(19) United States
(12) Patent Application Publication
(10) Pub. No.: US 2025/0188419 A1
(43) Pub. Date: Jun. 12, 2025

(54) METHODS AND COMPOSITIONS FOR STEM CELL DIFFERENTIATION

(71) Applicant: GC Therapeutics, Inc., Cambridge, MA (US)

(72) Inventors: Katelyn A. CABRAL, Cambridge, MA (US); Amanda M. MACKIE, Cambridge, MA (US); Cory J. SMITH, Brookline, MA (US); Parastoo KHOSHAKHLAGH, Boston, MA (US); Hon Man Alex NG, Quincy, MA (US); Jeremy VELAZQUEZ, Somerville, MA (US)

(21) Appl. No.: 18/980,842

(22) Filed: Dec. 13, 2024

Related U.S. Application Data
(63) Continuation of application No. PCT/US2023/068544, filed on Jun. 15, 2023.
(60) Provisional application No. 63/352,529, filed on Jun. 15, 2022.

(51) Int. Cl. C12N 5/071

(52) U.S. Cl. CPC C12N 5/067; C12N 2506/45; C12N 2510/00

(57) ABSTRACT

The present disclosure provides transcription factors and methods for stem cell differentiation into hepatocytes or hepatic cells. In some examples, a pluripotent stem cell (PSC) comprises a nucleic acid. In some examples, the nucleic acid comprises an open reading frame encoding one or more transcription factors, one or more transcription factors, or an activator of transcription of the open reading frame encoding one or more transcription factors. In some examples, the nucleic acid comprising an open reading frame encoding one or more transcription factors, the one or more transcription factors, or the activator of transcription of the open reading frame encoding one or more transcription factors induces differentiation of the PSC into a hepatocyte or a hepatic cell.

### CROSS REFERENCE

This application is a continuation of International Application No. PCT/US2023/068544, filed on Jun. 15, 2023, which claims benefit of priority to U.S. Provisional Application No. 63/352,529, filed Jun. 15, 2022, which are incorporated by reference herein in their entireties.

### BACKGROUND

Hepatocytes serve an important role in the liver: they can be involved in the synthesis or storage of proteins; transformation of carbohydrates; the synthesis of cholesterol, bile salts, or phospholipids; and detoxification. Therapies to improve certain functions of the liver may involve the introduction or modulation of hepatocytes or hepatic cells. Stem cells can be programmed into various phenotypes via the introduction of one or more transcription factors, or one or more molecules that modulate transcription or transcription factors. For example, stem cells can be programmed into hepatocytes or hepatic cells.

### SUMMARY

An aspect of the present disclosure is a pluripotent stem cell (PSC) engineered to express a nucleic acid comprising an open reading frame encoding one or more transcription factors, one or more transcription factors, or activator of transcription of the open reading frame encoding one or more transcription factors, wherein the one or more transcription factors comprising: one or more hepatocyte nuclear factor (“HNF”) family members; one or more forkhead box (“FOX”) family members; and one or more additional transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the one or more transcription factors comprises 2 or more HNF family members. In some embodiments, the one or more transcription factors comprises 3 or more HNF family members. In some embodiments, the one or more additional transcription factors comprises CEBP, and the one or more transcription factors further comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the one or more additional transcription factors comprises RBPJ, and the one or more transcription factors further comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the PSC further comprises at least 2 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the PSC further comprises at least 3 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the PSC further comprises at least 4 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the PSC further comprises at least 5 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the PSC further comprises at least 6 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the PSC further comprises at least 7 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the PSC further comprises at least 8 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the one or more HNF family members comprises HNF4A, HNF1A, ONECUTI/HNF6, or any combination thereof. In some embodiments, the one or more FOX family members comprises FOXA1. In some embodiments, the one or more transcription factors comprises HNF4A, HNF1A, ONECUT1/HNF6, and FOXA1. In some embodiments, the one or more transcription factors comprises CEBPA, FOXA1, HNF1A, HNF4A, ONECUTI/HNF6, and RBPJ.

Another aspect of the present disclosure is a method of generating a population of hepatocytes or hepatic cells comprising: providing one or more pluripotent stem cells (PSCs); delivering to the one or more PSCs at least one nucleic acid comprising an open reading frame encoding one or more transcription factors, one or more transcription factors, or activator of transcription of the open reading frame encoding one or more transcription factors, wherein the one or more transcription factors comprise: one or more hepatocyte nuclear factor (“HNF”) family members; one or more forkhead box (“FOX”) family members; and one or more additional transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12; generating the population of hepatocytes or hepatic cells from the one or more PSCs. In some embodiments, the one or more transcription factors comprises 2 or more HNF family members. In some embodiments, the one or more transcription factors comprises 3 or more HNF family members. In some embodiments, the one or more additional transcription factors comprises CEBPA, and the one or more transcription factors further comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the one or more additional transcription factors comprises RBPJ, and the one or more transcription factors further comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 2 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 3 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 4 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 5 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 6 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 7 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 8 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the one or more HNF family members comprises HNF4A, HNF1A, ONECUTI/HNF6, or any combination thereof. In some embodiments, the one or more FOX family members comprises FOXA1. In some embodiments, the one or more transcription factors comprises HNF4A, HNF1A, ONECUT1/HNF6, and FOXA1. In some embodiments, the one or more transcription factors comprises CEBPA, FOXA1, HNF1A, HNF4A, ONECUT1/HNF6, and RBPJ. In some embodiments, the at least one or more transcription factors induce the expression of the one or more PSCs into a population of hepatocytes or hepatic cells in 30 days or less. In some embodiments, the one or more PSCs are provided in a media. In some embodiments, the media is not altered during the differentiation of the one or more PSCs into the population of hepatocytes or hepatic cells. In some embodiments, the media is not altered during the delivering to the one or more PSCs the at least one exogenous expression cassette. In some embodiments, at least one of the one or more hepatocytes or the hepatic cells of the population of hepatocytes or hepatic cells expresses ASGR-1. In some embodiments, at least one of the one or more hepatocytes or the hepatic cells of the population of hepatocytes or hepatic cells expresses CXCR-4. In some embodiments, at least 2% of the population of hepatocytes or hepatic cells express ASGR-1. In some embodiments, at least 3% of the population of hepatocytes or hepatic cells express ASGR-1. In some embodiments, at least 5% of the population of hepatocytes or hepatic cells express ASGR-1. In some embodiments, at least 6% of the population of hepatocytes or hepatic cells express ASGR-1. In some embodiments, at least 10% of the population of hepatocytes or hepatic cells express CXCR-4. In some embodiments, at least 20% of the population of hepatocytes or hepatic cells express CXCR-4. In some embodiments, at least 30% of the PSCs express CXCR-4.

Another aspect of the present disclosure is an exogenous expression cassette that induces differentiation of a pluripotent stem cell (PSC) into a hepatocyte or a hepatic cell, wherein the exogenous expression cassette comprises: one or more hepatocyte nuclear factor (“HNF”) family member genes; one or more forkhead box (“FOX”) family member genes; and one or more additional transcription factor genes comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the one or more transcription factor genes comprises 2 or more HNF family members. In some embodiments, the one or more transcription factor genes comprises 3 or more HNF family members. In some embodiments, the one or more additional transcription factor genes comprises CEBPA, and the one or more transcription factor genes further comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the one or more additional transcription factor genes comprises RBPJ, and the one or more transcription factors further comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the exogenous expression cassette further comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the exogenous expression cassette comprises at least 3 or more transcription factor genes comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the exogenous expression cassette comprises at least 4 or more transcription factor genes comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the exogenous expression cassette comprises at least 5 or more transcription factor genes comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the exogenous expression cassette comprises at least 6 or more transcription factor genes comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the exogenous expression cassette comprises at least 7 or more transcription factor genes comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the exogenous expression cassette comprises at least 8 or more transcription factor genes comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the one or more HNF family members comprises HNF4A, HNF1A, ONECUT1/HNF6, or any combination thereof. In some embodiments, the one or more FOX family members comprises FOXA1, or FOXA3, or any combination thereof. In some embodiments, the one or more transcription factor genes comprises HNF4A, HNF1A, ONECUT1/HNF6, and FOXA1. In some embodiments, the one or more transcription factors genes comprises CEBPA, FOXA1, HNF1A, HNF4A, ONECUT1/HNF6, and RBPJ. In some embodiments, the PSC is provided in a media. In some embodiments, the media is not altered during the differentiation of the PSC into the hepatocyte or hepatic cell. In some embodiments, the exogenous expression cassette induces differentiation of the PSC into the hepatocyte or hepatic cell in 30 days or less. In some embodiments, the exogenous expression cassette induces differentiation of the PSC into the hepatocyte or hepatic cell in 96 hours or less.

Another aspect of the present disclosure is a method of generating a population of hepatocytes or hepatic cells comprising: providing one or more pluripotent stem cells (PSCs); delivering to the one or more PSCs a nucleic acid comprising an open reading frame encoding one or more transcription factors, one or more transcription factors, or activator of transcription of the open reading frame encoding one or more transcription factors; and generating the population of hepatocytes or hepatic cells from the one or more PSCs in 30 days or less. In some embodiments, the one or more PSCs is differentiated into the population of hepatocytes or hepatic cells in 96 hours or less. In some embodiments, the one or more PSCs are provided in a media. In some embodiments, the media is not altered during the differentiation of the one or more PSCs into the population of hepatocytes. In some embodiments, the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the one or more transcription factors are of a transcription factor family, for example: FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, two or more transcription factors are of a transcription factor family, for example: FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, three or more transcription factors are of a transcription factor family, for example: FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, four or more transcription factors are of a transcription factor family, for example: FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the at least one or more transcription factors comprise SPI1, HNF1A, FoxA2, CEBPA, HNF6, HNF4a, and HNF6B. In some embodiments, the one or more transcription factors comprise HNF1A, Atf3, HNF6, HNF6B, Fos, CEBPA, and FoxM1. In some embodiments, the one or more transcription factors comprise GATA2 ISOFORM P23769-2, HNF4a, RXRB, NFE2L2, Myc, TP73, and HNF6. In some embodiments, the one or more transcription factors comprise FOXA1, HNF1A, FOXA2, CEBPA, ONECUT1/HNF6, HNF4A, RBPJ. In some embodiments, the one or more transcription factors comprise CEBPA, ATF5, HHEX, SOX17, HNF4A, ONECUT2/HNF6B, and HNF1A. In some embodiments, the one or more transcription factors comprise HNF1A, CEBPA, ONECUT1/HNF6, ONECUT2/HNF6B, Fos, HIFIA, and TBX3. In some embodiments, the one or more PSCs are provided in a media. In some embodiments, the media is not altered during the differentiation of the one or more PSCs into the population of hepatocytes or hepatic cells. In some embodiments, the media is not altered during the delivering to the one or more PSCs the nucleic acid comprising an open reading frame encoding one or more transcription factors, the one or more transcription factors, or the activator of transcription of the open reading frame encoding one or more transcription factors. In some embodiments, at least one of the one or more hepatocytes or the hepatic cells of the population of hepatocytes or hepatic cells expresses ASGR-1. In some embodiments, at least one of the one or more hepatocytes or the hepatic cells of the population of hepatocytes or hepatic cells does not express CXCR-4. In some embodiments, at least one of the one or more hepatocytes or the hepatic cells of the population of hepatocytes or hepatic cells expresses CXCR-4. In some embodiments, the population of hepatocytes or hepatic cells comprise at least 2% of the population of hepatocytes or hepatic cells express ASGR-1. In some embodiments, the population of hepatocytes or hepatic cells comprise at least 3% of the population of hepatocytes or hepatic cells express ASGR-1. In some embodiments, the population of hepatocytes or hepatic cells comprise at least 5% of the population of hepatocytes or hepatic cells express ASGR-1. In some embodiments, the population of hepatocytes or hepatic cells comprise at least 6% of the population of hepatocytes or hepatic cells express ASGR-1. In some embodiments, the population of hepatocytes or hepatic cells comprise at least 10% of the population of hepatocytes or hepatic cells express CXCR-4. In some embodiments, the population of hepatocytes or hepatic cells comprise at least 20% of the population of hepatocytes or hepatic cells express CXCR-4. In some embodiments, the population of hepatocytes or hepatic cells comprise wherein at least 30% of the PSCs express CXCR-4. A pluripotent stem cell (PSC) comprising a nucleic acid comprising an open reading frame encoding one or more transcription factors, one or more transcription factors, or an activator of transcription of the open reading frame encoding one or more transcription factors, wherein the nucleic acid comprising an open reading frame encoding one or more transcription factors, the one or more transcription factors, or the activator of transcription of the open reading frame encoding one or more transcription factors induces differentiation of the PSC into a hepatocyte or a hepatic cell in 30 days or less. In some embodiments, the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the one or more transcription factors are of a transcription factor family, for example: FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the one or more transcription factors comprise ONECUT1/HNF6 or ONECUT2/HNF6B. In some embodiments, the one or more transcription factors comprise ONECUT1/HNF6 and ONECUT2/HNF6B. In some embodiments, the one or more transcription factors are, for example, SPI1, HNF1A, FoxA2, CEBPA, HNF6, HNF4a, and HNF6B. In some embodiments, the one or more transcription factors are, for example, HNF1A, Atf3, HNF6, HNF6B, Fos, CEBPA, and FoxM1. In some embodiments, the one or more transcription factors are, for example, SPI1, FoxA2, a GATA2 transcription factor, GATA4, JUN/AP-1, and NFIX. In some embodiments, the one or more transcription factors are, for example, HNF4a, CEBPA, GATA6, TBX3, Fos, and a GATA2 transcription factor. In some embodiments, the one or more transcription factors are, for example, GATA2 transcription factor, HNF4a, RXRB, NFE2L2, Myc, TP73, and HNF6. In some embodiments, the one or more transcription factors are, for example, GATA2 transcription factor, CEBPA, ATF3, Fos, GATA3, NFIX. In some embodiments, the one or more transcription factors are, for example, HHEX, SOX17, HNF4A, HNF1A, ONECUTI/HNF6, FoxA2, and FoxA1. In some embodiments, the one or more transcription factors are, for example, HHEX, SOX17, and HNF4A. In some embodiments, the one or more transcription factors are, for example, CEBPA, ATF5, HHEX, SOX17, HNF4A, ONECUT2/HNF6B, and HNF1A. In some embodiments, the one or more transcription factors are, for example, FoxA1, HNF1A, FoxA2, CEBPA, ONECUT1/HNF6, HNF4a, and RBPJ. In some embodiments, the one or more transcription factors are, for example, Smad3, HHEX, ATF5, NR5A2, GATA2 transcription factor, CEBPB, and NR1I3. In some embodiments, the one or more transcription factors are, for example, HNF1A, CEBPA, ONECUT1/HNF6, ONECUT2/HNF6B, Fos, HIFIA, and TBX3. In some embodiments, the hepatocyte or the hepatic cell of expresses ASGR-1. In some embodiments, the hepatocyte or the hepatic cell, does not express CXCR-4. In some embodiments, the hepatocyte or the hepatic cell expresses CXCR-4. In some embodiments, the PSC is provided in a media. In some embodiments, the media is not altered during the differentiation of the PSC into the hepatocyte or hepatic cell. In some embodiments, the population of hepatocytes or hepatic cells comprise two or more of the hepatocyte or the hepatic cell. In some embodiments, the population of hepatocytes or hepatic cells comprise at least 2% of the hepatocytes or the hepatic cells that express ASGR-1. In some embodiments, the population of hepatocytes or hepatic cells comprise at least 3% of the hepatocytes or the hepatic cells that express ASGR-1. In some embodiments, the population of hepatocytes or hepatic cells comprise at least 5% of the hepatocytes or the hepatic cells that express ASGR-1. In some embodiments, the population of hepatocytes or hepatic cells comprise at least 6% of the hepatocytes or the hepatic cells that express ASGR-1. In some embodiments, the population of hepatocytes or hepatic cells comprise at least 10% of the hepatocytes or the hepatic cells that express CXCR-4. In some embodiments, the population of hepatocytes or hepatic cells comprise at least 20% of the hepatocytes or the hepatic cells that express CXCR-4. In some embodiments, the population of hepatocytes or hepatic cells comprise at least 30% of the hepatocytes or the hepatic cells that express CXCR-4. In some embodiments, the population of hepatocytes or hepatic cells comprising no nutrient, or growth factor or microenvironmental/matrix optimizations are performed.

Another aspect of the present disclosure is a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: SPI1, HNF1A, FOXA2, CEBPA, ONECUT1, HNF4A, and ONECUT2.

Another aspect of the present disclosure is a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: HHEX, SOX17, HNF4A, HNF1A, ONECUT1/HNF6, FOXA2, and FOXA1.

Another aspect of the present disclosure is a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: CEBPA, ATF5, HHEX, SOX17, HNF4A, ONECUT2/HNF6B, and HNF1A.

Another aspect of the present disclosure is a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: FOXA1, HNF1A, FOXA2, CEBPA, ONECUT1/HNF6, HNF4A, and RBPJ.

Another aspect of the present disclosure is a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: HNF1A, CEBPA, ONECUTI, ONECUT2, FOS, HIFIA, and TBX3.

Another aspect of the present disclosure is a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: HNF4A, HNF1A, ONECUT1/HNF6, and FOXA1.

Another aspect of the present disclosure is a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: FOXA1, HNF1A, CEBPA, ONECUT1/HNF6, and HNF4A.

Another aspect of the present disclosure is a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: FOXA1, HNF4A, ONECUTI/HNF6, and RBPJ.

Another aspect of the present disclosure is a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: CEBPA, FOXA1, HNF1A, HNF4A, ONECUT1/HNF6, and RBPJ.

Another aspect of the present disclosure is a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: CEBPA, FOXA1, HNF1A, and HNF4A.

Another aspect of the present disclosure is a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: CEBPA, HNF1A, HNF4A, FOXA3, FOXA2, PXR, and RXRA.

Another aspect of the present disclosure is a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: CEBPB, FOXA1, FOXA3, HLF, HNF1A, HNF4A, NR1I2/PXR, NR113/CAR, ONECUT1/HNF6, PROX1, RBPJ, RORC, and SALL4.

In another aspect of the present disclosure is provided a kit for inducing differentiation of a pluripotent stem cell (PSC) into a hepatocyte or a hepatic cell, the kit comprising: one or more hepatocyte nuclear factor (“HNF”) family members; one or more forkhead box (“FOX”) family members; and one or more additional transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the one or more transcription factor genes comprises 2 or more HNF family members. In some embodiments, the one or more transcription factor genes comprises 3 or more HNF family members. In some embodiments, the one or more additional transcription factors comprise CEBPA, and the one or more transcription factor further comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the one or more additional transcription factors comprise RBPJ and the one or more transcription factors further comprises FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 2 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 3 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 4 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 5 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 6 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 7 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the method further comprises at least 8 or more transcription factors comprising FOS, JUN, FOX, PROX, GATA, HHEX, HIF, HNF, MYC, CEBP, NFE, NFI, NR, RBPJ, RXR, SMAD, SOX, SPI, TBX, TP, PPAR, SALL, EGR, ONECUT, SKI or HLF family members, or one or more additional transcription factors from Table 12. In some embodiments, the one or more HNF family members comprise HNF4A, HNF1A, ONECUT1/HNF6, or any combination thereof. In some embodiments, the one or more FOX family members comprise FOXA1, or FOXA3, or any combination thereof. In some embodiments, the one or more transcription factors comprise HNF4A, HNF1A, ONECUT1/HNF6, and FOXA1. In some embodiments, the one or more transcription factors comprise CEBPA, FOXA1, HNF1A, HNF4A, ONECUT1/HNF6, and RBPJ. In some embodiments, the PSC is provided in a media. In some embodiments, the media is not altered during the differentiation of the PSC into the hepatocyte or hepatic cell. In some embodiments, the exogenous expression cassette induces differentiation of the PSC into the hepatocyte or hepatic cell in 30 days or less. In some embodiments, the exogenous expression cassette induces differentiation of the PSC into the hepatocyte or hepatic cell in 96 hours or less.

Another aspect of the present disclosure is a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: ATF5, CEBPA, CEBPB, FOXA1, FOXA2, FOXA3, GATA6, HHEX, HIFIA, HLF, HNF1A, HNF4A, NR1H4/FXR, NR112/PXR, NR113/CAR, NR5A2, ONECUT1/HNF6, ONECUT2/HNF6B, NR1C1/PPARA, PROX1, RBPJ, RORC, RXRA, TBX3, and SALL4.

Another aspect of the present disclosure provides a pluripotent stem cell (PSC) comprising a nucleic acid comprising an open reading frame encoding one or more transcription factors, one or more transcription factors, or an activator of transcription of the open reading frame encoding one or more transcription factors, wherein the nucleic acid comprising an open reading frame encoding one or more transcription factors, the one or more transcription factors, or the activator of transcription of the open reading frame encoding one or more transcription factors induces differentiation of the PSC into a hepatocyte or a hepatic cell in 30 days or less. In some embodiments, the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments wherein two or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, wherein four or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the one or more transcription factors are of a transcription factor family, for example, ATF, CEBP, FOS, FOX, GATA, HHEX, HIF, HNF, AP-1/JUN, MYC, NFE, NFI, NR, ONECUT, RBPJ, RXR, SMAD, SOX, SPI, TBX, PPAR, FOS, JUN, SKI, SALL, EGR, and TP family. In some embodiments, the one or more transcription factors comprise ONECUT1/HNF6 or ONECUT2/HNF6B. In some embodiments, the one or more transcription factors comprise ONECUT1/HNF6 and ONECUT2/HNF6B. In some embodiments, the one or more transcription factors are, for example, SPI1, HNF1A, FoxA2, CEBPA, HNF6, HNF4a, and HNF6B. In some embodiments, the one or more transcription factors are, for example, HNF1A, Atf3, HNF6, HNF6B, Fos, CEBPA, and FoxM1. In some embodiments, the one or more transcription factors are, for example, SPI1, FoxA2, a GATA2 transcription factor, GATA4, JUN/AP-1, and NFIX. In some embodiments, wherein the one or more transcription factors are, for example, HNF4a, CEBPA, GATA6, TBX3, Fos, and a GATA2 transcription factor. In some embodiments, wherein the one or more transcription factors are GATA2 transcription factor, HNF4a, RXRB, NFE2L2, Myc, TP73, and HNF6. In some embodiments, wherein the one or more transcription factors are, for example, GATA2 transcription factor, CEBPA, ATF3, Fos, GATA3, NFIX. In some embodiments, wherein the one or more transcription factors are, for example, HHEX, SOX17, HNF4A, HNF1A, ONECUTI/HNF6, FoxA2, and FoxA1. In some embodiments, wherein the one or more transcription factors are, for example, HHEX, SOX17, and HNF4A. In some embodiments, wherein the one or more transcription factors are, for example, CEBPA, ATF5, HHEX, SOX17, HNF4A, ONECUT2/HNF6B, and HNF1A. In some embodiments, wherein the one or more transcription factors are, for example, FoxA1, HNF1A, FoxA2, CEBPA, ONECUT1/HNF6, HNF4a, and RBPJ. In some embodiments, wherein the one or more transcription factors are, for example, Smad3, HHEX, ATF5, NR5A2, GATA2 transcription factor, CEBPB, and NR1I3. In some embodiments, wherein the one or more transcription factors are, for example, HNF1A, CEBPA, ONECUTI/HNF6, ONECUT2/HNF6B, Fos, HIFIA, and TBX3.

The present disclosure provides a hepatocyte or hepatic cell wherein the hepatocyte or the hepatic cell expresses ASGR-1. In some embodiments, the hepatocyte or the hepatic cell does not express CXCR-4. In some embodiments, wherein the hepatocyte or the hepatic cell expresses CXCR-4. In some embodiments, wherein the PSC is provided in a media. In some embodiments, the media is not altered during the differentiation of the PSC into the hepatocyte or hepatic cell.

The present disclosure provides a population of hepatocytes or hepatic cells comprising two or more of the hepatocyte or the hepatic cell as disclosed herein. In some embodiments, at least 2% of the hepatocytes or the hepatic cells express ASGR-1. In some embodiments, at least 3% of the hepatocytes or the hepatic cells express ASGR-1. In some embodiments, at least 5% of the hepatocytes or the hepatic cells express ASGR-1. In some embodiments, at least 6% of the hepatocytes or the hepatic cells express ASGR-1. In some embodiments, at least 10% of the hepatocytes or the hepatic cells express CXCR-4. In some embodiments, at least 20% of the hepatocytes or the hepatic cells express CXCR-4. In some embodiments, at least 30% of the hepatocytes or the hepatic cells express CXCR-4. In some embodiments, no nutrient, growth factor or microenvironmental/matrix optimizations are performed.

Another aspect of the present disclosure provides a pluripotent stem cell (PSC) comprising a nucleic acid comprising an open reading frame encoding one or more transcription factors, one or more transcription factors, or activator of transcription of the open reading frame encoding one or more transcription factors, wherein the nucleic acid comprising an open reading frame encoding one or more transcription factors, the one or more transcription factors, or the activator of transcription of the open reading frame encoding one or more transcription factors induce differentiation of the PSC into a hepatocyte or a hepatic cell, wherein the PSC is provided in a media, and wherein the media is not altered during the differentiation of the PSC into the hepatocyte or the hepatic cell. In some embodiments, the one or more transcription factors induce the differentiation of the PSC into the hepatocyte or the hepatic cell in 30 days or less. In some embodiments, the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the one or more transcription factors are selected from the group of transcription factor families consisting of: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, the one or more transcription factors comprise ONECUT1/HNF6 or ONECUT2/HNF6B. In some embodiments, the one or more transcription factors comprise ONECUT1/HNF6 and ONECUT2/HNF6B. In some embodiments, the one or more transcription factors are, for example, SPI1, HNF1A, FoxA2, CEBPA, HNF6, HNF4a, and HNF6B. In some embodiments, the one or more transcription factors are, for example, HNF1A, Atf3, HNF6, HNF6B, Fos, CEBPA, and FoxM1. In some embodiments, the one or more transcription factors are, for example, SPI1, FoxA2, a GATA2 transcription factor, GATA4, JUN/AP-1, and NFIX. In some embodiments, the one or more transcription factors are, for example, HNF4a, CEBPA, GATA6, TBX3, Fos, and a GATA2 transcription factor. In some embodiments, the one or more transcription factors are, for example, GATA2 transcription factor, HNF4a, RXRB, NFE2L2, Myc, TP73, and HNF6. In some embodiments, the one or more transcription factors are, for example, GATA2 transcription factor, CEBPA, ATF3, Fos, GATA3, NFIX. In some embodiments, the one or more transcription factors are, for example, HHEX, SOX17, HNF4A, HNF1A, ONECUT1/HNF6, FoxA2, and FoxA1. In some embodiments, the one or more transcription factors are, for example, HHEX, SOX17, and HNF4A. In some embodiments, the one or more transcription factors are, for example, CEBPA, ATF5, HHEX, SOX17, HNF4A, ONECUT2/HNF6B, and HNF1A. In some embodiments, the one or more transcription factors are, for example, FoxA1, HNF1A, FoxA2, CEBPA, ONECUT1/HNF6, HNF4a, and RBPJ. In some embodiments, the one or more transcription factors are, for example, Smad3, HHEX, ATF5, NR5A2, GATA2 transcription factor, CEBPB, and NR1I3. In some embodiments, the one or more transcription factors are, for example, HNF1A, CEBPA, ONECUTI/HNF6, ONECUT2/HNF6B, Fos, HIFIA, and TBX3. In some embodiments, wherein the hepatocyte or the hepatic cell expresses ASGR-1. In some embodiments, wherein the hepatocyte or the hepatic cell does not express CXCR-4. In some embodiments, wherein the hepatocyte or the hepatic cell expresses CXCR-4. In some embodiments, the PSC is provided in a media. In some embodiments, the media is not altered during the differentiation of the PSC into the hepatocyte or hepatic cell.

The present disclosure provides a population of hepatocytes or hepatic cells comprising two or more of the hepatocyte or the hepatic cell as disclosed herein. In some embodiments, at least 2% of the hepatocytes or the hepatic cells express ASGR-1. In some embodiments, at least 3% of the hepatocytes or the hepatic cells express ASGR-1. In some embodiments, at least 5% of the hepatocytes or the hepatic cells express ASGR-1. In some embodiments, at least 6% of the hepatocytes or the hepatic cells express ASGR-1. In some embodiments, at least 10% of the hepatocytes or the hepatic cells express CXCR-4. In some embodiments, at least 20% of the hepatocytes or the hepatic cells express CXCR-4. In some embodiments, at least 30% of the hepatocytes or the hepatic cells express CXCR-4. In some embodiments, no nutrient, growth factor or microenvironmental/matrix optimizations are performed.

Another aspect of the present disclosure provides a pluripotent stem cell (PSC) comprising a nucleic acid comprising an open reading frame encoding one or more transcription factors, one or more transcription factors, or activator of transcription of the open reading frame encoding one or more transcription factors, wherein the one or more transcription factors comprise: an HNF6 gene, and one or more additional hepatocyte programming factor genes, for example, FoxA1, SPI1, FoxA2, CEBPA, HNF4a, Atf3, Fos, CEBPB, FoxM1, GATA2 transcription factor, GATA4, JUN/FOS, JUN, NFIX, GATA6, TBX3, RXRB, NFE2L2, Myc, TP73, HHEX, SOX17, ATF5, RBPJ, Smad3, NR5A2, NR113, and HIF1A. In some embodiments, the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more transcription factors are, for example, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, or TP53 family members. In some embodiments, two or more transcription factors are selected from the group of transcription factor families consisting of: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, three or more transcription factors are selected from the group of transcription factor families consisting of: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, four or more transcription factors are selected from the group of transcription factor families consisting of: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, JUN, SKI, SALL, EGR, and TP. In some embodiments, the nucleic acid comprising an open reading frame encoding one or more transcription factors, the one or more transcription factors, or the activator of transcription of the open reading frame encoding one or more transcription factors induces differentiation of the PSC into a hepatocyte or a hepatic cell. In some embodiments, the nucleic acid comprising an open reading frame encoding one or more transcription factors, the one or more transcription factors, or the activator of transcription of the open reading frame encoding one or more transcription factors induce the differentiation of the PSC into the hepatocyte or hepatic cell in 30 days or less. In some embodiments, the one or more transcription factors are, for example, SPI1, HNF1A, FoxA2, CEBPA, HNF6, HNF4a, and HNF6B. In some embodiments, the one or more transcription factors are, for example, HNF1A, Atf3, HNF6, HNF6B, Fos, CEBPA, and FoxM1. In some embodiments, the one or more transcription factors are, for example, SPI1, FoxA2, a GATA2 transcription factor, GATA4, JUN/AP-1, and NFIX. In some embodiments, the one or more transcription factors are, for example, HNF4a, CEBPA, GATA6, TBX3, Fos, and a GATA2 transcription factor. In some embodiments, the one or more transcription factors are, for example, GATA2 transcription factor, HNF4a, RXRB, NFE2L2, Myc, TP73, and HNF6. In some embodiments, the one or more transcription factors are, for example, GATA2 transcription factor, CEBPA, ATF3, Fos, GATA3, NFIX. In some embodiments, the one or more transcription factors are, for example, HHEX, SOX17, HNF4A, HNF1A, ONECUT1/HNF6, FoxA2, and FoxA1. In some embodiments, the one or more transcription factors are, for example, HHEX, SOX17, and HNF4A. In some embodiments, the one or more transcription factors are, for example, CEBPA, ATF5, HHEX, SOX17, HNF4A, ONECUT2/HNF6B, and HNF1A. In some embodiments, the one or more transcription factors are, for example, FoxA1, HNF1A, FoxA2, CEBPA, ONECUT1/HNF6, HNF4a, and RBPJ. In some embodiments, the one or more transcription factors are, for example, Smad3, HHEX, ATF5, NR5A2, GATA2 transcription factor, CEBPB, and NR1I3. In some embodiments, the one or more transcription factors are, for example, HNF1A, CEBPA, ONECUT1/HNF6, ONECUT2/HNF6B, Fos, HIFIA, and TBX3. In some embodiments, the PSC is provided in a media. In some embodiments, the media is not altered during the differentiation of the PSC into the hepatocyte or hepatic cell.

The present disclosure provides a hepatocyte or hepatic cell, wherein the hepatocyte or the hepatic cell expresses ASGR-1. In some embodiments, wherein the hepatocyte or the hepatic cell does not express CXCR-4. In some embodiments, wherein the hepatocyte or the hepatic cell expresses CXCR-4.

The present disclosure provides a population of cells comprising two or more of the hepatocyte or the hepatic cell as disclosed herein. In some embodiments, wherein at least 2% of the hepatocytes or the hepatic cells express ASGR-1. In some embodiments, wherein at least 3% of the hepatocytes or the hepatic cells express ASGR-1. In some embodiments, wherein at least 5% of the hepatocytes or the hepatic cells express ASGR-1. In some embodiments, wherein at least 6% of the hepatocytes or the hepatic cells express ASGR-1. In some embodiments, wherein at least 10% of the hepatocytes or the hepatic cells express CXCR-4. In some embodiments, wherein at least 20% of the hepatocytes or the hepatic cells express CXCR-4. In some embodiments, wherein at least 30% of the hepatocytes or the hepatic cells express CXCR-4. In some embodiments, wherein the population of cells is provided in a media. In some embodiments, wherein no nutrient, growth factor, or microenvironmental or matrix optimizations are performed.

Another aspect of the present disclosure provides a method of generating a population of hepatocytes or hepatic cells comprising: providing one or more pluripotent stem cells (PSCs); delivering to the one or more PSCs at least one nucleic acid comprising an open reading frame encoding one or more transcription factors, one or more transcription factors, or activator of transcription of the open reading frame encoding one or more transcription factors, wherein the one or more transcription factors comprise an HNF6 gene; and generating the population of hepatocytes or hepatic cells from the one or more PSCs. In some embodiments, the method further comprises one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the method further comprises two or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the method further comprises three or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the method further comprises four or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, three or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, four or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, five or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, the at least one or more transcription factors comprise SPI1, HNF1A, FoxA2, CEBPA, HNF6, HNF4a, and HNF6B. In some embodiments, the at least one or more transcription factors comprise HNF1A, Atf3, HNF6, HNF6B, Fos, CEBPA, and FoxM1. In some embodiments, the at least one or more transcription factors comprise GATA2 ISOFORM P23769-2, HNF4a, RXRB, NFE2L2, Myc, TP73, and HNF6. In some embodiments, the at least one or more transcription factors comprise FOXA1, HNF1A, FOXA2, CEBPA, ONECUTI/HNF6, HNF4A, RBPJ. In some embodiments, the at least one or more transcription factors comprise CEBPA, ATF5, HHEX, SOX17, HNF4A, ONECUT2/HNF6B, and HNF1A. In some embodiments, the at least one or more transcription factors comprise HNF1A, CEBPA, ONECUTI/HNF6, ONECUT2/HNF6B, Fos, HIFIA, and TBX3. In some embodiments, the at least one or more transcription factors induce the expression of the one or more PSCs into the population of hepatocytes or hepatic cells in 30 days or less. In some embodiments, the one or more PSCs are provided in a media. In some embodiments, the media is not altered during the differentiation of the one or more PSCs into the population of hepatocytes or hepatic cells. In some embodiments, the media is not altered during the delivering to the one or more PSCs the at least one exogenous expression cassette. In some embodiments, at least one of the one or more hepatocytes or the hepatic cells of the population of hepatocytes or hepatic cells expresses ASGR-1. In some embodiments, at least one of the one or more hepatocytes or the hepatic cells of the population of hepatocytes or hepatic cells does not express CXCR-4. In some embodiments, at least one of the one or more hepatocytes or the hepatic cells of the population of hepatocytes or hepatic cells expresses CXCR-4. In some embodiments, at least 2% of the population of hepatocytes or hepatic cells express ASGR-1. In some embodiments, at least 3% of the population of hepatocytes or hepatic cells express ASGR-1. In some embodiments, at least 5% of the population of hepatocytes or hepatic cells express ASGR-1. In some embodiments, at least 6% of the population of hepatocytes or hepatic cells express ASGR-1. In some embodiments, at least 10% of the population of hepatocytes or hepatic cells express CXCR-4. In some embodiments, at least 20% of the population of hepatocytes or hepatic cells express CXCR-4. In some embodiments, at least 30% of the PSCs express CXCR-4.

Another aspect of the present disclosure provides a method of generating a population of hepatocytes or hepatic cells comprising: providing one or more pluripotent stem cells (PSCs); delivering to the one or more PSCs a nucleic acid comprising an open reading frame encoding one or more transcription factors, one or more transcription factors, or activator of transcription of the open reading frame encoding one or more transcription factors, wherein the one or more transcription factors induce differentiation of the one or more PSCs into the population of hepatocytes or hepatic cells; additional inducing the expression of the one or more transcription factors in the one or more PSCs in 30 days or less; and generating the population of hepatocytes or hepatic cells from the one or more PSCs. In some embodiments, the one or more PSCs is differentiated into the population of hepatocytes or hepatic cells in 96 hours or less. In some embodiments, the one or more PSCs are provided in a media. In some embodiments, the media is not altered during the differentiation of the one or more PSCs into the population of hepatocytes. In some embodiments, the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the one or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, two or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, three or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, four or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, JUN, SKI, SALL, EGR, and TP. In some embodiments, the at least one or more transcription factors comprise SPI1, HNF1A, FoxA2, CEBPA, HNF6, HNF4a, and HNF6B. In some embodiments, the one or more transcription factors comprise HNF1A, Atf3, HNF6, HNF6B, Fos, CEBPA, and FoxM1. In some embodiments, the one or more transcription factors comprise GATA2 ISOFORM P23769-2, HNF4a, RXRB, NFE2L2, Myc, TP73, and HNF6. In some embodiments, the one or more transcription factors comprise FOXA1, HNF1A, FOXA2, CEBPA, ONECUTI/HNF6, HNF4A, RBPJ. In some embodiments, the one or more transcription factors comprise CEBPA, ATF5, HHEX, SOX17, HNF4A, ONECUT2/HNF6B, and HNF1A. In some embodiments, the one or more transcription factors comprise HNF1A, CEBPA, ONECUT1/HNF6, ONECUT2/HNF6B, Fos, HIFIA, and TBX3. In some embodiments, the one or more PSCs are provided in a media. In some embodiments, the media is not altered during the differentiation of the one or more PSCs into the population of hepatocytes or hepatic cells. In some embodiments, the media is not altered during the delivering to the one or more PSCs the nucleic acid comprising an open reading frame encoding one or more transcription factors, the one or more transcription factors, or the activator of transcription of the open reading frame encoding one or more transcription factors. In some embodiments, at least one of the one or more hepatocytes or the hepatic cells of the population of hepatocytes or hepatic cells expresses ASGR-1. In some embodiments, at least one of the one or more hepatocytes or the hepatic cells of the population of hepatocytes or hepatic cells does not express CXCR-4. In some embodiments, at least one of the one or more hepatocytes or the hepatic cells of the population of hepatocytes or hepatic cells expresses CXCR-4.

The present disclosure provides a population of hepatocytes or hepatic cells as disclosed herein, wherein at least 2% of the population of hepatocytes or hepatic cells express ASGR-1. In some embodiments, at least 3% of the population of hepatocytes or hepatic cells express ASGR-1. In some embodiments, at least 5% of the population of hepatocytes or hepatic cells express ASGR-1. In some embodiments, at least 6% of the population of hepatocytes or hepatic cells express ASGR-1. In some embodiments, at least 10% of the population of hepatocytes or hepatic cells express CXCR-4. In some embodiments, at least 20% of the population of hepatocytes or hepatic cells express CXCR-4. In some embodiments, at least 30% of the PSCs express CXCR-4.

Another aspect of the present disclosure provides a method of generating a population of hepatocytes or hepatic cells comprising: providing one or more pluripotent stem cells (PSCs) in a media; delivering to the one or more PSCs a nucleic acid comprising an open reading frame encoding one or more transcription factors, one or more transcription factors, or activator of transcription of the open reading frame encoding one or more transcription factors, wherein the one or more transcription factors additional induce differentiation of the one or more PSCs into the population of hepatocytes or hepatic cells; and generating the population of hepatocytes or hepatic cells from the one or more PSCs, wherein the media is not altered during the differentiation of the one or more PSCs into the population of hepatocytes or hepatic cells. In some embodiments, the media is not altered during the delivering to the one or more PSCs the one or more transcription factors. In some embodiments, the one or more transcription factors comprises HNF6. In some embodiments, the one or more transcription factors induce the expression of the one or more PSCs into the population of hepatocytes or hepatic cells in 30 days or less. In some embodiments, the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, three or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, four or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, the at least one or more transcription factors comprise SPI1, HNF1A, FoxA2, CEBPA, HNF6, HNF4a, and HNF6B. In some embodiments, the one or more transcription factors comprise HNF1A, Atf3, HNF6, HNF6B, Fos, CEBPA, and FoxM1. In some embodiments, the one or more transcription factors comprise GATA2 ISOFORM P23769-2, HNF4a, RXRB, NFE2L2, Myc, TP73, and HNF6. In some embodiments, the one or more transcription factors comprise FOXA1, HNF1A, FOXA2, CEBPA, ONECUTI/HNF6, HNF4A, RBPJ. In some embodiments, the one or more transcription factors comprise CEBPA, ATF5, HHEX, SOX17, HNF4A, ONECUT2/HNF6B, and HNF1A. In some embodiments, the one or more transcription factors comprise HNF1A, CEBPA, ONECUTI/HNF6, ONECUT2/HNF6B, Fos, HIFIA, and TBX3. In some embodiments, the at least one or more transcription factors induce the expression of the one or more PSCs into the population of hepatocytes or hepatic cells in 30 days or less. In some embodiments, the one or more PSCs are provided in a media. In some embodiments, the media is not altered during the differentiation of the one or more PSCs into the population of hepatocytes or hepatic cells. In some embodiments, the media is not altered during the delivering to the one or more PSCs the nucleic acid comprising an open reading frame encoding one or more transcription factors, the one or more transcription factors, or the activator of transcription of the open reading frame encoding one or more transcription factors. In some embodiments, at least one of the one or more hepatocytes or the hepatic cells of the population of hepatocytes or hepatic cells expresses ASGR-1. In some embodiments, at least one of the one or more hepatocytes or the hepatic cells of the population of hepatocytes or hepatic cells does not express CXCR-4. In some embodiments, at least one of the one or more hepatocytes or the hepatic cells of the population of hepatocytes or hepatic cells expresses CXCR-4. In some embodiments, at least 2% of the population of hepatocytes or hepatic cells express ASGR-1. In some embodiments, at least 3% of the population of hepatocytes or hepatic cells express ASGR-1. In some embodiments, at least 5% of the population of hepatocytes or hepatic cells express ASGR-1. In some embodiments, at least 6% of the population of hepatocytes or hepatic cells express ASGR-1. In some embodiments, at least 10% of the population of hepatocytes or hepatic cells express CXCR-4. In some embodiments, at least 20% of the population of hepatocytes or hepatic cells express CXCR-4. In some embodiments, at least 30% of the PSCs express CXCR-4.

Another aspect of the present disclosure provides an exogenous expression cassette comprising one or more transcription factors that induce differentiation of a pluripotent stem cell (PSC) into a hepatocyte or a hepatic cell, wherein exogenous expression cassette induces the expression of the PSC into the hepatocyte or the hepatic cell in 30 days or less. In some embodiments, the PSC is provided in a media. In some embodiments, the media is not altered during the differentiation of the PSC into the hepatocyte or hepatic cell. In some embodiments, the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the one or more transcription factors comprise ONECUT1/HNF6 or ONECUT2/HNF6B. In some embodiments, the one or more transcription factors comprise ONECUT1/HNF6 and ONECUT2/HNF6B. In some embodiments, two or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, three or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, four or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, the one or more transcription factors are, for example, SPI1, HNF1A, FoxA2, CEBPA, HNF6, HNF4a, and HNF6B. In some embodiments, the one or more transcription factors are, for example, HNF1A, Atf3, HNF6, HNF6B, Fos, CEBPA, and FoxM1. In some embodiments, the one or more transcription factors are, for example, FoxA1, FoxA2, GATA2 ISOFORM P23769-2, GATA4, GATA2 ISOFORM P23769-1, JUN/AP-1, and NFIX. In some embodiments, the one or more transcription factors are, for example, HNF4a, CEBPA, GATA6, TBX3, Fos, GATA2 ISOFORM P23769-2, and GATA2 ISOFORM P23769-1. In some embodiments, the one or more transcription factors are, for example, GATA2 ISOFORM P23769-2, HNF4a, RXRB, NFE2L2, Myc, TP73, and HNF6. In some embodiments, the one or more transcription factors are, for example, additional FOXA1, HNF1A, FOXA2, CEBPA, ONECUTI/HNF6, HNF4A, RBPJ. In some embodiments, the one or more transcription factors are, for example, HHEX, SOX17, HNF4A, HNF1A, ONECUTI/HNF6, FoxA2, and FoxA1. In some embodiments, the one or more transcription factors are, for example, HHEX, SOX17, and HNF4A. In some embodiments, the one or more transcription factors are, for example, CEBPA, ATF5, HHEX, SOX17, HNF4A, ONECUT2/HNF6B, and HNF1A. In some embodiments, the one or more transcription factors are, for example, FoxA1, HNF1A, FoxA2, CEBPA, ONECUTI/HNF6, HNF4a, and RBPJ. In some embodiments, the one or more transcription factors are, for example, Smad3, HHEX, ATF5, NR5A2, GATA2 ISOFORM P23769-1, CEBPB, and NR1I3. In some embodiments, the one or more transcription factors are, for example, HNF1A, CEBPA, ONECUT1/HNF6, ONECUT2/HNF6B, Fos, HIFIA, and TBX3.

The present disclosure provides a hepatocyte or hepatic cell as disclosed herein, wherein the hepatocyte or the hepatic cell expresses ASGR-1. In some embodiments, the hepatocyte or the hepatic cell does not express CXCR-4. In some embodiments, the hepatocyte or the hepatic cell expresses CXCR-4.

The present disclosure provides a population of hepatocytes or hepatic cells comprising two or more of a hepatocyte or hepatic cell as disclosed herein. In some embodiments, at least 2% of the hepatocytes or the hepatic cell express ASGR-1. In some embodiments, at least 3% of the hepatocytes or the hepatic cell express ASGR-1. In some embodiments, at least 5% of the hepatocytes or the hepatic cell express ASGR-1. In some embodiments, at least 6% of the hepatocytes or the hepatic cell express ASGR-1. In some embodiments, at least 10% of the hepatocytes or the hepatic cell express CXCR-4. In some embodiments, at least 20% of the hepatocytes or the hepatic cell express CXCR-4. In some embodiments, at least 30% of the hepatocytes or the hepatic cell express CXCR-4. In some embodiments, no nutrient, growth factor or microenvironmental/matrix optimizations are performed.

Another aspect of the present disclosure provides an exogenous expression cassette comprising one or more transcription factors that induce differentiation of a pluripotent stem cell (PSC) into a hepatocyte or a hepatic cell, wherein the PSC is provided in a media and wherein the media is not altered during the differentiation of the PSC into the hepatocyte or hepatic cell. In some embodiments, the exogenous expression cassette induces differentiation of the PSC into the hepatocyte or hepatic cell in 30 days or less. In some embodiments, the exogenous expression cassette induces differentiation of the PSC into the hepatocyte or hepatic cell in 96 hours or less. In some embodiments, the one or more transcription factors comprise ONECUT1/HNF6 or ONECUT2/HNF6B. In some embodiments, the one or more transcription factors comprise ONECUTI/HNF6 and ONECUT2/HNF6B. In some embodiments, the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, three or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, four or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, five or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, the one or more transcription factors are, for example, SPI1, HNF1A, FoxA2, CEBPA, HNF6, HNF4a, and HNF6B. In some embodiments, the one or more transcription factors are, for example, HNF1A, Atf3, HNF6, HNF6B, Fos, CEBPA, and FoxM1. In some embodiments, the one or more transcription factors are, for example, SPI1, FoxA2, GATA2 ISOFORM P23769-2, GATA4, GATA2 ISOFORM P23769-1, JUN/AP-1, and NFIX. In some embodiments, the one or more transcription factors are, for example, HNF4a, CEBPA, GATA6, TBX3, Fos, GATA2 ISOFORM P23769-2, and GATA2 ISOFORM P23769-1. In some embodiments, the one or more transcription factors are, for example, GATA2 ISOFORM P23769-2, HNF4a, RXRB, NFE2L2, Myc, TP73, and HNF6. In some embodiments, the one or more transcription factors are, for example, GATA2 ISOFORM P23769-1, CEBPA, ATF3, GATA6, Fos, GATA3, NFIX. In some embodiments, the one or more transcription factors are, for example, HHEX, SOX17, HNF4A, HNF1A, ONECUT1/HNF6, FoxA2, and FoxA1. In some embodiments, the one or more transcription factors are, for example, HHEX, SOX17, and HNF4A. In some embodiments, the one or more transcription factors are, for example, CEBPA, ATF5, HHEX, SOX17, HNF4A, ONECUT2/HNF6B, and HNF1A. In some embodiments, the one or more transcription factors are, for example, FoxA1, HNF1A, FoxA2, CEBPA, ONECUT1/HNF6, HNF4a, and RBPJ. In some embodiments, the one or more transcription factors are, for example, Smad3, HHEX, ATF5, NR5A2, GATA2 ISOFORM P23769-1, CEBPB, and NR1I3. In some embodiments, the one or more transcription factors are, for example, HNF1A, CEBPA, ONECUTI/HNF6, ONECUT2/HNF6B, Fos, HIFIA, and TBX3. In some embodiments, the hepatocyte or the hepatic cell expresses ASGR-1. In some embodiments, the hepatocyte or the hepatic cell does not express CXCR-4. In some embodiments, the hepatocyte or the hepatic cell expresses CXCR-4.

The present disclosure provides a population of hepatocytes or hepatic cells comprising two or more of the hepatocyte or the hepatic cell as disclosed herein. In some embodiments, at least 2% of the hepatocytes or the hepatic cell express ASGR-1. In some embodiments, at least 3% of the hepatocytes or the hepatic cell express ASGR-1. In some embodiments, at least 5% of the hepatocytes or the hepatic cell express ASGR-1. In some embodiments, at least 6% of the hepatocytes or the hepatic cell express ASGR-1. In some embodiments, at least 10% of the hepatocytes or the hepatic cell express CXCR-4. In some embodiments, at least 20% of the hepatocytes or the hepatic cell express CXCR-4. In some embodiments, at least 30% of the hepatocytes or the hepatic cell express CXCR-4. In some embodiments, no nutrient, growth factor or microenvironmental/matrix optimizations are performed.

Another aspect of the present disclosure provides an exogenous expression cassette that induces differentiation of a pluripotent stem cell (PSC) into a hepatocyte or a hepatic cell, wherein the exogenous expression cassette comprises an HNF6 transcription factor and one or more additional transcription factors. In some embodiments, the PSC is provided in a media. In some embodiments, the media is not altered during the differentiation of the PSC into the hepatocyte or hepatic cell. In some embodiments, the exogenous expression cassette induces differentiation of the PSC into the hepatocyte or hepatic cell in 30 days or less. In some embodiments, the exogenous expression cassette induces differentiation of the PSC into the hepatocyte or hepatic cell in 96 hours or less. In some embodiments, the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, three or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, four or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, five or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, the one or more additional transcription factors are, for example, SPI1, HNF1A, FoxA2, CEBPA, HNF6, HNF4a, and HNF6B. In some embodiments, the one or more additional transcription factors are, for example, HNF1A, Atf3, HNF6, HNF6B, Fos, CEBPA, and FoxM1. In some embodiments, the one or more additional transcription factors are, for example, SPI1, FoxA2, GATA2 ISOFORM P23769-2, GATA4, GATA2 ISOFORM P23769-1, JUN/AP-1, and NFIX. In some embodiments, the one or more additional transcription factors are, for example, HNF4a, CEBPA, GATA6, TBX3, Fos, GATA2 ISOFORM P23769-2, and GATA2 ISOFORM P23769-1. In some embodiments, the one or more additional transcription factors are, for example, GATA2 ISOFORM P23769-2, HNF4a, RXRB, NFE2L2, Myc, TP73, and HNF6. In some embodiments, the one or more additional transcription factors are, for example, additional GATA2 ISOFORM P23769-1, CEBPA, ATF3, GATA6, Fos, GATA3, NFIX. In some embodiments, the one or more additional transcription factors are, for example, HHEX, SOX17, HNF4A, HNF1A, ONECUTI/HNF6, FoxA2, and FoxA1. In some embodiments, the one or more additional transcription factors are, for example, HHEX, SOX17, and HNF4A. In some embodiments, the one or more additional transcription factors are, for example, CEBPA, ATF5, HHEX, SOX17, HNF4A, ONECUT2/HNF6B, and HNF1A. In some embodiments, the one or more additional transcription factors are, for example, FoxA1, HNF1A, FoxA2, CEBPA, ONECUT1/HNF6, HNF4a, and RBPJ. In some embodiments, the one or more additional transcription factors are, for example, Smad3, HHEX, ATF5, NR5A2, GATA2 ISOFORM P23769-1, CEBPB, and NR1I3. In some embodiments, the one or more additional transcription factors are, for example, HNF1A, CEBPA, ONECUTI/HNF6, ONECUT2/HNF6B, Fos, HIF1A, and TBX3. In some embodiments, the hepatocyte or the hepatic cell expresses ASGR-1. In some embodiments, the hepatocyte or the hepatic cell does not express CXCR-4. In some embodiments, the hepatocyte or the hepatic cell expresses CXCR-4.

The present disclosure provides a population of hepatocytes or hepatic cells comprising two or more hepatocytes or the hepatic cells as disclosed herein. In some embodiments, at least 2% of the hepatocytes or the hepatic cell express ASGR-1. In some embodiments, at least 3% of the hepatocytes or the hepatic cell express ASGR-1. In some embodiments, at least 5% of the hepatocytes or the hepatic cell express ASGR-1. In some embodiments, at least 6% of the hepatocytes or the hepatic cell express ASGR-1. In some embodiments, at least 10% of the hepatocytes or the hepatic cell express CXCR-4. In some embodiments, at least 20% of the hepatocytes or the hepatic cell express CXCR-4. In some embodiments, at least 30% of the hepatocytes or the hepatic cell express CXCR-4. In some embodiments, no nutrient, growth factor or microenvironmental/matrix optimizations are performed.

Another aspect of the present disclosure provides a hepatocyte or hepatic cell comprising one or more nucleic acids comprising an open reading frame encoding one or more transcription factors, one or more transcription factors, or one or more activators of transcription of the open reading frame encoding one or more transcription factors, wherein the one or more transcription factors induce the differentiation of a pluripotent stem cell (PSC) into the hepatocyte or hepatic cell in 30 days or less. In some embodiments, the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the one or more transcription factors induce the differentiation of the PSC into the hepatocyte or hepatic cell in 96 hours or less. In some embodiments, the PSC is provided in a media. In some embodiments, the media is not altered during the differentiation of the PSC into the hepatocyte or hepatic cell. In some embodiments, two or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, three or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, four or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, five or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, the one or more transcription factors are, for example, HNF1A, SPI1, FoxA2, CEBPA, HNF6, HNF4a, and HNF6B. In some embodiments, the one or more additional transcription factors are, for example, HNF1A, Atf3, HNF6, HNF6B, Fos, CEBPA, and FoxM1. In some embodiments, the one or more transcription factors are, for example, FoxA1, FoxA2, GATA2 ISOFORM P23769-2, GATA4, GATA2 ISOFORM P23769-1, JUN/AP-1, and NFIX. In some embodiments, the one or more transcription factors are, for example, HNF4a, CEBPA, GATA6, TBX3, Fos, GATA2 ISOFORM P23769-2, and GATA2 ISOFORM P23769-1. In some embodiments, the one or more transcription factors are, for example, GATA2 ISOFORM P23769-2, HNF4a, RXRB, NFE2L2, Myc, TP73, and HNF6. In some embodiments, the one or more transcription factors are, for example, additional GATA2 ISOFORM P23769-1, CEBPA, ATF3, GATA6, Fos, GATA3, NFIX. In some embodiments, the one or more transcription factors are, for example, HHEX, SOX17, HNF4A, HNF1A, ONECUT1/HNF6, FoxA2, and FoxA1. In some embodiments, the one or more transcription factors are, for example, HHEX, SOX17, and HNF4A. In some embodiments, the one or more transcription factors are, for example, CEBPA, ATF5, HHEX, SOX17, HNF4A, ONECUT2/HNF6B, and HNF1A. In some embodiments, the one or more transcription factors are, for example, FoxA1, HNF1A, FoxA2, CEBPA, ONECUT1/HNF6, HNF4a, and RBPJ. In some embodiments, the one or more transcription factors are, for example, Smad3, HHEX, ATF5, NR5A2, GATA2 ISOFORM P23769-1, CEBPB, and NR1I3. In some embodiments, the one or more additional transcription factors are, for example, HNF1A, CEBPA, ONECUTI/, ONECUT2/HNF6B, Fos, HIFIA, and TBX3. In some embodiments, the hepatocyte or the hepatic cell expresses ASGR-1. In some embodiments, the hepatocyte or the hepatic cell does not express CXCR-4. In some embodiments, the hepatocyte or the hepatic cell expresses CXCR-4.

The present disclosure provides a population of hepatocytes or hepatic cells comprising two or more hepatocytes or the hepatic cells as disclosed herein. In some embodiments, at least 2% of the hepatocytes or the hepatic cell express ASGR-1. In some embodiments, at least 3% of the hepatocytes or the hepatic cell express ASGR-1. In some embodiments, at least 5% of the hepatocytes or the hepatic cell express ASGR-1. In some embodiments, at least 6% of the hepatocytes or the hepatic cell express ASGR-1. In some embodiments, at least 10% of the hepatocytes or the hepatic cell express CXCR-4. In some embodiments, at least 20% of the hepatocytes or the hepatic cell express CXCR-4. In some embodiments, at least 30% of the hepatocytes or the hepatic cell express CXCR-4. In some embodiments, no nutrient, growth factor or microenvironmental/matrix optimizations are performed.

Another aspect of the present disclosure provides a hepatocyte or hepatic cell comprising one or more nucleic acids comprising an open reading frame encoding one or more transcription factors, one or more transcription factors, or one or more activators of transcription of the open reading frame encoding one or more transcription factors, wherein the one or more transcription factors induce the differentiation of a pluripotent stem cell (PSC) into the hepatocyte or hepatic cell, and wherein the media is not altered during the differentiation of the PSC into the hepatocyte or hepatic cell. In some embodiments, the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the one or more exogenous expression cassettes induce differentiation of the PSC into the hepatocyte or the hepatic cell in 30 days or less. In some embodiments, two or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, three or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, four or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, five or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, the one or more transcription factors are, for example, SPI1, HNF1A, FoxA2, CEBPA, HNF6, HNF4a, and HNF6B. In some embodiments, the one or more additional transcription factors are, for example, HNF1A, Atf3, HNF6, HNF6B, Fos, CEBPA, and FoxM1. In some embodiments, the one or more transcription factors are, for example, SPI1, FoxA2, GATA2 ISOFORM P23769-2, GATA4, GATA2 ISOFORM P23769-1, JUN/AP-1, and NFIX. In some embodiments, the one or more transcription factors are, for example, HNF4a, CEBPA, GATA6, TBX3, Fos, GATA2 ISOFORM P23769-2, and GATA2 ISOFORM P23769-1. In some embodiments, the one or more transcription factors are, for example, GATA2 ISOFORM P23769-2, HNF4a, RXRB, NFE2L2, Myc, TP73, and HNF6. In some embodiments, the one or more transcription factors are, for example, additional GATA2 ISOFORM P23769-1, CEBPA, ATF3, GATA6, Fos, GATA3, NFIX. In some embodiments, the one or more transcription factors are, for example, HHEX, SOX17, HNF4A, HNF1A, ONECUT1/HNF6, FoxA2, and FoxA1. In some embodiments, the one or more transcription factors are, for example, HHEX, SOX17, and HNF4A. In some embodiments, the one or more transcription factors are, for example, CEBPA, ATF5, HHEX, SOX17, HNF4A, ONECUT2/HNF6B, and HNF1A. In some embodiments, the one or more transcription factors are, for example, FoxA1, HNF1A, FoxA2, CEBPA, ONECUT1/HNF6, HNF4a, and RBPJ. In some embodiments, the one or more transcription factors are, for example, Smad3, HHEX, ATF5, NR5A2, GATA2 ISOFORM P23769-1, CEBPB, and NR1I3. In some embodiments, the one or more additional transcription factors are, for example, HNF1A, CEBPA, ONECUT1/, ONECUT2/HNF6B, Fos, HIFIA, and TBX3. In some embodiments, the hepatocyte or the hepatic cell expresses ASGR-1. In some embodiments, the hepatocyte or the hepatic cell does not express CXCR-4. In some embodiments, the hepatocyte or the hepatic cell expresses CXCR-4.

The present disclosure provides a population of hepatocytes or hepatic cells comprising two or more hepatocytes or the hepatic cells as disclosed herein. In some embodiments, at least 2% of the hepatocytes or the hepatic cell express ASGR-1. In some embodiments, at least 3% of the hepatocytes or the hepatic cell express ASGR-1. In some embodiments, at least 5% of the hepatocytes or the hepatic cell express ASGR-1. In some embodiments, at least 6% of the hepatocytes or the hepatic cell express ASGR-1. In some embodiments, at least 10% of the hepatocytes or the hepatic cell express CXCR-4. In some embodiments, at least 20% of the hepatocytes or the hepatic cell express CXCR-4. In some embodiments, at least 30% of the hepatocytes or the hepatic cell express CXCR-4. In some embodiments, no nutrient, growth factor or microenvironmental/matrix optimizations are performed.

Another aspect of the present disclosure provides an ASGR-1 expressing cell comprising one or more nucleic acids comprising an open reading frame encoding one or more transcription factors, one or more transcription factors, or one or more activators of transcription of the open reading frame encoding one or more transcription factors, wherein the one or more transcription factors induce the differentiation of a pluripotent stem cell (PSC) into the ASGR-1 expressing cell, and wherein the media is not altered during the differentiation of the PSC into the hepatocyte or hepatic cell. In some embodiments, the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the one or more exogenous expression cassettes induce differentiation of the PSC into the hepatocyte or the hepatic cell in 30 days or less. In some embodiments, two or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, three or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, four or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, five or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, the one or more transcription factors are, for example, SPI1, HNF1A, FoxA2, CEBPA, HNF6, HNF4a, and HNF6B. In some embodiments, the one or more additional transcription factors are, for example, HNF1A, Atf3, HNF6, HNF6B, Fos, CEBPA, and FoxM1. In some embodiments, the one or more transcription factors are, for example, SPI1, FoxA2, GATA2 ISOFORM P23769-2, GATA4, GATA2 ISOFORM P23769-1, JUN/AP-1, and NFIX. In some embodiments, the one or more transcription factors are, for example, HNF4a, CEBPA, GATA6, TBX3, Fos, GATA2 ISOFORM P23769-2, and GATA2 ISOFORM P23769-1. In some embodiments, the one or more transcription factors are, for example, GATA2 ISOFORM P23769-2, HNF4a, RXRB, NFE2L2, Myc, TP73, and HNF6. In some embodiments, the one or more transcription factors are, for example, additional GATA2 ISOFORM P23769-1, CEBPA, ATF3, GATA6, Fos, GATA3, NFIX. In some embodiments, the one or more transcription factors are, for example, HHEX, SOX17, HNF4A, HNF1A, ONECUT1/HNF6, FoxA2, and FoxA1. In some embodiments, the one or more transcription factors are, for example, HHEX, SOX17, and HNF4A. In some embodiments, the one or more transcription factors are, for example, CEBPA, ATF5, HHEX, SOX17, HNF4A, ONECUT2/HNF6B, and HNF1A. In some embodiments, the one or more transcription factors are, for example, FoxA1, HNF1A, FoxA2, CEBPA, ONECUT1/HNF6, HNF4a, and RBPJ. In some embodiments, the one or more transcription factors are, for example, Smad3, HHEX, ATF5, NR5A2, GATA2 ISOFORM P23769-1, CEBPB, and NR1I3. In some embodiments, the one or more additional transcription factors are, for example, HNF1A, CEBPA, ONECUTI/, ONECUT2/HNF6B, Fos, HIFIA, and TBX3. In some embodiments, the hepatocyte or the hepatic cell expresses ASGR-1. In some embodiments, the hepatocyte or the hepatic cell does not express CXCR-4. In some embodiments, the hepatocyte or the hepatic cell expresses CXCR-4.

The present disclosure provides a population of hepatocytes or hepatic cells comprising two or more hepatocytes or the hepatic cells as disclosed herein. In some embodiments, at least 2% of the hepatocytes or the hepatic cell express ASGR-1. In some embodiments, at least 3% of the hepatocytes or the hepatic cell express ASGR-1. In some embodiments, at least 5% of the hepatocytes or the hepatic cell express ASGR-1. In some embodiments, at least 6% of the hepatocytes or the hepatic cell express ASGR-1. In some embodiments, at least 10% of the hepatocytes or the hepatic cell express CXCR-4. In some embodiments, at least 20% of the hepatocytes or the hepatic cell express CXCR-4. In some embodiments, at least 30% of the hepatocytes or the hepatic cell express CXCR-4. In some embodiments, no nutrient, growth factor or microenvironmental/matrix optimizations are performed.

The present disclosure provides an ASGR-1 expressing cell comprising one or more nucleic acids comprising an open reading frame encoding one or more transcription factors, one or more transcription factors, or one or more activators of transcription of the open reading frame encoding one or more transcription factors, wherein the nucleic acid comprising an open reading frame encoding one or more transcription factors, the one or more transcription factors, or the activator of transcription of the open reading frame encoding one or more transcription factors induces differentiation of a pluripotent stem cell (PSC) into the ASGR-1 expressing cell in 30 days or less.

The present disclosure provides a population of hepatocytes or hepatic cells comprising two or more hepatocytes or the hepatic cells as disclosed herein. In some embodiments, the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the one or more exogenous expression cassettes induce differentiation of the PSC into the hepatocyte or the hepatic cell in 30 days or less. In some embodiments, two or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, three or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, four or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, five or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, the one or more transcription factors are, for example, SPI1, HNF1A, FoxA2, CEBPA, HNF6, HNF4a, and HNF6B. In some embodiments, the one or more additional transcription factors are, for example, HNF1A, Atf3, HNF6, HNF6B, Fos, CEBPA, and FoxM1. In some embodiments, the one or more transcription factors are, for example, SPI1, FoxA2, GATA2 ISOFORM P23769-2, GATA4, GATA2 ISOFORM P23769-1, JUN/AP-1, and NFIX. In some embodiments, the one or more transcription factors are, for example, HNF4a, CEBPA, GATA6, TBX3, Fos, GATA2 ISOFORM P23769-2, and GATA2 ISOFORM P23769-1. In some embodiments, the one or more transcription factors are, for example, GATA2 ISOFORM P23769-2, HNF4a, RXRB, NFE2L2, Myc, TP73, and HNF6. In some embodiments, the one or more transcription factors are, for example, additional GATA2 ISOFORM P23769-1, CEBPA, ATF3, GATA6, Fos, GATA3, NFIX. In some embodiments, the one or more transcription factors are, for example, HHEX, SOX17, HNF4A, HNF1A, ONECUT1/HNF6, FoxA2, and FoxA1. In some embodiments, the one or more transcription factors are, for example, HHEX, SOX17, and HNF4A. In some embodiments, the one or more transcription factors are, for example, CEBPA, ATF5, HHEX, SOX17, HNF4A, ONECUT2/HNF6B, and HNF1A. In some embodiments, the one or more transcription factors are, for example, FoxA1, HNF1A, FoxA2, CEBPA, ONECUT1/HNF6, HNF4a, and RBPJ. In some embodiments, the one or more transcription factors are, for example, Smad3, HHEX, ATF5, NR5A2, GATA2 ISOFORM P23769-1, CEBPB, and NR1I3. In some embodiments, the one or more additional transcription factors are, for example, HNF1A, CEBPA, ONECUT1/, ONECUT2/HNF6B, Fos, HIFIA, and TBX3. In some embodiments, the hepatocyte or the hepatic cell expresses ASGR-1. In some embodiments, the hepatocyte or the hepatic cell does not express CXCR-4. In some embodiments, the hepatocyte or the hepatic cell expresses CXCR-4.

The present disclosure provides a population of hepatocytes or hepatic cells comprising two or more of the hepatocyte or the hepatic cell as disclosed herein. In some embodiments, at least 2% of the hepatocytes or the hepatic cell express ASGR-1. In some embodiments, at least 3% of the hepatocytes or the hepatic cell express ASGR-1. In some embodiments, at least 5% of the hepatocytes or the hepatic cell express ASGR-1. In some embodiments, at least 6% of the hepatocytes or the hepatic cell express ASGR-1. In some embodiments, at least 10% of the hepatocytes or the hepatic cell express CXCR-4. In some embodiments, at least 20% of the hepatocytes or the hepatic cell express CXCR-4. In some embodiments, at least 30% of the hepatocytes or the hepatic cell express CXCR-4. In some embodiments, no nutrient, growth factor or microenvironmental/matrix optimizations are performed.

Another aspect of the present disclosure provides a CXCR-4 expressing cell comprising one or more nucleic acids comprising an open reading frame encoding one or more transcription factors, one or more transcription factors, or one or more activators of transcription of the open reading frame encoding one or more transcription factors, wherein the one or more transcription factors induce the differentiation of a pluripotent stem cell (PSC) into the CXCR-4 expressing cell, and wherein the media is not altered during the differentiation of the PSC into the hepatocyte or hepatic cell. In some embodiments, the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, two or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more additional transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the one or more exogenous expression cassettes induce differentiation of the PSC into the hepatocyte or the hepatic cell in 30 days or less. In some embodiments, two or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, three or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, four or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, five or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, the one or more transcription factors are, for example, for example, SPI1, HNF1A, FoxA2, CEBPA, HNF6, HNF4a, and HNF6B. In some embodiments, the one or more additional transcription factors, for example, HNF1A, Atf3, HNF6, HNF6B, Fos, CEBPA, and FoxM1 or any combination thereof. In some embodiments, the one or more transcription factors are, for example, for example, SPI1, FoxA2, GATA2 ISOFORM P23769-2, GATA4, GATA2 ISOFORM P23769-1, JUN/AP-1, and NFIX or any combination thereof. In some embodiments, the one or more transcription factors are, for example, for example HNF4a, CEBPA, GATA6, TBX3, Fos, GATA2 ISOFORM P23769-2, and GATA2 ISOFORM P23769-1 or any combination thereof. In some embodiments, the one or more transcription factors are, for example, for example, GATA2 ISOFORM P23769-2, HNF4a, RXRB, NFE2L2, Myc, TP73, and HNF6 or any combination thereof. In some embodiments, the one or more transcription factors are, for example, GATA2 ISOFORM P23769-1, CEBPA, ATF3, GATA6, Fos, GATA3, NFIX or any combination thereof. In some embodiments, the one or more transcription factors are, for example, HHEX, SOX17, HNF4A, HNF1A, ONECUT1/HNF6, FoxA2, and FoxA1 or any combination thereof. In some embodiments, the one or more transcription factors are, for example, for example HHEX, SOX17, and HNF4A or any combination thereof. In some embodiments, the one or more transcription factors are, for example, CEBPA, ATF5, HHEX, SOX17, HNF4A, ONECUT2/HNF6B, and HNF1A or any combination thereof. In some embodiments, the one or more transcription factors are, for example, FoxA1, HNF1A, FoxA2, CEBPA, ONECUT1/HNF6, HNF4a, and RBPJ or any combination thereof. In some embodiments, the one or more transcription factors are, for example, Smad3, HHEX, ATF5, NR5A2, GATA2 ISOFORM P23769-1, CEBPB, and NR113 or any combination thereof. In some embodiments, the one or more additional transcription factors are, for example, HNF1A, CEBPA, ONECUTI/, ONECUT2/HNF6B, Fos, HIFIA, and TBX3 or any combination thereof. In some embodiments, the hepatocyte or the hepatic cell expresses CXCR-4. In some embodiments, the hepatocyte or the hepatic cell does not express CXCR-4. In some embodiments, the hepatocyte or the hepatic cell expresses CXCR-4.

The present disclosure provides a population of hepatocytes or hepatic cells comprising two or more of the hepatocyte or the hepatic cell as disclosed herein. In some embodiments, at least 2% of the hepatocytes or the hepatic cell express CXCR-4. In some embodiments, at least 3% of the hepatocytes or the hepatic cell express CXCR-4. In some embodiments, at least 5% of the hepatocytes or the hepatic cell express CXCR-4. In some embodiments, at least 6% of the hepatocytes or the hepatic cell express CXCR-4. In some embodiments, at least 10% of the hepatocytes or the hepatic cell express CXCR-4. In some embodiments, at least 20% of the hepatocytes or the hepatic cell express CXCR-4. In some embodiments, at least 30% of the hepatocytes or the hepatic cell express CXCR-4. In some embodiments, no nutrient, growth factor or microenvironmental/matrix optimizations are performed.

Another aspect of the present disclosure provides a CXCR-4 expressing cell comprising: (a) one or more nucleic acids, where the one or more nucleic acids each comprise an open reading frame encoding one or more transcription factors, (b) one or more transcription factors, and/or (c) one or more activators of transcription of the open reading frame encoding one or more transcription factors; wherein the nucleic acid comprising the open reading frame encoding the one or more transcription factors, the one or more transcription factors, or the activators of transcription of the open reading frame encoding the one or more transcription factors induces differentiation of a pluripotent stem cell (PSC) into the CXCR-4 expressing cell in 30 days or less.

In some embodiments, the nucleic acid comprising an open reading frame encoding one or more transcription factors, the one or more transcription factors, or the activator of transcription of the open reading frame encoding one or more transcription factors induces differentiation of a pluripotent stem cell (PSC) into the CXCR-4 expressing cell in 96 hours or less. In some embodiments, the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, TP53 family members, or any combination thereof. In some embodiments, two or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, three or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, four or more of the one or more transcription factors are, for example, basic leucine zipper transcription factors, forkhead box (FOX) transcription factors, HNF hepatocyte nuclear factor transcription factors, GATA zinc finger transcription factors, NKL subclass homeobox and pseudogene transcription factors, basic helix-loop-helix transcription factors, CUT homeobox transcription factors, NR nuclear receptor family members, IPT (Ig-like, Plexin, Transcription factors) domain, Nuclear Factor I (NFI) family transcription factors, SMAD proteins, SRY-box family members, Helix-turn-helix ETS family members, T-box family members, and TP53 family members. In some embodiments, the one or more exogenous expression cassettes induce differentiation of the PSC into the hepatocyte or the hepatic cell in 30 days or less. In some embodiments, two or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, three or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, four or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, five or more transcription factors are of a transcription factor family, for example: ATF, CEBP, FOS, FOX, GATA, HHEX, HIF, HNF, JUN, MYC, NFE, NFI, NR, ONECUT, RBPJ, RXR, SMAD, SOX, SPI, TBX, PPAR, FOS, JUN, SKI, SALL, EGR, and TP. In some embodiments, the one or more transcription factors are, for example, SPI1, HNF1A, FoxA2, CEBPA, HNF6, HNF4a, and HNF6B. In some embodiments, the one or more additional transcription factors are, for example, HNF1A, Atf3, HNF6, HNF6B, Fos, CEBPA, and FoxM1. In some embodiments, the one or more transcription factors are, for example, SPI1, FoxA2, GATA2 ISOFORM P23769-2, GATA4, GATA2 ISOFORM P23769-1, JUN/AP-1, and NFIX. In some embodiments, the one or more transcription factors are, for example, HNF4a, CEBPA, GATA6, TBX3, Fos, GATA2 ISOFORM P23769-2, and GATA2 ISOFORM P23769-1. In some embodiments, the one or more transcription factors are, for example, GATA2 ISOFORM P23769-2, HNF4a, RXRB, NFE2L2, Myc, TP73, and HNF6. In some embodiments, the one or more transcription factors are, for example, additional GATA2 ISOFORM P23769-1, CEBPA, ATF3, GATA6, Fos, GATA3, NFIX. In some embodiments, the one or more transcription factors are for example, HHEX, SOX17, HNF4A, HNF1A, ONECUT1/HNF6, FoxA2, and FoxA1. In some embodiments, the one or more transcription factors are for example, HHEX, SOX17, and HNF4A. In some embodiments, the one or more transcription factors are, for example, CEBPA, ATF5, HHEX, SOX17, HNF4A, ONECUT2/HNF6B, and HNF1A. In some embodiments, the one or more transcription factors are, for example, FoxA1, HNF1A, FoxA2, CEBPA, ONECUTI/HNF6, HNF4a, and RBPJ. In some embodiments, the one or more transcription factors are, for example, Smad3, HHEX, ATF5, NR5A2, GATA2 ISOFORM P23769-1, CEBPB, and NR1I3. In some embodiments, the transcription factors are, for example, HNF1A, CEBPA, ONECUTI/, ONECUT2/HNF6B, Fos, HIFIA, and TBX3. In some embodiments, the hepatocyte or the hepatic cell expresses CXCR-4. In some embodiments, the hepatocyte or the hepatic cell does not express CXCR-4. In some embodiments, the hepatocyte or the hepatic cell expresses CXCR-4.

The present disclosure provides a population of hepatocytes or hepatic cells comprising two or more of the hepatocyte or the hepatic cell as disclosed herein. In some embodiments, at least 2% of the hepatocytes or the hepatic cell express CXCR-4. In some embodiments, at least 3% of the hepatocytes or the hepatic cell express CXCR-4. In some embodiments, at least 5% of the hepatocytes or the hepatic cell express CXCR-4. In some embodiments, at least 6% of the hepatocytes or the hepatic cell express CXCR-4. In some embodiments, at least 10% of the hepatocytes or the hepatic cell express CXCR-4. In some embodiments, at least 20% of the hepatocytes or the hepatic cell express CXCR-4. In some embodiments, at least 30% of the hepatocytes or the hepatic cell express CXCR-4. In some embodiments, no nutrient, growth factor or microenvironmental/matrix optimizations are performed.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts an exemplary screen showing the ability of pools of transcription factors to induce hepatocyte formation. iPSCs were transfected, and then specific antibiotics were used to select for integrants.

FIG. 2 depicts an exemplary experiment showing the changes in morphology after differentiation in thirteen unique cell lines.

FIG. 3 depicts the expression of CK18, albumin, and ASGR-1 by cells with no transcription factor induction and by cells with transcription factor induction.

FIG. 4A-FIG. 4C depicts expression of hepatocyte-specific markers and/or definitive endoderm markers or the lack thereof for various cocktails. Shown is the percent cells that positively expressed the hepatocyte-specific marker, ASGR-1 (FIG. 4A), definitive endoderm marker, CXCR4 (FIG. 4B) and the stem-cell marker, TRA-1-60 (FIG. 4C).

FIG. 5A-FIG. 5B depicts the expression of different cell markers, ASGR-1 and albumin. The percent cells that were positive for CK18, albumin, and ASGR-1 in cell lines that expressed transcription factor (TF) recipes or combinations H0008 (FIG. 5A), H0011 (FIG. 5B) and the percentage of cells expressing both CK18 and albumin (FIG. 5C) by cell lines expressing both TF recipes is shown.

FIG. 6 depicts staining for CK18 and albumin for two independent experiments.

FIG. 7 depicts the results of immunofluorescence imaging for H0002.

FIG. 8 depicts the results of immunofluorescence imaging for H0003.

FIG. 9 depicts the results of immunofluorescence imaging for H0004.

FIG. 10 depicts the results of immunofluorescence imaging for H0005.

FIG. 11 depicts the results of immunofluorescence imaging for H0006.

FIG. 12 depicts the results of immunofluorescence imaging for H0008.

FIG. 13 depicts the results of immunofluorescence imaging for H0009.

FIG. 14 depicts the results of immunofluorescence imaging for H0010.

FIG. 15 depicts the results of immunofluorescence imaging for H0011.

FIG. 16 depicts the results of immunofluorescence imaging for H0012.

FIG. 17 depicts the results of immunofluorescence imaging for H0013.

FIG. 18A-FIG. 18B depicts primary hepatocytes and induced cell line expressing H0001 cocktail that differentiate into hepatocyte. The ability to store lipids is depicted with lipid droplets, which are stained red as expected in primary cells (FIG. 18A) and recapitulated by induced cells (FIG. 18B).

FIG. 19A-FIG. 19C illustrates ability of cells to store lipids when iPSCs are modified to express specific cocktail H0011. Storage of lipids was detected using Oil Red O staining in the primary cells (positive control; FIG. 19A) and the induced iPSCs that differentiated into hepatocytes and stained lipid droplets (FIG. 19B), but no signs of lipid accumulation as expected, was detected in hiPSCs without any TF induction (negative control; FIG. 19C).

FIG. 20A-FIG. 20B depicts the percent cells that differentiated to hepatocytes and therefore positively expressed ASGR-1, CK18, albumin markers and had ability to store lipids. The percent cells that positively expressed markers and stored lipid was confirmed for cells modified to express specific cocktail H0001 (FIG. 20A) and H0011 (FIG. 20B).

FIG. 21 illustrates an experimental scheme to screen transcription factors (TFs) critical for hepatocyte differentiation.

FIG. 22A-FIG. 22B illustrates use of flow cytometry to assess the percentage of differentiated cells that expressed the hepatocyte marker ASGR-1 on their surface. An example plot for transcription factor cocktails that were induced in modified cells is shown on the x-axis (FIG. 22A); run is consistent for transcription factor cocktail, (e.g., run 29 is cell line H0064). Expression of ASGR-1 was assessed in the run 29/H0064 cell line by flow cytometry; undifferentiated cells remained positive for the TRA-1-60 marker (FIG. 22B).

FIG. 23 illustrates an assay to measure the amount of albumin secreted by the differentiated cells was by ELISA.

FIG. 24A-FIG. 24C illustrates uniform manifold approximation and projection (“UMAP”) of induced H0011 cells which revealed two clusters of cells expressed hepatocyte genes and had down regulated expression of pluripotent genes. Clustering of the induced H0011 cells revealed six clusters of gene expression profiles (FIG. 24A). Heat map of gene expression showed clusters 4 and 5 had upregulated expression of hepatocyte genes, including APOA2, SERPINA1, APOA1, RBP4, CEBPA, HP, AND APOB (FIG. 24B). Mapping gene expression of selected genes onto the UMAP showed clusters 4 and 5 had low expression of the stem cell marker POU5F1 and higher expression of hepatocyte markers e.g., APOA2, fibrinogen (FGA), vitronectin (VTN), and albumin (ALB) (FIG. 24C).

FIG. 25A-FIG. 25C illustration of machine learning classification of hepatocyte-like cells in induced H0011 cells. A UMAP showed four samples that were studied: primary human hepatocytes, iPSCs, induced H0011, and uninduced H0011. The uninduced H0011 cells clustered more closely with the iPSCs than with the induced H0011 (FIG. 25A). Hepatocyte-like cells categorized by the classification algorithm included primary hepatocytes and iPSC-derived hepatocytes (FIG. 25B). A graph showing the number of hepatocyte-like and iPSC-like cells in each sample (FIG. 25C).

FIG. 26A-FIG. 26.B illustration showing that predicted hepatocyte-like cells in induced H0011 cells expressed specific immature and mature hepatocyte markers. The predicted hepatocyte-like cells are clustered together in the UMAP of the induced H0011 cells (FIG. 26A). The relative gene expression of a selected group of hepatocyte markers across all samples was assessed; predicted hepatocyte-like cells from the induced H0011 population had enriched expression of many key mature hepatocyte genes, including albumin, ASGR-1, apolipoproteins, and SERPINA1 (alpha-1-antitrypsin) (FIG. 26B). The predicted hepatocyte-like cells also expressed some immature hepatocyte markers such as DLK1 and AFP.

FIG. 27 illustration showing that predicted hepatocyte-like cells in induced H0011 had enriched expression of many hepatocyte-associated genes, including CYP3A5, CYP2B6, CYP2A7, SULT1A1, ABCC3, ABCC6, FAH, ALDH6A1, HAL, TAT, ABCC2, LEPR, ASL, HPR, CP, DEFB1, ACSL1, PLIN1, MTTP, APOB, APOM.

FIG. 28 illustration depicting an assay that was used to identify transcription factor combinations that lead to high proportion of hepatocyte-like cells in induced H0011 cells.

FIG. 29 illustrates induced cells from engineered lines H0014-16 that were stained with antibodies for alpha-fetoprotein (AFP), cytokeratin 18 (CK18), albumin, as well as DAPI to label nuclei. Scale bar: 50 μm.

FIG. 30 illustrates induced cells from engineered lines H0020, H0021, and H0025 that were stained with antibodies for AFP, CK18, albumin, as well as DAPI to label nuclei. Scale bar: 100 μm.

FIG. 31 illustrates induced cells from engineered lines H0015C and H0026A-30A that were stained with antibodies for A1AT, AFP, CK18, and albumin. Scale bar: 100 μm.

FIG. 32 illustrates induced cells from engineered lines H0011C and H0031A-35A were stained with antibodies for A1AT, AFP, CK18, and albumin. Scale bar: 100 μm.

FIG. 33A-FIG. 33C illustrates quantification of immunofluorescent staining on lines H0011C and H0031A-35A that were stained with antibodies for albumin, CK18, and ASGR-1. The percentage of cells that were positive for albumin (FIG. 33A), CK18 (FIG. 33B) or ASGR-1 (FIG. 33C), as indicated by an average object intensity over a set threshold, were calculated.

FIG. 34 illustrates transcription factors that were induced in H0014-35 cells for 96 hours and then Tra-1-60 expression (a marker of pluripotency) was measured via flow cytometry. The isotype shows background fluorescence. Non-engineered iPSCs are a positive control and HepG2 cells are a negative control.

FIG. 35 illustrates transcription factors that were induced in H0014-35 cells for 96 hours and then ASGR1 expression (a mature hepatocyte marker) was measured via flow cytometry. The isotype shows background fluorescence. Non-engineered iPSCs are a negative control and HepG2 cells are a positive control.

FIG. 36 illustrates transcription factors that were induced in H0014-35 cells for 96 hours and then ASGR1 and TRA-1-60 expression was measured via flow cytometry. This graph shows the percentage of ASGR1+ cells of the TRA-1-60-population. The isotype shows background fluorescence.

FIG. 37 illustrates transcription factors that were induced in H0014-35 cells for 96 hours and then CXCR4 expression (marker of definitive endoderm) was measured via flow cytometry. The isotype shows background fluorescence. Non-engineered iPSCs are a negative control and HepG2 cells are a positive control.

FIG. 38A-FIG. 38C illustrates RT-qPCR data showing increase in liver specific markers transcription factor induction. RT-qPCR was performed on uninduced and induced engineered hepatocyte-like cells, as well as primary human hepatocytes normalized by a housekeeping gene. Fold-change expression for ALB (FIG. 38A), CYP3A7 (FIG. 38B) and CYP3A4 (FIG. 38C) genes was calculated relative to the median value of the uninduced control cells. Significance determined by unpaired t-test; * p<0.5, ** p<0.01.

FIG. 39 illustrates the amount of albumin produced by uninduced and induced engineered cell lines H0027B, H0028B, H0031A, and H0033A as measured by ELISA and normalized for time and total number of cells.

FIG. 40 illustrates the amount of A1AT produced by uninduced and induced engineered cell lines H0027B, H0028B, H0031A, and H0033A as measured by ELISA and normalized for time and total number of cells.

FIG. 41 illustrates schematic depicting cells of H0028B and H0033A that were isolated and differentiated for four days (96 hours) and seven days and analyzed by several metrics, including immunofluorescent staining (for A1AT, CK18, and albumin), ELISAs to detect secreted A1AT and albumin, chromogenic assay was used to detect secreted urea, and luminogenic substrate cleavage rate measurements of cytochrome p450 enzymatic activity. The top performers that exhibited the most hepatocyte-like phenotype as assessed by these metrics were selected for expansion and further validation.

FIG. 42A-FIG. 42B illustrates top performers from H0028 (H28B) and H0033 (H33A) cell lines that were cultured for 4 days (96 hours) in mTeSR Plus stem cell medium with transcription factor induction in 0.5 μM A83-01. Cells were next stained with antibodies to detect A1AT, CK18, and albumin. Several performers functioned well across multiple assays, suggesting that they are hepatocyte-like (FIGS. 42A-42B). When best performers were compared for A1AT, CK18, and albumin to the original cells, higher percentages of cells expressed A1AT, CK18 and albumin compared to uninduced and parental cells (FIGS. 42A-42B). The numbers below the images represent the percentage of cells that express each marker, averaged across nine fields of view. Scale bar: 100 μm.

FIG. 43A-FIG. 43B illustrates cell lines that expressed H0028B and H0033A transcription factors that were also cultured for 4 days (96 hours) in mTeSR Plus with transcription factor induction. Cells were next stained using antibodies for A1AT, CK18, and albumin after 96 hours of staining. Several performers functioned well across multiple assays, suggesting that they are hepatocyte-like (FIGS. 43A-42B). When best performers were compared for A1AT, CK18, and albumin to the original cells, higher percentages of cells expressed A1AT, CK18 and albumin compared to uninduced and parental cells (FIG. 43A-34B). The numbers below the images represent the percentage of cells that express each marker, averaged across nine fields of view. Scale bar: 100 μm.

FIG. 44A-FIG. 44B illustrates cell lines that expressed H0028B and H0033A that were also cultured for 96 hours in mTeSR Plus without transcription factor induction. They were then stained using antibodies for A1AT, CK18, and albumin. Several performers functioned well across multiple assays, suggesting that they are hepatocyte-like (FIGS. 44A-42B). When best performers were compared for A1AT, CK18, and albumin to the original cells, higher percentages of cells expressed A1AT, CK18 and albumin compared to uninduced and parental cells (FIG. 44A-44B). The numbers below the images represent the percentage of cells that express each marker, averaged across nine fields of view. Scale bar: 100 μm.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Pluripotent stem cells (PSCs) are characterized by their ability to self-renew, while maintaining potency for therapeutic applications. PSCs also have the ability to differentiate into a vast number of distinct phenotypes (e.g., hepatocytes). Disclosed herein are various compositions, formulations, and methods that facilitate efficient differentiation of PSCs to hepatocytes (and/or hepatic cells).

The hepatocytes or hepatic cells generated from the utilization of the compositions, formulations, and methods disclosed herein can be used for therapeutic applications wherein classical lineage hepatocytes have been conventionally used. Thus, the various compositions, formulations, and methods disclosed herein provide robust improvements to hepatocyte-based therapeutics as the compositions, formulations, and methods disclosed herein enable efficient and repeatable PSC differentiation into potent hepatocytes and/or hepatic cells.

PSCs are capable of dividing indefinitely and producing identical daughter cells. When provided with a signal, PSCs may differentiate into various phenotypes. In an example, a PSC is an embryonic stem cell (ESC). In another example, a PSC is an induced pluripotent stem cell (iPSC).

In some aspects, the present disclosure provides methods and compositions for efficient, repeatable differentiation of PSCs into hepatocytes or hepatic cells (e.g., defined by cell-surface marker expression signatures, function, etc.). These methods and compositions can provide robust improvements to current cell therapies that utilize hepatocyte or hepatic cell administration (e.g., availability, dosage, potency etc.).

### Pluripotent Stem Cells

Pluripotent stem cells (PSCs) may be characterized by self-renewal and potency. PSCs are capable of dividing indefinitely and producing identical daughter cells. When provided with a signal, PSCs may differentiate into various phenotypes. In an example, a PSC is an embryonic stem cell (ESC). In another example, a PSC is an induced pluripotent stem cell (iPSC). PSCs (e.g., ESCs and iPSCs) express TRA-1-60. TRA-1-60 expression is indicative of the cells' ability to differentiate.

iPSCs are a type of pluripotent stem cell derived from adult somatic cells that have been genetically reprogrammed to an embryonic stem (ES) cell-like state through the expression of genes and factors important for maintaining the defining properties of ES cells. iPSCs are like ES cells in many aspects, including the expression of ES cell markers, chromatin methylation patterns, embryoid body formation, teratoma formation, viable chimera formation, pluripotency, and the ability to contribute to many different tissues in vitro. Studies have reported a directed differentiation of iPSCs into a variety of functional cell types in vitro, and cell therapy effects of implanted iPSCs have been demonstrated in several animal models of disease. Directed differentiation is a bioengineering method that harnesses the potential of stem cells by constraining their differentiation in vitro toward a specific cell type or tissue of interest. Directed differentiation may be primarily applied to PSCs of mammalian origin, for example, mouse and human cells for biomedical research applications. Cell differentiation may involve a transformation from a proliferative mode toward differentiation mode. Directed differentiation may comprise mimicking developmental cultures in controlled conditions involving specific substrate or extracellular matrices promoting cell adhesion and differentiation and define culture media compositions. Signaling factors, such as growth factors or small molecules may be applied sequentially or in a combinatorial manner, at varying dosage and exposure time, to modulate differentiate. Direct reprogramming, also known as trans-differentiation or direct conversion, may comprise overexpressing one or several factors, introduced in the cells. In an example, the one or several factors may be transcription factors. Proper differentiation of the cell type of interest may be verified by analyzing cell type specific markers, gene expression profile, and functional assays.

The present disclosure provides pluripotent stem cells that may be induced to differentiate into one or more hepatocytes or hepatic cells. The pluripotent stem cells may be induced to differentiate into one or more hepatocytes or hepatic cells through various methods, including being induced to express one or more transcription factors, being contacted with one or more transcription factor proteins or nucleic acids encoding transcription factor proteins, or through other means as disclosed herein. The one or more nucleic acids may be expressed in one or more exogenous expression cassettes. The one or more exogenous expression cassettes may comprise one or more transcription factors. The one or more transcription factors may induce differentiation of the PSC. The one or more transcription factors may induce differentiation of the PSC into a hepatocyte or a hepatic cell. The one or more transcription factors may induce differentiation of the PSC into a hepatocyte, or a hepatic cell, or a cell that expresses ASGR-1, or a cell that expresses CXCR-4, or a cell that expresses cytokeratin-18 (CK18), or a cell that expresses alpha-fetoprotein (AFP), or a cell that expresses alpha-1-antitrypsin (A1AT), or a cell that expresses albumin, or a cell that expresses cytokine p450 subfamily CYP3A7 or a cell that expresses cytokine p450 subfamily CYP3A4 or any combination thereof. The pluripotent stem cells may be induced to differentiate into one or more hepatocytes or hepatic cells in 30 days or less. The pluripotent stem cells may be induced to differentiate into one or more hepatocytes or hepatic cells in 96 hours or less. The exogenous expression cassettes may induce the differentiation of the PSC into the hepatocyte or hepatic cell in 30 days or less. The exogenous expression cassettes may induce the differentiation of the PSC into the hepatocyte or hepatic cell in 96 hours or less. The PSC may be provided in a media. The media may not have to be altered during the differentiation of the PSC into the hepatocyte or hepatic cell. The media may be altered during the differentiation of the PSC into the hepatocyte or hepatic cell. A population of cells comprising one or more hepatocytes or hepatic cells may be generated. A population of cells comprising one or more hepatocytes, or hepatic cells, cells that express ASGR-1, cells that express CXCR-4, or cells that express cytokeratin-18 (CK18), or cells that express alpha-fetoprotein (AFP), or cells that express alpha-1-antitrypsin (A1AT), or cells that express albumin, or cells that express cytokine p450. The population of cells may comprise adherent cells. The population of cells may comprise suspension cells. The population of cells may comprise adherent cells and suspension cells. The population of cells may be provided in a media. The media may not have to be altered during the differentiation of the PSCs into hepatocytes or hepatic cells. The media may not need any nutrients, growth factors, or microenvironmental or matrix optimizations. The media may need any nutrients, growth factors, or microenvironmental or matrix optimizations. In some embodiments, no nutrient, growth factor or microenvironmental/matrix optimizations are performed. In some embodiments, nutrient, growth factor or microenvironmental/matrix optimizations are performed. In some embodiments, nutrient, growth factor, and microenvironmental/matrix optimizations are performed. In some embodiments, no nutrient optimizations are performed. In some embodiments, no growth factor optimizations are performed. In some embodiments, no microenvironmental/matrix optimizations are performed. In some embodiments, no microenvironmental optimizations are performed. In some embodiments, no matrix optimizations are performed. In some embodiments, no nutrient or growth factor optimizations are performed. In some embodiments, no nutrient or microenvironmental/matrix optimizations are performed. In some embodiments, no growth factor or microenvironmental/matrix optimizations are performed. In some embodiments, no nutrient or microenvironmental optimizations are performed. In some embodiments, no growth factor or microenvironmental optimizations are performed. In some embodiments, no nutrient or matrix optimizations are performed. In some embodiments, no growth factor or matrix optimizations are performed.

### Hepatocytes

Hepatocytes can exist in the liver. Hepatocytes can be involved in synthesis of proteins, storage of proteins, transformation of carbohydrates, synthesis of cholesterol, synthesis of bile salts, synthesis of phospholipids, detoxification, and secretion of bile. Hepatocytes can be cubical. In an example, hepatocytes can have side lengths of 20-30 microns. In another example, a hepatocyte can have a volume of 3.4×10⁻⁹ cm³. Hepatocytes can be used to explore the mechanisms of drug metabolism. Hepatocytes can be used to predict in vivo drug metabolism. Hepatocytes can be isolated by collagenase digestion.

In some embodiments, hepatocytes may express the protein asialoglycoprotein receptor 1 (ASGR-1), which is also known as ASGR1, ASGPR, ASGPR1, CLEC4H1, HL-1, or asialoglycoprotein receptor 1. ASGR-1 (e.g., a subunit thereof) can be encoded by the ASGR-1 gene. The ASGR-1 protein can act as a receptor protein. ASGR-1 can be a transmembrane protein. ASGR-1 can play a role in serum glycoprotein homeostasis. ASGR-1 can mediate the endocytosis of glycoproteins. ASGR-1 can mediate the endocytosis of glycoproteins with exposed terminal galactose. ASGR-1 can mediate the endocytosis of glycoproteins with N-acetylgalactosamine residues. ASGR-1 can facilitate hepatic infection. ASGR-1 can facilitate hepatic infection by viruses. ASGR-1 can facilitate hepatic infection by hepatitis B. ASGR-1 can be a target for liver-specific drug delivery. ASGR-1 can be a heterooligomeric protein. ASGR-1 can comprise major and minor subunits. The major and minor subunits can be encoded by different genes. Multiple isoforms of ASGR-1 can be encoded via alternative splicing.

In some embodiments, hepatocytes may express the chemokine receptor type 4 protein CXCR-4, which is also known as for example, fusin, CXCR4, CD184, D2S201E, FB22, HM89, HSY3RR, LAP-3, LAP3, LCR1, LESTR, NPY3R, NPYR, NPYRL, NPYY3R, WHIM, WHIMS, C-X-C motif chemokine receptor 4, orWHIMS1. CXCR-4 can be classified as a CXC chemokine receptor. CXCR-4 can be considered an alpha-chemokine receptor. CXCR-4 can be specific for stromal-derived-factor-1 (SDF-1). SDF-1 can exhibit chemotaxis towards lymphocytes. SDF-1 can be important in hematopoietic stem cell homing to the bone marrow. SDF-1 can be important in hematopoietic stem cell quiescence. CXCR-4 can aid in liver regeneration. CXCR-4 can be profibrotic. CXCR-4 can be profibrotic through activation of hepatic stellate cells. CXCR-4 signaling can be detrimental to liver recovery and regeneration. Clinical therapy with a CXCR-4 antagonist may improve hepatic recovery following acute livery injury. In liver disease, aberrant CXCR-4 expression can be related to migration and movement of liver-specific cells. In liver disease, aberrant CXCR-4 expression can be related to migration and movement of liver-specific cells through crosstalk with other pathways. CXCR-4 and its ligand may play a role in hepatitis. The mechanisms of inflammatory responses mediated by CXCR-4 signaling may impact (e.g., adversely) the chemotaxis of inflammatory cells (e.g., lymphocytes, neutrophils, and monocytes). CXCR-4 can be used by HIV to infect T cells. CXCR-4 receptors can be implicated in the adhesion phase of human implantation. CXCR-4 signaling can regulate expression of CD20 on B cells. Ubiquitin can be a natural ligand of CXCR-4. CXCR-4 dimerization can be dynamic. CXCR-4 dimerization can increase with concentration. CXCR-4 can be expressed in cancer, including breast cancer, ovarian cancer, melanoma, or prostate cancer.

In an aspect, the present disclosure provides one or more hepatocytes, or one or more hepatic cells, or one or more cells that express ASGR-1, or one or more cells that express CXCR-4, or one or more cells that express cytokeratin-18 (CK18), or one or more cells that express alpha-fetoprotein (AFP), or one or more cells that express alpha-1-antitrypsin (A1AT), or one or more cells that express albumin, or one or more cells that express cytokine p450 subfamily CYP3A7 or cytokine p450 subfamily CYP3A4, or a cell that expresses any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory. In some embodiments, the hepatocytes, or hepatic cells, or cells that expresses ASGR-1, cells that expresses CXCR-4, cells that express cytokeratin-18 (CK18), or cell that expresses alpha-fetoprotein (AFP), or cells that express alpha-1-antitrypsin (A1AT), or cells that express albumin, or cells that express cytokine p450 subfamily CYP3A7 or cells that express cytokine p450 subfamily CYP3A4 may comprise one or more exogenous expression cassettes. In some embodiments, the one or more exogeneous expression cassettes comprise one or more transcription factors that have induced the differentiation of one or more PSCs. The exogenous expression cassettes may induce the differentiation of the PSC into the hepatocytes, hepatic cells, cells that express ASGR-1, cells that express CXCR-4, cells that express cytokeratin-18 (CK18), or cells that express alpha-fetoprotein (AFP), or cells that express alpha-1-antitrypsin (A1AT), or cells that express albumin, or cells that express cytokine p450 subfamily CYP3A7 or cells that express cytokine p450 subfamily CYP3A4 or a cell that expresses any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory. In some embodiments, the PSC may be provided in a media. The media may not have to be altered during the differentiation of the PSC into the hepatocyte, hepatic cell, or cell that expresses ASGR-1, cell that expresses CXCR-4, or cell that expresses cytokeratin-18 (CK18), or cell that expresses alpha-fetoprotein (AFP), or cell that expresses alpha-1-antitrypsin (A1AT), or cell that expresses albumin, or cell that expresses cytokine p450 subfamily CYP3A7 or cell that expresses cytokine p450 subfamily CYP3A4 or a cell that expresses any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory. In some embodiments, the PSC may be provided in a media.

The present disclosure provides PSCs that may be induced to differentiate into one or more hepatocytes or hepatic cells. The pluripotent stem cells may be induced to differentiate into one or more hepatocytes or hepatic cells through various methods, including being induced to express one or more transcription factors, being contacted with one or more transcription factor proteins or nucleic acids encoding transcription factor proteins, or through other means as disclosed herein. The PSCs may be induced to differentiate into one or more hepatocytes or hepatic cells in 30 days or less. The PSCs may be induced to differentiate into one or more hepatocytes or hepatic cells in at most about 11, 10, 9, 8, 7, 6, 5, 4, 3, 2 days or less. The PSCs may be induced to differentiate into one or more hepatocytes or hepatic cells in at most about 1 day. The PSCs may be induced to differentiate into one or more hepatocytes or hepatic cells in at least most 4 days. The PSCs may be induced to differentiate into one or more hepatocytes or hepatic cells in at most about 96 hours. The PSCs may be induced to differentiate into one or more hepatocytes or hepatic cells in at most about 96, 90, 80, 70, 60, 50, 40, 30, 20, 10 hours or less. The one or more nucleic acids may be expressed in one or more exogenous expression cassettes. The one or more exogenous expression cassettes may comprise one or more transcription factors. The one or more transcription factors may induce differentiation of the PSC. The one or more transcription factors may induce differentiation of the PSC into a hepatocyte or a hepatic cell. The PSC may comprise one or more exogenous expressions cassettes. The one or more exogenous cassettes may comprise one or more transcription factors that induce differentiation of the PSC into a hepatocyte, hepatic cell, or a cell that expresses ASGR-1, cell that expresses CXCR-4, or a cell that expresses any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory. The exogenous expression cassettes may induce the differentiation of the PSC into the hepatocyte, hepatic cell, cell that expresses ASGR-1, cell that expresses CXCR-4, cell that does not express ASGR-1, or cell that does not express CXCR-4 in 30 days or less. The exogenous expression cassettes may induce the differentiation of the PSC into the hepatocyte, hepatic cell, cell that expresses ASGR-1, cell that expresses CXCR-4, cell that does not express ASGR-1, or cell that does not express CXCR-4 in 96 hours or less. The PSC may be provided in a media. The media may not have to be altered during the differentiation of the PSC into the hepatocyte, hepatic cell, cell that expresses ASGR-1, cell that expresses CXCR-4, cell that does not express ASGR-1, or cell that does not express CXCR-4.

The present disclosure provides a population of cells comprising one or more hepatocyte, hepatic cell, cell that expresses ASGR-1, cell that expresses CXCR-4, or a cell that expresses or any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory. The population of cells may comprise adherent cells. The population of cells may comprise suspension cells. The population of cells may comprise adherent cells and suspension cells. The population of cells may be provided in a media. The media may not have to be altered during the differentiation of the PSCs into hepatocytes, hepatic cells, cells that express ASGR-1, cells that express CXCR-4, or cells that express or any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory. The population of cells may be provided in a media. The media may not need any nutrients, growth factors, or microenvironmental or matrix optimizations. At least 5% of the cells may express ASGR-1. At least about 1%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, or at least about 40% of the cells may express ASGR-1 or a cell that may express any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory. At least 5% of the cells may express CXCR-1 or a cell that may express any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory. At least about 1%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, or at least about 40% of the cells may express ASGR-1.

In some embodiments, at least 0.01% of the hepatocytes or the hepatic cell express ASGR-1. In some embodiments, at least 0.1% of the hepatocytes or the hepatic cell express ASGR-1. In some embodiments, at least 0.5% of the hepatocytes or the hepatic cell expresses ASGR-1 or a cell that expresses any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory. In some embodiments, at least 1% of the hepatocytes or the hepatic cell express ASGR-1. In some embodiments, at least 1.5% of the hepatocytes or the hepatic cell express ASGR-1. In some embodiments, at least 2% of the hepatocytes or the hepatic cell express ASGR-1. In some embodiments, at least 3% of the hepatocytes or the hepatic cell express ASGR-1. In some embodiments, at least 4% of the hepatocytes or the hepatic cell express ASGR-1. In some embodiments, at least 5% of the hepatocytes or the hepatic cell express ASGR-1. In some embodiments, at least 6% of the hepatocytes or the hepatic cell express ASGR-1. In some embodiments, at least 7% of the hepatocytes or the hepatic cell express ASGR-1. In some embodiments, at least 8% of the hepatocytes or the hepatic cell express ASGR-1. In some embodiments, at least 9% of the hepatocytes or the hepatic cell express ASGR-1. In some embodiments, at least 10% of the hepatocytes or the hepatic cell express ASGR-1. In some embodiments, at least 12% of the hepatocytes or the hepatic cell express ASGR-1. In some embodiments, at least 15% of the hepatocytes or the hepatic cell express ASGR-1. In some embodiments, at least 17% of the hepatocytes or the hepatic cell express ASGR-1. In some embodiments, at least 20% of the hepatocytes or the hepatic cell express ASGR-1. In some embodiments, at least 25% of the hepatocytes or the hepatic cell express ASGR-1. In some embodiments, at least 30% of the hepatocytes or the hepatic cell express ASGR-1. In some embodiments, at least 35% of the hepatocytes or the hepatic cell express ASGR-1. In some embodiments, at least 40% of the hepatocytes or the hepatic cell express ASGR-1. In some embodiments, at least 45% of the hepatocytes or the hepatic cell express ASGR-1. In some embodiments, at least 50% of the hepatocytes or the hepatic cell express ASGR-1. In some embodiments, at least 60% of the hepatocytes or the hepatic cell express ASGR-1. In some embodiments, at least 70% of the hepatocytes or the hepatic cell express ASGR-1. In some embodiments, at least 80% of the hepatocytes or the hepatic cell express ASGR-1. In some embodiments, at least 90% of the hepatocytes or the hepatic cell express ASGR-1. In some embodiments, at least 95% of the hepatocytes or the hepatic cell express ASGR-1. In some embodiments, at least 99% of the hepatocytes or the hepatic cell express ASGR-1.

In some embodiments, the percentage of the hepatocytes or hepatic cells that express ASGR-1, or a cell that expresses any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory is at least about 0.01%, at least about 0.1%, at least about 0.5%, at least about 1%, at least about 1.5%, at least about 2%, at least about 3%, at least about 4%, at least about 5%, at least about 6%, at least about 7%, at least about 8%, at least about 9%, at least about 10%, at least about 12%, at least about 15%, at least about 17%, at least about 20%, at least about 25%, at least about 30%, at least about 35%, at least about 40%, at least about 45%, at least about 50%, at least about 60%, at least about 70%, at least about 80%, at least about 90%, at least about 95%, or at least about 99%. In some embodiments, In some embodiments, the percentage of the hepatocytes or hepatic cells that express ASGR-1 is at most about 0.01%, at most about 0.1%, at most about 0.5%, at most about 1%, at most about 1.5%, at most about 2%, at most about 3%, at most about 4%, at most about 5%, at most about 6%, at most about 7%, at most about 8%, at most about 9%, at most about 10%, at most about 12%, at most about 15%, at most about 17%, at most about 20%, at most about 25%, at most about 30%, at most about 35%, at most about 40%, at most about 45%, at most about 50%, at most about 60%, at most about 70%, at most about 80%, at most about 90%, at most about 91%, at most about 92%, at most about 93%, at most about 94%, at most about 95%, at most about 96%, at most about 97%, at most about 98%, at most about 99%, at most about 99.55, or at most about 99.9%.

In some embodiments, the percentage of the hepatocytes or the hepatic cells that express ASGR-1 is about 1% to about 90%. In some embodiments, the percentage of the hepatocytes or the hepatic cells that express ASGR-1 or cells that express any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory is at least about 1%. In some embodiments, the percentage of the hepatocytes or the hepatic cells that express ASGR-1 is at most about 90%. In some embodiments, the percentage of the hepatocytes or the hepatic cells that express ASGR-1 is about 1% to about 2%, about 1% to about 3%, about 1% to about 5% about 1% to about 6%, about 1% to about 7%, about 1% to about 8%, about 1% to about 10%, about 1% to about 20%, about 1% to about 30%, about 1% to about 50%, about 1% to about 90%, about 2% to about 3%, about 2% to about 5%, about 2% to about 6%, about 2% to about 7%, about 2% to about 8%, about 2% to about 10%, about 2% to about 20%, about 2% to about 30%, about 2% to about 50%, about 2% to about 90%, about 3% to about 5%, about 3% to about 6%, about 3% to about 7%, about 3% to about 8%, about 3% to about 10%, about 3% to about 20%, about 3% to about 30%, about 3% to about 50%, about 3% to about 90%, about 5% to about 6%, about 5% to about 7%, about 5% to about 8%, about 5% to about 10%, about 5% to about 20%, about 5% to about 30%, about 5% to about 50%, about 5% to about 90%, about 6% to about 7%, about 6% to about 8%, about 6% to about 10%, about 6% to about 20%, about 6% to about 30%, about 6% to about 50%, about 6% to about 90%, about 7% to about 8%, about 7% to about 10%, about 7% to about 20%, about 7% to about 30%, about 7% to about 50%, about 7% to about 90%, about 8% to about 10%, about 8% to about 20%, about 8% to about 30%, about 8% to about 50%, about 8% to about 90%, about 10% to about 20%, about 10% to about 30%, about 10% to about 50%, about 10% to about 90%, about 20% to about 30%, about 20% to about 50%, about 20% to about 90%, about 30% to about 50%, about 30% to about 90%, or about 50% to about 90%. In some embodiments, the percentage of the hepatocytes or the hepatic cells that express ASGR-1 is about 1%, about 2%, about 3%, about 5%, about 6%, about 7%, about 8%, about 10%, about 20%, about 30%, about 50%, or about 90%.

In some embodiments, at least 0.01% of the hepatocytes or the hepatic cell express CXCR-4. In some embodiments, at least 0.1% of the hepatocytes or the hepatic cell express CXCR-4 or a cell that expresses any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory. In some embodiments, at least 0.5% of the hepatocytes or the hepatic cells express CXCR-4 or cells that express any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory. In some embodiments, at least 1% of the hepatocytes or the hepatic cell express CXCR-4. In some embodiments, at least 1.5% of the hepatocytes or the hepatic cell express CXCR-4. In some embodiments, at least 2% of the hepatocytes or the hepatic cell express CXCR-4. In some embodiments, at least 3% of the hepatocytes or the hepatic cell express CXCR-4. In some embodiments, at least 4% of the hepatocytes or the hepatic cell express CXCR-4. In some embodiments, at least 5% of the hepatocytes or the hepatic cell express CXCR-4. In some embodiments, at least 6% of the hepatocytes or the hepatic cell express CXCR-4. In some embodiments, at least 7% of the hepatocytes or the hepatic cell express CXCR-4. In some embodiments, at least 8% of the hepatocytes or the hepatic cell express CXCR-4. In some embodiments, at least 9% of the hepatocytes or the hepatic cell express CXCR-4. In some embodiments, at least 10% of the hepatocytes or the hepatic cell express CXCR-4. In some embodiments, at least 12% of the hepatocytes or the hepatic cell express CXCR-4. In some embodiments, at least 15% of the hepatocytes or the hepatic cell express CXCR-4. In some embodiments, at least 17% of the hepatocytes or the hepatic cell express CXCR-4. In some embodiments, at least 20% of the hepatocytes or the hepatic cell express CXCR-4. In some embodiments, at least 25% of the hepatocytes or the hepatic cell express CXCR-4. In some embodiments, at least 30% of the hepatocytes or the hepatic cell express CXCR-4. In some embodiments, at least 35% of the hepatocytes or the hepatic cell express CXCR-4. In some embodiments, at least 40% of the hepatocytes or the hepatic cell express CXCR-4. In some embodiments, at least 45% of the hepatocytes or the hepatic cell express CXCR-4. In some embodiments, at least 50% of the hepatocytes or the hepatic cell express CXCR-4. In some embodiments, at least 60% of the hepatocytes or the hepatic cell express CXCR-4. In some embodiments, at least 70% of the hepatocytes or the hepatic cell express CXCR-4. In some embodiments, at least 80% of the hepatocytes or the hepatic cell express CXCR-4. In some embodiments, at least 90% of the hepatocytes or the hepatic cell express CXCR-4. In some embodiments, at least 95% of the hepatocytes or the hepatic cell express CXCR-4. In some embodiments, at least 99% of the hepatocytes or the hepatic cell express CXCR-4.

In some embodiments, the percentage of the hepatocytes or hepatic cells that express CXCR-4 is at least about 0.01%, at least about 0.1%, at least about 0.5%, at least about 1%, at least about 1.5%, at least about 2%, at least about 3%, at least about 4%, at least about 5%, at least about 6%, at least about 7%, at least about 8%, at least about 9%, at least about 10%, at least about 12%, at least about 15%, at least about 17%, at least about 20%, at least about 25%, at least about 30%, at least about 35%, at least about 40%, at least about 45%, at least about 50%, at least about 60%, at least about 70%, at least about 80%, at least about 90%, at least about 95%, or at least about 99%. In some embodiments, In some embodiments, the percentage of the hepatocytes or hepatic cells that express CXCR-4 or cells that express any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory is at most about 0.01%, at most about 0.1%, at most about 0.5%, at most about 1%, at most about 1.5%, at most about 2%, at most about 3%, at most about 4%, at most about 5%, at most about 6%, at most about 7%, at most about 8%, at most about 9%, at most about 10%, at most about 12%, at most about 15%, at most about 17%, at most about 20%, at most about 25%, at most about 30%, at most about 35%, at most about 40%, at most about 45%, at most about 50%, at most about 60%, at most about 70%, at most about 80%, at most about 90%, at most about 91%, at most about 92%, at most about 93%, at most about 94%, at most about 95%, at most about 96%, at most about 97%, at most about 98%, at most about 99%, at most about 99.55, or at most about 99.9%.

In some embodiments, the percentage of the hepatocytes or the hepatic cells that express CXCR-4 is about 1% to about 90%. In some embodiments, the percentage of the hepatocytes or the hepatic cells that express CXCR-4 or cells that express any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory is at least about 1%. In some embodiments, the percentage of the hepatocytes or the hepatic cells that express CXCR-4 or cells that express any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory is at most about 90%. In some embodiments, the percentage of the hepatocytes or the hepatic cells that express CXCR-4 is about 1% to about 2%, about 1% to about 3%, about 1% to about 5%, about 1% to about 6%, about 1% to about 7%, about 1% to about 8%, about 1% to about 10%, about 1% to about 20%, about 1% to about 30%, about 1% to about 50%, about 1% to about 90%, about 2% to about 3%, about 2% to about 5%, about 2% to about 6%, about 2% to about 7%, about 2% to about 8%, about 2% to about 10%, about 2% to about 20%, about 2% to about 30%, about 2% to about 50%, about 2% to about 90%, about 3% to about 5%, about 3% to about 6%, about 3% to about 7%, about 3% to about 8%, about 3% to about 10%, about 3% to about 20%, about 3% to about 30%, about 3% to about 50%, about 3% to about 90%, about 5% to about 6%, about 5% to about 7%, about 5% to about 8%, about 5% to about 10%, about 5% to about 20%, about 5% to about 30%, about 5% to about 50%, about 5% to about 90%, about 6% to about 7%, about 6% to about 8%, about 6% to about 10%, about 6% to about 20%, about 6% to about 30%, about 6% to about 50%, about 6% to about 90%, about 7% to about 8%, about 7% to about 10%, about 7% to about 20%, about 7% to about 30%, about 7% to about 50%, about 7% to about 90%, about 8% to about 10%, about 8% to about 20%, about 8% to about 30%, about 8% to about 50%, about 8% to about 90%, about 10% to about 20%, about 10% to about 30%, about 10% to about 50%, about 10% to about 90%, about 20% to about 30%, about 20% to about 50%, about 20% to about 90%, about 30% to about 50%, about 30% to about 90%, or about 50% to about 90%. In some embodiments, the percentage of the hepatocytes or the hepatic cells that express CXCR-4 is about 1%, about 2%, about 3%, about 5%, about 6%, about 7%, about 8%, about 10%, about 20%, about 30%, about 50%, or about 90%.

The present disclosure provides one or more hepatic cells. A hepatic cell can resemble a hepatocyte. The hepatic cell may be polygonal. For example, the hepatic cell may be hexagonal. The hepatic cell may be cuboidal. The stem cell may be a pluripotent stem cell. The pluripotent stem cell may be an induced pluripotent stem cell. The hepatic cell may express ASGR-1. The hepatic cell may express CXCR-4. A hepatic cell may express both ASGR-1 and CXCR-4. In a population of two or more hepatic cells, at least 2% of the hepatic cells may express ASGR-1 or any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory. In a population of two or more hepatic cells, at least 10% of the hepatocytes or the hepatic cell express CXCR-4 or any other hepatocyte-like, hepatocyte lineage or hepatocyte-associated markers disclosed herein, or any other markers disclosed herein, that are associated with the (TF recipe) non-natural, exogenous induction of iPSC differentiation along the differentiation trajectory.

### Transcription Factors

The present disclosure provides transcription factors (TFs), the expression of which may induce differentiation of one or more PSCs into one or more hepatocytes or hepatic cells. The transcription factors may induce differentiation through various methods, such as expression of one or more nucleic acid encoding one or more transcription factors in a PSC, contacting a PSC with one or more transcription factor proteins or nucleic acids encoding transcription factor proteins, or through other means as disclosed herein. The PSCs may be induced to differentiate into one or more hepatocytes or hepatic cells in 30 days or less. The PSCs may be induced to differentiate into one or more hepatocytes or hepatic cells in at most about 11, 10, 9, 8, 7, 6, 5, 4, 3, 2 days or less. The PSCs may be induced to differentiate into one or more hepatocytes or hepatic cells in at most about 1 day. The PSCs may be induced to differentiate into one or more hepatocytes or hepatic cells in at least most 4 days. The PSCs may be induced to differentiate into one or more hepatocytes or hepatic cells in at most about 96 hours. The PSCs may be induced to differentiate into one or more hepatocytes or hepatic cells in at most about 96, 90, 80, 70, 60, 50, 40, 30, 20, 10 hours or less. The one or more nucleic acids encoding one or more transcription factors may be expressed in one or more exogenous expression cassettes.

TFs may be used to trigger differentiation programs. Certain transcription factors may be able to induce stem cells to differentiate to particular lineages, such as hepatocytes or hepatic cells. In some aspects, combinations of transcription factors may be used to achieve differentiation to a particular cell lineage. The combinations may achieve a cell type or a cell sub-type that is not achieved by either transcription factor alone. The combination may achieve the same cell type as one of the transcription factors alone but may achieve it more efficiently.

The present disclosure provides cells, exogenous expression cassettes, and methods comprising the use of one or more transcription factors, or molecules that increase transcription or increase activation of transcription factors. The one or more transcription factors may induce differentiation of one or more PSCs. The one or more transcription factors may induce differentiation of the one or more PSCs into one or more hepatocytes or hepatic cells. A combination comprising one or more transcription factors may be created. The combination of transcription factors may be introduced in one or more expression cassettes. The one or more expression cassettes may be induced in one or more PSCs. The induction of the one or more expression cassettes in one or more PSCs may cause the one or more PSCs to differentiate into hepatocytes or hepatic cells. Some transcription factors may require a critical amount of expression to effectively induce differentiation, such as the equivalent of at least 5, 10, 15, 20, 25, or 50 copies of the open reading frame (ORF) per cell. Other factors may require less than a certain threshold of expression due to possible toxicity at high levels, such as less than 20, 10, or 5 copies per cell. Increased levels of expression may also be achieved by increasing the copy number of the ORF, for example, by using a higher copy number vector or by using a transposon.

In some embodiments, the one or more transcription factors are, for example, FoxA1, HNF1A, SPI1, FoxA2, CEBPA, ONECUTI/HNF6, ONECUT2/HNF6B, HNF4a, Atf3, Fos, CEBPB, FoxM1, GATA2 isoform P23769-2, GATA4, GATA2 isoform P23769-1, JUN/AP-1, NFIX, GATA6, TBX3, RXRB, NFE2L2, Myc, TP73, HHEX, SOX17, ATF5, RBPJ, Smad3, NR5A2, NR113, PROX, FOXA3, EGR1, NR1I2/PXR, SKI or HIF1A. In some embodiments, the one or more transcription factors comprise FoxA1. In some embodiments, the one or more transcription factors comprise one or more members, are, for example, FoxA1, HNF1A, SPI1, FoxA2, CEBPA, ONECUTI/HNF6, ONECUT2/HNF6B, HNF4a, Atf3, Fos, CEBPB, FoxM1, GATA2 isoform P23769-2, GATA4, GATA2 isoform P23769-1, JUN/AP-1, NFIX, GATA6, TBX3, RXRB, NFE2L2, Myc, TP73, HHEX, SOX17, ATF5, RBPJ, Smad3, NR5A2, NR113, PROX, FOXA3, EGR1, NR1I2/PXR, SKI or HIF1A. In some embodiments, the one or more transcription factors comprise FoxA1. In some embodiments, the one or more transcription factors comprise HNF1A. In some embodiments, the one or more transcription factors comprise SPI1. In some embodiments, the one or more transcription factors comprise FoxA2. In some embodiments, the one or more transcription factors comprise CEBPA. In some embodiments, the one or more transcription factors comprise ONECUT1/HNF6. In some embodiments, the one or more transcription factors comprise ONECUT 2/HNF6B. In some embodiments, the one or more transcription factors comprise HNF4a. In some embodiments, the one or more transcription factors comprise Atf3. In some embodiments, the one or more transcription factors comprise Fos. In some embodiments, the one or more transcription factors comprise CEBPB. In some embodiments, the one or more transcription factors comprise FoxM1. In some embodiments, the one or more transcription factors comprise GATA2 isoform P23769-2. In some embodiments, the one or more transcription factors comprise GATA4. In some embodiments, the one or more transcription factors comprise GATA2 isoform P23769-1. In some embodiments, the one or more transcription factors comprise JUN/AP-1. In some embodiments, the one or more transcription factors comprise NFIX. In some embodiments, the one or more transcription factors comprise GATA6. In some embodiments, the one or more transcription factors comprise TBX3. In some embodiments, the one or more transcription factors comprise RXRB. In some embodiments, the one or more transcription factors comprise NFE2L2. In some embodiments, the one or more transcription factors comprise Myc. In some embodiments, the one or more transcription factors comprise TP73. In some embodiments, the one or more transcription factors comprise HHEX. In some embodiments, the one or more transcription factors comprise SOX17. In some embodiments, the one or more transcription factors comprise ATF5. In some embodiments, the one or more transcription factors comprise RBPJ. In some embodiments, the one or more transcription factors comprise Smad3. In some embodiments, the one or more transcription factors comprise NR5A2. In some embodiments, the one or more transcription factors comprise HIF1A. In some embodiments, the one or more transcription factors comprise PROX. In some embodiments, the one or more transcription factors comprise FOXA3. In some embodiments, the one or more transcription factors comprise EGR1. In some embodiments, the one or more transcription factors comprise NR112/PXR. In some embodiments, the one or more transcription factors comprise SKI.

In some embodiments, the one or more transcription factors comprise ONECUT1/HNF6 or ONECUT2/HNF6B. In some embodiments, the one or more transcription factors comprise ONECUT1/HNF6 and ONECUT2/HNF6B.

In some embodiments, one or more of the one or more transcription factors are, for example, FoxA1, HNF1A, SPI1, FoxA2, CEBPA, ONECUT1/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR113, PROX, FOXA3, EGR1, NR112/PXR, SKI or HIF1A. In some embodiments, two or more of the one or more transcription factors are, for example, FoxA1, HNF1A, SPI1, FoxA2, CEBPA, ONECUTI/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR1I3, PROX, FOXA3, EGR1, NR112/PXR, SKI or HIF1A. In some embodiments, three or more of the one or more transcription factors are, for example, FoxA1, HNF1A, SPI1, FoxA2, CEBPA, ONECUT1/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR113, PROX, FOXA3, EGR1, NR112/PXR, SKI or HIF1A. In some embodiments, four or more of the one or more transcription factors are, for example, FoxA1, FoxA1, HNF1A, SPI1, FoxA2, CEBPA, ONECUTI/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR113, or PROX, FOXA3, EGR1, NR112/PXR, SKI HIF1A. In some embodiments, five or more of the one or more transcription factors are, for example, selected from the group consisting of, for example, FoxA1, HNF1A, SPI1, FoxA2, CEBPA, ONECUT1/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR113, PROX, FOXA3, EGR1, NR1I2/PXR, SKI or HIF1A. In some embodiments, six or more of the one or more transcription factors are, for example, FoxA1, HNF1A, SPI1, FoxA2, CEBPA, ONECUTI/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR113, or HIF1A. In some embodiments, seven or more of the one or more transcription factors are, for example, FoxA1, HNF1A, SPI1, FoxA2, CEBPA, ONECUTI/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR113, PROX, FOXA3, EGR1, NR112/PXR, SKI or HIF1A. In some embodiments, eight or more of the one or more transcription factors are, for example, FoxA1, HNF1A, SPI1, FoxA2, CEBPA, ONECUTI/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR113, PROX, FOXA3, EGR1, NR1I2/PXR, SKI or HIF1A.

In some embodiments, the one or more transcription factors are, for example, SPI1, HNF1A, FoxA2, CEBPA, HNF6, HNF4a, or HNF6B. In some embodiments, the one or more transcription factors are, for example HNF1A, Atf3, HNF6, HNF6B, Fos, CEBPA, and FoxM1. In some embodiments, the one or more transcription factors are, for example, SPI1, FoxA2, GATA2 isoform P23769-2, GATA4, GATA2 isoform P23769-1, JUN/AP-1, or NFIX. In some embodiments, the one or more transcription factors are, for example, HNF4a, CEBPA, HNF1A, TBX3, Fos, GATA2 isoform P23769-2, or GATA2 isoform P23769-1. In some embodiments, the one or more transcription factors are, for example, GATA2 isoform P23769-2, HNF4a, RXRB, NFE2L2, Myc, TP73, or HNF6. In some embodiments, the one or more transcription factors are, for example, GATA2 isoform P23769-1, CEBPA, ATF3, GATA6, Fos, GATA3, NFIX. In some embodiments, the one or more transcription factors are, for example, HHEX, SOX17, HNF4A, HNF1A, ONECUT1/HNF6, FoxA2, or FoxA1. In some embodiments, the one or more transcription factors are, for example, HHEX, SOX17, or HNF4A. In some embodiments, the one or more transcription factors are, for example, CEBPA, ATF5, HHEX, SOX17, HNF4A, ONECUT2/HNF6B, or HNF1A. In some embodiments, the one or more transcription factors are, for example, FoxA1, HNF1A, FoxA2, CEBPA, ONECUTI/HNF6, HNF4a, or RBPJ. In some embodiments, the one or more transcription factors are, for example, Smad3, HHEX, ATF5, NR5A2, GATA2 isoform P23769-1, CEBPB, or NR1I3. In some embodiments, the one or more transcription factors are, for example, HNF1A, CEBPA, ONECUT1/HNF6, ONECUT2/HNF6B, Fos, HIFIA, or TBX3.

### Forkhead Box (FOX) Transcription Factors

The present disclosure provides cells, expression cassettes, and methods using transcription factors, or molecules that increase transcription or increase activation of transcription factors. One or more transcription factors may be used. A transcription factor may be a member of the FOX family of transcription factors. A FOX transcription factor may be, for example, FoxA1, FoxA2, FoxA3, FoxM1.

### FoxA1

The present disclosure provides cells, expression cassettes, and methods using transcription factors, or molecules that increase transcription or increase activation of transcription factors. In some embodiments, one or more transcription factors may be used. In some embodiments, a transcription factor may be FoxA1, which may also be known as HNF3A; Hepatocyte Nuclear Factor 3-Alpha, Forkhead Box Protein A1, Transcription Factor 3A, HNF-3-Alpha, HNF-3A, TCF-3A, TCF3A, or Hepatocyte Nuclear Factor 3, Alpha.

The present disclosure provides an expression cassette comprising one or more transcription factors. The one or more transcription factors may comprise FoxA1. FoxA1 may be introduced in an expression cassette. An expression cassette comprising FoxA1 may be introduced in a PSC. An expression cassette comprising FoxA1 may be expressed in a PSC. An expression cassette comprising FoxA1 may induce differentiation of the PSC into a hepatocyte or hepatic cell.

FoxA1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising FoxA1 and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise FoxA1. An expression cassette comprising FoxA1 may be expressed in a PSC. An expression cassette comprising FoxA1 may induce the differentiation of the PSC into a hepatocyte or a hepatic cell. Different amounts of FoxA1 may be introduced into the PSC. At least about 5, about 10, about 15, about 20, about 25, or about 50 copies of the open reading frame (ORF) for FoxA1 per cell may be introduced. Alternatively, or in addition to, at most about 50, about 25, about 20, about 15, about 10, or about 5 copies of the open reading frame (ORF) for FoxA1 per cell may be introduced. Increased levels of expression may also be achieved by increasing the copy number of the ORF, for example, by using a higher copy number vector or by using a transposon.

In some embodiments, the number of copies of the ORF for FoxA1 introduced per cell can be about 1 to about 90. In some embodiments, the number of copies of the ORF for FoxA1 introduced per cell can be at least about 1. In some embodiments, the number of copies of the ORF for FoxA1 introduced per cell can be at most about 90. In some embodiments, the number of copies of the ORF for FoxA1 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for FoxA1 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise FoxA1. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### FoxM1

The present disclosure provides cells, expression cassettes, and methods using transcription factors, or molecules that increase transcription or increase activation of transcription factors. One or more transcription factors may be used. In some embodiments, a transcription factor may be FoxM1, which may also be known as Forkhead Box M1; HFH-11; MPP2; M-Phase Phosphoprotein 2; MPHOSPH2; FKHL16; HNF-3; INS-1; Hepatocyte Nuclear Factor 3 Forkhead Homolog 11; Winged-Helix Factor From INS-1 Cells; MPM-2 Reactive Phosphoprotein 2; Forkhead-Related Protein FKHL16; Transcription Factor Trident; HNF-3/Fork-Head Homolog 11; Forkhead Box Protein M1; Trident; HFH11; TGT3; Forkhead, Drosophila, Homo log-Like 16; Forkhead Box M1-D; TRIDENT; FOXM1A; FOXM1B; FOXM1C; MPP-2; PIG29; or WIN.

The present disclosure provides an expression cassette comprising one or more transcription factors. The one or more transcription factors may comprise FoxM1. FoxM1 may be introduced in an expression cassette. FoxM1 may be expressed in an expression cassette. An expression cassette comprising FoxM1 may be introduced in a PSC. An expression cassette comprising FoxM1 may be expressed in a PSC. An expression cassette comprising FoxM1 may induce differentiation of the PSC into a hepatocyte or hepatic cell.

FoxM1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising FoxM1, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise FoxM1. An expression cassette comprising FoxM1 may be expressed in a PSC. An expression cassette comprising FoxM1 may induce the differentiation of the PSC into a hepatocyte or a hepatic cell. Different amounts of FoxM1 may be introduced into the PSC. Different amounts of FoxM1 may be introduced into the PSC. At least about 5, about 10, about 15, about 20, about 25, or about 50 copies of the open reading frame (ORF) for FoxM1 per cell may be introduced. Alternatively, or in addition to, at most about 50, about 25, about 20, about 15, about 10, or about 5 copies of the open reading frame (ORF) for FoxM1 per cell may be introduced. Increased levels of expression may also be achieved by increasing the copy number of the ORF, for example, by using a higher copy number vector or by using a transposon.

In some embodiments, the number of copies of the ORF for FoxM1 introduced per cell can be about 1 to about 90. In some embodiments, the number of copies of the ORF for FoxM1 introduced per cell can be at least about 1. In some embodiments, the number of copies of the ORF for FoxM1 introduced per cell can be at most about 90. In some embodiments, the number of copies of the ORF for FoxM1 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for FoxM1 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise FoxM1. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### FoxA2

The present disclosure provides cells, expression cassettes, and methods using transcription factors, or molecules that increase transcription or increase activation of transcription factors. One or more transcription factors may be used. In some embodiments, a transcription factor may be FoxA2, which may also be known as Forkhead Box A2; HNF3B; Hepatocyte Nuclear Factor 3-Beta; Forkhead Box Protein A2; Transcription Factor 3B; HNF-3-Beta; TCF3B; Hepatocyte Nuclear Factor 3, Beta; Hepatic Nuclear Factor-3-Beta; HNF-3B; or TCF-3B.

The present disclosure provides an expression cassette comprising one or more transcription factors. The one or more transcription factors may comprise FoxA2. FoxA2 may be introduced in an expression cassette. An expression cassette comprising FoxA2 may be introduced in a PSC. FoxA2 may be introduced in an expression cassette. FoxA2 may be expressed in an expression cassette. An expression cassette comprising FoxA2 may be introduced in a PSC. An expression cassette comprising FoxA2 may be expressed in a PSC. An expression cassette comprising FoxA2 may induce differentiation of the PSC into a hepatocyte or hepatic cell.

FoxA2 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising FoxA2, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise FoxA2. An expression cassette comprising FoxA2 may be expressed in a PSC. An expression cassette comprising FoxA2 may induce the differentiation of the PSC into a hepatocyte or a hepatic cell. Different amounts of FoxA2 may be introduced into the PSC. At least about 5, about 10, about 15, about 20, about 25, or about 50 copies of the open reading frame (ORF) for FoxA2 per cell may be introduced. Alternatively, or in addition to, at most about 50, about 25, about 20, about 15, about 10, or about 5 copies of the open reading frame (ORF) for FoxA2 per cell may be introduced. Increased levels of expression may also be achieved by increasing the copy number of the ORF, for example, by using a higher copy number vector or by using a transposon.

In some embodiments, the number of copies of the ORF for FoxA2 introduced per cell can be about 1 to about 90. In some embodiments, the number of copies of the ORF for FoxA2 introduced per cell can be at least about 1. In some embodiments, the number of copies of the ORF for FoxA2 introduced per cell can be at most about 90. In some embodiments, the number of copies of the ORF for FoxA2 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for FoxA2 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise FoxA2. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### FOXA3

FOXA3 gene encodes a member of the forkhead class of DNA-binding proteins. These hepatocyte nuclear factors are transcriptional activators for liver-specific transcripts such as albumin and transthyretin, and they also interact with chromatin. The present disclosure provides cells, expression cassettes, and methods using transcription factors, or molecules that increase transcription or increase activation of transcription factors. In some embodiments, one or more transcription factors may be used. In some embodiments, a transcription factor may be FoxA3 which may also be known as Forkhead Box A3 HNF3G, Hepatocyte Nuclear Factor 3-Gamma, Fork Head-Related Protein FKH H3, Forkhead Box Protein A3, or Transcription Factor 3G.

The present disclosure provides an expression cassette comprising one or more transcription factors. The one or more transcription factors may comprise FoxA3. FoxA3 may be introduced in an expression cassette. An expression cassette comprising FoxA3 may be introduced in a PSC. FoxA3 may be introduced in an expression cassette. FoxA3 may be expressed in an expression cassette. An expression cassette comprising FoxA3 may be introduced in a PSC. An expression cassette comprising FoxA3 may be expressed in a PSC. An expression cassette comprising FoxA3 may induce differentiation of the PSC into a hepatocyte or hepatic cell.

FoxA3 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising FoxA3, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise FoxA3. An expression cassette comprising FoxA3 may be expressed in a PSC. An expression cassette comprising FoxA3 may induce the differentiation of the PSC into a hepatocyte or a hepatic cell. Different amounts of FoxA3 may be introduced into the PSC. At least about 5, about 10, about 15, about 20, about 25, or about 50 copies of the open reading frame (ORF) for FoxA3 per cell may be introduced. Alternatively, or in addition to, at most about 50, about 25, about 20, about 15, about 10, or about 5 copies of the open reading frame (ORF) for FoxA3 per cell may be introduced. Increased levels of expression may also be achieved by increasing the copy number of the ORF, for example, by using a higher copy number vector or by using a transposon.

In some embodiments, the number of copies of the ORF for FoxA3 introduced per cell can be about 1 to about 90. In some embodiments, the number of copies of the ORF for FoxA3 introduced per cell can be at least about 1. In some embodiments, the number of copies of the ORF for FoxA3 introduced per cell can be at most about 90. In some embodiments, the number of copies of the ORF for FoxA3 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for FoxA3 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise FoxA3. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### HNF Hepatocyte Nuclear Factor Transcription Factors

The present disclosure provides cells, expression cassettes, and methods using transcription factors, or molecules that increase transcription or increase activation of transcription factors. One or more transcription factors may be used. A transcription factor may be a member of the HNF Hepatocyte Nuclear Factor family of transcription factors. An HNF Hepatocyte Nuclear Factor transcription factor may be, for example, HNF1A or HNF4A.

### HNF1A

The present disclosure provides cells, expression cassettes, and methods using transcription factors, or molecules that increase transcription or increase activation of transcription factors. One or more transcription factors may be used. A transcription factor may be a member of the HNF family. In some embodiments, the transcription factor may be HNF1A, which may also be known as HNF1 Homeobox A; LFB1; HNF1; TCF1; Liver-Specific Transcription Factor LF-B1; Hepatocyte Nuclear Factor 1-Alpha; HNF-1-Alpha; HNF-1A; MODY3; TCF-1; Transcription Factor 1, Hepatic; LF-B1, Hepatic Nuclear Factor (HNF1), Albumin Proximal Factor; Truncated Hepatocyte Nuclear Factor 1 Alpha; Interferon Production Regulator Factor; Transcription Factor 1, Hepatic; Hepatic Nuclear Factor 1; Albumin Proximal Factor; Transcription Factor 1; HNF1alpha; IDDM20; HNF1Î±; HNF1α; or HNF1A.

The present disclosure provides an expression cassette comprising one or more transcription factors. The one or more transcription factors may comprise HNF1A. HNF1A may be introduced in an expression cassette. HNF1A may be expressed in an expression cassette. An expression cassette comprising HNF1A may be introduced in a PSC. An expression cassette comprising HNF1A may be expressed in a PSC. An expression cassette comprising HNF1A may induce differentiation of the PSC into a hepatocyte or hepatic cell.

HNF1A may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising HNF1A, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise HNF1A. An expression cassette comprising HNF1A may be expressed in a PSC. An expression cassette comprising HNF1A may induce the differentiation of the PSC into a hepatocyte or a hepatic cell. Different amounts of HNF 1 Ac may be introduced into the PSC. At least about 5, about 10, about 15, about 20, about 25, or about 50 copies of the open reading frame (ORF) for HNF1A per cell may be introduced. Alternatively, or in addition to, at most about 50, about 25, about 20, about 15, about 10, or about 5 copies of the open reading frame (ORF) for HNF1A per cell may be introduced. Increased levels of expression may also be achieved by increasing the copy number of the ORF, for example, by using a higher copy number vector or by using a transposon.

In some embodiments, the number of copies of the ORF for HNF1A introduced per cell can be about 1 to about 90. In some embodiments, the number of copies of the ORF for HNF1A introduced per cell can be at least about 1. In some embodiments, the number of copies of the ORF for HNF1A introduced per cell can be at most about 90. In some embodiments, the number of copies of the ORF for HNF1A introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for HNF1A introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise HNF1A. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### HNF4α

The present disclosure provides cells, expression cassettes, and methods using transcription factors, or molecules that increase transcription or increase activation of transcription factors. One or more transcription factors may be used. A transcription factor may be a member of the HNF family. In some embodiments, the transcription factor may be HNF4a, which may also be known as Hepatocyte Nuclear Factor 4 Alpha; NR2A1; HNF4; TCF14; Nuclear Receptor Subfamily 2 Group A Member 1; Hepatocyte Nuclear Factor 4-Alpha; Transcription Factor HNF-4; Transcription Factor 14; TCF-14; MODY1; MODY; Hepatic Nuclear Factor 4 Alpha; HNF4alpha10/11/12; HNF-4-Alpha; HNF4alpha; HNF4a7; HNF4a8; HNF4a9; NR2A21; FRTS4; or TCF.

The present disclosure provides an expression cassette comprising one or more transcription factors. The one or more transcription factors may comprise HNF4a. HNF4a may be introduced in an expression cassette. HNF4a may be expressed in an expression cassette. An expression cassette comprising HNF4a may be introduced in a PSC. An expression cassette comprising HNF4a may be expressed in a PSC. An expression cassette comprising HNF4a may induce differentiation of the PSC into a hepatocyte or hepatic cell.

HNF4a may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising HNF4a, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise HNF4a. An expression cassette comprising HNF4a may be expressed in a PSC. An expression cassette comprising HNF4a may induce the differentiation of the PSC into a hepatocyte or a hepatic cell. Different amounts of HNF4a may be introduced into the PSC. At least about 5, about 10, about 15, about 20, about 25, or about 50 copies of the open reading frame (ORF) for HNF4a per cell may be introduced. Alternatively, or in addition to, at most about 50, about 25, about 20, about 15, about 10, or about 5 copies of the open reading frame (ORF) for HNF4a per cell may be introduced. Increased levels of expression may also be achieved by increasing the copy number of the ORF, for example, by using a higher copy number vector or by using a transposon.

In some embodiments, the number of copies of the ORF for HNF4a introduced per cell can be about 1 to about 90. In some embodiments, the number of copies of the ORF for HNF4a introduced per cell can be at least about 1. In some embodiments, the number of copies of the ORF for HNF4a introduced per cell can be at most about 90. In some embodiments, the number of copies of the ORF for HNF4a introduced per cell can be from about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for HNF4a introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise HNF4a. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### CUT Homeobox Transcription Factors

The present disclosure provides cells, expression cassettes, and methods using transcription factors, or molecules that increase transcription or increase activation of transcription factors. One or more transcription factors may be used. A transcription factor may be a member of the CUT homeobox family of transcription factors. The transcription factor may be, for example, HNF6A/ONECUT1 or HNF6B/ONECUT2.

### ONECUT1/HNF6

The present disclosure provides cells, expression cassettes, and methods using transcription factors, or molecules that increase transcription or increase activation of transcription factors. One or more transcription factors may be used. In some embodiments, a transcription factor may be a ONECUT transcription factor, which may also be known as ONECUT1; One Cut Homeobox 1; HNF-6; HNF6A; HNF6; One Cut Domain Family Member 1; Hepatocyte Nuclear Factor 6; Hepatocyte Nuclear Factor 6, Alpha; or One Cut Domain, Family Member 1. The present disclosure provides an expression cassette comprising one or more transcription factors. The one or more transcription factors may comprise ONECUT1/HNF6. ONECUTI/HNF6 may be introduced in an expression cassette. ONECUT1/HNF6 may be expressed in an expression cassette. An expression cassette comprising ONECUTI/HNF6 may be introduced in a PSC. An expression cassette comprising ONECUTI/HNF6 may be expressed in a PSC. An expression cassette comprising ONECUT1/HNF6 may induce differentiation of the PSC into a hepatocyte or hepatic cell.

ONECUTI/HNF6 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising ONECUT1/HNF6, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise ONECUTI/HNF6. An expression cassette comprising ONECUT1/HNF6 may be expressed in a PSC. An expression cassette comprising ONECUTI/HNF6 may induce the differentiation of the PSC into a hepatocyte or a hepatic cell. Different amounts of ONECUTI/HNF6 may be introduced into the PSC. At least about 5, about 10, about 15, about 20, about 25, or about 50 copies of the open reading frame (ORF) for ONECUTI/HNF6 per cell may be introduced. Alternatively, or in addition to, at most about 50, about 25, about 20, about 15, about 10, or about 5 copies of the open reading frame (ORF) for ONECUTI/HNF6 per cell may be introduced. Increased levels of expression may also be achieved by increasing the copy number of the ORF, for example, by using a higher copy number vector or by using a transposon.

In some embodiments, the number of copies of the ORF for ONECUT1/HNF6 introduced per cell can be about 1 to about 90. In some embodiments, the number of copies of the ORF for ONECUTI/HNF6 introduced per cell can be at least about 1. In some embodiments, the number of copies of the ORF for ONECUT1/HNF6 introduced per cell can be at most about 90. In some embodiments, the number of copies of the ORF for ONECUT1/HNF6 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for ONECUT1/HNF6 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise ONECUT1/HNF6. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### ONECUT2/HNF6B

The present disclosure provides cells, expression cassettes, and methods using transcription factors, or molecules that increase transcription or increase activation of transcription factors. One or more transcription factors may be used. In some embodiments, a transcription factor may be a ONECUT transcription factor, which may also be known as ONECUT2; One Cut Homeobox 2; OC-2; Hepatocyte Nuclear Factor 6-Beta; One Cut Domain Family Member 2; Transcription Factor ONECUT-2; HNF-6-Beta; ONECUT-2 Homeodomain Transcription Factor; One Cut Domain, Family Member 2; Onecut 2; HNF6B; or OC2.

The present disclosure provides an expression cassette comprising one or more transcription factors. The one or more transcription factors may comprise ONECUT2/HNF6B. ONECUT2/HNF6B may be introduced in an expression cassette. ONECUT2/HNF6B may be expressed in an expression cassette. An expression cassette comprising ONECUT2/HNF6B may be introduced in a PSC. An expression cassette comprising ONECUT2/HNF6B may be expressed in a PSC. An expression cassette comprising ONECUT2/HNF6B may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise ONECUT2/HNF6B. ONECUT2/HNF6B may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising ONECUT2/HNF6B, and one or more other transcription factors may induce differentiation of the PSC into a hepatocyte or hepatic cell.

Different amounts of ONECUT2/HNF6B may be introduced into the PSC. At least about 5, about 10, about 15, about 20, about 25, or about 50 copies of the open reading frame (ORF) for ONECUT2/HNF6B per cell may be introduced. Alternatively, or in addition to, at most about 50, about 25, about 20, about 15, about 10, or about 5 copies of the open reading frame (ORF) for ONECUT2/HNF6B per cell may be introduced. Increased levels of expression may also be achieved by increasing the copy number of the ORF, for example, by using a higher copy number vector or by using a transposon.

In some embodiments, the number of copies of the ORF for ONECUT2/HNF6B introduced per cell can be about 1 to about 90. In some embodiments, the number of copies of the ORF for ONECUT2/HNF6B introduced per cell can be at least about 1. In some embodiments, the number of copies of the ORF for ONECUT2/HNF6B introduced per cell can be at most about 90. In some embodiments, the number of copies of the ORF for ONECUT2/HNF6B introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for ONECUT2/HNF6B introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise ONECUT2/HNF6B. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### Basic Leucine Zipper Transcription Factors

The present disclosure provides cells, expression cassettes, and methods using transcription factors, or molecules that increase transcription or increase activation of transcription factors. One or more transcription factors may be used. A transcription factor may be a basic leucine zipper transcription factor. A basic leucine zipper transcription factor may be, for example, ATF3, ATF5, CEBPA, CEBPB, FOS, JUN, or NFE2L2.

### CEBPA

The present disclosure provides cells, expression cassettes, and methods using transcription factors, or molecules that increase transcription or increase activation of transcription factors. One or more transcription factors may be used. In some embodiments, the transcription factor may be a CCAAT enhancer binding protein (CEBP), which may also be known as CCAAT Enhancer Binding Protein Alpha; C/EBP-Alpha; CEBP; CCAAT/Enhancer Binding Protein (C/EBP), Alpha; CCAAT/Enhancer-Binding Protein Alpha; CCAAT/Enhancer Binding Protein Alpha; or C/EBP Alpha.

The present disclosure provides an expression cassette comprising one or more transcription factors. The one or more transcription factors may comprise CEBPA. CEBPA may be introduced in an expression cassette. CEBPA may be expressed in an expression cassette. An expression cassette comprising CEBPA may be introduced in a PSC. An expression cassette comprising CEBPA may be expressed in a PSC. An expression cassette comprising CEBPA may induce differentiation of the PSC into a hepatocyte or hepatic cell.

CEBPA may be introduced in an expression cassette with one or more other transcription factors. A combination comprising one or more transcription factors may be created. The combination of transcription factors may be introduced in an expression cassette.

The combination of transcription factors may be expressed in an expression cassette. The expression cassette may be introduced in a PSC. The expression cassette comprising CEBPA, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise CEBPA. An expression cassette comprising CEBPA may be expressed in a PSC. An expression cassette comprising CEBPA may induce the differentiation of the PSC into a hepatocyte or a hepatic cell. Different amounts of CEBPA may be introduced into the PSC. At least about 5, about 10, about 15, about 20, about 25, or about 50 copies of the open reading frame (ORF) for CEBPA per cell may be introduced. Alternatively, or in addition to, at most about 50, about 25, about 20, about 15, about 10, or about 5 copies of the open reading frame (ORF) for CEBPA per cell may be introduced. Increased levels of expression may also be achieved by increasing the copy number of the ORF, for example, by using a higher copy number vector or by using a transposon.

In some embodiments, the number of copies of the ORF for CEBPA introduced per cell can be about 1 to about 90. In some embodiments, the number of copies of the ORF for CEBPA introduced per cell can be at least about 1. In some embodiments, the number of copies of the ORF for CEBPA introduced per cell can be at most about 90. In some embodiments, the number of copies of the ORF for CEBPA introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for CEBPA introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise CEBPA. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### Atf3

The present disclosure provides cells, expression cassettes, and methods using transcription factors, or molecules that increase transcription or increase activation of transcription factors. One or more transcription factors may be used. The transcription factor may be an activating transcription factor (ATF). In some embodiments, the ATF may be Atf3, which may also be known as Activating Transcription Factor 3, Cyclic AMP-Dependent Transcription Factor ATF-3, or CAMP-Dependent Transcription Factor ATF-3.

The present disclosure provides an expression cassette comprising one or more transcription factors. The one or more transcription factors may comprise Atf3. Atf3 may be expressed in an expression cassette. An expression cassette comprising Atf3 may be introduced in a PSC. An expression cassette comprising Atf3 may be expressed in a PSC. An expression cassette comprising Atf3 may induce differentiation of the PSC into a hepatocyte or hepatic cell.

Atf3 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising Atf3, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise Atf3. An expression cassette comprising Atf3 may be expressed in a PSC. An expression cassette comprising Atf3 may induce the differentiation of the PSC into a hepatocyte or a hepatic cell. Different amounts of Atf3 may be introduced into the PSC. At least about 5, about 10, about 15, about 20, about 25, or about 50 copies of the open reading frame (ORF) for Atf3 per cell may be introduced. Alternatively, or in addition to, at most about 50, about 25, about 20, about 15, about 10, or about 5 copies of the open reading frame (ORF) for Atf3 per cell may be introduced. Increased levels of expression may also be achieved by increasing the copy number of the ORF, for example, by using a higher copy number vector or by using a transposon.

In some embodiments, the number of copies of the ORF for Atf3 introduced per cell can be about 1 to about 90. In some embodiments, the number of copies of the ORF for Atf3 introduced per cell can be at least about 1. In some embodiments, the number of copies of the ORF for Atf3 introduced per cell can be at most about 90. In some embodiments, the number of copies of the ORF for Atf3 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for Atf3 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise Atf3. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### Fos

The present disclosure provides cells, expression cassettes, and methods using transcription factors, or molecules that increase transcription or increase activation of transcription factors. One or more transcription factors may be used. In some embodiments, a transcription factor may be a member of the Fos family. The Fos family member may be Fos, which may also be known as Fos Proto-Oncogene, AP-1 Transcription Factor Subunit; AP-1; FBJ Murine Osteosarcoma Viral Oncogene Homolog; G0/G1 Switch Regulatory Protein 7; Proto-Oncogene C-Fos; C-Fos; FBJ Murine; Osteosarcoma Viral (V-Fos) Oncogene Homolog (Oncogene FOS); V-Fos FBJ Murine Osteosarcoma Viral Oncogene Homolog; Fos Proto-Oncogene, AP-1 Transcription Factor Subunit; Cellular Oncogene C-Fos; Cellular Oncogene Fos; Activator Protein 1; C-FOS; G0S7; or P55.

The present disclosure provides an expression cassette comprising one or more transcription factors. The one or more transcription factors may comprise Fos. Fos may be introduced in an expression cassette. Fos may be expressed in an expression cassette. An expression cassette comprising Fos may be introduced in a PSC. An expression cassette comprising Fos may be expressed in a PSC. An expression cassette comprising Fos may induce differentiation of the PSC into a hepatocyte or hepatic cell.

Fos may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising Fos and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise Fos. Different amounts of Fos may be introduced into the PSC. An expression cassette comprising Fos may induce the differentiation of the PSC into a hepatocyte or a hepatic cell. Different amounts of Fos may be introduced into the PSC. At least about 5, about 10, about 15, about 20, about 25, or about 50 copies of the open reading frame (ORF) for Fos per cell may be introduced. Alternatively, or in addition to, at most about 50, about 25, about 20, about 15, about 10, or about 5 copies of the open reading frame (ORF) for Fos per cell may be introduced. Increased levels of expression may also be achieved by increasing the copy number of the ORF, for example, by using a higher copy number vector or by using a transposon.

In some embodiments, the number of copies of the ORF for Fos introduced per cell can be about 1 to about 90. In some embodiments, the number of copies of the ORF for Fos introduced per cell can be at least about 1. In some embodiments, the number of copies of the ORF for Fos introduced per cell can be at most about 90. In some embodiments, the number of copies of the ORF for Fos introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for Fos introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise Fos. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### CEBPB

The present disclosure provides cells, expression cassettes, and methods using transcription factors, or molecules that increase transcription or increase activation of transcription factors. One or more transcription factors may be used. In some embodiments, the transcription factor may be a CCAAT enhancer binding protein (CEBP), which may also be known as CCAAT Enhancer Binding Protein Beta; C/EBP-Beta; IL6DBP; TCF5; LAP; CCAAT/Enhancer Binding Protein (C/EBP), Beta; Interleukin 6-Dependent DNA-Binding Protein; CCAAT/Enhancer-Binding Protein Beta; Nuclear Factor Of Interleukin 6; Transcription Factor 5; Nuclear Factor NF-IL6; NFIL6; CRP2; Liver-Enriched Transcriptional Activator Protein; CCAAT/Enhancer Binding Protein Beta; Liver-Enriched Inhibitory Protein; Transcription Factor C/EBP Beta; Liver Activator Protein; C/EBP Beta; NF-IL6; TCF-5; or LIP.

The present disclosure provides an expression cassette comprising one or more transcription factors. The one or more transcription factors may comprise CEBPB. CEBPB may be expressed in an expression cassette. An expression cassette comprising CEBPB may be introduced in a PSC. An expression cassette comprising CEBPB may be expressed in a PSC. An expression cassette comprising CEBPB may induce differentiation of the PSC into a hepatocyte or hepatic cell.

CEBPB may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising CEBPB, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise CEBPB. An expression cassette comprising CEBPB may be expressed in a PSC. An expression cassette comprising CEBPB may induce the differentiation of the PSC into a hepatocyte or a hepatic cell. Different amounts of CEBPB may be introduced into the PSC. At least about 5, about 10, about 15, about 20, about 25, or about 50 copies of the open reading frame (ORF) for CEBPB per cell may be introduced. Alternatively, or in addition to, at most about 50, about 25, about 20, about 15, about 10, or about 5 copies of the open reading frame (ORF) for CEBPB per cell may be introduced. Increased levels of expression may also be achieved by increasing the copy number of the ORF, for example, by using a higher copy number vector or by using a transposon.

In some embodiments, the number of copies of the ORF for CEBPB introduced per cell can be about 1 to about 90. In some embodiments, the number of copies of the ORF for CEBPB introduced per cell can be at least about 1. In some embodiments, the number of copies of the ORF for CEBPB introduced per cell can be at most about 90. In some embodiments, the number of copies of the ORF for CEBPB introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for CEBPB introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise CEBPB. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### JUN/AP-1

The present disclosure provides cells, expression cassettes, and methods using transcription factors, or molecules that increase transcription or increase activation of transcription factors. One or more transcription factors may be used. A transcription factor may be a member of the JUN family. In some embodiments, the transcription factor may be JUN/AP-1. JUN/AP-1, which may also be known as Jun Proto-Oncogene, AP-1 Transcription Factor Subunit; V-Jun Avian Sarcoma Virus 17 Oncogene Homolog; C-Jun; AP-1; Transcription Factor AP-1; Proto-Oncogene C-Jun; Activator Protein 1; Jun Oncogene; AP1; P39; V-Jun Sarcoma Virus 17 Oncogene Homolog; Jun Activation Domain Binding Protein; Enhancer-Binding Protein AP1; Proto-Oncogene CJun; or CJUN.

The present disclosure provides an expression cassette comprising one or more transcription factors. The one or more transcription factors may comprise JUN/AP-1. JUN/AP-1 may be expressed in an expression cassette. An expression cassette comprising JUN/AP-1 may be introduced in a PSC. An expression cassette comprising JUN/AP-1 may be expressed in a PSC. An expression cassette comprising JUN/AP-1 may induce differentiation of the PSC into a hepatocyte or hepatic cell.

JUN/AP-1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising JUN/AP-1 and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise JUN/AP-1. An expression cassette comprising JUN/AP-1 may be expressed in a PSC. An expression cassette comprising JUN/AP-1 may induce the differentiation of the PSC into a hepatocyte or a hepatic cell. Different amounts of JUN/AP-1 may be introduced into the PSC. At least about 5, about 10, about 15, about 20, about 25, or about 50 copies of the open reading frame (ORF) for JUN/AP-1 per cell may be introduced. Alternatively, or in addition to, at most about 50, about 25, about 20, about 15, about 10, or about 5 copies of the open reading frame (ORF) for JUN/AP-1 per cell may be introduced. Increased levels of expression may also be achieved by increasing the copy number of the ORF, for example, by using a higher copy number vector or by using a transposon.

In some embodiments, the number of copies of the ORF for JUN/AP-1 introduced per cell can be about 1 to about 90. In some embodiments, the number of copies of the ORF for JUN/AP-1 introduced per cell can be at least about 1. In some embodiments, the number of copies of the ORF for JUN/AP-1 introduced per cell can be at most about 90. In some embodiments, the number of copies of the ORF for JUN/AP-1 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for JUN/AP-1 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise JUN/AP-1. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### NFE2L2

The present disclosure provides cells, expression cassettes, and methods using transcription factors, or molecules that increase transcription or increase activation of transcription factors. One or more transcription factors may be used. A transcription factor may be a member of the NFE family. In some embodiments, the transcription factor may be NFE2L2, which may also be known as NFE2 Like BZIP Transcription Factor 2; NRF2; Nuclear Factor, Erythroid 2 Like 2; Nuclear Factor Erythroid 2-Related Factor 2; NF-E2-Related Factor 2; HEBP1; Nrf-2; Nuclear Factor (Erythroid-Derived 2)-Like 2; Nuclear Factor, Erythroid Derived 2, Like 2; Nuclear Factor Erythroid-Derived 2-Like 2; Nuclear Factor, Erythroid 2-Like 2; NFE2-Related Factor 2; IMDDHH; or NRF-2.

The present disclosure provides an expression cassette comprising one or more transcription factors. The one or more transcription factors may comprise NFE2L2. NFE2L2 may be introduced in an expression cassette. NFE2L2 may be expressed in an expression cassette. An expression cassette comprising NFE2L2 may be introduced in a PSC. An expression cassette comprising NFE2L2 may be expressed in a PSC. An expression cassette comprising NFE2L2 may induce differentiation of the PSC into a hepatocyte or hepatic cell.

NFE2L2 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising NFE2L2, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise NFE2L2. An expression cassette comprising NFE2L2 may be expressed in a PSC. An expression cassette comprising NFE2L2 may induce the differentiation of the PSC into a hepatocyte or a hepatic cell. Different amounts of NFE2L2 may be introduced into the PSC. At least about 5, about 10, about 15, about 20, about 25, or about 50 copies of the open reading frame (ORF) for NFE2L2 per cell may be introduced. Alternatively, or in addition to, at most about 50, about 25, about 20, about 15, about 10, or about 5 copies of the open reading frame (ORF) for NFE2L2 per cell may be introduced. Increased levels of expression may also be achieved by increasing the copy number of the ORF, for example, by using a higher copy number vector or by using a transposon.

In some embodiments, the number of copies of the ORF for NFE2L2 introduced per cell can be about 1 to about 90. In some embodiments, the number of copies of the ORF for NFE2L2 introduced per cell can be at least about 1. In some embodiments, the number of copies of the ORF for NFE2L2 introduced per cell can be at most about 90. In some embodiments, the number of copies of the ORF for NFE2L2 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for NFE2L2 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise NFE2L2. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### GATA Zinc Finger Transcription Factors

The present disclosure provides cells, expression cassettes, and methods using transcription factors, or molecules that increase transcription or increase activation of transcription factors. One or more transcription factors may be used. In some embodiments, a transcription factor may be a member of the GATA zinc finger family of transcription factors, which may be, for example, GATA2, GATA4, or GATA6, ODAG, RGO83M05.2, GATA Zinc Finger Domain-Containing Protein 1, Ocular Development-Associated Gene Protein, FJ22489, Ocular Development-Associated Gene, CMD2B. The GATA2 transcription factor may be, for example, isoform P23769-1 or P23769-2.

GATA2 isoform P23769-2

The present disclosure provides cells, expression cassettes, and methods using transcription factors, or molecules that increase transcription or increase activation of transcription factors. One or more transcription factors may be used. In some embodiments, a transcription factor may be GATA2 isoform P23769-2, which may also be known as GATA Binding Protein 2; NFE1B; Endothelial Transcription Factor GATA-2; GATA-Binding Protein 2; MONOMAC; IMD21; or DCML.

The present disclosure provides an expression cassette comprising one or more transcription factors. The one or more transcription factors may comprise GATA2 isoform P23769-2. GATA2 isoform P23769-2 may be introduced in an expression cassette. GATA2 isoform P23769-2 may be expressed in an expression cassette. An expression cassette comprising GATA2 isoform P23769-2 may be introduced in a PSC. An expression cassette comprising GATA2 isoform P23769-2 may be expressed in a PSC. An expression cassette comprising GATA2 isoform P23769-2 may induce differentiation of the PSC into a hepatocyte or hepatic cell.

GATA2 isoform P23769-2 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising GATA2 isoform P23769-2 and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise GATA2 isoform P23769-2. An expression cassette comprising GATA2 isoform P23769-2 may be expressed in a PSC. An expression cassette comprising GATA2 isoform P23769-2 may induce the differentiation of the PSC into a hepatocyte or a hepatic cell. Different amounts of GATA2 isoform P23769-2 may be introduced into the PSC. At least about 5, about 10, about 15, about 20, about 25, or about 50 copies of the open reading frame (ORF) for GATA2 isoform P23769-2 per cell may be introduced. Alternatively, or in addition to, at most about 50, about 25, about 20, about 15, about 10, or about 5 copies of the open reading frame (ORF) for GATA2 isoform P23769-2 per cell may be introduced. Increased levels of expression may also be achieved by increasing the copy number of the ORF, for example, by using a higher copy number vector or by using a transposon.

In some embodiments, the number of copies of the ORF for GATA2 isoform P23769-2 introduced per cell can be about 1 to about 90. In some embodiments, the number of copies of the ORF for GATA2 isoform P23769-2 introduced per cell can be at least about 1. In some embodiments, the number of copies of the ORF for GATA2 isoform P23769-2 introduced per cell can be at most about 90. In some embodiments, the number of copies of the ORF for GATA2 isoform P23769-2 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for GATA2 isoform P23769-2 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise GATA2 isoform P23769-2. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### GATA4

The present disclosure provides cells, expression cassettes, and methods using transcription factors, or molecules that increase transcription or increase activation of transcription factors. One or more transcription factors may be used. In some embodiments, a transcription factor may be GATA4, which may also be known as GATA Binding Protein 4; Transcription Factor GATA-4; GATA-Binding Factor 4; GATA-Binding Protein 4; TACHD; ASD2; VSD1; or TOF.

The present disclosure provides an expression cassette comprising one or more transcription factors. The one or more transcription factors may comprise GATA4. GATA4 may be introduced in an expression cassette. GATA4 may be expressed in an expression cassette. An expression cassette comprising GATA4 may be introduced in a PSC. An expression cassette comprising GATA4 may be expressed in a PSC. An expression cassette comprising GATA4 may induce differentiation of the PSC into a hepatocyte or hepatic cell.

GATA4 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising GATA4, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise GATA4. An expression cassette comprising GATA4 may be expressed in a PSC. An expression cassette comprising GATA4 may induce the differentiation of the PSC into a hepatocyte or a hepatic cell. Different amounts of GATA4 may be introduced into the PSC. At least about 5, about 10, about 15, about 20, about 25, or about 50 copies of the open reading frame (ORF) for GATA4 per cell may be introduced. Alternatively, or in addition to, at most about 50, about 25, about 20, about 15, about 10, or about 5 copies of the open reading frame (ORF) for GATA4 per cell may be introduced. Increased levels of expression may also be achieved by increasing the copy number of the ORF, for example, by using a higher copy number vector or by using a transposon.

In some embodiments, the number of copies of the ORF for GATA4 introduced per cell can be about 1 to about 90. In some embodiments, the number of copies of the ORF for GATA4 introduced per cell can be at least about 1. In some embodiments, the number of copies of the ORF for GATA4 introduced per cell can be at most about 90. In some embodiments, the number of copies of the ORF for GATA4 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for GATA4 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise GATA4. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### GATA2 Isoform P23769-1

The present disclosure provides cells, expression cassettes, and methods using transcription factors, or molecules that increase transcription or increase activation of transcription factors. One or more transcription factors may be used. In some embodiments, a transcription factor may be GATA2 isoform P23769-2, which may also be known as GATA Binding Protein 2; NFE1B; Endothelial Transcription Factor GATA-2; GATA-Binding Protein 2; MONOMAC; IMD21; or DCML.

The present disclosure provides an expression cassette comprising one or more transcription factors. The one or more transcription factors may comprise GATA2 isoform P23769-1. GATA2 isoform P23769-1 may be introduced in an expression cassette. GATA2 isoform P23769-1 may be expressed in an expression cassette. An expression cassette comprising GATA2 isoform P23769-1 may be introduced in a PSC. An expression cassette comprising GATA2 isoform P23769-1 may be expressed in a PSC. An expression cassette comprising GATA2 isoform P23769-1 may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise GATA2 isoform P23769-1. GATA2 isoform P23769-1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising GATA2 isoform P23769-1 and one or more other transcription factors may induce differentiation of the PSC into a hepatocyte or hepatic cell. Different amounts of GATA2 isoform P23769-1 may be introduced into the PSC. At least about 5, about 10, about 15, about 20, about 25, or about 50 copies of the open reading frame (ORF) for GATA2 isoform P23769-1 per cell may be introduced. Alternatively, or in addition to, at most about 50, about 25, about 20, about 15, about 10, or about 5 copies of the open reading frame (ORF) for GATA2 isoform P23769-1per cell may be introduced. Increased levels of expression may also be achieved by increasing the copy number of the ORF, for example, by using a higher copy number vector or by using a transposon.

In some embodiments, the number of copies of the ORF for GATA2 isoform P23769-1 introduced per cell can be about 1 to about 90. In some embodiments, the number of copies of the ORF for GATA2 isoform P23769-1 introduced per cell can be at least about 1. In some embodiments, the number of copies of the ORF for GATA2 isoform P23769-1 introduced per cell can be at most about 90. In some embodiments, the number of copies of the ORF for GATA2 isoformP23769-1 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for GATA2 isoform P23769-1 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise GATA2 isoform P23769-1. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### GATA6

The present disclosure provides cells, expression cassettes, and methods using transcription factors, or molecules that increase transcription or increase activation of transcription factors. One or more transcription factors may be used. In some embodiments, a transcription factor may be GATA6, which may also be known as GATA Binding Protein 6; Transcription Factor GATA-6; GATA-Binding Factor 6; or GATA-Binding Protein 6.

The present disclosure provides an expression cassette comprising one or more transcription factors. The one or more transcription factors may comprise GATA6. GATA6 may be introduced in an expression cassette. GATA6 may be expressed in an expression cassette. An expression cassette comprising GATA6 may be introduced in a PSC. An expression cassette comprising GATA6 may be expressed in a PSC. An expression cassette comprising GATA6 may induce differentiation of the PSC into a hepatocyte or hepatic cell.

GATA6 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising GATA6, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise GATA6. An expression cassette comprising GATA6 may be expressed in a PSC. An expression cassette comprising GATA6 may induce the differentiation of the PSC into a hepatocyte or a hepatic cell. Different amounts of GATA6 may be introduced into the PSC. At least about 5, about 10, about 15, about 20, about 25, or about 50 copies of the open reading frame (ORF) for GATA6 per cell may be introduced. Alternatively, or in addition to, at most about 50, about 25, about 20, about 15, about 10, or about 5 copies of the open reading frame (ORF) for GATA6 per cell may be introduced. Increased levels of expression may also be achieved by increasing the copy number of the ORF, for example, by using a higher copy number vector or by using a transposon.

In some embodiments, the number of copies of the ORF for GATA6 introduced per cell can be about 1 to about 90. In some embodiments, the number of copies of the ORF for GATA6 introduced per cell can be at least about 1. In some embodiments, the number of copies of the ORF for GATA6 introduced per cell can be at most about 90. In some embodiments, the number of copies of the ORF for GATA6 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for GATA6 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise GATA6. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### Nuclear Factor I (NFI) Family

The present disclosure provides cells, expression cassettes, and methods using transcription factors, or molecules that increase transcription or increase activation of transcription factors. One or more transcription factors may be used. A transcription factor may be a member of the NFI family of transcription factors. The NFI transcription factor may be, for example, NFIX.

### NFIX

The present disclosure provides cells, expression cassettes, and methods using transcription factors, or molecules that increase transcription or increase activation of transcription factors. One or more transcription factors may be used. A transcription factor may be a member of the NFI family. In some embodiments, the transcription factor may be NFIX, which may also be known as Nuclear Factor I X; NF1A; CCAAT-Box-Binding Transcription Factor; Nuclear Factor 1 X-Type; TGGCA-Binding Protein; Nuclear Factor 1/X; NF-I/X; NF1-X; CTF; Nuclear Factor I/X (CCAAT-Binding Transcription Factor); CCAAT-Binding Transcription Factor; Nuclear Factor I/X; MRSHSS; SOTOS2; or NFI-X.

The present disclosure provides an expression cassette comprising one or more transcription factors. The one or more transcription factors may comprise NFIX. NFIX may be introduced in an expression cassette. NFIX may be expressed in an expression cassette. An expression cassette comprising NFIX may be introduced in a PSC. An expression cassette comprising NFIX may be expressed in a PSC. An expression cassette comprising NFIX may induce differentiation of the PSC into a hepatocyte or hepatic cell.

NFIX may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising NFIX, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise NFIX. An expression cassette comprising NFIX may be expressed in a PSC. An expression cassette comprising NFIX may induce the differentiation of the PSC into a hepatocyte or a hepatic cell. Different amounts of NFIX may be introduced into the PSC. At least about 5, about 10, about 15, about 20, about 25, or about 50 copies of the open reading frame (ORF) for NFIX per cell may be introduced. Alternatively, or in addition to, at most about 50, about 25, about 20, about 15, about 10, or about 5 copies of the open reading frame (ORF) for NFIX per cell may be introduced. Increased levels of expression may also be achieved by increasing the copy number of the ORF, for example, by using a higher copy number vector or by using a transposon.

In some embodiments, the number of copies of the ORF for NFIX introduced per cell can be about 1 to about 90. In some embodiments, the number of copies of the ORF for NFIX introduced per cell can be at least about 1. In some embodiments, the number of copies of the ORF for NFIX introduced per cell can be at most about 90. In some embodiments, the number of copies of the ORF for NFIX introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for NFIX introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise NFIX. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### T-Box Family

The present disclosure provides cells, expression cassettes, and methods using transcription factors, or molecules that increase transcription or increase activation of transcription factors. One or more transcription factors may be used. A transcription factor may be a member of the T-box family of transcription factors. The transcription factor may be a member of the TBX family. The T-box transcription factor may be, for example, TBX3.

### TBX3

The present disclosure provides cells, expression cassettes, and methods using transcription factors, or molecules that increase transcription or increase activation of transcription factors. One or more transcription factors may be used. In some embodiments, a transcription factor may be TBX3, which may also be known as T-Box Transcription Factor 3; TBX3-ISO; XHL; T-Box Transcription Factor TBX3; T-Box Protein 3; T-Box 3; UMS; Bladder Cancer Related Protein XHL; or Ulnar Mammary Syndrome.

The present disclosure provides an expression cassette comprising one or more transcription factors. The one or more transcription factors may comprise TBX3. TBX3 may be introduced in an expression cassette. TBX3 may be expressed in an expression cassette. An expression cassette comprising TBX3 may be introduced in a PSC. An expression cassette comprising TBX3 may be expressed in a PSC. An expression cassette comprising TBX3 may induce differentiation of the PSC into a hepatocyte or hepatic cell.

TBX3 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising TBX3, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise TBX3. An expression cassette comprising TBX3 may be expressed in a PSC. An expression cassette comprising TBX3 may induce the differentiation of the PSC into a hepatocyte or a hepatic cell. Different amounts of TBX3 may be introduced into the PSC. Different amounts of TBX3 may be introduced into the PSC. At least about 5, about 10, about 15, about 20, about 25, or about 50 copies of the open reading frame (ORF) for TBX3 per cell may be introduced. Alternatively, or in addition to, at most about 50, about 25, about 20, about 15, about 10, or about 5 copies of the open reading frame (ORF) for TBX3 per cell may be introduced. Increased levels of expression may also be achieved by increasing the copy number of the ORF, for example, by using a higher copy number vector or by using a transposon.

In some embodiments, the number of copies of the ORF for TBX3 introduced per cell can be about 1 to about 90. In some embodiments, the number of copies of the ORF for TBX3 introduced per cell can be at least about 1. In some embodiments, the number of copies of the ORF for TBX3 introduced per cell can be at most about 90. In some embodiments, the number of copies of the ORF for TBX3 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for TBX3 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise TBX3. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### NR Nuclear Receptor Family

The present disclosure provides cells, expression cassettes, and methods using transcription factors, or molecules that increase transcription or increase activation of transcription factors. One or more transcription factors may be used. A transcription factor may be a member of the NR Nuclear Receptor of transcription factors. The NR Nuclear Receptor transcription factor may be, for example, NR1I3, NR5A1, and RXRB.

### RXRB

The present disclosure provides cells, expression cassettes, and methods using transcription factors, or molecules that increase transcription or increase activation of transcription factors. One or more transcription factors may be used. A transcription factor may be a member of the RXR family. In some embodiments, the transcription factor may be RXRB, which may also be known as Retinoid X Receptor Beta; NR2B2; Nuclear Receptor Subfamily 2 Group B Member 2; H-2RIIBP; RCoR-1; Retinoic Acid Receptor RXR-Beta; RXR-Beta; RXRbeta; MHC Class I Promoter Binding Protein; Retinoid X Receptor, Beta; or DAUDI6.

The present disclosure provides an expression cassette comprising one or more transcription factors. The one or more transcription factors may comprise RXRB. RXRB may be introduced in an expression cassette. RXRB may be expressed in an expression cassette. An expression cassette comprising RXRB may be introduced in a PSC. An expression cassette comprising RXRB may be expressed in a PSC. An expression cassette comprising RXRB may induce differentiation of the PSC into a hepatocyte or hepatic cell.

RXRB may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising RXRB, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise RXRB. An expression cassette comprising RXRB may be expressed in a PSC. An expression cassette comprising RXRB may induce the differentiation of the PSC into a hepatocyte or a hepatic cell. Different amounts of RXRB may be introduced into the PSC. Different amounts of RXRB may be introduced into the PSC. At least about 5, about 10, about 15, about 20, about 25, or about 50 copies of the open reading frame (ORF) for RXRB per cell may be introduced. Alternatively, or in addition to, at most about 50, about 25, about 20, about 15, about 10, or about 5 copies of the open reading frame (ORF) for RXRB per cell may be introduced. Increased levels of expression may also be achieved by increasing the copy number of the ORF, for example, by using a higher copy number vector or by using a transposon.

In some embodiments, the number of copies of the ORF for RXRB introduced per cell can be about 1 to about 90. In some embodiments, the number of copies of the ORF for RXRB introduced per cell can be at least about 1. In some embodiments, the number of copies of the ORF for RXRB introduced per cell can be at most about 90. In some embodiments, the number of copies of the ORF for RXRB introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for RXRB introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise RXRB. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### NR5A2

The present disclosure provides cells, expression cassettes, and methods using transcription factors, or molecules that increase transcription or increase activation of transcription factors. One or more transcription factors may be used. In some embodiments, a transcription factor may be NR5A2, which may also be known as Nuclear Receptor Subfamily 5 Group A Member 2; LRH-1; FTZ-F1beta; FTZ-F1; HB1F-2; HB1F; B1F2; LRH1; FTF; CYP7A Promoter-Binding Factor; BIF; CPF; B1-Binding Factor, Hepatocyte Transcription Factor Which Activates Enhancer II Of Hepatitis B Virus; Nuclear Receptor Subfamily 5, Group A, Member 2; Fetoprotein-Alpha 1 (AFP) Transcription Factor; Liver Nuclear Receptor Homolog-1 Variant 2; Alpha-1-Fetoprotein Transcription Factor; Hepatocytic Transcription Factor HB1F-3; Hepatocytic Transcription Factor; Liver Receptor Homolog-1; Liver Receptor Homolog 1; or B1-Binding Factor.

The present disclosure provides an expression cassette comprising one or more transcription factors. The one or more transcription factors may comprise NR5A2. NR5A2 may be introduced in an expression cassette. NR5A2 may be expressed in an expression cassette. An expression cassette comprising NR5A2 may be introduced in a PSC. An expression cassette comprising NR5A2 may be expressed in a PSC. An expression cassette comprising NR5A2 may induce differentiation of the PSC into a hepatocyte or hepatic cell.

NR5A2 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising NR5A2, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise NR5A2. An expression cassette comprising NR5A2 may be expressed in a PSC. An expression cassette comprising NR5A2 may induce the differentiation of the PSC into a hepatocyte or a hepatic cell. Different amounts of NR5A2 may be introduced into the PSC. At least about 5, about 10, about 15, about 20, about 25, or about 50 copies of the open reading frame (ORF) for NR5A2 per cell may be introduced. Alternatively, or in addition to, at most about 50, about 25, about 20, about 15, about 10, or about 5 copies of the open reading frame (ORF) for NR5A2 per cell may be introduced. Increased levels of expression may also be achieved by increasing the copy number of the ORF, for example, by using a higher copy number vector or by using a transposon.

In some embodiments, the number of copies of the ORF for NR5A2 introduced per cell can be about 1 to about 90. In some embodiments, the number of copies of the ORF for NR5A2 introduced per cell can be at least about 1. In some embodiments, the number of copies of the ORF for NR5A2 introduced per cell can be at most about 90. In some embodiments, the number of copies of the ORF for NR5A2 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for NR5A2 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise NR5A2. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### NR5A1

The present disclosure provides cells, expression cassettes, and methods using transcription factors, or molecules that increase transcription or increase activation of transcription factors. One or more transcription factors may be used. A transcription factor may be NR5A1. NR5A1 may also be known as Nuclear Receptor Subfamily 5 Group A Member 1, AD4BP, HSF-1, SF-1, SF1, Steroidogenic Factor 1, FTZF1, FTZ1, ELP, Steroid Hormone Receptor Ad4BP, Fushi Tarazu Factor Homolog 1, STF-1, Nuclear Receptor Subfamily 5, Group A, Member 1, Steroidogenic Factor 1 Nuclear Receptor, Adrenal 4 Binding Protein, Adrenal 4-Binding Protein, Nuclear Receptor AdBP4, Steroidogenic Factor-1, SPGF8, SRXX4, SRXY3, or POF7.

The present disclosure provides an expression cassette comprising one or more transcription factors. The one or more transcription factors may comprise NR5A1. NR5A1 may be introduced in an expression cassette. NR5A1 may be expressed in an expression cassette. An expression cassette comprising NR5A1 may be introduced in a PSC. An expression cassette comprising NR5A1 may be expressed in a PSC. An expression cassette comprising NR5A1 may induce differentiation of the PSC into a hepatocyte or hepatic cell.

NR5A1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising NR5A1, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise NR5A1. An expression cassette comprising NR5A1 may be expressed in a PSC. An expression cassette comprising NR5A1 may induce the differentiation of the PSC into a hepatocyte or a hepatic cell. Different amounts of NR5A1 may be introduced into the PSC. At least about 5, about 10, about 15, about 20, about 25, or about 50 copies of the open reading frame (ORF) for NR5A1 per cell may be introduced. Alternatively, or in addition to, at most about 50, about 25, about 20, about 15, about 10, or about 5 copies of the open reading frame (ORF) for NR5A1 per cell may be introduced. Increased levels of expression may also be achieved by increasing the copy number of the ORF, for example, by using a higher copy number vector or by using a transposon.

In some embodiments, the number of copies of the ORF for NR5A1 introduced per cell can be about 1 to about 90. In some embodiments, the number of copies of the ORF for NR5A1 introduced per cell can be at least about 1. In some embodiments, the number of copies of the ORF for NR5A1 introduced per cell can be at most about 90. In some embodiments, the number of copies of the ORF for NR5A1 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for NR5A1 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise NR5A1. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### NR1I3

The present disclosure provides cells, expression cassettes, and methods using transcription factors, or molecules that increase transcription or increase activation of transcription factors. One or more transcription factors may be used. A transcription factor may be NR1I3. NR113 may also be known as Nuclear Receptor Subfamily 1 Group I Member 3; CAR; Constitutive Androstane Receptor; MB67; CAR1; Constitutive Activator Of Retinoid Response; Constitutive Active Response; Orphan Nuclear Receptor MB67; Constitutive Androstane Nuclear Receptor Variant 2; Constitutive Androstane Nuclear Receptor Variant 3; Constitutive Androstane Nuclear Receptor Variant 4; Constitutive Androstane Nuclear Receptor Variant 5; Nuclear Receptor Subfamily 1, Group I, Member 3; Orphan Nuclear Hormone Receptor; or Constitutive Active Receptor.

The present disclosure provides an expression cassette comprising one or more transcription factors. The one or more transcription factors may comprise NR1I3. NR113 may be introduced in an expression cassette. NR113 may be expressed in an expression cassette. An expression cassette comprising NR113 may be introduced in a PSC. An expression cassette comprising NR113 may be expressed in a PSC. An expression cassette comprising NR113 may induce differentiation of the PSC into a hepatocyte or hepatic cell.

NR113 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising NR113, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise NR1I3. An expression cassette comprising NR113 may be expressed in a PSC. An expression cassette comprising NR113 may induce the differentiation of the PSC into a hepatocyte or a hepatic cell. Different amounts of NR113 may be introduced into the PSC. At least about 5, about 10, about 15, about 20, about 25, or about 50 copies of the open reading frame (ORF) for NR113 per cell may be introduced. Alternatively, or in addition to, at most about 50, about 25, about 20, about 15, about 10, or about 5 copies of the open reading frame (ORF) for NR113 per cell may be introduced. Increased levels of expression may also be achieved by increasing the copy number of the ORF, for example, by using a higher copy number vector or by using a transposon.

In some embodiments, the number of copies of the ORF for NR1I3 introduced per cell can be about 1 to about 90. In some embodiments, the number of copies of the ORF for NR113 introduced per cell can be at least about 1. In some embodiments, the number of copies of the ORF for NR113 introduced per cell can be at most about 90. In some embodiments, the number of copies of the ORF for NR113 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for NR113 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise NR1I3. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### NKL Subclass Homeoboxes and Pseudogenes

The present disclosure provides cells, expression cassettes, and methods using transcription factors, or molecules that increase transcription or increase activation of transcription factors. One or more transcription factors may be used. A transcription factor may be a member of the transcription factor family of NKL subclass homeoboxes and pseudogenes. The transcription factor may be, for example, HHEX.

### HHEX

The present disclosure provides cells, expression cassettes, and methods using transcription factors, or molecules that increase transcription or increase activation of transcription factors. One or more transcription factors may be used. A transcription factor may be a member of the HHEX family. The transcription factor may be HHEX. HHEX may also be known as Haematopoietically Expressed Homeobox; HEX; HOX11L-PEN; PRHX; Haematopoietically-Expressed Homeobox Protein HHEX; Homeobox Protein HEX; Homeobox Protein PRH; PRH; Proline-Rich Homeodomain-Containing Transcription Factor; Homeobox, Haematopoietically Expressed; or HMPH.

The present disclosure provides an expression cassette comprising one or more transcription factors. HHEX may be introduced in an expression cassette. HHEX may be expressed in an expression cassette. An expression cassette comprising HHEX may be introduced in a PSC. An expression cassette comprising HHEX may be expressed in a PSC. An expression cassette comprising HHEX may induce differentiation of the PSC into a hepatocyte or hepatic cell.

HHEX may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising HHEX, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise HHEX. An expression cassette comprising HHEX may be expressed in a PSC. An expression cassette comprising HHEX may induce the differentiation of the PSC into a hepatocyte or a hepatic cell. Different amounts of NFIX may be introduced into the PSC. Different amounts of HHEX may be introduced into the PSC. At least about 5, about 10, about 15, about 20, about 25, or about 50 copies of the open reading frame (ORF) for HHEX per cell may be introduced. Alternatively, or in addition to, at most about 50, about 25, about 20, about 15, about 10, or about 5 copies of the open reading frame (ORF) for HHEX per cell may be introduced. Increased levels of expression may also be achieved by increasing the copy number of the ORF, for example, by using a higher copy number vector or by using a transposon.

In some embodiments, the number of copies of the ORF for HHEX introduced per cell can be about 1 to about 90. In some embodiments, the number of copies of the ORF for HHEX introduced per cell can be at least about 1. In some embodiments, the number of copies of the ORF for HHEX introduced per cell can be at most about 90. In some embodiments, the number of copies of the ORF for HHEX introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for HHEX introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise HHEX. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### SRY-Box Transcription Factor

The present disclosure provides cells, expression cassettes, and methods using transcription factors, or molecules that increase transcription or increase activation of transcription factors. One or more transcription factors may be used. A transcription factor may be a SRY-box transcription factor. A transcription factor may be a member of the SOX family. The transcription factor may be, for example, SOX17.

### SOX17

The present disclosure provides cells, expression cassettes, and methods using transcription factors, or molecules that increase transcription or increase activation of transcription factors. One or more transcription factors may be used. A transcription factor may be a member of the SOX family. The transcription factor may be SOX17. SOX17 may also be known as SRY-Box Transcription Factor 17; SRY (Sex Determining Region Y)-Box 17; Transcription Factor SOX-17; SRY-Box 17; SRY-Related HMG-Box Transcription Factor SOX17; or VUR3.

The present disclosure provides an expression cassette comprising one or more transcription factors. SOX17 may be introduced in an expression cassette. SOX17 may be expressed in an expression cassette. An expression cassette comprising SOX17 may be introduced in a PSC. An expression cassette comprising SOX17 may be expressed in a PSC. An expression cassette comprising SOX17 may induce differentiation of the PSC into a hepatocyte or hepatic cell.

SOX17 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising SOX17, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise SOX17. An expression cassette comprising SOX17 may be expressed in a PSC. An expression cassette comprising SOX17 may induce the differentiation of the PSC into a hepatocyte or a hepatic cell. Different amounts of SOX17 may be introduced into the PSC. At least about 5, about 10, about 15, about 20, about 25, or about 50 copies of the open reading frame (ORF) for SOX17 per cell may be introduced. Alternatively, or in addition to, at most about 50, about 25, about 20, about 15, about 10, or about 5 copies of the open reading frame (ORF) for SOX17 per cell may be introduced. Increased levels of expression may also be achieved by increasing the copy number of the ORF, for example, by using a higher copy number vector or by using a transposon.

In some embodiments, the number of copies of the ORF for SOX17 introduced per cell can be about 1 to about 90. In some embodiments, the number of copies of the ORF for SOX17 introduced per cell can be at least about 1. In some embodiments, the number of copies of the ORF for SOX17 introduced per cell can be at most about 90. In some embodiments, the number of copies of the ORF for SOX17 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for SOX17 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise SOX17. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### Ig-Like, Plexins, Transcription Factors (IPT) Domain

The present disclosure provides cells, expression cassettes, and methods using transcription factors, or molecules that increase transcription or increase activation of transcription factors. One or more transcription factors may be used. A transcription factor may have an Ig-like, Plexins, Transcription factors (IPT) domain. The transcription factor may be, for example, RBPJ.

### RBPJ

The present disclosure provides cells, expression cassettes, and methods using transcription factors, or molecules that increase transcription or increase activation of transcription factors. One or more transcription factors may be used. A transcription factor may be a member of the RBPJ family. The transcription factor may be RBPJ. RBPJ may also be known as Recombination Signal Binding Protein For Immunoglobulin Kappa J Region; IGKJRB; RBPJK; RBP-J; IGKJRB1; RBPSUH; KBF2; CBF1; SUH; Recombining Binding Protein Suppressor Of Hairless; Renal Carcinoma Antigen NY-REN-30; RBP-J Kappa; RBP-JK; CBF-1; Immunoglobulin Kappa J Region Recombination Signal Binding Protein 1; Recombining Binding Protein Suppressor Of Hairless (Drosophila); J Kappa-Recombination Signal-Binding Protein; Suppressor Of Hairless Homolog (Drosophila); Suppressor Of Hairless Homolog; H-2K Binding Factor-2; AOS3; or Csl.

The present disclosure provides an expression cassette comprising one or more transcription factors. The one or more transcription factors may comprise RBPJ. RBPJ may be introduced in an expression cassette. RBPJ may be expressed in an expression cassette. An expression cassette comprising RBPJ may be introduced in a PSC. An expression cassette comprising RBPJ may be expressed in a PSC. An expression cassette comprising RBPJ may induce differentiation of the PSC into a hepatocyte or hepatic cell.

RBPJ may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising RBPJ, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise RBPJ. An expression cassette comprising RBPJ may be expressed in a PSC. An expression cassette comprising RBPJ may induce the differentiation of the PSC into a hepatocyte or a hepatic cell. Different amounts of NFIX may be introduced into the PSC. Different amounts of RBPJ may be introduced into the PSC. At least about 5, about 10, about 15, about 20, about 25, or about 50 copies of the open reading frame (ORF) for RBPJ per cell may be introduced. Alternatively, or in addition to, at most about 50, about 25, about 20, about 15, about 10, or about 5 copies of the open reading frame (ORF) for RBPJ per cell may be introduced. Increased levels of expression may also be achieved by increasing the copy number of the ORF, for example, by using a higher copy number vector or by using a transposon.

In some embodiments, the number of copies of the ORF for RBPJ introduced per cell can be about 1 to about 90. In some embodiments, the number of copies of the ORF for RBPJ introduced per cell can be at least about 1. In some embodiments, the number of copies of the ORF for RBPJ introduced per cell can be at most about 90. In some embodiments, the number of copies of the ORF for RBPJ introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for RBPJ introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise RBPJ. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### SMAD

The present disclosure provides cells, expression cassettes, and methods using transcription factors, or molecules that increase transcription or increase activation of transcription factors. One or more transcription factors may be used. A transcription factor may be a SMAD transcription factor. The transcription factor may be, for example, Smad3.

### Smad3

The present disclosure provides cells, expression cassettes, and methods using transcription factors, or molecules that increase transcription or increase activation of transcription factors. One or more transcription factors may be used. A transcription factor may be Smad3. Smad3 may also be known as SMAD Family Member 3; JV15-2; HsT17436; MADH3; Mothers Against Decapentaplegic Homolog 3; Mothers Against DPP Homolog 3; MAD Homolog 3; HMAD-3; HSMAD3; Mad3; MAD, Mothers Against Decapentaplegic Homolog 3 (Drosophila); SMAD, Mothers Against DPP Homolog 3 (Drosophila); MAD, Mothers Against Decapentaplegic Homolog 3; SMAD, Mothers Against DPP Homolog 3; SMA- And MAD-Related Protein 3; Mad Protein Homolog; Mad Homolog JV15-2; HSPC193; SMAD 3; LDS1C; Smad3; or LDS3.

The present disclosure provides an expression cassette comprising one or more transcription factors. Smad3 may be introduced in an expression cassette. Smad3 may be expressed in an expression cassette. An expression cassette comprising Smad3 may be introduced in a PSC. An expression cassette comprising Smad3 may be expressed in a PSC. An expression cassette comprising Smad3 may induce differentiation of the PSC into a hepatocyte or hepatic cell.

Smad3 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising Smad3, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise Smad3. An expression cassette comprising Smad3 may be expressed in a PSC. An expression cassette Smad3 NFIX may induce the differentiation of the PSC into a hepatocyte or a hepatic cell. Different amounts of Smad3 may be introduced into the PSC. At least about 5, about 10, about 15, about 20, about 25, or about 50 copies of the open reading frame (ORF) for Smad3 per cell may be introduced. Alternatively, or in addition to, at most about 50, about 25, about 20, about 15, about 10, or about 5 copies of the open reading frame (ORF) for Smad3 per cell may be introduced. Increased levels of expression may also be achieved by increasing the copy number of the ORF, for example, by using a higher copy number vector or by using a transposon.

In some embodiments, the number of copies of the ORF for Smad3 introduced per cell can be about 1 to about 90. In some embodiments, the number of copies of the ORF for Smad3 introduced per cell can be at least about 1. In some embodiments, the number of copies of the ORF for Smad3 introduced per cell can be at most about 90. In some embodiments, the number of copies of the ORF for Smad3 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for Smad3 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise Smad3. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### Basic Helix-Loop-Helix

The present disclosure provides cells, expression cassettes, and methods using transcription factors, or molecules that increase transcription or increase activation of transcription factors. One or more transcription factors may be used. A transcription factor may be a member of the basic helix-loop-helix family of transcription factors. The transcription factor may be, for example, HIFIA or MYC.

### Myc

The present disclosure provides cells, expression cassettes, and methods using transcription factors, or molecules that increase transcription or increase activation of transcription factors. One or more transcription factors may be used. A transcription factor may be a member of the Myc family. The transcription factor may be Myc. Myc may also be known as MYC Proto-Oncogene, BHLH Transcription Factor; BHLHe39; C-Myc; MYCC; V-Myc Avian Myelocytomatosis Viral Oncogene Homolog; Class E Basic Helix-Loop-Helix Protein 39; Myc Proto-Oncogene Protein; Transcription Factor P64; Proto-Oncogene C-Myc; Myc-Related Translation/Localization Regulatory Factor; Avian Myelocytomatosis Viral Oncogene Homolog; V-Myc Myelocytomatosis Viral Oncogene Homolog; BHLHE39; or MRTL.

The present disclosure provides an expression cassette comprising one or more transcription factors. The one or more transcription factors may comprise Myc. Myc may be introduced in an expression cassette. Myc may be expressed in an expression cassette. An expression cassette comprising Myc may be introduced in a PSC. An expression cassette comprising Myc may be expressed in a PSC. An expression cassette comprising Myc may induce differentiation of the PSC into a hepatocyte or hepatic cell.

Myc may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising Myc and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise Myc. An expression cassette comprising Myc may be expressed in a PSC. An expression cassette comprising Myc may induce the differentiation of the PSC into a hepatocyte or a hepatic cell. Different amounts of Myc may be introduced into the PSC. At least about 5, about 10, about 15, about 20, about 25, or about 50 copies of the open reading frame (ORF) for Myc per cell may be introduced. Alternatively, or in addition to, at most about 50, about 25, about 20, about 15, about 10, or about 5 copies of the open reading frame (ORF) for Myc per cell may be introduced. Increased levels of expression may also be achieved by increasing the copy number of the ORF, for example, by using a higher copy number vector or by using a transposon.

In some embodiments, the number of copies of the ORF for Myc introduced per cell can be about 1 to about 90. In some embodiments, the number of copies of the ORF for Myc introduced per cell can be at least about 1. In some embodiments, the number of copies of the ORF for Myc introduced per cell can be at most about 90. In some embodiments, the number of copies of the ORF for Myc introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for Myc introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise Myc. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### HIFIA

The present disclosure provides cells, expression cassettes, and methods using transcription factors, or molecules that increase transcription or increase activation of transcription factors. One or more transcription factors may be used. A transcription factor may be a member of the HIF family. The member of the HIF family may be HIF1A. HIFIA may also be known as Hypoxia Inducible Factor 1 Subunit Alpha; BHLHe78; PASD8; MOP1; Class E Basic Helix-Loop-Helix Protein 78; PAS Domain-Containing Protein 8; Member Of PAS Protein 1; HIF-1alpha; HIF1; Hypoxia Inducible Factor 1, Alpha Subunit (Basic Helix-Loop-Helix Transcription Factor); Basic-Helix-Loop-Helix-PAS Protein MOP1; Hypoxia-Inducible Factor 1-Alpha; HIF-1-Alpha; Hypoxia Inducible Factor 1 Alpha Subunit; Hypoxia-Inducible Factor1alpha; Member Of PAS Superfamily 1; ARNT Interacting Protein; ARNT-Interacting Protein; HIF1-ALPHA; HIF1-Alpha; BHLHE78; or HIF-1A.

The present disclosure provides an expression cassette comprising one or more transcription factors. HIFIA may be introduced in an expression cassette. HIFIA may be expressed in an expression cassette. An expression cassette comprising HIFIA may be introduced in a PSC. An expression cassette comprising HIFIA may be expressed in a PSC. An expression cassette comprising HIFIA may induce differentiation of the PSC into a hepatocyte or hepatic cell.

HIF1A may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising HIF1A, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise HIF1A. An expression cassette comprising HIFIA may be expressed in a PSC. An expression cassette comprising HIFIA may induce the differentiation of the PSC into a hepatocyte or a hepatic cell. Different amounts of HIFIA may be introduced into the PSC. At least about 5, about 10, about 15, about 20, about 25, or about 50 copies of the open reading frame (ORF) for HIFIA per cell may be introduced. Alternatively, or in addition to, at most about 50, about 25, about 20, about 15, about 10, or about 5 copies of the open reading frame (ORF) for HIFIA per cell may be introduced. Increased levels of expression may also be achieved by increasing the copy number of the ORF, for example, by using a higher copy number vector or by using a transposon.

In some embodiments, the number of copies of the ORF for HIFIA introduced per cell can be about 1 to about 90. In some embodiments, the number of copies of the ORF for HIF1A introduced per cell can be at least about 1. In some embodiments, the number of copies of the ORF for HIFIA introduced per cell can be at most about 90. In some embodiments, the number of copies of the ORF for HIF1A introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for HIFIA introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise Myc. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### PROX1

The present disclosure provides cells, expression cassettes, and methods using transcription factors, or molecules that increase transcription or increase activation of transcription factors. In some embodiments, one or more transcription factors may be used. In some embodiments, a transcription factor may be PROX1, a highly conserved transcription factor, expressed in hepatocytes from the earliest stages of development into adulthood and over-expressed in hepatoma cell lines. PROX1 may also be known as prospero homeobox 1, Homeobox Prospero-Like Protein PROX1, Prospero-Related Homeobox 1, Prospero Homeobox Protein 1. The present disclosure provides cells, expression cassettes, and methods using transcription factors, or molecules that increase transcription or increase activation of transcription factors. In some embodiments, one or more transcription factors may be used. In some embodiments, a transcription factor may be PROX1, a highly conserved transcription factor, expressed in hepatocytes from the earliest stages of development into adulthood and over-expressed in hepatoma cell lines. PROX1 may also be known as prospero homeobox 1, Homeobox Prospero-Like Protein PROX1, Prospero-Related Homeobox 1, Prospero Homeobox Protein 1. The present disclosure provides an expression cassette comprising one or more transcription factors. The one or more transcription factors may comprise PROX1. PROX1 may be introduced in an expression cassette. An expression cassette comprising PROX1 may be introduced in a PSC. PROX1 may be introduced in an expression cassette. PROX1 may be expressed in an expression cassette. An expression cassette comprising PROX1 may be introduced in a PSC. An expression cassette comprising PROX1may be expressed in a PSC. An expression cassette comprising PROX1 may induce differentiation of the PSC into a hepatocyte or hepatic cell.

PROX1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising PROX1, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise PROX1. An expression cassette comprising PROX1 may be expressed in a PSC. An expression cassette comprising PROX1 may induce the differentiation of the PSC into a hepatocyte or a hepatic cell. Different amounts of PROX1 may be introduced into the PSC. At least about 5, about 10, about 15, about 20, about 25, or about 50 copies of the open reading frame (ORF) for PROX1 per cell may be introduced. Alternatively, or in addition to, at most about 50, about 25, about 20, about 15, about 10, or about 5 copies of the open reading frame (ORF) for PROX1 per cell may be introduced. Increased levels of expression may also be achieved by increasing the copy number of the ORF, for example, by using a higher copy number vector or by using a transposon.

In some embodiments, the number of copies of the ORF for PROX1 introduced per cell can be about 1 to about 90. In some embodiments, the number of copies of the ORF for PROX1 introduced per cell can be at least about 1. In some embodiments, the number of copies of the ORF for PROX1 introduced per cell can be at most about 90. In some embodiments, the number of copies of the ORF for PROX1 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for PROX1 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise PROX1. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### EGR1

EGR1 early growth response 1 (human) TF regulates the expression of the clock gene PER2 under IL-4 stimulated human keratinocytes. The present disclosure provides cells, expression cassettes, and methods using transcription factors, or molecules that increase transcription or increase activation of transcription factors. One or more transcription factors may be used. In some embodiments, the one or more transcription factors may be EGR1. EGR1 may also be known as NGFI-A, AT225, Nerve Growth Factor-Induced Protein A, Early Growth Response Protein 1, Transcription Factor ETR103, KROX-24, ZIF-268, GOS30, zenk.

The present disclosure provides an expression cassette comprising one or more transcription factors. EGR1 may be introduced in an expression cassette. EGR1 may be expressed in an expression cassette. An expression cassette comprising EGR1 may be introduced in a PSC. An expression cassette comprising EGR1 may be expressed in a PSC. An expression cassette comprising EGR1may induce differentiation of the PSC into a hepatocyte or hepatic cell.

EGR1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising EGR1, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise EGR1. An expression cassette comprising EGR1 may be expressed in a PSC. An expression cassette comprising EGR1 may induce the differentiation of the PSC into a hepatocyte or a hepatic cell. Different amounts of EGR1 may be introduced into the PSC. At least about 5, about 10, about 15, about 20, about 25, or about 50 copies of the open reading frame (ORF) for EGR1 per cell may be introduced. Alternatively, or in addition to, at most about 50, about 25, about 20, about 15, about 10, or about 5 copies of the open reading frame (ORF) for EGR1 per cell may be introduced. Increased levels of expression may also be achieved by increasing the copy number of the ORF, for example, by using a higher copy number vector or by using a transposon.

In some embodiments, the number of copies of the ORF for EGR1 introduced per cell can be about 1 to about 90. In some embodiments, the number of copies of the ORF for EGR1 introduced per cell can be at least about 1. In some embodiments, the number of copies of the ORF for EGR1 introduced per cell can be at most about 90. In some embodiments, the number of copies of the ORF for EGR1 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for EGR1 introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise Myc. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### SKI

SKI functions as a repressor of TGF-beta signaling. The present disclosure provides cells, expression cassettes, and methods using transcription factors, or molecules that increase transcription or increase activation of transcription factors. One or more transcription factors may be used. In some embodiments, the one or more transcription factors may be SKI. SKI may also be known synonyms SKI Proto-Oncogene, V-Ski Avian Sarcoma Viral Oncogene Homolog, Sloan-Kettering Institute Proto-Oncogene, Proto-Oncogene C-Ski, Ski Oncogene, Ski Oncoprotein, SGS, SKV.

The present disclosure provides an expression cassette comprising one or more transcription factors. SKI may be introduced in an expression cassette. SKI may be expressed in an expression cassette. An expression cassette comprising SKI may be introduced in a PSC. An expression cassette comprising SKI may be expressed in a PSC. An expression cassette comprising SKI may induce differentiation of the PSC into a hepatocyte or hepatic cell.

SKI may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising SKI and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise SKI. An expression cassette comprising SKI may be expressed in a PSC. An expression cassette comprising SKI may induce the differentiation of the PSC into a hepatocyte or a hepatic cell. Different amounts of SKI may be introduced into the PSC. At least about 5, about 10, about 15, about 20, about 25, or about 50 copies of the open reading frame (ORF) for SKI per cell may be introduced. Alternatively, or in addition to, at most about 50, about 25, about 20, about 15, about 10, or about 5 copies of the open reading frame (ORF) for SKI per cell may be introduced. Increased levels of expression may also be achieved by increasing the copy number of the ORF, for example, by using a higher copy number vector or by using a transposon.

In some embodiments, the number of copies of the ORF for SKI introduced per cell can be about 1 to about 90. In some embodiments, the number of copies of the ORF for SKI introduced per cell can be at least about 1. In some embodiments, the number of copies of the ORF for SKI introduced per cell can be at most about 90. In some embodiments, the number of copies of the ORF for SKI introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for SKI introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise Myc. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### NR1I2/PXR

NR112/PXR nuclear receptor subfamily 1 group I member 2 gene product belongs to the nuclear receptor superfamily, members of which are transcription factors characterized by a ligand-binding domain and a DNA-binding domain. The encoded protein is a transcriptional regulator of the cytochrome P450 gene CYP3A4, binding to the response element of the CYP3A4 promoter as a heterodimer with the 9-cis retinoic acid receptor RXR. It is activated by a range of compounds that induce CYP3A4, including dexamethasone and rifampicin. In some embodiments, NR112/PXR may comprise various alternatively spliced transcripts encoding different isoforms, some of which use non-AUG (CUG) translation initiation codon.

The present disclosure provides cells, expression cassettes, and methods using transcription factors, or molecules that increase transcription or increase activation of transcription factors. One or more transcription factors may be used. In some embodiments, the one or more transcription factors may be NR112/PXR. NR112/PXR may also be known as BXR; PAR; PRR; PXR (pregnane X receptor); SAR; SXR; ONR1; PAR1; PAR2; PARq.

The present disclosure provides an expression cassette comprising one or more transcription factors. NR112/PXR may be introduced in an expression cassette. NR112/PXR may be expressed in an expression cassette. An expression cassette comprising NR112/PXR may be introduced in a PSC. An expression cassette comprising NR112/PXR may be expressed in a PSC. An expression cassette comprising NR112/PXR may induce differentiation of the PSC into a hepatocyte or hepatic cell.

NR112/PXR may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising NR112/PXR, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise NR112/PXR. An expression cassette comprising NR112/PXR may be expressed in a PSC. An expression cassette comprising NR112/PXR may induce the differentiation of the PSC into a hepatocyte or a hepatic cell. Different amounts of NR112/PXR may be introduced into the PSC. At least about 5, about 10, about 15, about 20, about 25, or about 50 copies of the open reading frame (ORF) for NR112/PXR per cell may be introduced. Alternatively, or in addition to, at most about 50, about 25, about 20, about 15, about 10, or about 5 copies of the open reading frame (ORF) for NR112/PXR per cell may be introduced. Increased levels of expression may also be achieved by increasing the copy number of the ORF, for example, by using a higher copy number vector or by using a transposon.

In some embodiments, the number of copies of the ORF for NR1I2/PXR introduced per cell can be about 1 to about 90. In some embodiments, the number of copies of the ORF for NR112/PXR introduced per cell can be at least about 1. In some embodiments, the number of copies of the ORF for NR112/PXR introduced per cell can be at most about 90. In some embodiments, the number of copies of the ORF for NR112/PXR introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for NR112/PXR introduced per cell can be about 1, about 2, about 4, about 5, about 10, about 15, about 20, about 25, about 40, about 50, about 60, or about 90.

The present disclosure provides one or more transcription factors that may induce differentiation of one or more PSCs into one or more hepatocytes or hepatic cells. The one or more transcription factors may comprise Myc. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### Helix-Turn-Helix ETS Family

The present disclosure provides cells, expression cassettes, and methods using transcription factors, or molecules that increase transcription or increase activation of transcription factors. One or more transcription factors may be used. A transcription factor may be a member of the helix-turn-helix ETS family of transcription factors. The transcription factor may be a member of the SPI family. The transcription factor may be, for example, SPI1.

### SPII

The present disclosure provides cells, expression cassettes, and methods using transcription factors, or molecules that increase transcription or increase activation of transcription factors. One or more transcription factors may be used. A transcription factor may be a member of the SPI family. The transcription factor may be SPI1. SPI1 may also be known as Spi-1 Proto-Oncogene; SPI-A; SFPI1; SPI-1; PU.1; OF; Hematopoietic Transcription Factor PU.1; 31 KDa Transforming Protein; Transcription Factor PU.1; Spleen Focus Forming Virus (SFFV) Proviral Integration Oncogene Spi1; Spleen Focus Forming Virus (SFFV) Proviral Integration Oncogene; or 31 KDa-Transforming Protein.

The present disclosure provides an expression cassette comprising one or more transcription factors. The one or more transcription factors may comprise SPI1. SPI1 may be introduced in an expression cassette. SPI1 may be expressed in an expression cassette. An expression cassette comprising SPI1 may be introduced in a PSC. An expression cassette comprising SPI1 may be expressed in a PSC. An expression cassette comprising SPI1 may induce differentiation of the PSC into a hepatocyte or hepatic cell.

SPI1 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising SPI1, and one or more other transcription factors may induce differentiation of the PSC into a hepatocyte or hepatic cell.

The present disclosure provides one or more PSCs comprising one or more exogenous expression cassettes. The one or more exogenous expression cassettes may comprise one or more transcription factors. The one or more transcription factors may comprise SPI1. An expression cassette comprising SPI1 may be expressed in a PSC. An expression cassette comprising SPI1 may induce the differentiation of the PSC into a hepatocyte or a hepatic cell. Different amounts of SPI1 may be introduced into the PSC. At least about 5, about 10, about 15, about 20, about 25, or about 50 copies of the open reading frame (ORF) for SPI1 per cell may be introduced. Alternatively, or in addition to, at most about 50, about 25, about 20, about 15, about 10, or about 5 copies of the open reading frame (ORF) for SPI1 per cell may be introduced. Increased levels of expression may also be achieved by increasing the copy number of the ORF, for example, by using a higher copy number vector or by using a transposon.

In some embodiments, the number of copies of the ORF for SPI1 introduced per cell can be about 1 to about 90. In some embodiments, the number of copies of the ORF for SPI1 introduced per cell can be at least about 1. In some embodiments, the number of copies of the ORF for SPI1 introduced per cell can be at most about 90. In some embodiments, the number of copies of the ORF for SPI1 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for SPI1 introduced per cell can be about 1, about 2, about 4, about 5, about 10, about 15, about 20, about 25, about 40, about 50, about 60, or about 90.

The present disclosure provides one or more hepatocytes or hepatic cells. The one or more hepatocytes or hepatic cells may comprise an exogenous expression cassette. The exogenous expression cassette may comprise one or more transcription factors. The one or more transcription factors may comprise SPI1. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### TP53 Family

The present disclosure provides cells, expression cassettes, and methods using transcription factors, or molecules that increase transcription or increase activation of transcription factors. One or more transcription factors may be used. A transcription factor may be a member of the TP53 family of transcription factors. The transcription factor may be a member of the TP family. The transcription factor may be, for example, TP73.

### TP73

The present disclosure provides cells, expression cassettes, and methods using transcription factors, or molecules that increase transcription or increase activation of transcription factors. One or more transcription factors may be used. The transcription factor may be a member of the TP family. A transcription factor may be TP73. TP73 may also be known as Tumor Protein P73; P73; P53-Like Transcription Factor; P53-Related Protein; or CILD47.

The present disclosure provides an expression cassette comprising one or more transcription factors. TP73 may be introduced in an expression cassette. TP73 may be expressed in an expression cassette. An expression cassette comprising TP73 may be introduced in a PSC. An expression cassette comprising TP73 may be expressed in a PSC. An expression cassette comprising TP73 may induce differentiation of the PSC into a hepatocyte or hepatic cell.

TP73 may be introduced in an expression cassette with one or more other transcription factors. A combination comprising one or more transcription factors may be created. The combination of transcription factors may be introduced in an expression cassette. The combination of transcription factors may be expressed in an expression cassette. The expression cassette may be introduced in a PSC. The expression cassette comprising TP73, and one or more other transcription factors may induce differentiation of the PSC into a hepatocyte or hepatic cell.

The present disclosure provides one or more transcription factors that may induce differentiation of one or more PSCs into one or more hepatocytes or hepatic cells. The one or more transcription factors may comprise TP73. An expression cassette comprising TP73 may be expressed in a PSC. An expression cassette comprising TP73 may induce the differentiation of the PSC into a hepatocyte or a hepatic cell. Different amounts of TP73 may be introduced into the PSC. At least about 5, about 10, about 15, about 20, about 25, or about 50 copies of the open reading frame (ORF) for TP73 per cell may be introduced. Alternatively, or in addition to, at most about 50, about 25, about 20, about 15, about 10, or about 5 copies of the open reading frame (ORF) for TP73 per cell may be introduced. Increased levels of expression may also be achieved by increasing the copy number of the ORF, for example, by using a higher copy number vector or by using a transposon.

In some embodiments, the number of copies of the ORF for TP73 introduced per cell can be about 1 to about 90. In some embodiments, the number of copies of the ORF for TP73 introduced per cell can be at least about 1. In some embodiments, the number of copies of the ORF for TP73 introduced per cell can be at most about 90. In some embodiments, the number of copies of the ORF for TP73 introduced per cell can be about 1 to about 2, about 1 to about 4, about 1 to about 5, about 1 to about 10, about 1 to about 15, about 1 to about 20, about 1 to about 25, about 1 to about 40, about 1 to about 50, about 1 to about 60, about 1 to about 90, about 2 to about 4, about 2 to about 5, about 2 to about 10, about 2 to about 15, about 2 to about 20, about 2 to about 25, about 2 to about 40, about 2 to about 50, about 2 to about 60, about 2 to about 90, about 4 to about 5, about 4 to about 10, about 4 to about 15, about 4 to about 20, about 4 to about 25, about 4 to about 40, about 4 to about 50, about 4 to about 60, about 4 to about 90, about 5 to about 10, about 5 to about 15, about 5 to about 20, about 5 to about 25, about 5 to about 40, about 5 to about 50, about 5 to about 60, about 5 to about 90, about 10 to about 15, about 10 to about 20, about 10 to about 25, about 10 to about 40, about 10 to about 50, about 10 to about 60, about 10 to about 90, about 15 to about 20, about 15 to about 25, about 15 to about 40, about 15 to about 50, about 15 to about 60, about 15 to about 90, about 20 to about 25, about 20 to about 40, about 20 to about 50, about 20 to about 60, about 20 to about 90, about 25 to about 40, about 25 to about 50, about 25 to about 60, about 25 to about 90, about 40 to about 50, about 40 to about 60, about 40 to about 90, about 50 to about 60, about 50 to about 90, or about 60 to about 90. In some embodiments, the number of copies of the ORF for TP73 introduced per cell can be about 1, about 2, about 4, about 5, about 10, about 15, about 20, about 25, about 40, about 50, about 60, or about 90.

The present disclosure provides one or more hepatocytes or hepatic cells. The one or more hepatocytes or hepatic cells may comprise an exogenous expression cassette. The exogenous expression cassette may comprise one or more transcription factors. The one or more transcription factors may comprise TP73. The one or more hepatocytes or hepatic cells may be differentiated from one or more PSCs. The one or more hepatocytes or hepatic cells may comprise one or more transcription factors that may induce differentiation of the one or more PSCs into hepatocytes or hepatic cells.

### Induced Differentiation

The present disclosure provides methods for generating one or more hepatocytes or hepatic cells from one or more PSCs. The method may comprise providing one or more pluripotent stem cells (PSCs). The method can comprise expressing one or more proteins or nucleic acids encoding one or more transcription factors. The method may comprise directly contacting one or more proteins encoding one or more transcription factors with the PSC to differentiate the one or more PSCs into one or more hepatocytes or hepatic cells. The method may comprise delivering the one or more PSCs with at least one exogenous expression cassette. The at least one exogenous expression cassette can comprise one or more transcription factors. The one or more transcription factors can be, for example, FoxA1, HNF1A, SPI1, FoxA2, CEBPA, ONECUTI/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR113, and HIF1A. The expression cassette may comprise two or more transcription factors, for example, FoxA1, HNF1A, SPI1, FoxA2, CEBPA, ONECUTI/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR113, and HIF1A. The expression cassette may comprise three or more transcription factors, for example, FoxA1, HNF1A, SPI1, FoxA2, CEBPA, ONECUT1/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR113, and HIF1A. The expression cassette may comprise four or more transcription factors, for example, FoxA1, HNF1A, SPI1, FoxA2, CEBPA, ONECUT1/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR113, and HIF1A. The expression cassette may comprise five or more transcription factors, for example, FoxA1, HNF1A, SPI1, FoxA2, CEBPA, ONECUTI/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR113, and HIF1A. The expression cassette may comprise six or more transcription factors, for example, FoxA1, HNF1A, SPI1, FoxA2, CEBPA, ONECUTI/HNF6, ONECUT2/HNF6B, HNF4a, Atf3, Fos, CEBPB, FoxM1, a GATA2 transcription factor, GATA4, JUN/AP-1, NFIX, GATA6, TBX3, RXRB, NFE2L2, Myc, TP73, HHEX, SOX17, ATF5, RBPJ, Smad3, NR5A2, NR113, and HIF1A.

### Transcription Factor Recipes or Combinations

In some embodiments as disclosed herein, e.g., one or more, two or more, three or more, four or more, five or more, six or more and so forth, transcription factors (TFs) may be expressed by an exogenous expression cassette to induce or differentiate one or more PSCs into one or more hepatocytes or hepatic cells. The exogenous expression cassettes may induce the expression of the one or more PSCs into the population of hepatocytes or hepatic cells in 96 hours or less. The PSC may be provided in a media. The media may not need any alterations during the transfection of the one or more PSCs into one or more hepatocytes or hepatic cells. In some embodiments, expression cassettes comprising any number of transcription factor (TF) combination disclosed herein including for example, at least one or more TF combinations (also known as TF recipes or TF cocktails, singular/plural inclusive) may induce the expression of one or more PSCs into population of hepatocytes or hepatocyte-like cells, or hepatic cells. For example, the TF recipe or TF combination may comprise one or more transcription factors or at least one transcription factor. In some embodiments, the TF recipe(s) or TF combination(s) may comprise at least one or more transcription factors in any combination of TFs disclosed herein. In the present disclosure, the TF recipe(s) or TF combination(s) is not restricted to any particular transcription factors, TF recipes or TF combinations. As disclosed herein, in some embodiment, the TF recipe or combination may comprise for example, without any limitations, TF recipes including any transcription factor (TF) disclosed in a TF recipe in the present disclosure. For example, the at least one or more TF may be obtained from TF recipe disclosed herein such as for example TF recipe H0001 (SPI1, HNF1A, FOXA2, CEBPA, ONECUT1, HNF4A, ONECUT2), H0002 (HNF1A, ATF3, ONECUTI, ONECUT2, FOS, CEBPA, FOXM1), H0003 (SPI1, FOXA2, GATA2 #1, GATA2 #2, GATA4, JUN, NFIX), H0004 (HNF4A, CEBPA, GATA6, TBX3, FOS, GATA2 #1, GATA2 #2), H0005 (GATA2 #1, HNF4A, RXRB, NFE2L2, MYC, TP73, ONECUT1), H0006 (GATA2 #2, CEBPA, ATF3, GATA6, FOS, GATA3, NFIX), H0008 (HHEX, SOX17, HNF4A, HNF1A, ONECUTI/HNF6, FOXA2, FOXA1), H0009 (HHEX, SOX17, HNF4A), H0010 (CEBPA, ATF5, HHEX, SOX17, HNF4A, ONECUT2, HNF1A), H0011 (FOXA1, HNF1A, FOXA2, CEBPA, ONECUT1, HNF4A, RBPJ), H0011C (FOXA1, HNF1A, FOXA2, CEBPA, ONECUT1, HNF4A, RBPJ), H0012 (SMAD3, HHEX, ATF5, NR5A2, GATA2, CEBPB, NR113), H0013 (HNF1A, CEBPA, ONECUTI, ONECUT2, FOS, HIFIA, TBX3), H0014 (HHEX, SOX17, HNF4A, HNF1A, ONECUT1, FOXA2, FOXA1, CEBPA, RBPJ, ATF5), H0015 (HNF4A, HNF1A, ONECUT1, FOXA2, FOXA1), H0015C (HNF4A, HNF1A, ONECUT1/HNF6, FOXA2, FOXA1), H0015D (HNF4A, HNF1A, ONECUT1/HNF6, FOXA2, FOXA1), H0016 (FOXA1, HNF1A, CEBPA, HNF4A, GATA2 #1, GATA6, PROX1), H0017 (PROX1, FOXA3, SMAD3, ATF5, SOX17, EGR1, NR112/PXR), H0018 (SKI, RXRG, SOX17, RXRB, GATA3, ONECUT2, FOXA1), H0019 (ATF5, PROX1, CEBPA, FOXA3, FOXA2, HNF4A, GATA6), H0020 (FOXA1, HNF1A, FOXA2, CEBPA, ONECUT1, HNF4A, RBPJ), H0021 (HNF4A, HNF1A, ONECUT1, FOXA2, FOXA1), H0022 (GATA6, FOXA2, PROX1, HNF1A, HNF4A, ATF5, FOXA3), H0023 (GATA4, FOXA2, HHEX, PROX1, HNF4A), H0024 (HNF4A, PROX1), H0025 (HNF4A, HNF1A, FOXA2), H0026A (HNF1A, ONECUT1/HNF6, FOXA2, FOXA1), H0026B (HNF1A, ONECUT1/HNF6, FOXA2, FOXA1), H0027A (HNF4A, HNF1A, FOXA2, FOXA1), H0027B (HNF4A, HNF1A, FOXA2, FOXA1), H0028A (HNF4A, HNF1A, ONECUT1/HNF6, FOXA1), H0028B (HNF4A, HNF1A, ONECUT1/HNF6, FOXA1), H0029A (HNF4A, ONECUT1/HNF6, FOXA2, FOXA1), H0029B (HNF4A, ONECUTI/HNF6, FOXA2, FOXA1), H0030A (HNF4A, HNF1A, ONECUT1/HNF6, FOXA2), H0030B (HNF4A, HNF1A, ONECUT1/HNF6, FOXA2), H0031A (FOXA1, HNF1A, CEBPA, ONECUT1/HNF6, HNF4A), H0032A (FOXA1, HNF4A, ONECUT1/HNF6, RBPJ), H0033A (CEBPA, FOXA1, HNF1A, HNF4A, ONECUT1/HNF6, RBPJ), H0034A (CEBPA, FOXA1, HNF1A, HNF4A), H0035A (CEBPA, HNF1A, HNF4A, FOXA3, FOXA2, PXR, RXRA), H0036 (ONECUTI/HNF6, ONECUT2/HNF6B, NR1C1/PPARA, TBX3, SALL4), H0037 (ATF5, FOXA3, HIFIA, HLF, HNF4A, NR1H4/FXR, NR112/PXR, NR5A2, RBPJ), H0038 (CEBPA, FOXA3, GATA6, HNF1A, HNF4A, NR1H4/FXR, NR113/CAR, NR5A2, PROX1, RXRA, TBX3, SALL4), H0039 (ATF5, CEBPA, GATA6, HIFIA, HLF, HNF1A, NR112/PXR, NR113/CAR, ONECUT1/HNF6, ONECUT2/HNF6B, NR1C1/PPARA, RORC), H0040 (CEBPB, FOXA3, GATA6, HHEX, HIFIA, NR1I2/PXR, NR113/CAR, NR5A2, NR1C1/PPARA, RORC, TBX3, SALL4), H0041 (ATF5, CEBPB, GATA6, HHEX, HLF, HNF4A, NR1H4/FXR, NR113/CAR, ONECUT1/HNF6, ONECUT2/HNF6B, PROX1, RXRA), H0042 CEBPA, CEBPB, HHEX, HIFIA, HNF1A, HNF4A, NR1H4/FXR, NR1I2/PXR, ONECUTI/HNF6, ONECUT2/HNF6B, RBPJ, TBX3, SALL4), H0043 (ATF5, CEBPA, CEBPB, FOXA3, HHEX, HLF, HNF1A, NR5A2, NR1C1/PPARA, PROX1, RBPJ, RORC, RXRA), H0044 (FOXA1, GATA6, HHEX, HIFIA, HLF, HNF1A, HNF4A, NR5A2, ONECUT2/HNF6B, RORC, RXRA, SALL4), H0045 (ATF5, FOXA1, FOXA3, GATA6, HHEX, HNF1A, NR1H4/FXR, NR112/PXR, NR113/CAR, ONECUTI/HNF6, NR1C1/PPARA, PROX1, TBX3), H0046 (CEBPA, FOXA1, FOXA3, HHEX, HIFIA, HLF, NR1H4/FXR, NR113/CAR, ONECUTI/HNF6, NR1C1/PPARA, RBPJ, RXRA, SALL4), H0047 (ATF5, CEBPA, FOXA1, HHEX, HNF4A, NR112/PXR, NR113/CAR, NR5A2, ONECUT2/HNF6B, PROX1, RBPJ, RORC, TBX3), H0048 (CEBPB, FOXA1, FOXA3, HLF, HNF1A, HNF4A, NR112/PXR, NR113/CAR, ONECUTI/HNF6, PROX1, RBPJ, RORC, SALL4), H0049 (ATF5, CEBPB, FOXA1, HIFIA, HNF1A, NR1H4/FXR, NR113/CAR, NR5A2, ONECUT2/HNF6B, NR1CI/PPARA, RBPJ, RXRA, TBX3), H0050 (CEBPA, CEBPB, FOXA1, GATA6, HLF, NR1H4/FXR, NR112/PXR, NR5A2, ONECUT2/HNF6B, NR1C1/PPARA, PROX1, SALL4), H0051 (ATF5, CEBPA, CEBPB, FOXA1, FOXA3, GATA6, HIFIA, HNF4A, ONECUTI/HNF6, RORC, RXRA, TBX3), H0052 (FOXA2, HHEX, HLF, HNF1A, NR1H4/FXR, NR112/PXR, NR113/CAR, NR5A2, ONECUT1/HNF6, RORC, RXRA, TBX3), H0053 (ATF5, FOXA2, FOXA3, HHEX, HIFIA, HNF1A, HNF4A, NR113/CAR, ONECUT2/HNF6B, NR1C1/PPARA, PROX1, SALL4), H0054 (CEBPA, FOXA2, FOXA3, GATA6, HHEX, HLF, HNF4A, NR1I2/PXR, ONECUT2/HNF6B, NR1C1/PPARA, RBPJ, RXRA, TBX3), H0055 (ATF5, CEBPA, FOXA2, GATA6, HHEX, HIFIA, NR1H4/FXR, NR5A2, ONECUT1/HNF6, PROX1, RBPJ, RORC, SALL4), H0056 (CEBPB, FOXA2, FOXA3, GATA6, HIFIA, HLF, HNF1A, NR1H4/FXR, ONECUT2/HNF6B, PROX1, RBPJ, RORC, TBX3), H0057 (ATF5, CEBPB, FOXA2, GATA6, HNF1A, HNF4A, NR1I2/PXR, NR5A2, ONECUTI/HNF6, NR1C1/PPARA, RBPJ, RXRA, SALL4), H0058 (CEBPA, CEBPB, FOXA2, HIFIA, HNF4A, NR113/CAR, NR5A2, ONECUT1/HNF6, NR1C1/PPARA, PROX1, TBX3), H0059 (ATF5, CEBPA, CEBPB, FOXA2, FOXA3, HLF, NR1H4/FXR, NR112/PXR, NR113/CAR, ONECUT2/HNF6B, RORC, RXRA, SALL4), H0060 (FOXA1, FOXA2, GATA6, HIFIA, HNF4A, NR1H4/FXR, NR1I2/PXR, NR113/CAR, NR1C1/PPARA, PROX1, RBPJ, RORC, RXRA), H0061 (ATF5, FOXA1, FOXA2, FOXA3, GATA6, HLF, NR113/CAR, NR5A2, ONECUTI/HNF6, ONECUT2/HNF6B, RBPJ, TBX3, SALL4), H0062 (CEBPA, FOXA1, FOXA2, FOXA3, HIF1A, HNF1A, NR112/PXR, NR5A2, ONECUTI/HNF6, ONECUT2/HNF6B, PROX1, RXRA), H0063 (ATF5, CEBPA, FOXA1, FOXA2, HLF, HNF1A, HNF4A, NR1H4/FXR, NR1C1/PPARA, RORC, TBX3, SALL4), H0064 (CEBPB, FOXA1, FOXA2, FOXA3, HHEX, HNF4A, NR1H4/FXR, NR5A2, ONECUT1/HNF6, ONECUT2/HNF6B, NR1C1/PPARA, RORC), H0065 (ATF5, CEBPB, FOXA1, FOXA2, HHEX, HIFIA, HLF, NR1I2/PXR, PROX1, RXRA, TBX3, SALL4), H0066 (CEBPA, CEBPB, FOXA1, FOXA2, GATA6, HHEX, HNF1A, NR113/CAR, RBPJ), H0067 (ATF5, CEBPA, CEBPB, FOXA1, FOXA2, FOXA3, GATA6, HHEX, HIFIA, HLF, HNF1A, HNF4A, NR1H4/FXR, NR1I2/PXR, NR113/CAR, NR5A2, ONECUT1/HNF6, ONECUT2/HNF6B, NR1C1/PPARA, PROX1, RBPJ, RORC, RXRA, TBX3, SALL4). In some embodiments, the exogenous expression cassettes of the present disclosure may induce the expression of the one or more PSCs into the population of hepatocytes or hepatic cells in 96 hours or less. In some embodiments, provided herein are additional transcription factors (TFs) which can be used to differentiate pluripotent stem cells (PSCs) into hepatocytes or hepatocyte-like cells or hepatic cells. For example, such transcriptional factors include any TFs provided herein including for example, in Table 1 to Table 12. In some embodiments, the PSC may be provided in a media. The media may need alterations. In some instances, the media may not need alternation. For example, the media may not need any alterations during the transfection of the one or more PSCs into one or more hepatocytes or hepatic cells. In some embodiments, expression cassettes comprising any number of transcription factor (TF) combination and/or combinations of TF families disclosed herein including for example, at least one or more TF combinations (also known as TF recipes or TF cocktails, singular/plural inclusive) from any of the TF families disclosed herein and which may induce the expression of one or more PSCs into population of hepatocytes or hepatocyte-like cells, or hepatic cells.

Methods as described herein may be used to generate a population of one or more hepatocytes or hepatic cells. The method can comprise generating the population of hepatocytes or hepatic cells from the one or more PSCs. The method can comprise providing one or more pluripotent stem cells (PSCs). The method can comprise delivering the one or more PSCs with at least one exogenous expression cassette. The at least one exogenous expression cassette can comprise a transcription factor. The transcription factor can induce differentiation of the one or more PSCs into the population of hepatocytes or hepatic cells. The method can comprise inducing the expression of the at least one exogenous expression cassette in the one or more PSCs in 96 hours or less. The method can comprise generating the population of hepatocytes or hepatic cells from the one or more PSCs. The population of cells may comprise adherent cells. The population of cells may comprise suspension cells. The population of cells may comprise adherent cells and suspension cells. The population of cells may be provided in a media. The media may not need any nutrients, growth factors, or microenvironmental or matrix optimizations. At least 5% of the cells may express ASGR-1 or CXCR-4. At least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, or at least about 40% of the cells may express ASGR-1 or CXCR-4.

The present disclosure provides methods for expressing, or for increasing expression of, a transcription factor. A transcription factor may be expressed or have increased expression in a cell through various methods. This may involve delivery of either a nucleic acid comprising an open reading frame encoding the transcription factor, delivery of the transcription factor itself, or delivery of an activator of the transcription factor or its expression. Any technique known in the art, for such delivery may be used. For example, for delivery of a cDNA, a viral or plasmid vector may be used. The open reading frame (encoding any isoform of the TF) may be inducible or repressible for control, to achieve a suitable level of expression. The nucleic acid comprising the open reading frame may be a complementary DNA (cDNA), a messenger RNA (mRNA), or a synthetic or engineered nucleic acid. Some transcription factors may require a critical amount of expression to effectively induce differentiation, such as the equivalent of at least 5, 10, 15, 20, 25, or 50 copies of the ORF per cell. Other factors may require less than a certain threshold of expression due to possible toxicity at high levels, such as less than 20, 10, or 5 copies per cell. Increased levels of expression may also be achieved by increasing the copy number of the ORF, for example, by using a higher copy number vector or by using a transposon. Modified RNAs, RNAs that encode the transcription factor but use synthetic nucleotides that improve stability and reduce degradation, may be used. Use of culture media adapted for a particular cell type may increase the expression of the ORF that induces expression of that cell type. Expression of an ORF can be increased from a non-expressed gene, from a gene expressed at a low level, or from a gene expressed at a robust level. The present disclosure provides methods for expressing, or for increasing expression of, a transcription factor as described in Ng, Alex H M, et al. “A comprehensive library of human transcription factors for cell fate engineering.” Nature biotechnology 39.4 (2021): 510-519; PCT Application Number PCT/US2017/051122; and PCT Application Number PCT/US2018/030216, which are entirely incorporated herein by reference.

It will be understood that methods for increasing the expression of the transcription factors in the cells to be programmed into hepatic cells may include any method known in the art, for example, by induction of expression of one or more expression cassettes previously introduced into the cells, or by introduction of nucleic acids (such as DNA or RNA), polypeptides, or small molecules to the cells. Increasing the expression of certain endogenous but transcriptionally repressed genes may also reverse the silencing or inhibitory effect on the expression of these genes by regulating the upstream transcription factor expression or epigenetic modulation. Therefore, methods of the invention may involve culturing the cell population under conditions to artificially increase the expression level of one or more of the transcription factors described herein.

Contact with Transcription Factors

The present disclosure provides methods for expressing, or for increasing expression of, a transcription factor. The expression of the transcription factors may be increased by contacting at least one cell with one or more proteins encoding transcription factors. The at least one cell may be a stem cell. The stem cell may be a pluripotent stem cell. The pluripotent stem cell may be an induced pluripotent stem cell (iPSC). Delivery of the transcription factors may occur using direct electroporation of transcription factor proteins to the cells.

Direct Contact with Agents

The present disclosure provides methods for expressing, or for increasing expression of, a transcription factor. The expression of the transcription factors may be increased by contacting at least one cell with one or more agents that activate or increase the expression or amount of the transcription factors. The at least one cell may be a stem cell. The stem cell may be a pluripotent stem cell. The pluripotent stem cell may be an induced pluripotent stem cell (iPSC). Delivery of the transcription factors may occur using direct electroporation of transcription factor proteins to the cells. The agent may be a nucleic acid (i.e., polynucleotide, e.g., messenger RNA (mRNA), coding DNA sequence), a protein, an aptamer and small molecule, ribosome, RNAi agent, guide RNA (gRNA) and peptide-nucleic acid (PNA) and analogues or variants thereof. In one embodiment, the agent is a transcriptional activation system (e.g., a gRNA for use in a gene activation system such as CRISPR/Cas9 or TALEN) for increasing the expression of the one or more endogenous transcription factors.

### Open Reading Frame

The present disclosure provides methods for expressing, or for increasing expression of, a transcription factor. The expression of the transcription factors may be increased by inducing differentiation of at least one cell by delivering to the at least one cell a nucleic acid comprising an open reading frame encoding one or more of the transcription factors, the transcription factor protein, or an activator of transcription of the open reading frame encoding one or more transcription factors. The one or more transcription factors may be delivered in an expression cassette. The at least one cell may be a stem cell. The stem cell may be a pluripotent stem cell. The pluripotent stem cell may be an induced pluripotent stem cell (iPSC).

### Exogenous Expression Cassette

The present disclosure provides methods for expressing, or for increasing expression of, a transcription factor. The expression of the transcription factors may be increased by delivering one or more nucleic acids encoding a transcription factor or an activator of transcription of the open reading frame encoding one or more transcription factors. The nucleic acid may comprise a recombinant or exogenous expression cassette encoding one or more transcription factors. The recombinant or exogenous expression cassette may induce the cell to differentiate into a hepatocyte or hepatic cell. The at least one cell may be a stem cell. The stem cell may be a pluripotent stem cell. The pluripotent stem cell may be an induced pluripotent stem cell (iPSC). One or more recombinant or exogenous expression cassettes may be used. The exogenous expression cassette may comprise an externally inducible transcriptional regulatory element for inducible expression of the one or more transcription factors, such as an inducible promoter, e.g., comprising a tetracycline response element or variant thereof. Any suitable system for delivering the sequence may be used. The gene delivery system may be a transposon system; a viral gene delivery system; an episomal gene delivery system; or a homologous recombination system such as utilizing a zinc finger nuclease, a transcription activator-like effector nuclease (TALENs), or a meganuclease, or a CRISPR/Cas9, or the like.

The exogenous expression cassette may include cleavable sequences. Such sequences are sequences that are recognized by an entity capable of specifically cutting DNA, and include restriction sites, which are the target sequences for restriction enzymes or sequences for recognition by other DNA cleaving entities, such as nucleases, recombinases, ribozymes or artificial constructs. At least one cleavable sequence may be included, but preferably two or more are present. These cleavable sequences may be at any suitable point in the cassette, such that a selected portion of the cassette, or the entire cassette, can be selectively removed if desired. The cleavable sites may thus flank the part/all of the genetic sequence that it may be desired to remove. The method may therefore also comprise removal of the expression cassette and/or the genetic material.

### Direct Delivery of Nucleic Acids

The present disclosure provides methods for expressing, or for increasing expression of, a transcription factor. The expression of the transcription factors may be increased by directly delivering one or more nucleic acids to a cell. The at least one cell may be a stem cell. The stem cell may be a pluripotent stem cell. The pluripotent stem cell may be an induced pluripotent stem cell (iPSC). The nucleic acid may be a DNA or RNA (or nucleic acid in any form, e.g., single stranded, double stranded, etc.). The one or more nucleic acids may be delivered into cells using any suitable methods for nucleic acid delivery for transformation of a cell known in the art. Such methods include, but are not limited to, direct delivery of DNA such as by ex vivo transfection, by injection (including microinjection), by electroporation, by calcium phosphate precipitation, by using DEAE-dextran followed by polyethylene glycol, by direct sonic loading, by liposome mediated transfection, by receptor-mediated transfection, by microprojectile bombardment, by agitation with silicon carbide fibers, by Agrobacterium-mediated transformation, and any combination of such methods.

### Vectors

The present disclosure provides methods for expressing, or for increasing expression of, a transcription factor. The expression of the transcription factors may be increased by delivering a nucleic acid comprising an open reading frame encoding one or more of the transcription factors, the transcription factor protein, or an activator of transcription of the open reading frame encoding one or more transcription factors to at least one cell. The at least one cell may be a stem cell. The stem cell may be a pluripotent stem cell. The pluripotent stem cell may be an induced pluripotent stem cell (iPSC). The nucleic acids may be introduced into the cell using a vector. A vector can be constructed through standard techniques known in the art. Vectors include but are not limited to plasmids, cosmids, viruses (bacteriophage, animal viruses, and plant viruses), and artificial chromosomes (e.g., YACs).

The vector may be a viral vector. The viral gene delivery system may be an RNA-based or DNA-based viral vector. Viral vectors include retroviral vectors, lentiviral vectors (e.g., derived from HIV-1, HIV-2, SIV, BIV, FIV etc.), gammaretroviral vectors, adenoviral (Ad) vectors (including replication competent, replication deficient and gutless forms thereof), adeno-associated virus-derived (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors and Sendai virus vectors. The viral vector may be selected from a lentiviral vector, an adeno-associated virus vector or a Sendai virus vector. In a yet further embodiment, the viral vector is a lentiviral vector. In one embodiment, the viral vector is used at a high multiplicity of infection (MOI). A high MOI helps to ensure that more than one transcription factor is introduced into the source cell. In one embodiment, the MOI is greater than 0.5, such as 1.0 or above.

### Plasmids

The present disclosure provides methods for expressing, or for increasing expression of, a transcription factor. The expression of the transcription factors may be increased by delivering cell a nucleic acid comprising an open reading frame encoding one or more of the transcription factors, the transcription factor protein, or an activator of transcription of the open reading frame encoding one or more transcription factors to at least one cell. The at least one cell may be a stem cell. The stem cell may be a pluripotent stem cell. The pluripotent stem cell may be an induced pluripotent stem cell (iPSC). The nucleic acids may be introduced into the cell using a plasmid.

The plasmid may be episomal. Episomal vectors are able to introduce large fragments of DNA into a cell but are maintained extra-chromosomally, replicated once per cell cycle, partitioned to daughter cells efficiently, and elicit substantially no immune response. In alternative embodiments, an Epstein-Barr virus (EBV)-based episomal vector, a yeast-based vector, an adenovirus-based vector, a simian virus 40 (SV40)-based episomal vector, or a bovine papilloma virus (BPV)-based vector may be used.

### Site-Specific Delivery

Any suitable technique for insertion of a nucleic acid sequence into a specific sequence may be used, and several are described in the art. Suitable techniques include any method which introduces a break at the desired location and permits recombination of the vector into the gap. Thus, a crucial first step for targeted site-specific genomic modification is the creation of a double-strand DNA break (DSB) at the genomic locus to be modified. Distinct cellular repair mechanisms can be exploited to repair the DSB and to introduce the desired sequence, and these are non-homologous end joining repair (NHEJ), which is more prone to error; and homologous recombination repair (HR) mediated by a donor DNA template, that can be used to insert inducible cassettes.

Several techniques exist to allow customized site-specific generation of DSB in the genome. Many of these involve the use of customized endonucleases, such as zinc finger nucleases, TALENs or the clustered regularly interspaced short palindromic repeats/CRISPR associated protein (CRISPR/Cas9) system.

Zinc finger nucleases are artificial enzymes which are generated by fusion of a zinc-finger DNA-binding domain to the nuclease domain of the restriction enzyme FokI. The latter has a non-specific cleavage domain which must dimerize in order to cleave DNA. This means that two zinc finger nuclease monomers are required to allow dimerization of the FokI domains and to cleave the DNA. The DNA binding domain may be designed to target any genomic sequence of interest, is a tandem array of Cys2His2 zinc fingers, each of which recognizes three contiguous nucleotides in the target sequence. The two binding sites are separated by 5-7 bp to allow optimal dimerization of the FokI domains. The enzyme thus is able to cleave DNA at a specific site, and target specificity is increased by ensuring that two proximal DNA-binding events must occur to achieve a double-strand break.

Transcription activator-like effector nucleases, or TALENs, are dimeric transcription factor/nucleases. They are made by fusing a TAL effector DNA-binding domain to a DNA cleavage domain (a nuclease). Transcription activator-like effectors (TALEs) can be engineered to bind practically any desired DNA sequence, so when combined with a nuclease, DNA can be cut at specific locations. TAL effectors are proteins that are secreted by Xanthomonas bacteria, the DNA binding domain of which contains a repeated highly conserved 33-34 amino acid sequence with divergent 12th and 13th amino acids. These two positions are highly variable and show a strong correlation with specific nucleotide recognition. This straightforward relationship between amino acid sequence and DNA recognition has allowed for the engineering of specific DNA-binding domains by selecting a combination of repeat segments containing appropriate residues at the two variable positions. TALENs are thus built from arrays of 33 to 35 amino acid modules, each of which targets a single nucleotide. By selecting the array of the modules, almost any sequence may be targeted. Again, the nuclease used may be FokI or a derivative thereof.

Three types of CRISPR mechanisms have been identified, of which type II is the most studied. The CRISPR/Cas9 system (type II) utilizes the Cas9 nuclease to make a double-stranded break in DNA at a site determined by a short guide RNA. The CRISPR/Cas system is a prokaryotic immune system that confers resistance to foreign genetic elements. CRISPR are segments of prokaryotic DNA containing short repetitions of base sequences. Each repetition is followed by short segments of “protospacer DNA” from previous exposures to foreign genetic elements. CRISPR spacers recognize and cut the exogenous genetic elements using RNA interference. The CRISPR immune response occurs through two steps: CRISPR-RNA (crRNA) biogenesis and crRNA-guided interference. CrRNA molecules are composed of a variable sequence transcribed from the protospacer DNA and a CRISPR repeat. Each crRNA molecule then hybridizes with a second RNA, known as the trans-activating CRISPR RNA (tracrRNA) and together these two eventually form a complex with the nuclease Cas9. The protospacer DNA encoded section of the crRNA directs Cas9 to cleave complementary target DNA sequences, if they are adjacent to short sequences known as protospacer adjacent motifs (PAMs). This natural system has been engineered and exploited to introduce DSB breaks in specific sites in genomic DNA, amongst many other applications. In particular, the CRISPR type II system from Streptococcus pyogenes may be used. At its simplest, the CRISPR/Cas9 system comprises two components that are delivered to the cell to provide genome editing: the Cas9 nuclease itself and a gRNA. The gRNA is a fusion of a customized, site-specific crRNA (directed to the target sequence) and a standardized tracrRNA.

Once a DSB has been made, a donor template with homology to the targeted locus is supplied; the DSB may be repaired by the homology-directed repair (HDR) pathway allowing for precise insertions to be made.

Derivatives of this system are also possible. Mutant forms of Cas9 are available, such as Cas9D10A, with only nickase activity. This means it cleaves only one DNA strand and does not activate NHEJ. Instead, when provided with a homologous repair template, DNA repairs are conducted via the high-fidelity HDR pathway only. Cas9D10A may be used in paired Cas9 complexes designed to generate adjacent DNA nicks in conjunction with two sgRNAs complementary to the adjacent area on opposite strands of the target site, which may be particularly advantageous.

The elements for making the double-strand DNA break may be introduced in one or more vectors, such as plasmids, for expression in the cell.

Thus, any method of making specific, targeted double strand breaks in the genome in insert a gene or inducible cassette may be used in the method of the invention. It may be preferred that the method for inserting the gene/inducible cassette utilizes any one or more of zinc finger nucleases, TALENs and/or CRISPR/Cas9 systems or any derivative thereof.

Once the DSB has been made by any appropriate means, the gene/inducible cassette for insertion may be supplied in any suitable fashion as described below. The gene/inducible cassette and associated genetic material form the donor DNA for repair of the DNA at the DSB and are inserted using standard cellular repair machinery/pathways. How the break is initiated will alter which pathway is used to repair the damage, as noted above.

### Controlled Expression

In one embodiment, expression of the transcription factors is under controlled transcription. In this aspect of the invention, the transcription and translation (expression) of the transcription factors may be controlled within the cell. This permits overexpression of the transcription factor(s), if required.

An exogenous expression cassette carrying the transcription factors may comprise an externally inducible transcriptional regulatory element (i.e., an inducible promoter) for inducible expression of the transcription factors. The inducible expression cassette may be controlled by addition of an exogenous substance. Whatever culturing conditions are used, the exogenous substance will control expression of the genetic sequence within the inducible expression cassette; and may either be supplied continuously and then withdrawn in order to induce transcription or supplied as transcription is required, dependent upon its mode of action.

Expression of the transcription factors described herein may be increased using a dual cassette expression system. This system targets genetic safe harbor (GSH) sites which provides a reduced risk of epigenetic silencing of the inserted genetic material.

A GSH site is a locus within the genome wherein a gene or other genetic material may be inserted without any deleterious effects on the cell or on the inserted genetic material. Most beneficial is a GSH site in which expression of the inserted gene sequence is not perturbed by any read-through expression from neighboring genes and expression of the inducible cassette minimizes interference with the endogenous transcription program. More formal criteria have been proposed that assist in the determination of whether a particular locus is a GSH site in future (Papapetrou et al., (2011)) These criteria include a site that is (i) 50 kb or more from the 5′ end of any gene, (ii) 300 kb or more from any gene related to cancer, (iii) 300 kb or more from any microRNA (miRNA), (iv) located outside a transcription unit and (v) located outside ultra-conserved regions (UCR). It may not be necessary to satisfy all of these proposed criteria, since GSH already identified do not fulfil all of the criteria. It is thought that a suitable GSH will satisfy at least 2, 3, 4 or all of these criteria. Any suitable GSH site may be used in the method of the invention, on the basis that the site allows insertion of genetic material without deleterious effects to the cell and permits transcription of the inserted genetic material. Those skilled in the art may use these simplified criteria to identify a suitable GSH, and/or the more formal criteria set out above.

Specific insertion of genetic material into the particular GSH based upon customized site-specific generation of DNA double-strand breaks at the GSH may be achieved. The genetic material may then be introduced using any suitable mechanism, such as homologous recombination. Any method of making a specific DSB in the genome may be used, but preferred systems include CRISPR/Cas9 and modified versions thereof, zinc finger nucleases and the TALEN system.

One or more genetic sequences may be controllably transcribed from within the second and/or further GSH. Indeed, the inducible cassette may contain 1, 2, 3, 4, 5, 6, 7, 8, 9 or 10 genetic sequences (e.g., transcription factor sequences) which it is desired to insert into the GSH and the transcription of which be controllably induced. Therefore, the transcription factors required by the present invention may be included within the same cassette introduced into the second genetic safe harbor site. For example, the three or more transcription factors may be included in, for example, three mono-cistronic constructs, one mono-cistronic and one bi-cistronic construct or one tri-cistronic construct. It will be understood that similar combinations of constructs may be used to achieve higher orders of transcription factor expression.

Alternatively, if a combination of transcription factors is used, the individual transcription factors may be introduced into separate GSHs and/or under the control of different inducible promoters. Therefore, in one embodiment, the at least three or more transcription factors are introduced into separate GSHs. This may be achieved by utilizing three or more different GSH sites for the three or more transcription factors (i.e., wherein the transcription factors are introduced as mono-cistronic cassettes). Alternatively, this may be achieved by utilizing the fact that a GSH exists at the same genetic loci on both chromosomes of diploid organisms, e.g., introducing one transcription factor into the GSH on one chromosome and a different transcription factor into the same GSH on the other chromosome. This embodiment is advantageous if different expression levels or timing of expression of the transcription factors is desired. In one embodiment, the method comprises targeted insertion of the at least three or more transcription factors, each operably linked to an inducible promoter into a second, third and fourth genetic safe harbor site of the source cell. The inducible promoter may be the same of each transcription factor and therefore are all regulated by the transcriptional regulator protein.

A transcriptional regulator protein is a protein that binds to DNA, preferably sequence-specifically to a DNA site located in or near a promoter, and either facilitating the binding of the transcription machinery to the promoter, and thus transcription of the DNA sequence (a transcriptional activator) or blocks this process (a transcriptional repressor).

The DNA sequence that a transcriptional regulator protein binds to is called a transcription factor-binding site or response element, and these are found in or near the promoter of the regulated DNA sequence. Transcriptional activator proteins bind to the response element and promote gene expression. Such proteins are preferred in the methods of the present invention for controlling inducible cassette expression. Transcriptional repressor proteins bind to the response element and prevent gene expression.

Transcriptional regulator proteins may be activated or deactivated by a number of mechanisms including binding of a substance, interaction with other transcription factors (e.g., homo- or hetero-dimerization) or coregulatory proteins, phosphorylation, and/or methylation. The transcriptional regulator protein may be controlled by activation or deactivation. Any suitable transcriptional regulator protein may be used, preferably one that may be activated or deactivated. It is preferred that an exogenous substance may be supplied to control the transcriptional regulator protein. Such transcriptional regulator proteins are also called inducible transcriptional regulator proteins.

### Tetracycline-Controlled Transcriptional Activation

Tetracycline-Controlled Transcriptional Activation is a method of inducible gene expression where transcription is reversibly turned on or off in the presence of the antibiotic tetracycline or one of its derivatives (e.g., doxycycline which is more stable). In this system, the transcriptional activator protein is tetracycline-responsive transcriptional activator protein (rtTa) or a derivative thereof. The rtTA protein is able to bind to DNA at specific TetO operator sequences. Several repeats of such TetO sequences are placed upstream of a minimal promoter (such as the CMV promoter), which together form a tetracycline response element (TRE). There are two forms of this system, depending on whether the addition of tetracycline or a derivative activates (Tet-On) or deactivates (Tet-Off) the rtTA protein.

In a Tet-Off system, tetracycline or a derivative thereof binds rtTA and deactivates the rtTA, rendering it incapable of binding to TRE sequences, thereby preventing transcription of TRE-controlled genes. This system was first described in Gossen et al., (1992).

The Tet-On system is composed of two components; (1) the constitutively expressed tetracycline-responsive transcriptional activator protein (rtTa) and the rtTa-sensitive inducible promoter (Tet Responsive Element, TRE). This may be bound by tetracycline or its more stable derivatives, including doxycycline (dox), resulting in activation of rtTa, allowing it to bind to TRE sequences and inducing expression of TRE-controlled genes. The use of this may be preferred in the method of the invention.

Thus, the transcriptional regulator protein may thus be tetracycline-responsive transcriptional activator protein (rtTa) protein, which can be activated or deactivated by the antibiotic tetracycline or one of its derivatives, which are supplied exogenously. If the transcriptional regulator protein is rtTA, then the inducible promoter inserted into the second GSH site includes the tetracycline response element (TRE). The exogenously supplied substance is the antibiotic tetracycline or one of its derivatives. Variants and modified rtTa proteins may also be used in the methods of the invention, these include Tet-On Advanced transactivator (also known as rtTA2S-M2) and Tet-On 3G (also known as rtTA-V16, derived from rtTA2S-52).

The tetracycline response element (TRE) generally consists of 7 repeats of the 19 bp bacterial TetO sequence separated by spacer sequences, together with a minimal promoter. Variants and modifications of the TRE sequence are possible since the minimal promoter can be any suitable promoter. Preferably the minimal promoter shows no or minimal expression levels in the absence of rtTa binding. The inducible promoter inserted into the second GSH may thus comprise a TRE.

A modified system based upon tetracycline control is the T-REX System (Thermo-Fisher Scientific), in which the transcriptional regulator protein is a transcriptional repressor protein, TetR. The components of this system include (i) an inducible promoter comprising a strong human cytomegalovirus immediate-early (CMV) promoter and two tetracycline operator 2 (TetO2) sites, and a Tet repressor (TetR). In the absence of tetracycline, the Tet repressor forms a homodimer that binds with extremely high affinity to each TetO2 sequence in the inducible promoter and prevent transcription from the promoter. Once added, tetracycline binds with high affinity to each Tet repressor homodimer rendering it unable to bind to the Tet operator. The Tet repressor: tetracycline complex then dissociates from the Tet operator and allows induction of expression. In this instance, the transcriptional regulator protein is TetR and the inducible promoter comprises two TetO2 sites. The exogenously supplied substance is tetracycline or a derivative thereof. Other inducible expression systems are known and can be used in the method of the invention. These include the Complete Control Inducible system from Agilent Technologies. This is based upon the insect hormone ecdysone or its analogue ponasterone A (ponA) which can activate transcription in mammalian cells which are transfected with both the gene for the Drosophila melanogaster ecdysone receptor (EcR) and an inducible promoter comprising a binding site for the ecdysone receptor. The EcR is a member of the retinoid-X-receptor (RXR) family of nuclear receptors. In humans, EcR forms a heterodimer with RXR that binds to the ecdysone-responsive element (EcRE). In the absence of PonA, transcription is repressed by the heterodimer.

### Applications

The differentiated cells that can be produced using the methods described in the present disclosure may have multiple applications. They can be used for regenerative medicine, such as transplanting the cells into a recipient in need of a certain type of cell. They can be used for drug testing, both in cell culture as well as after transplantation. The cells may be used to deliver a product to a part of a body, for example, if they naturally produce or are engineered to produce and secrete the product.

Drug testing in the cells may use substances that are known or unknown to have a certain biological activity. The substances may be elements, compounds, or mixtures, whether natural or synthetic. The cells may be used to determine a desirable activity of a potential drug or conversely to determine undesirable effects of a substance or lack of such effects. The contacting of the substance with the cells may be in culture or in a human or animal body. The activity or side effects of the substance may be determined in vitro or in vivo, irrespective of where the contacting occurred.

The cells can be observed for effects on cell growth, apoptosis, secreted products, expression of particular products, etc. The genome of these cells may be edited to match mutations found in patients with disease. Any type of assay known in the art for such changes may be used, including but not limited to immunological assays, morphological observations, histochemical stains, reverse transcription polymerase chain reaction, protein blots, mass spectrometry, hybridization assays, electrophysiology, etc.

The stem cells may be obtained from source. One particularly useful source is human induced pluripotent stem cells. Mouse induced pluripotent stem cells and mouse embryonic stem cells may also be used, as well as such cells from other animals. The use of human embryonic stem cells may be regulated or ethically undesirable, but these may be used as well. Differentiated cells may be identified by any property or set of properties that is characteristic or defining of that type of differentiated cell. For example, different cell types have a unique transcriptome. The transcriptome may be used as a means of matching and identifying an unknown cell type to a known cell type. The transcriptome may be used qualitatively or quantitatively. Similarly, a proteome may be used a means of identifying an unknown differentiated cell type. Some cell types may be identifiable based on morphology, growth habit, secretion products, enzymatic activity, cellular function, and the like. Any means known in the art for identifying cells may be used.

### Definitions

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Wherever the term “cells” disclosed in the methods of the present disclosure e.g., modified cells, expressed cells, or in cell lines etcetera, shall be taken to mean non-naturally occurring cells or cell lines. Alternatively, the term “cells” in this disclosure, e.g., modified cells, expressed cells, or cell lines (as understood by those of skill in the art) shall be taken to mean isolated cells or in addition such cells can be isolated e.g., from any contaminants or removed from a composition, a cell culture etcetera.

Whenever the term “deliver,” is disclosed in the methods of the present disclosure e.g. delivering to the one or more PSCs at least one nucleic acid comprising an open reading frame encoding one or more transcription factors, one or more transcription factors, or activator of transcription of the open reading frame encoding one or more transcription factors, the term “deliver,” shall mean any way of inducing a cell to express the described transcription factors e.g. transfection with DNA and/or RNA, viral infection, and supplementing the cell culture media with the one or more transcription factor proteins.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Overexpression is expression at level that is higher than the level that is expressed before induction from a gene that is expressed at a low, medium, or high level. An exogenous open reading frame is typically an open reading frame that differs from the similar gene or mRNA in the cell. It may be engineered to have a different control sequence or sequences, such as promoter, operator, enhancer, terminator, etc. It may be engineered to have no introns. It may be engineered to be fused to a second open reading frame to which it is not fused in the human genome.

As used herein, a “hepatic cell” refers to a cell that is related to parenchymal cells of the liver. Hepatic cells may refer to cells that have one or more characteristic of an adult hepatocyte, adult and fetal hepatic progenitor cells (including hepatobiliary bipotential progenitors), hepatoblasts, and fetal hepatocytes. This term includes further cells with the capacity to engraft liver tissue when transplanted in vivo. The hepatic cells produced by this method may be at least as functional as the hepatic cells produced by directed differentiation to date. Hepatic cells may express ASGR-1. Hepatic cells may express CXCR-4. Hepatic cells may express both ASGR-1 and CXCR-4. In a population of two or more hepatic cells, at least 2% of the hepatic cells may express ASGR-1. In a population of two or more hepatic cells, at least 10% of the hepatocytes or the hepatic cell express CXCR-4.

The above disclosure generally describes the present invention. All references disclosed herein are expressly incorporated by reference. A more complete understanding can be obtained by reference to the following specific example, which are provided herein for purposes of illustration only, and is not intended to limit the scope of the invention.

### Examples

### Example 1: Expression of Hepatocyte-Specific Markers

In order to deliberately induce hepatocyte formation, sub-libraries of transcription factors (TFs), or “recipes”, H0001 to H0065, were generated (Tables 1-11). These TFs are not known to be naturally expressed in pluripotent stem cells; they are typically only expressed in restricted progenitor cells. For these experiments, the changes in cell morphology were monitored using various assays, e.g., as depicted in Table A below.

TABLE A
Morphology and ASGR-1 expression in iPSCs after
induction with transcription factor recipes

| TF | Differentiated Morphology After | Percent of ASGR1 |
|---|---|---|
| Recipe | Induction | positive cells |
| H0001 | Individual flattened cells and epithelial- | 3% |
| | like cells | |
| H0002 | Individual flattened cells and epithelial- | n.s. |
| | like cells | |
| H0003 | Fibroblast-like cells and elongated cells | n.s. |
| H0004 | Epithelial-like cells | n.s. |
| H0005 | iPSC morphology | n.s. |
| H0006 | Epithelial-like cells and fibroblast-like | n.s. |
| | cells | |
| H0008 | Large, flat, polygonal cells | 5% |
| H0009 | Epithelial-like cells and fibroblast-like | n.s. |
| | cells | |
| H0010 | Large, flat, polygonal cells; epithelial | 2% |
| | cells | |
| H0011 | Large, flat, polygonal cells | 6% |
| H0012 | iPSC morphology; fibroblast-like cells | n.s. |
| H0013 | Granular cells; epithelial-like cells | n.s. |

TABLE 1
Libraries of transcription factors
recipes (or cocktails) H0001-H0006

| H0001 | H0002 | H0003 | H0004 | H0005 | H0006 |
|---|---|---|---|---|---|
| SPI1 | HNF1A | SPI1 | HNF4a | GATA2 | GATA2 |
| | | | | isoform | isoform |
| | | | | P23769-2 | P23769-1 |
| HNF1A | Atf3 | FoxA2 | CEBPA | HNF4a | CEBPA |
| FoxA2 | HNF6 | GATA2 | GATA6 | RXRB | Atf3 |
| | | isoform | | | |
| | | P23769-2 | | | |
| CEBPA | HNF6B | GATA4 | TBX3 | NFE2L2 | GATA6 |
| HNF6 | Fos | GATA2 | Fos | Myc | Fos |
| | | isoform | | | |
| | | P23769-1 | | | |
| HNF4a | CEBPA | JUN/AP-1 | GATA2 | TP73 | GATA3 |
| | | | isoform | | |
| | | | P23769-2 | | |
| HNF6B | FoxM1 | NFIX | GATA2 | HNF6 | NFIX |
| | | | isoform | | |
| | | | P23769-1 | | |

TABLE 2
Libraries of transcription factors recipes (or cocktails) H0008-H0013

| H0008 | H0009 | H0010 | H0011 | H0012 | H0013 |
|---|---|---|---|---|---|
| HHEX | HHEX | CEBPA | FoxA1 | Smad3 | HNF1A |
| SOX17 | SOX17 | ATF5 | HNF1A | HHEX | CEBPA |
| HNF4A | HNF4A | HHEX | FoxA2 | ATF5 | ONECUT1/HNF6 |
| HNF1A | | SOX17 | CEBPA | NR5A2 | ONECUT2/HNF6B |
| ONECUT1/HNF6 | | HNF4A | ONECUT1/HNF6 | GATA2 isoform | Fos |
| | | | | P23769-1 | |
| FoxA2 | | ONECUT2/HNF6B | HNF4a | CEBPB | HIF1A |
| FoxA1 | | HNF1A | RBPJ | NR1I3 | TBX3 |

Each TF was cloned into an expression plasmid vector then transfected into iPSCs. The plasmid was designed to be antibiotic resistant and small molecule-inducible in order to allow for the selection of TF-integrated cells and to express the TFs on demand, respectively. Other methods of inducing cell conversion may include non-integrating DNA transfection, mRNA transfection or protein delivery.

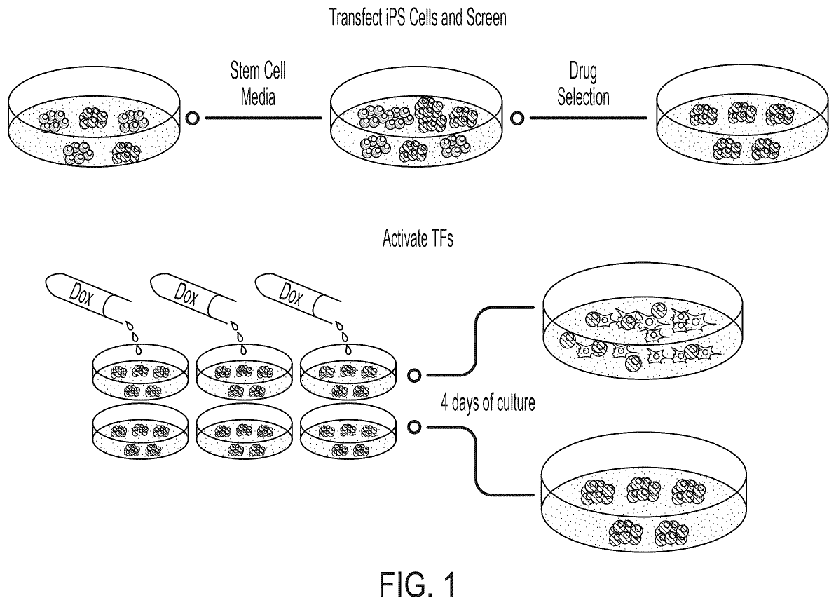

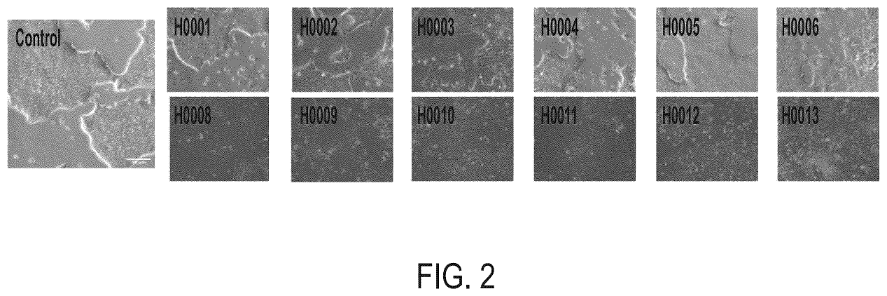

Twelve different pools of TFs combinations (also known as TF cocktails or TF recipes) were selected for transfection. In order to screen the ability of these pools for hepatocyte formation, iPSCs were transfected, and then specific antibiotics were used to select for integrants (FIG. 1). TF expression was induced by addition of small molecules to the stem cell media for four days (96 hours). Change in cell morphology was observed throughout the process (FIG. 2 and Table A). Growth was conducted with no nutrient, no growth factor, or any microenvironmental/matrix optimizations to assess and improve differentiation efficiency. The most effective TF candidates were elected.

Induced pluripotent stem cells (iPSCs) with the combination of TFs from Table 1 were transfected. Cells with no TF induction and cells with TF induction were compared by adding or excluding an inducible small molecule reagent to the media to activate the inducible promoter for a period of 96 hours. After 96 hours of TF induction and without altering any media or matrix components, cells were evaluated for expression of three hepatocyte markers (CK18 (cytokeratin 18), Albumin and ASGR-1) using fluorescently labeled antibodies targeting each marker. Fluorescence was assessed by flow cytometry.

### Example 2: H0001 Differentiate iPSCs into Hepatocytes as Confirmed by Flow Cytometry

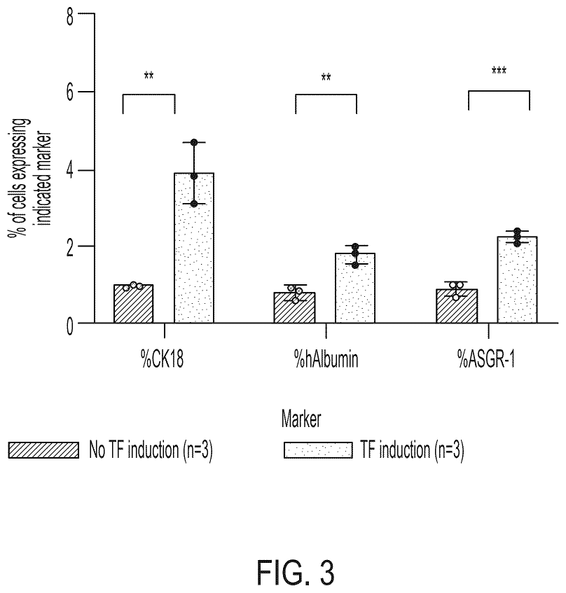

H0001 showed an increase in the percentage of cells expressing each marker compared to cells without TF induction. Specifically, 4% of TF-induced cells expressed CK18 compared to 1% of cells without TF induction; 2% of TF-induced cells expressed Albumin compared to 1% of cells without TF induction; 2.5% of TF-induced cells expressed ASGR-1 compared to 1% of cells without TF induction. These data indicated that TFs from the H0001 combination were able to induce differentiation of iPSCs into CK18+Albumin+ASGR-1+hepatocyte cells (FIG. 3).

### Example 3: H0008, H0010 and H0011 Differentiate iPSCs into Hepatocytes without Addition of any Nutrients, Growth Hormones or Matrix Components

Experiments were conducted to test whether TF combinations led to differentiation of iPSCs into hepatocyte lineage and the efficiency of that differentiation. To do this, induced PSC were either transfected using antibiotic selection and TF expression was induced (as described previously in Example 1 above) or there was no induction of TF expression (control group) following transfection of iPSC. Cells were trypsinized and stained using fluorescently labeled antibodies. Cells were evaluated for hepatocyte-like differentiation using flow cytometry to assess expression of hepatocyte-specific markers.

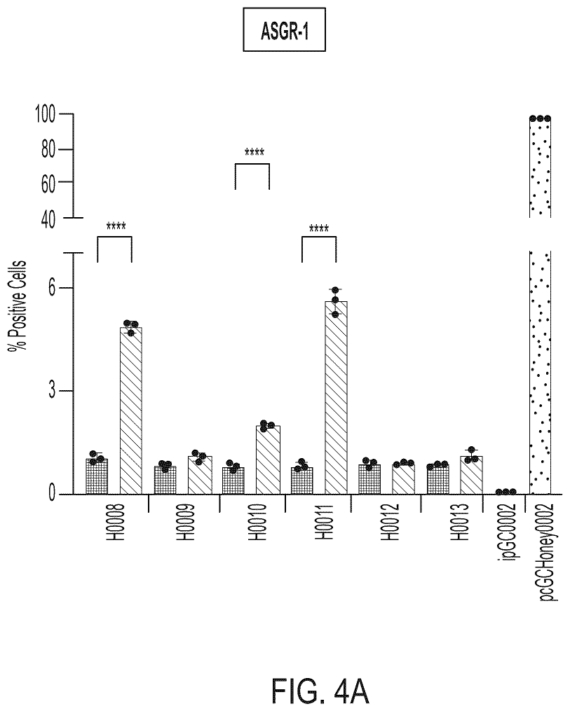

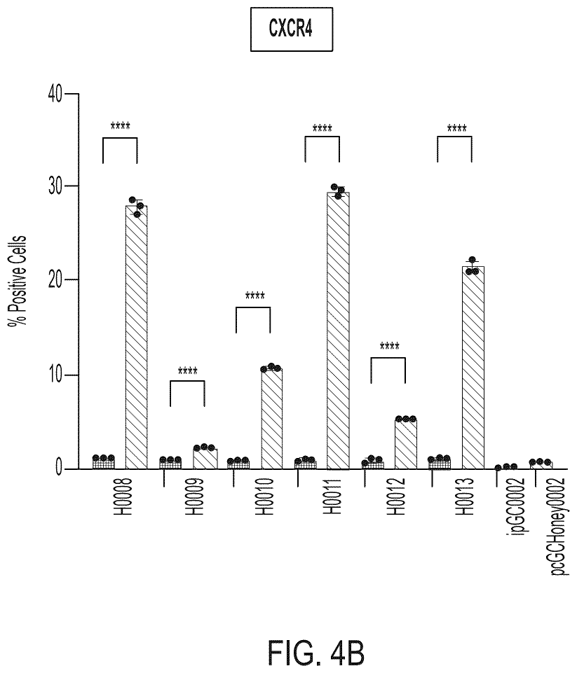

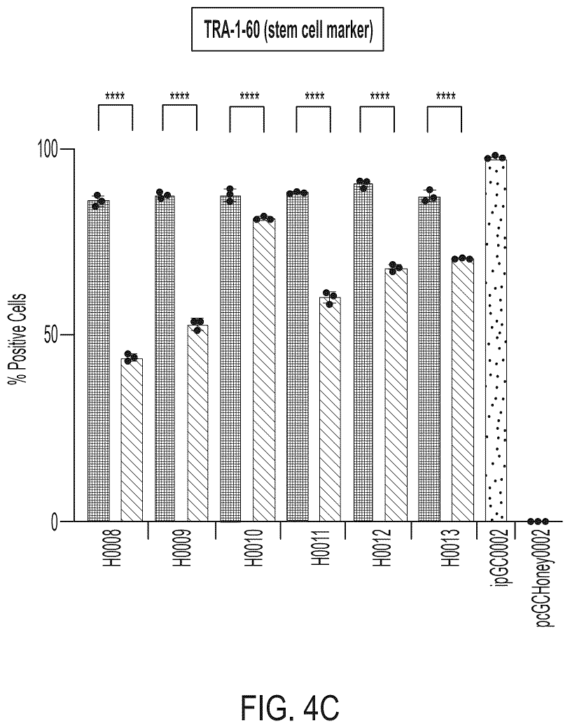

While cell lines modified to express H0008, H0010, and H0011 TF combinations expressed hepatocyte-specific markers ASGR-1 and CXCR4, cell lines modified to express H0012 & H0013 predominantly expressed definitive endoderm markers CXCR4 (but that hepatocyte-specific marker expression was not significantly different from control) (FIG. 4). Overall, there were differences in the percent of cells that showed expression of the hepatocyte marker ASGR-1 among cells modified to express the TF combinations e.g., for H0008˜5% of TF-induced cells expressed ASGR-1 marker compared to 1% in uninduced cells; for H0010, ˜2% of TF-induced cells expressed ASGR-1marker compared to 1% in uninduced; for H0011, ˜6% of TF-induced cells expressed the ASGR-1 marker compared to 1% in uninduced (FIG. 4A). No statistically significant increase in ASGR-1 expression was observed in H0009, H0012 or H0013; ipGC0002 represented non-transfected negative control iPSCs and did not express ASGR-1. pcGCHoney0002 were positive control cells that expressed ASGR-1. Overall, these results indicate that TF combinations H0008, H0010 and H0011 induced differentiation of iPSCs into hepatocytes in 96 hours without any changes made to the media (no growth factors, no nutrients) or matrix components.

### Example 4: H0008, H0009, H0010, H0011, H0012 and H0013 Differentiate iPSCs into Hepatocyte Lineages without Addition of any Nutrients, Growth Factors or Matrix Components

iPSCs were evaluated for differentiation into hepatocyte lineages using flow cytometry to assess expression of hepatocyte-lineage specific markers. To evaluate differentiation into hepatocyte lineages such as endoderms, for example, expression of the definitive endoderm marker CXCR4, was assessed.

Variation in CXCR4 expression was detected in cells modified with TF combinations, for example, while robust significant expression of CXCR4 was detected for cells modified to express H0011 (˜30%), H0008 (˜28% of TF-induced cells), and H0013 (˜20%), slightly lower, though significant expression was detected for cells modified to express H0010 (˜10%), H0009 (˜2%), and H0012 (˜5%) TF recipes (FIG. 4B). Cells modified to express H0008, H0010 and H0011TF combinations displayed significant expression of the CXCR4 marker compared to marker expression from uninduced cells, suggesting that these TF combinations induce differentiation of iPSCs into early hepatocytes that are expressing both a hepatocyte marker (ASGR-1; FIG. 4A) and definitive endoderm lineage marker (CXCR4; FIG. 4B). Collectively, these data show that cells modified to express H0009, H0012 and H0013 TF combinations, had much lower rates of iPSC differentiation since the expression of ASGR-1 hepatocyte marker was insignificant. Conversely, cell lines modified to express H009, H0012 and H0013 TF combinations did express CXCR4, a definitive endoderm marker, at 2%, 5%, and 20% respectively, suggesting these TF combinations induced iPSCs into endoderm lineage cells, either as definitive endoderm progenitors or other differentiated endoderm lineage cell types.

The expression of the pluripotency marker TRA-1-60 was also assessed. TF combinations that induce cells to lose TRA-1-60 expression are interpreted to be inducing differentiation (although without other specific markers, the exact differentiated cell type is unclear). Loss of TRA-1-60 in cells modified to express H0008 to H0013 was observed (FIG. 4C) about 50% of H0008 cells were TRA-1-60-negative differentiated cells. Additionally, ˜50% of H0009 cells were TRA-1-60-negative differentiated cells; ˜20% of H0010 cells were TRA-1-60-negative differentiated cells; ˜40% of H0010 were TRA-1-60-negative differentiated cells; ˜30% of H0011 were TRA-1-60-negative differentiated cells; ˜25% of H0012 were TRA-1-60-negative differentiated cells; and ˜25% of H0013 were TRA-1-60-negative differentiated cells. Positive control ipGC0002 were non-transfected iPSCs and ˜95% of cells were TRA-1-60-positive. Negative control pcGCHoney0002 are differentiated cells that did not express TRA-1-60.

Overall, four different cell recipes (H0001, H0008, H0010, and H0011) were successfully shown to drive differentiation at various efficiencies to produce hepatocytes or hepatocyte-like cells from iPSCs. Additionally, three cell recipes (H0009, H0012 and H0013) induced endoderm lineage cells from iPSCs as shown in FIG. 4B.

### Example 5: H0008 and H0011 Differentiate iPSCs into Hepatocytes

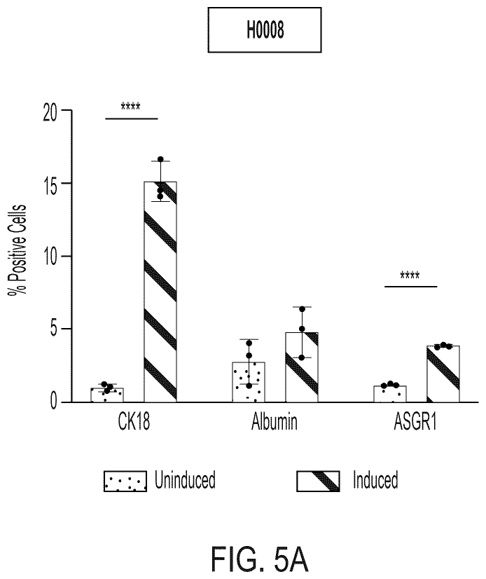

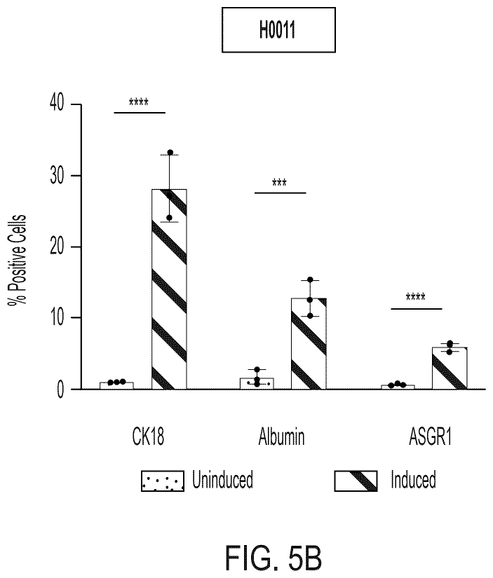

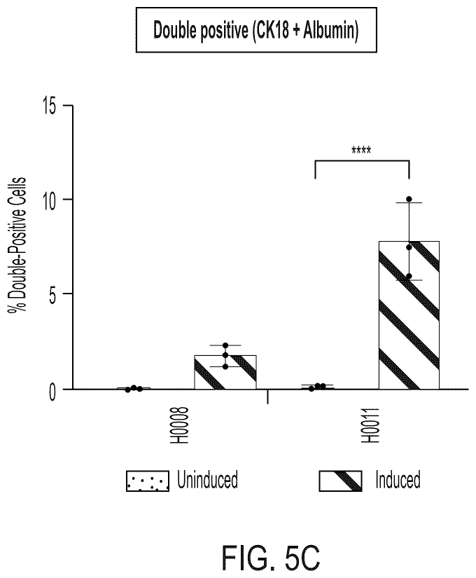

Two recipes (H0008 and H0011) were further investigated to assess expression of additional hepatocyte markers, CK18 and Albumin, and further confirm iPSC differentiation into hepatocytes. In H0008, ˜15% of TF-induced cells expressed CK18, ˜5% of TF-induced cells expressed albumin and reproduced ˜5% of TF-induced cells expressed ASGR1 (FIG. 5A). For H0011, ˜28% of TF-induced cells expressed CK18, ˜13% of TF-induced cells expressed Albumin and reproduced ˜5% of TF-induced cells expressed ASGR1 (FIG. 5B). The percentage of cells expressing both CK18 and Albumin were quantified, and in cells modified to express H0008, ˜3% CK18 and Albumin double positive cells were observed (FIG. 5C). In cells modified to express H0011, ˜8% CK18 and Albumin double positive cells were observed.

### Example 6: H0001 Differentiate iPSCs into Hepatocytes

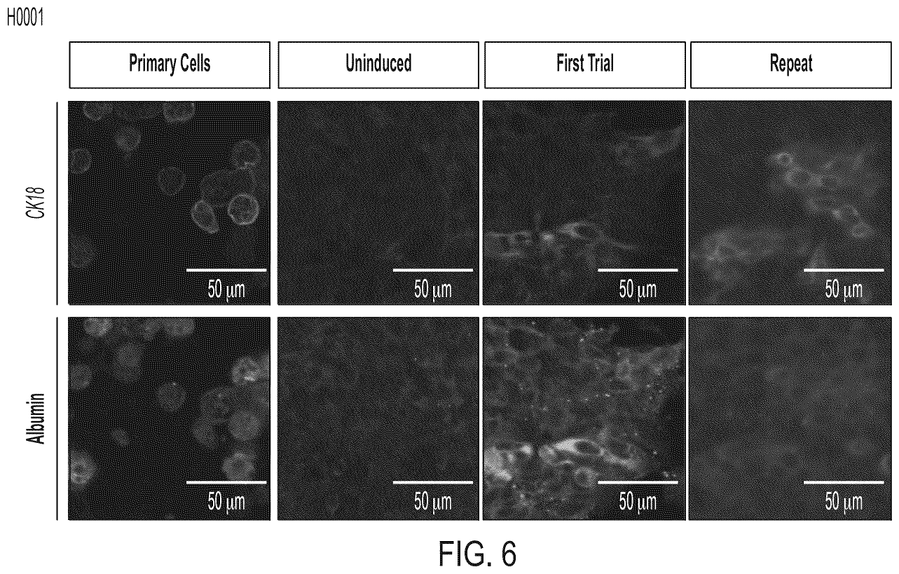

Marker expression was assessed by immunofluorescence microscopy. Differentiation into hepatocyte marker-positive cells was demonstrated, based on cytokeratin18 and albumin immunofluorescence for the top recipes. As shown in FIG. 6A (CK18 marker) and FIG. 6B (albumin marker), primary cells which served as a positive control, stained positive for both CK18 and Albumin markers. Conversely, the uninduced panel contained stem cells which were did not contain any transcription factor or were not induced to differentiate thereby served as a negative control stained negative for both markers. In the first trial panel, stem cells induced with Honey001, produced double positive cells or cells that stained positively for both the CK18 and Albumin markers, confirming differentiation of stem cells into hepatocytes. The experiment was repeated (Repeat panel) and result indicate stem cells differentiated to hepatocytes to produce double positive cells that stained positively for CK18 and Albumin markers. Overall, it was concluded that H0001 expresses both CK18 and Albumin, and that it is reproducible in independent differentiations and assessments.

### Example 7: Differentiation of iPSCs into Hepatocytes Driven by Specific TF Combinations

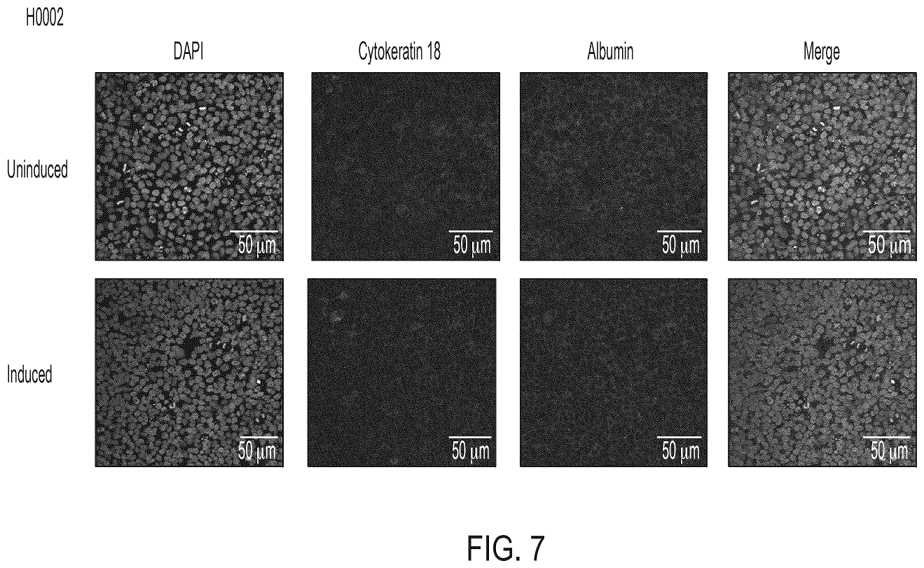

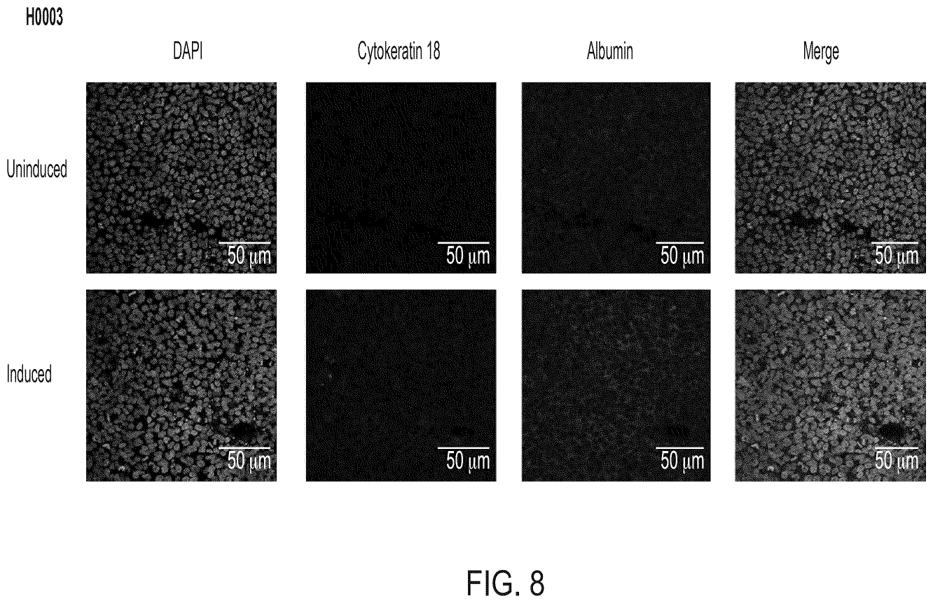

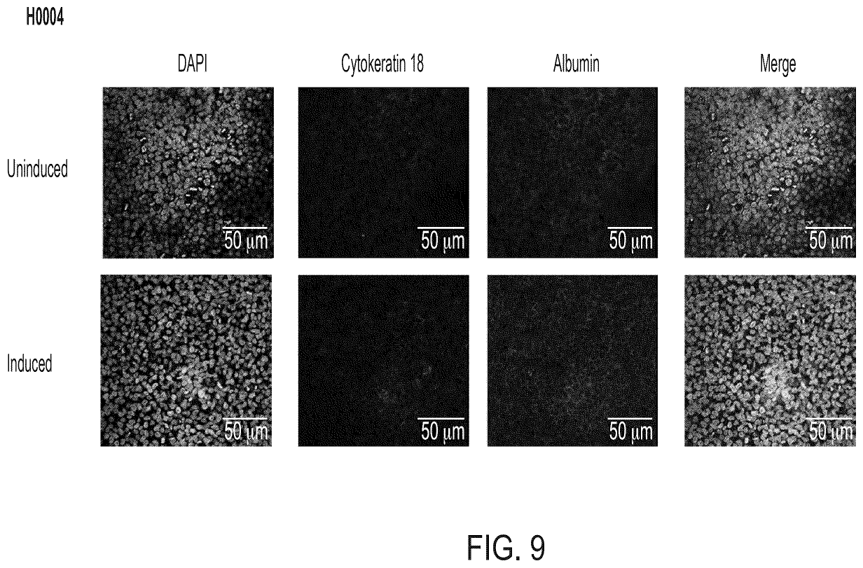

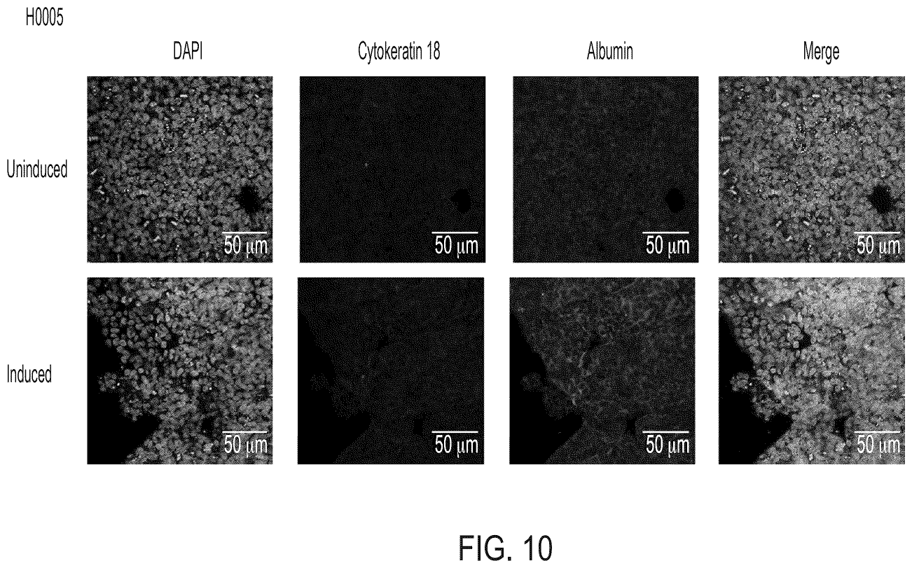

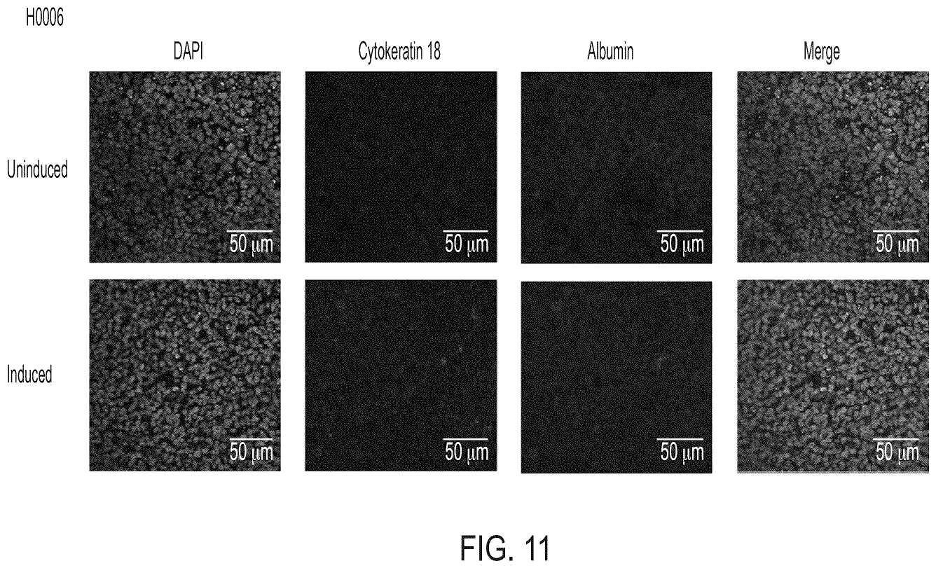

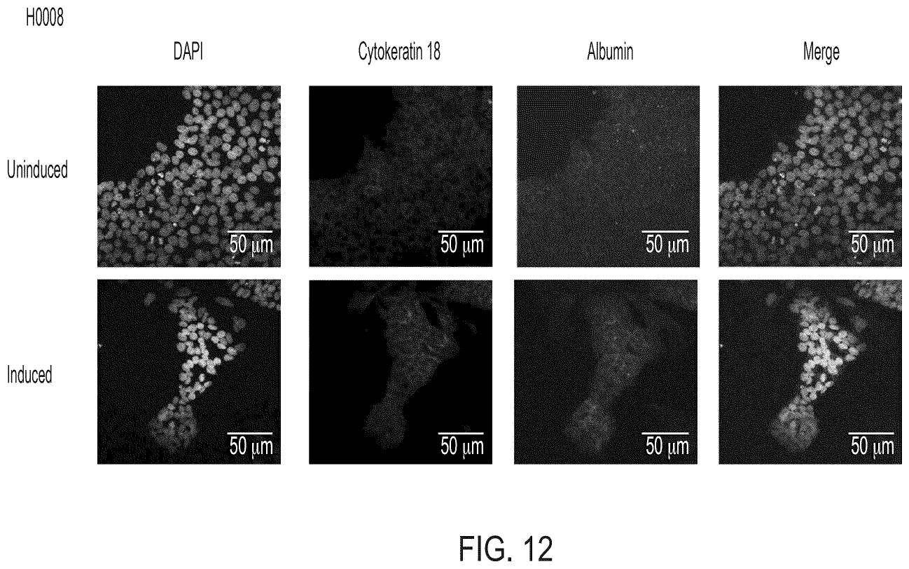

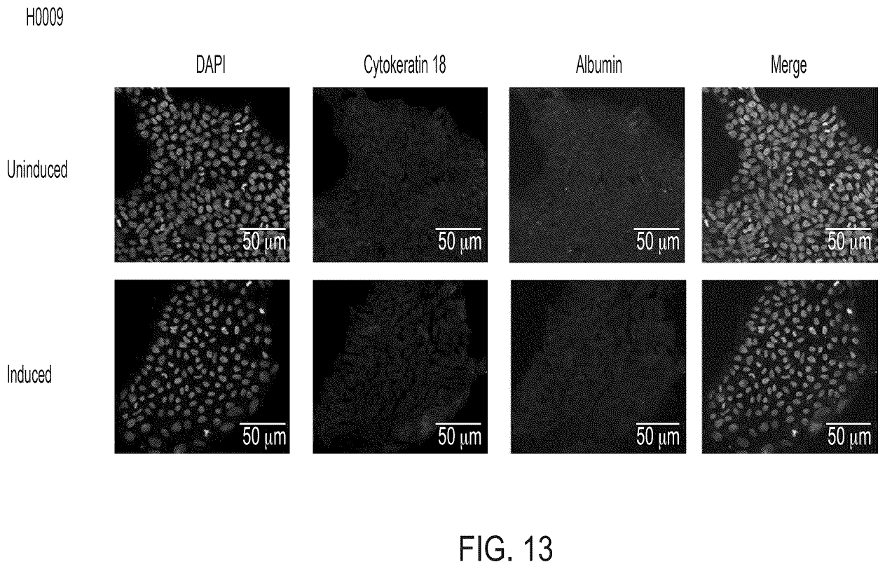

TF recipes or combinations were assessed as in prior experiments above, to evaluate efficacy in inducing iPSCs to differentiate into hepatocytes. Various TF combinations or cocktails were used for the differentiation experiment as can be seen in Tables 1 to Tables 11. It was determined that cells modified to express H0002 (FIG. 7), H0003 (FIG. 8), H0004 (FIG. 9), H0005 (FIG. 10), H0006 (FIG. 11) and H0009 (FIG. 13) TF combinations did not express appreciable levels of hepatocyte-specific markers when assessed using immunofluorescence imaging. These data indicate that these different TF combinations or recipes do not induce iPSCs to differentiate into hepatocytes.

### Example 8: H0008, H0010 and H0011 Express Other Hepatocyte-Specific Markers

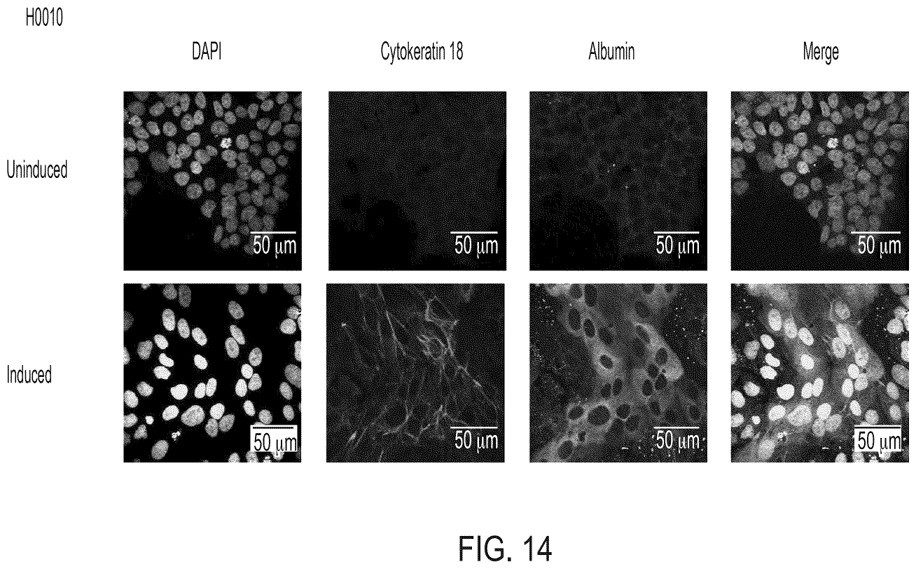

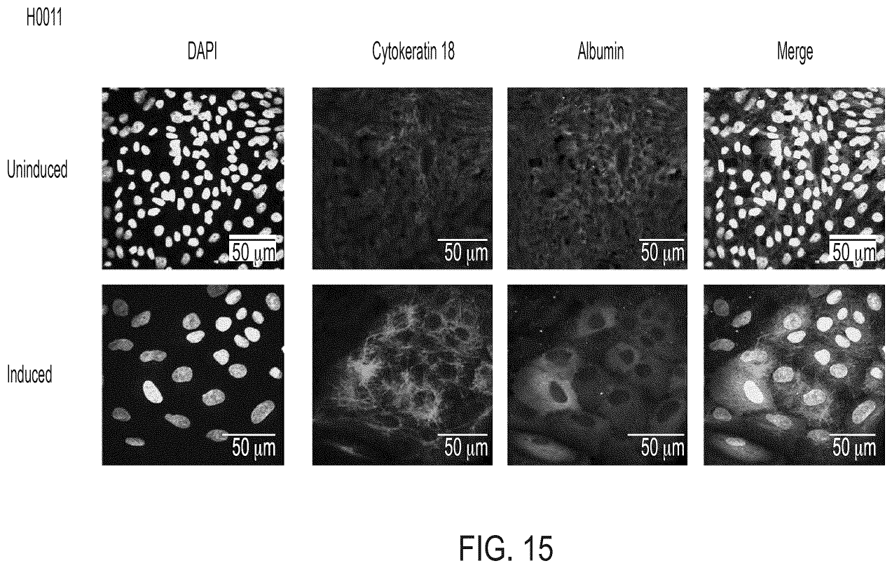

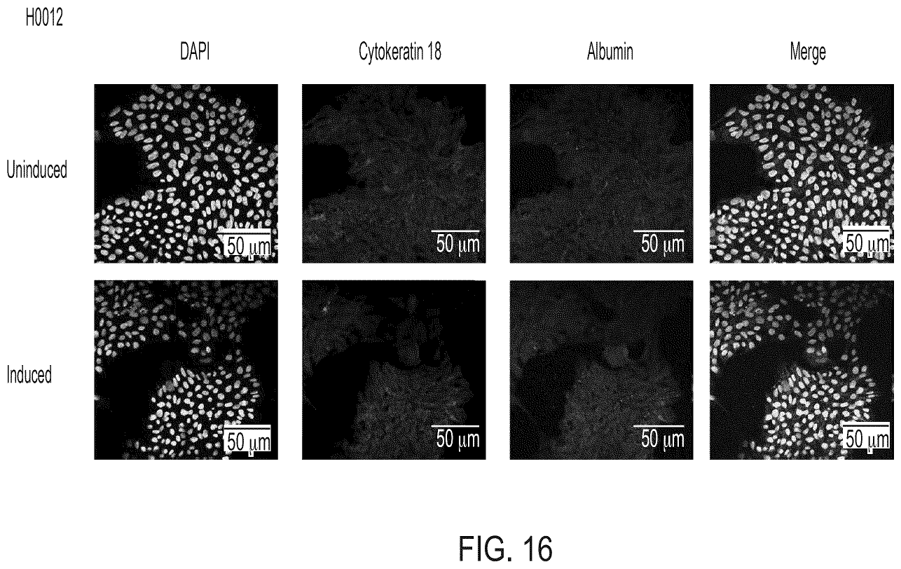

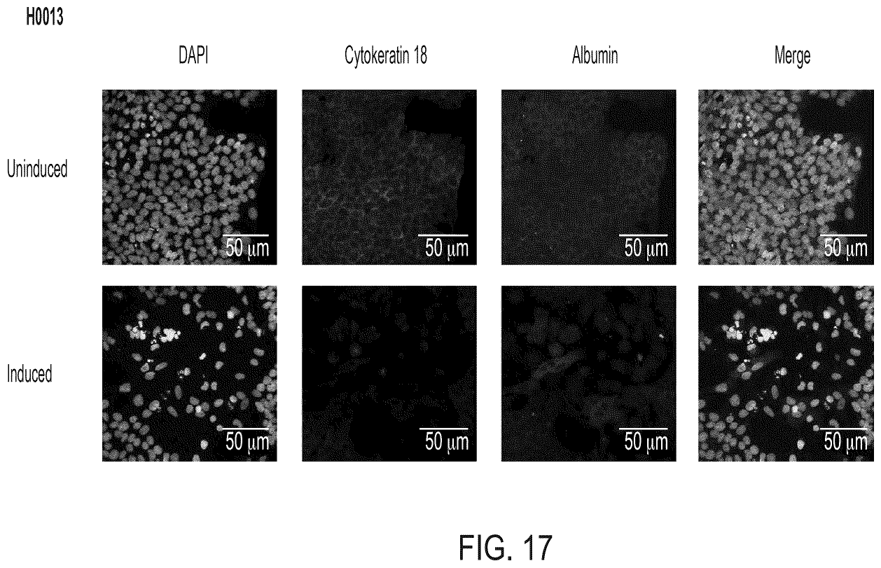

It was demonstrated previously using flow cytometry that cells modified and induced to express H0008 (FIG. 5A) and H0011 express CK18, ASGR1 (FIG. 5A-5B) and that in addition H0011-cells expressed significant levels of Albumin alone or in combination with CK18 (FIG. 5B-5C). Here, in this experiment cells were modified, and it was demonstrated that cells modified to express H0008 expressed hepatocyte-specific markers including CK18 and Albumin by immunofluorescence microscopy (FIG. 12), which reproduced the results from the previously referenced flow cytometry assay. Similarly, H0010 & H0011 cell lines both expressed hepatocyte-specific markers such as CK18 and Albumin and showed large, polygonal cells characteristic of hepatocytes (FIG. 14 and FIG. 15). This assay confirmed results obtained using the flow cytometry assay (in previous section). Immunofluorescence microscopy also confirmed the correct localization of these markers, and as expected, CK18 is an intermediate filament protein in the cytoplasm and Albumin is also expected to be observed in the cytoplasm of hepatocytes. While H0012 and H0013 cell lines expressed definitive endoderm markers in flow cytometry (FIG. 4), they did not express hepatocyte-specific markers such as CK18 and Albumin by immunofluorescence either (FIG. 16 and FIG. 17). This is consistent with the previous flow cytometry results. Based on our previous flow cytometry results, since H0012 and H0013-cell lines express endoderm markers but not hepatocyte markers, it was concluded that these cells are either still at the definitive endoderm stage or are producing cells from that lineage which are not hepatocytes.

### Example 9: H0001 Differentiated iPSCs can Accumulate and/or Synthesize Lipids

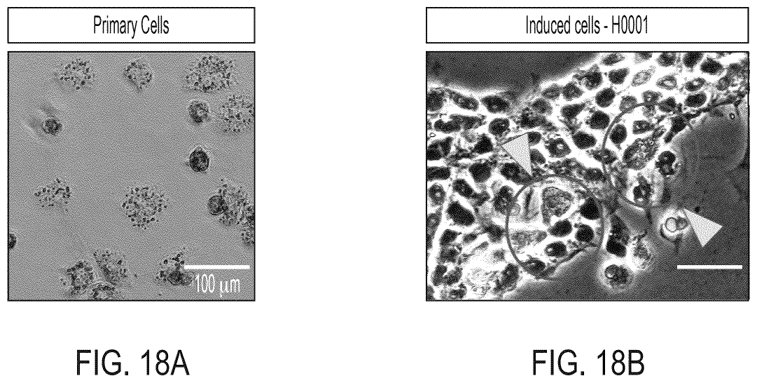

Beyond immunofluorescence and flow cytometry, an accepted functionality assay to assess the ability of differentiated cells to synthesize and accumulate lipids, expected of hepatocytes, was established. A test was conducted to evaluate the accumulation and/or synthesis of lipids using an Oil Red O dye assay. The dye, which stains for lipids, was introduced to differentiated cells. The cells were tested, and it was successfully shown that the cells modified to express H0001 TF combination are functional. As shown in FIG. 18A, primary hepatocytes with lipid droplets stained red as expected. When induced H0001 cells were stained under different conditions, a number of cells contained the red lipid droplets seen in the primary hepatocyte staining (FIG. 18B (arrows). These cells that stained red were not observed in uninduced TF controls or in the absence of the Oil Red O dye assay (data not shown). Overall, these datasets show that the H0001 TF candidate recipe is able to induce iPSCs to differentiate into hepatocytes and the hepatocytes in turn, expresses the proper hepatocyte markers based on flow cytometry, immunofluorescence microscopy, and the present lipid accumulation or synthesis functional assay (FIG. 18B). In summary, H0001 was able to induce expression of CK18, Albumin and ASGR1 markers, wherein CK18 was expressed in ˜7% of the cells. Programmed cells were confirmed to express the same markers by immunofluorescence microscopy and were shown to accumulate lipids by Oil Red O staining.

### Example 10: H0011 Differentiated iPSCs can Accumulate and/or Synthesize lipids Similarly, functionality of the H0011 induced hepatocytes was shown using the Oil Red

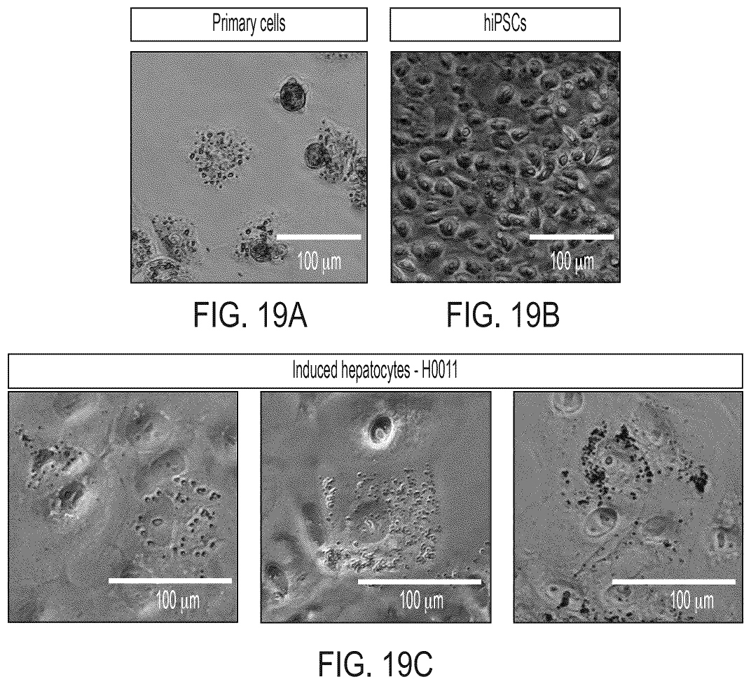

O assay. FIG. 19A, shows primary cells (primary hepatocytes) with stained lipid droplets. In human iPSCs (hiPSCs) or primary cells without any TF induction, there are no signs of lipid accumulation as expected (FIG. 19B). When H0011 induced cells were stained with Oil Red O, red lipid droplets were observed in multiple samples (FIG. 19C), which strongly suggested differentiated cells had the capability to store/accumulate or synthesize lipids. Overall, these datasets confirm that not only have cells been generated from iPSCs which are positive for hepatocyte specific markers both with flow cytometry and immunofluorescent imaging, but they also possess the initial hepatocyte-specific lipid storage functionality as confirmed here.

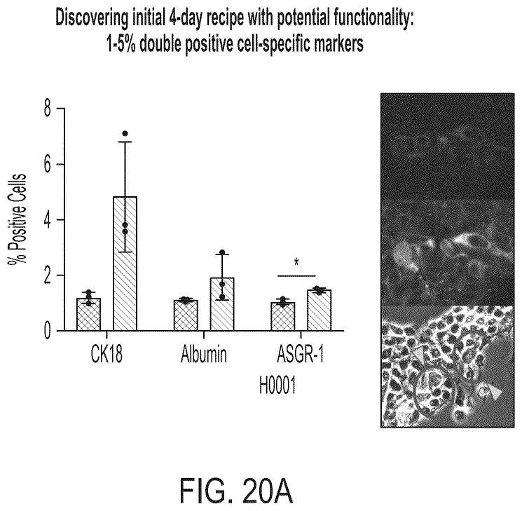

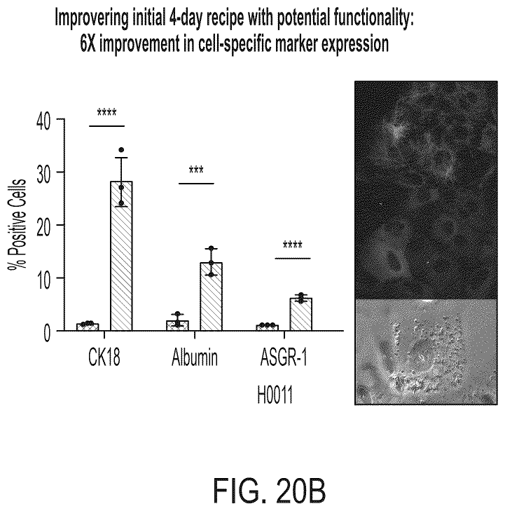

H0011 cell line showed improved characteristics. For example, FIG. 20B shows that H0011 cells achieved expression of up to 30% CK18 and 10% Albumin, which is about a 5-6X improvement from the initial H0001 recipe (FIG. 20A). Cells modified to express H0011 successfully induced hepatocytes and stained for the same markers by immunofluorescence (FIG. 15) and showed lipid storage functionality by Oil Red O staining (FIG. 20B).

### Example 11: Additional TFs Recipes that Differentiate iPSCs into Hepatocyte Discovered

TABLE 3
TF cocktails effecting differentiation of iPSCs to hepatocytes
or hepatocyte-like lineages or endoderm lineage

| H0014 | H0015 | H0015C | H0015D | H0016 | H0017 | H0018 |
|---|---|---|---|---|---|---|
| HHEX | HNF4A | HNF4A | HNF4A | FOXA1 | PROX1 | SKI |
| SOX17 | HNF1A | HNF1A | HNF1A | HNF1A | FOXA3 | RXRG |
| HNF4A | ONECUT1 | ONECUT1/HNF6 | ONECUT1/HNF6 | CEBPA | SMAD3 | SOX17 |
| HNF1A | FOXA2 | FOXA2 | FOXA2 | HNF4A | ATF5 | RXRB |
| ONECUT1 | FOXA1 | FOXA1 | FOXA1 | GATA2 | SOX17 | GATA3 |
| | | | | #1 | | |
| FOXA2 | | | | GATA6 | EGR1 | ONECUT2 |
| FOXA1 | | | | PROX1 | NR1I2/PXR | FOXA1 |
| CEBPA | | | | | | |
| RBPJ | | | | | | |
| ATF5 | | | | | | |

TABLE 4
TF cocktails effecting differentiation of iPSCs to hepatocytes
or hepatocyte-like lineages or endoderm lineage

| H0019 | H0020 | H0021 | H0022 | H0023 | H0024 | H0025 |
|---|---|---|---|---|---|---|
| ATF5 | FOXA1 | HNF4A | GATA6 | GATA4 | HNF4A | HNF4A |
| PROX1 | HNF1A | HNF1A | FOXA2 | FOXA2 | PROX1 | HNF1A |
| CEBPA | FOXA2 | ONECUT1 | PROX1 | HHEX | | FOXA2 |
| FOXA3 | CEBPA | FOXA2 | HNF1A | PROX1 | | |
| FOXA2 | ONECUT1 | FOXA1 | HNF4A | HNF4A | | |
| HNF4A | HNF4A | | ATF5 | | | |
| GATA6 | RBPJ | | FOXA3 | | | |

TABLE 5
TF cocktails effecting differentiation of iPSCs to hepatocytes or hepatocyte-like lineages or endoderm lineage

| H0026A | H0026B | H0027A | H0027B | H0028A | H0028B | H0029A | H0029B |
|---|---|---|---|---|---|---|---|
| HNF1A | HNF1A | HNF4A | HNF4A | HNF4A | HNF4A | HNF4A | HNF4A |
| ONECUT1/HNF6 | ONECUT1/HNF6 | HNF1A | HNF1A | HNF1A | HNF1A | ONECUT1/HNF6 | ONECUT1/HNF6 |
| FOXA2 | FOXA2 | FOXA2 | FOXA2 | ONECUT1/HNF6 | ONECUT1/HNF6 | FOXA2 | FOXA2 |
| FOXA1 | FOXA1 | FOXA1 | FOXA1 | FOXA1 | FOXA1 | FOXA1 | FOXA1 |

TABLE 6
TF cocktails effecting differentiation of iPSCs to hepatocytes
or hepatocyte-like lineages or endoderm lineage

| H0030A | H0030B | H0031A | H0032A | H0033A | H0034A | H0035A |
|---|---|---|---|---|---|---|
| HNF4A | HNF4A | FOXA1 | FOXA1 | CEBPA | CEBPA | CEBPA |
| HNF1A | HNF1A | HNF1A | HNF4A | FOXA1 | FOXA1 | HNF1A |
| ONECUT1/HNF6 | ONECUT1/HNF6 | CEBPA | ONECUT1/HNF6 | HNF1A | HNF1A | HNF4A |
| FOXA2 | FOXA2 | ONECUT1/HNF6 | RBPJ | HNF4A | HNF4A | FOXA3 |

| | | |
|---|---|---|
| HNF4A | ONECUT1/HNF6 | FOXA2 |

| | |
|---|---|
| RBPJ | PXR |
| | RXRA |

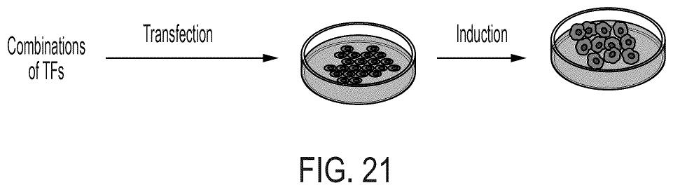

The effect of transcription factors (TFs) on hepatocyte differentiation was further investigated (Tables 3-Table 6). A group of TFs were selected for this experiment and assessed for their ability to induced iPSCs to hepatocyte or hepatocyte-like differentiation. To do this, iPSCs were transfected, selected and expanded. TF combinations were transfected, and induction was allowed to proceed for about 96 hours (FIG. 21) then differentiation of iPSCs was assessed for hepatocyte-like differentiation using flow cytometry for ASGR-1, a hepatocyte cell surface marker, and by measuring albumin production by the enzyme-linked immunosorbent assay (ELISA). Several recipes induced ASGR expression.

### Example 12: Effect of Additional TF Recipe on iPSCs Differentiation

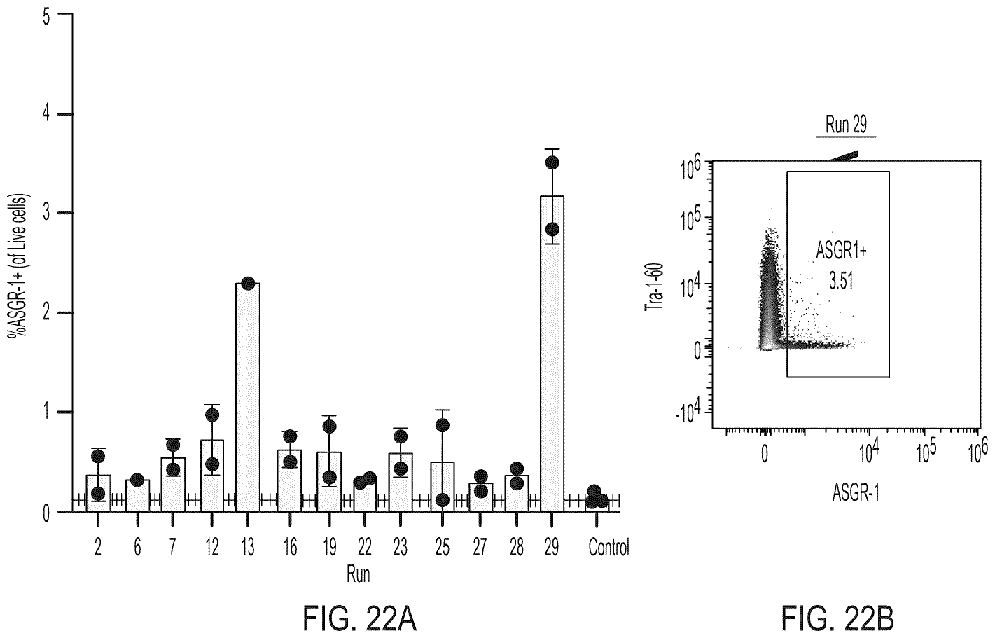

Additional TF cocktails or recipes were tested to evaluate their effect on induction of iPSC to differentiate into hepatocytes or hepatic cells. To do this, TF recipes or TF cocktails shown in Tables 7, 8, 9, 10 and Table 11, were administered to PSCs and induction to differentiate was allowed to proceed for at least 96 hours. Differentiated cells that expressed ASGR-1 hepatocyte marker were evaluated using flow cytometry. The percentage of cells that that differentiated into hepatocytes was detected by the percent cells that expressed the hepatocyte marker, ASGR-1, on their cells surface (FIG. 22A; live cells; runs 1-32). Runs 1-32 correspond to TF recipes H0036-H0067, depicted in Tables 7-11. Run 29 which received the H0064 TF cocktail had the highest percentage (3.5%) of ASGR-1 expressing cells (FIG. 22A). Run 29 comprised PSC cells received administered H0064 TF cocktails. An example plot for run 29, with the most ASGR1 positive cells, is shown FIG. 22B. The gray dotted line is the average +/−standard deviation for the uninduced control cells. The expression of ASGR-1 was evaluated among live cells, therefore among cells administered H0064, 3.5% of live cells expressed the ASGR-1 marker, confirming these cells had differentiated into hepatocytes or hepatic cells or hepatocyte-like lineage.

The second highest percentage (2.3%) of ASGR-1 expressing cells was found in TF recipe H0048 (run 13; FIG. 22A). The expression of ASGR-1 was evaluated among live cells, therefore, 2.3% of the live cells tested expressed ASGR-1 marker. Expression of ASGR-1 at varying percentages was also confirmed in other runs or TF recipes 2, 6, 7, 12, 16, 19, 22, 23, and 25, 27 and 28 (FIG. 22A). The corresponding TF recipes for the runs can be seen in Tables 7-11. Additional transcription factors used to generate induction of PSCs to hepatocytes, hepatocyte-like cells or hepatic cells can be located in Table 12.

TABLE 7
TF cocktails effecting differentiation of iPSCs to hepatocytes or hepatocyte-like lineages or endoderm lineage

| Run 1 | Run 2 | Run 3 | Run 4 | Run 5 | Run 6 | Run 7 |
|---|---|---|---|---|---|---|
| H0036 | H0037 | H0038 | H0039 | H0040 | H0041 | H0042 |
| ONECUT1/HNF6 | ATF5 | CEBPA | ATF5 | CEBPB | ATF5 | CEBPA |
| ONECUT2/HNF6B | FOXA3 | FOXA3 | CEBPA | FOXA3 | CEBPB | CEBPB |
| NR1C1/PPARA | HIF1A | GATA6 | GATA6 | GATA6 | GATA6 | HHEX |
| TBX3 | HLF | HNF1A | HIF1A | HHEX | HHEX | HIF1A |
| SALL4 | HNF4A | HNF4A | HLF | HIF1A | HLF | HNF1A |
| | NR1H4/FXR | NR1H4/FXR | HNF1A | NR1I2/PXR | HNF4A | HNF4A |
| | NR1I2/PXR | NR1I3/CAR | NR1I2/PXR | NR1I3/CAR | NR1H4/FXR | NR1H4/FXR |
| | NR5A2 | NR5A2 | NR1I3/CAR | NR5A2 | NR1I3/CAR | NR1I2/PXR |
| | RBPJ | PROX1 | ONECUT1/HNF6 | NR1C1/PPARA | ONECUT1/HNF6 | ONECUT1/HNF6 |
| | | RXRA | ONECUT2/HNF6B | RORC | ONECUT2/HNF6B | ONECUT2/HNF6B |
| | | TBX3 | NR1C1/PPARA | TBX3 | PROX1 | RBPJ |
| | | SALL4 | RORC | SALL4 | RXRA | TBX3 |
| | | | | | | SALL4 |

TABLE 8
TF cocktails effecting differentiation of iPSCs to hepatocytes or hepatocyte-like lineages or endoderm lineage

| Run 8 | Run 9 | Run 10 | Run 11 | Run 12 | Run 113 | Run 14 |
|---|---|---|---|---|---|---|
| H0043 | H0044 | H0045 | H0046 | H0047 | H0048 | H0049 |
| ATF5 | FOXA1 | ATF5 | CEBPA | ATF5 | CEBPB | ATF5 |
| CEBPA | GATA6 | FOXA1 | FOXA1 | CEBPA | FOXA1 | CEBPB |
| CEBPB | HHEX | FOXA3 | FOXA3 | FOXA1 | FOXA3 | FOXA1 |
| FOXA3 | HIF1A | GATA6 | HHEX | HHEX | HLF | HIF1A |
| HHEX | HLF | HHEX | HIF1A | HNF4A | HNF1A | HNF1A |
| HLF | HNF1A | HNF1A | HLF | NR1I2/PXR | HNF4A | NR1H4/FXR |
| HNF1A | HNF4A | NR1H4/FXR | NR1H4/FXR | NR1I3/CAR | NR1I2/PXR | NR1I3/CAR |
| NR5A2 | NR5A2 | NR1I2/PXR | NR1I3/CAR | NR5A2 | NR1I3/CAR | NR5A2 |
| NR1C1/PPARA | ONECUT2/HNF6B | NR1I3/CAR | ONECUT1/HNF6 | ONECUT2/HNF6B | ONECUT1/HNF6 | ONECUT2/HNF6B |
| PROX1 | RORC | ONECUT1/HNF6 | NR1C1/PPARA | PROX1 | PROX1 | NR1C1/PPARA |
| RBPJ | RXRA | NR1C1/PPARA | RBPJ | RBPJ | RBPJ | RBPJ |
| RORC | SALL4 | PROX1 | RXRA | RORC | RORC | RXRA |
| RXRA | | TBX3 | SALL4 | TBX3 | SALL4 | TBX3 |

TABLE 9
TF cocktails effecting differentiation of iPSCs to hepatocytes or hepatocyte-like lineages or endoderm lineage

| Run 15 | Run 16 | Run 17 | Run 18 | Run 19 | Run 20 |
|---|---|---|---|---|---|
| H0050 | H0051 | H0052 | H0053 | H0054 | H0055 |
| CEBPA | ATF5 | FOXA2 | ATF5 | CEBPA | ATF5 |
| CEBPB | CEBPA | HHEX | FOXA2 | FOXA2 | CEBPA |
| FOXA1 | CEBPB | HLF | FOXA3 | FOXA3 | FOXA2 |
| GATA6 | FOXA1 | HNF1A | HHEX | GATA6 | GATA6 |
| HLF | FOXA3 | NR1H4/FXR | HIF1A | HHEX | HHEX |
| NR1H4/FXR | GATA6 | NR1I2/PXR | HNF1A | HLF | HIF1A |
| NR1I2/PXR | HIF1A | NR1I3/CAR | HNF4A | HNF4A | NR1H4/FXR |
| NR5A2 | HNF4A | NR5A2 | NR1I3/CAR | NR1I2/PXR | NR5A2 |
| ONECUT2/HNF6B | ONECUT1/HNF6 | ONECUT1/HNF6 | ONECUT2/HNF6B | ONECUT2/HNF6B | ONECUT1/HNF6 |
| NR1C1/PPARA | RORC | RORC | NR1C1/PPARA | NR1C1/PPARA | PROX1 |
| PROX1 | RXRA | RXRA | PROX1 | RBPJ | RBPJ |
| SALL4 | TBX3 | TBX3 | SALL4 | RXRA | RORC |
| | | | | TBX3 | SALL4 |

TABLE 10
TF cocktails effecting differentiation of iPSCs to hepatocytes or hepatocyte-like lineages or endoderm lineage

| Run 21 | Run 22 | Run 23 | Run 24 | Run 25 | Run 26 |
|---|---|---|---|---|---|
| H0056 | H0057 | H0058 | H0059 | H0060 | H0061 |
| CEBPB | ATF5 | CEBPA | ATF5 | FOXA1 | ATF5 |
| FOXA2 | CEBPB | CEBPB | CEBPA | FOXA2 | FOXA1 |
| FOXA3 | FOXA2 | FOXA2 | CEBPB | GATA6 | FOXA2 |
| GATA6 | GATA6 | HIF1A | FOXA2 | HIF1A | FOXA3 |
| HIF1A | HNF1A | HNF4A | FOXA3 | HNF4A | GATA6 |
| HLF | HNF4A | NR1I3/CAR | HLF | NR1H4/FXR | HLF |
| HNF1A | NR1I2/PXR | NR5A2 | NR1H4/FXR | NR1I2/PXR | NR1I3/CAR |
| NR1H4/FXR | NR5A2 | ONECUT1/HNF6 | NR1I2/PXR | NR1I3/CAR | NR5A2 |
| ONECUT2/HNF6B | ONECUT1/HNF6 | NR1C1/PPARA | NR1I3/CAR | NR1C1/PPARA | ONECUT1/HNF6 |
| PROX1 | NR1C1/PPARA | PROX1 | ONECUT2/HNF6B | PROX1 | ONECUT2/HNF6B |
| RBPJ | RBPJ | TBX3 | RORC | RBPJ | RBPJ |
| RORC | RXRA | | RXRA | RORC | TBX3 |
| TBX3 | SALL4 | | SALL4 | RXRA | SALL4 |

TABLE 11
TF cocktails effecting differentiation of iPSCs to hepatocytes
or hepatocyte-like lineages or endoderm lineage

| Run 27 | Run 28 | Run 29 | Run 30 | Run 31 | Run 32 |
|---|---|---|---|---|---|
| H0062 | H0063 | H0064 | H0065 | H0066 | H0067 |
| CEBPA | ATF5 | CEBPB | ATF5 | CEBPA | ATF5 |
| FOXA1 | CEBPA | FOXA1 | CEBPB | CEBPB | CEBPA |
| FOXA2 | FOXA1 | FOXA2 | FOXA1 | FOXA1 | CEBPB |
| FOXA3 | FOXA2 | FOXA3 | FOXA2 | FOXA2 | FOXA1 |
| HIF1A | HLF | HHEX | HHEX | GATA6 | FOXA2 |
| HNF1A | HNF1A | HNF4A | HIF1A | HHEX | FOXA3 |
| NR1I2/ | HNF4A | NR1H4/ | HLF | HNF1A | GATA6 |
| PXR | | FXR | | | |
| NR5A2 | NR1H4/ | NR5A2 | NR1I2/ | NR1I3/ | HHEX |
| | FXR | | PXR | CAR | |
| ONECUT1/ | NR1C1/ | ONECUT1/ | PROX1 | RBPJ | HIF1A |
| HNF6 | PPARA | HNF6 | | | |
| ONECUT2/ | RORC | ONECUT2/ | RXRA | | HLF |
| HNF6B | | HNF6B | | | |
| PROX1 | TBX3 | NR1C1/ | TBX3 | | HNF1A |
| | | PPARA | | | |
| RXRA | SALL4 | RORC | SALL4 | | HNF4A |
| | | | | | NR1H4/ |
| | | | | | FXR |
| | | | | | NR1I2/ |
| | | | | | PXR |
| | | | | | NR1I3/ |
| | | | | | CAR |
| | | | | | NR5A2 |
| | | | | | ONECUT1/ |
| | | | | | HNF6 |
| | | | | | ONECUT2/ |
| | | | | | HNF6B |
| | | | | | NR1C1/ |
| | | | | | PPARA |
| | | | | | PROX1 |
| | | | | | RBPJ |
| | | | | | RORC |
| | | | | | RXRA |
| | | | | | TBX3 |
| | | | | | SALL4 |

TABLE 12
Additional transcription factors that may be used to differentiation of iPSCs to
hepatocytes or hepatocyte-like lineages or endoderm lineage can be found below

1: ENSG00000100987, 2: HGNC: 14209, 3: HGNC: 12920, 4: NM_006887, 5:
ENSG00000130584, 6: HGNC: 11593, 7: HGNC: 29365, 8: HGNC: 17521, 9:
ENSG00000126003, 10: ENSG00000281448, 11: HGNC: 6782, 12: ENSG00000141646, 13:
NM_012102, 14: FOXI1, 15: ENSG00000159885, 16: NM_001365805, 17: HGNC: 13112,
18: ENSG00000148200, 19: ENSG00000184436, 20: NM_001195753, 21: HGNC: 8633, 22:
HGNC: 25843, 23: ENSG00000172201, 24: NM_001126240, 25: ENSG00000187559, 26:
HGNC: 4318, 27: NM_001145097, 28: ENSG00000163064, 29: Entrez ID: 3236, 30:
ZNF653, 31: HGNC: 29010, 32: ENSG00000136327, 33: NM_005376, 34:
ENSG00000237900, 35: ESX1, 36: ZFP57, 37: ZFP92, 38: HGNC: 22265, 39: HGNC: 16759,
40: ENSG00000206366, 41: HGNC: 21735, 42: ENSG00000178764, 43: HGNC: 790, 44:
ENSG00000267041, 45: NHLH2, 46: NM_001204185, 47: NM_001126241, 48:
NM_001349608, 49: HGNC: 16744, 50: HGNC: 18078, 51: ENSG00000279552, 52: ZNF32,
53: ZBTB1, 54: ENSG00000160961, 55: HGNC: 11752, 56: HGNC: 18524, 57:
HGNC: 20696, 58: Entrez ID: 7181, 59: HGNC: 20813, 60: HGNC: 11716, 61:
ENSG00000113580, 62: ENSG00000234852, 63: NM_018471, 64: HGNC: 12930, 65:
ENSG00000206289, 66: ENSG00000174306, 67: HGNC: 17680, 68: ENSG00000119866, 69:
NM_013953, 70: Entrez ID: 147948, 71: HGNC: 13023, 72: HGNC: 9004, 73:
ENSG00000173120, 74: ENSG00000256463, 75: ENSG00000133937, 76:
ENSG00000004848, 77: ZBTB6, 78: ZNF804A, 79: HGNC: 8525, 80: ENSG00000181135,
81: ENSG00000144355, 82: ELF4, 83: HGNC: 9223, 84: ENSG00000125850, 85:
HGNC: 24108, 86: Entrez ID: 342933, 87: ENSG00000125285, 88: TSHZ1, 89:
HGNC: 14010, 90: HGNC: 11364, 91: SCAPER, 92: NM_001142588, 93:
ENSG00000225351, 94: E2F7, 95: HGNC: 18522, 96: NM_001367509, 97:
ENSG00000120690, 98: HGNC: 17150, 99: ZBTB26, 100: HGNC: 6120, 101:
ENSG00000198026, 102: ZNF827, 103: Entrez ID: 6670, 104: ENSG00000282684, 105:
HGNC: 26279, 106: NM_005598, 107: ENSG00000147421, 108: ENSG00000177425, 109:
ENSG00000206455, 110: ENSG00000115507, 111: HGNC: 25741, 112: TAL2, 113:
HGNC: 3317, 114: IRF3, 115: NM_001353130, 116: HGNC: 13139, 117: ENSG00000020256,
118: NM_014233, 119: HGNC: 4185, 120: HGNC: 26955, 121: ENSG00000233831, 122:
HGNC: 22788, 123: HGNC: 953, 124: Entrez ID: 65988, 125: NM_001261415, 126:
HGNC: 7188, 127: ZNF304, 128: HGNC: 23710, 129: NM_001009992, 130: NM_172225,
131: ZNF211, 132: HGNC: 12992, 133: ENSG00000074657, 134: HGNC: 24618, 135:
HGNC: 11242, 136: NM_198177, 137: HGNC: 31953, 138: NM_020235, 139: HGNC: 35123,
140: ENSG00000122691, 141: Entrez ID: 23036, 142: ETV5, 143: JUND, 144:
NM_001376591, 145: NM_001142589, 146: HGNC: 20144, 147: ENSG00000127903, 148:
ENSG00000169840, 149: HGNC: 21478, 150: ZNF862, 151: ENSG00000125968, 152:
ENSG00000198081, 153: HGNC: 17923, 154: ENSG00000174796, 155: ZNF487, 156:
Entrez ID: 162993, 157: ENSG00000181690, 158: ENSG00000152217, 159:
NM_001379420, 160: ENSG00000185122, 161: BOLA2B, 162: HGNC: 17908, 163:
ENSG00000215612, 164: NM_013992, 165: HGNC: 1805, 166: NM_013999, 167:
HGNC: 11645, 168: NM_024017, 169: ENSG00000177508, 170: ENSG00000124203, 171:
ZNF529, 172: NM_001313900, 173: ENSG00000285124, 174: NM_001365803, 175: THRA,
176: HGNC: 7135, 177: ZNF282, 178: ENSG00000124216, 179: ENSG00000167232, 180:
CREB3, 181: ENSG00000171940, 182: INSM2, 183: ENSG00000170549, 184:
HGNC: 28382, 185: HGNC: 10449, 186: HGNC: 3808, 187: ENSG00000235068, 188: PAX3,
189: NM_001384880, 190: ZNF571, 191: ENSG00000281897, 192: ENSG00000109445,
193: NR2F2, 194: HGNC: 30306, 195: NM_001261437, 196: ENSG00000233890, 197:
ZNF780B, 198: HGNC: 20164, 199: ENSG00000281894, 200: HGNC: 29416, 201: ZFP14,
202: NM_001387775, 203: HGNC: 9143, 204: ENSG00000152284, 205: ENSG00000129028,
206: ZNF789, 207: HGNC: 8920, 208: NM_001393987, 209: HGNC: 33258, 210:
NM_001291746, 211: FOXK2, 212: ENSG00000197779, 213: ENSG00000204946, 214:
HGNC: 7795, 215: ENSG00000182348, 216: NM_001313904, 217: ENSG00000005889, 218:
ZNF490, 219: HGNC: 7804, 220: ZNF248, 221: NM_001387776, 222: NM_001349625, 223:
ENSG00000182111, 224: NM_001126242, 225: Entrez ID: 3233, 226: NM_001379413, 227:
HGNC: 11137, 228: ZNF611, 229: NM_001142587, 230: HGNC: 30776, 231:
ENSG00000169260, 232: ENSG00000143995, 233: ELK3, 234: HGNC: 6117, 235:
HGNC: 29181, 236: HGNC: 6593, 237: ENSG00000278062, 238: ZNF398, 239:
ENSG00000283026, 240: ZNF449, 241: HGNC: 15830, 242: ENSG00000137834, 243:
ZNF525, 244: NM_001276286, 245: Entrez ID: 56676, 246: ENSG00000198807, 247:
ENSG00000063587, 248: ENSG00000157554, 249: HGNC: 1975, 250: HGNC: 14078, 251:
Entrez ID: 93624, 252: ENSG00000184221, 253: ENSG00000260596, 254: Entrez ID:
80317, 255: NM_001204189, 256: NM_001384882, 257: PITX3, 258: NM_001369846, 259:
ZFYVE26, 260: ENSG00000171532, 261: GCM1, 262: Entrez ID: 51027, 263:
NM_001349613, 264: NM_014223, 265: ZNF43, 266: NM_001379415, 267: GATA4, 268:
ZNF418, 269: ENSG00000196092, 270: HGNC: 23847, 271: NM_001354608, 272: Entrez
ID: 1406, 273: SMYD2, 274: ENSG00000101115, 275: ENSG00000283681, 276: Entrez ID:
22926, 277: NM_001367510, 278: HGNC: 6131, 279: Entrez ID: 1054, 280: Entrez ID:
503582, 281: FEZF2, 282: HGNC: 18316, 283: NM_002126, 284: ENSG00000186814, 285:
HAND2, 286: HGNC: 13017, 287: ENSG00000223858, 288: ENSG00000196628, 289:
HGNC: 23762, 290: ENSG00000102554, 291: ENSG00000170608, 292: NFE2L1, 293:
ENSG00000275499, 294: ENSG00000188786, 295: FOXD1, 296: ENSG00000170485, 297:
MYBL1, 298: HGNC: 3118, 299: ENSG00000150907, 300: HGNC: 9046, 301: ZNF587, 302:
ENSG00000276340, 303: HGNC: 23191, 304: ENSG00000235712, 305: NM_001376587,
306: NM_018953, 307: HGNC: 12981, 308: HGNC: 6595, 309: ENSG00000175387, 310:
ENSG00000188909, 311: ENSG00000230336, 312: HGNC: 37273, 313: ENSG00000198551,
314: ENSG00000285120, 315: NM_001304764, 316: HGNC: 1329, 317: HGNC: 26720, 318:
ENSG00000168267, 319: HGNC: 28323, 320: HGNC: 27845, 321: ENSG00000135374, 322:
ENSG00000256229, 323: ENSG00000215421, 324: SALL2, 325: Entrez ID: 161882, 326:
HGNC: 8014, 327: Entrez ID: 283150, 328: ENSG00000167785, 329: ENSG00000099326,
330: CXXC1, 331: HGNC: 16762, 332: ENSG00000234674, 333: NM_001127216, 334:
EBF3, 335: ZBED1, 336: NM_032827, 337: ZNF564, 338: HGNC: 20636, 339: Entrez ID:
3239, 340: Entrez ID: 64211, 341: ENSG00000177426, 342: ENSG00000274195, 343:
NM_001384747, 344: NM_001384886, 345: NM_012184, 346: ENSG00000083307, 347:
HGNC: 29657, 348: NM_001393986, 349: HGNC: 12953, 350: ENSG00000167074, 351:
NM_001134442, 352: Entrez ID: 3231, 353: ENSG00000165512, 354: HGNC: 12983, 355:
XBP1, 356: HGNC: 21767, 357: ENSG00000284923, 358: ENSG00000176679, 359:
ENSG00000057657, 360: SIM1, 361: ZNF674, 362: Entrez ID: 7561, 363: NR1D2, 364:
NM_001384888, 365: ZNF629, 366: ENSG00000205189, 367: ZNF552, 368:
ENSG00000235178, 369: ENSG00000204612, 370: HGNC: 11203, 371: ENSG00000121297,
372: ENSG00000083828, 373: ENSG00000164916, 374: ENSG00000203883, 375:
ENSG00000280661, 376: ENSG00000177599, 377: ENSG00000066827, 378:
ENSG00000164749, 379: ENSG00000196263, 380: HGNC: 7965, 381: HGNC: 700, 382:
NM_001330322, 383: HGNC: 13027, 384: NM_001195752, 385: HGNC: 1835, 386:
HGNC: 26049, 387: NM_001024598, 388: ZNF672, 389: SOX14, 390: NM_001287347, 391:
HGNC: 18273, 392: HGNC: 7780, 393: ENSG00000234196, 394: ENSG00000120149, 395:
HSF5, 396: HGNC: 33783, 397: ENSG00000131127, 398: DMRT1, 399: Entrez ID: 5449,
400: NM_001085487, 401: ZSCAN10, 402: ENSG00000213988, 403: HGNC: 16447, 404:
KLF10, 405: HGNC: 13060, 406: HGNC: 13099, 407: ENSG00000236353, 408: ZNF843,
409: ENSG00000281661, 410: HGNC: 13847, 411: HGNC: 35126, 412: HGNC: 4319, 413:
HGNC: 16660, 414: VENTX, 415: HGNC: 13138, 416: TLX3, 417: HGNC: 12897, 418:
HGNC: 24819, 419: ENSG00000129535, 420: NM_001004311, 421: ENSG00000196350,
422: ZNF583, 423: NM_001293228, 424: ZNF281, 425: NM_001204192, 426: ZNF341, 427:
ENSG00000167081, 428: ENSG00000101216, 429: NM_001293231, 430: NM_003466, 431:
Entrez ID: 728361, 432: HGNC: 7982, 433: HGNC: 9005, 434: NM_001172517, 435: PAX1,
436: HGNC: 6380, 437: HGNC: 9351, 438: NM_198158, 439: NM_001986, 440:
HGNC: 22448, 441: IRX6, 442: ENSG00000265763, 443: HGNC: 7977, 444:
ENSG00000281306, 445: NM_001040002, 446: HGNC: 15979, 447: NM_001079675, 448:
HGNC: 17922, 449: NM_001145412, 450: ENSG00000261787, 451: ZNF527, 452: HES1,
453: ZNF79, 454: ENSG00000125878, 455: ENSG00000156127, 456: HIC2, 457: ATF4,
458: ZXDA, 459: ENSG00000282712, 460: NM_005378, 461: ENSG00000275050, 462:
ENSG00000124766, 463: HGNC: 3033, 464: NM_001270943, 465: ENSG00000172468, 466:
HGNC: 5107, 467: NM_001384881, 468: NM_205768, 469: ENSG00000170577, 470:
SOX10, 471: NM_138281, 472: ENSG00000067646, 473: ENSG00000204524, 474:
HGNC: 28357, 475: ENSG00000102804, 476: FOXD4, 477: ENSG00000165655, 478:
NM_001349620, 479: ENSG00000234539, 480: HGNC: 11602, 481: HGNC: 16736, 482:
HGNC: 32291, 483: HGNC: 13091, 484: HGNC: 4170, 485: ENSG00000130818, 486:
HGNC: 28079, 487: ENSG00000285178, 488: HGNC: 25292, 489: ENSG00000120705, 490:
NM_001304763, 491: NM_001384750, 492: ZNF839, 493: ENSG00000204920, 494:
NM_001354604, 495: TEAD2, 496: ENSG00000165556, 497: ENSG00000168468, 498:
ZNF254, 499: ZNF546, 500: HGNC: 20868, 501: BATF3, 502: ENSG00000168152, 503:
HGNC: 7785, 504: ENSG00000151623, 505: ENSG00000059145, 506: ENSG00000279566,
507: HGNC: 25108, 508: ENSG00000188620, 509: NM_001394498, 510: HGNC: 27963, 511:
ENSG00000223687, 512: HGNC: 20225, 513: NM_001290404, 514: ZNF236, 515: Entrez
ID: 100505555, 516: ZNF3, 517: HGNC: 21029, 518: HGNC: 28495, 519: HGNC: 21806, 520:
HGNC: 32034, 521: ZBED2, 522: ENSG00000197372, 523: ENSG00000178919, 524:
ENSG00000285438, 525: HGNC: 21200, 526: NM_004502, 527: HGNC: 7545, 528: NPAS4,
529: NM_001369368, 530: ZNF646, 531: HGNC: 19088, 532: ENSG00000147117, 533:
ENSG00000223852, 534: HGNC: 30946, 535: HGNC: 3239, 536: ENSG00000143867, 537:
ENSG00000152804, 538: NM_001261414, 539: Entrez ID: 10725, 540: ENSG00000281709,
541: NM_002664, 542: HGNC: 13175, 543: ENSG00000285286, 544: NM_001127215, 545:
NM_001369367, 546: ENSG00000178665, 547: HGNC: 12931, 548: NM_001318005, 549:
HGNC: 6205, 550: STAT4, 551: Entrez ID: 333929, 552: ENSG00000197933, 553:
HGNC: 23025, 554: ENSG00000225987, 555: HGNC: 20878, 556: MYT1, 557:
ENSG00000283060, 558: ZNF34, 559: HGNC: 29653, 560: ENSG00000196867, 561:
ENSG00000186230, 562: ZNF133, 563: HGNC: 29238, 564: ZNF562, 565: HGNC: 26420,
566: NM_001033081, 567: HGNC: 13078, 568: ZIC1, 569: NM_001349624, 570:
ENSG00000185668, 571: ENSG00000109851, 572: NM_001349609, 573: Entrez ID: 92822,
574: NM_001122770, 575: NM_001278196, 576: NM_015215, 577: ENSG00000168062,
578: HGNC: 13507, 579: ENSG00000176222, 580: HGNC: 21696, 581: SRF, 582:
ENSG00000228333, 583: ENSG00000105866, 584: HGNC: 19311, 585: NM_003189, 586:
ENSG00000124232, 587: HGNC: 4979, 588: KLF12, 589: HGNC: 9221, 590: Entrez ID:
3394, 591: ENSG00000183072, 592: HGNC: 9213, 593: HGNC: 4909, 594:
ENSG00000229094, 595: ZFP1, 596: NM_001367508, 597: NM_001184968, 598:
HGNC: 19321, 599: ENSG00000036549, 600: HGNC: 4978, 601: HGNC: 24149, 602: Entrez
ID: 3235, 603: ZNF511, 604: HGNC: 13804, 605: ZNF623, 606: HGNC: 7968, 607:
ENSG00000147183, 608: HGNC: 11751, 609: HGNC: 26765, 610: ENSG00000083844, 611:
ENSG00000167034, 612: ENSG00000288335, 613: GCM2, 614: HGNC: 21629, 615:
HGNC: 17441, 616: NM_001363736, 617: ENSG00000106261, 618: ENSG00000277421,
619: NM_001204186, 620: NM_002145, 621: HGNC: 17409, 622: HGNC: 16763, 623: E2F2,
624: ENSG00000120963, 625: ENSG00000274577, 626: GLIS2, 627: ENSG00000288204,
628: Entrez ID: 8648, 629: ENSG00000196233, 630: HGNC: 11639, 631: HGNC: 27271, 632:
NM_001369366, 633: NM_001349610, 634: ENSG00000205927, 635: ENSG00000214652,
636: NM_032522, 637: HGNC: 4370, 638: HGNC: 5099, 639: HGNC: 4074, 640: MYF6, 641:
HGNC: 7794, 642: NM_000248, 643: MYOD1, 644: NM_001395202, 645: ZNF10, 646:
HGNC: 26166, 647: MYF5, 648: HGNC: 13854, 649: HESX1, 650: ENSG00000179772, 651:
NM_001031680, 652: Entrez ID: 3097, 653: NM_001349614, 654: HGNC: 9455, 655: DBX2,
656: HGNC: 25352, 657: ENSG00000006377, 658: HGNC: 7321, 659: NM_198178, 660:
TBX19, 661: NM_001204188, 662: ZNF334, 663: HGNC: 17197, 664: ZFP42, 665:
NM_005414, 666: HES7, 667: NM_147192, 668: ENSG00000281626, 669: ZNF358, 670:
ENSG00000232005, 671: DRAP1, 672: ENSG00000124151, 673: TBX1, 674:
ENSG00000169946, 675: ENSG00000196767, 676: HGNC: 31986, 677: NM_001346115,
678: NM_001134441, 679: HGNC: 26758, 680: HGNC: 12915, 681: ENSG00000166478, 682:
ENSG00000101412, 683: HGNC: 6761, 684: NFXL1, 685: HGNC: 18737, 686:
ENSG00000138083, 687: ENSG00000198783, 688: ENSG00000198554, 689:
ENSG00000171469, 690: SOX15, 691: ENSG00000148704, 692: HGNC: 12955, 693:
ENSG00000185252, 694: CTCFL, 695: EBF4, 696: ENSG00000181315, 697:
ENSG00000196109, 698: ENSG00000105967, 699: HGNC: 20856, 700: NM_001379419,
701: HGNC: 28510, 702: NM_033210, 703: ENSG00000183434, 704: HGNC: 14361, 705:
ZNF607, 706: ENSG00000273274, 707: HGNC: 20190, 708: ENSG00000288283, 709:
ENSG00000060138, 710: RAX2, 711: NM_001394500, 712: ZNF569, 713: CREB3L1, 714:
ENSG00000215397, 715: DACH1, 716: NM_001282858, 717: NM_001145664, 718:
NM_001003845, 719: TOX4, 720: TBX4, 721: NM_006940, 722: Entrez ID: 4205, 723:
NM_001025603, 724: HGNC: 12919, 725: HGNC: 13061, 726: NM_153633, 727:
ENSG00000124191, 728: Entrez ID: 162966, 729: ENSG00000227322, 730: Entrez ID:
3159, 731: ENSG00000224379, 732: NM_001384749, 733: HGNC: 33106, 734: Entrez ID:
3662, 735: ENSG00000113430, 736: HGNC: 30988, 737: NM_001204184, 738:
HGNC: 10560, 739: NM_001349616, 740: Entrez ID: 84450, 741: NM_001282860, 742:
NM_001290405, 743: NM_002479, 744: ENSG00000173145, 745: ENSG00000189299, 746:
ENSG00000215474, 747: TSC22D3, 748: ZNF250, 749: ENSG00000144331, 750: LBX1,
751: HGNC: 12957, 752: NM_001379417, 753: ENSG00000180613, 754: ZNF256, 755:
NM_001395201, 756: ENSG00000198911, 757: HGNC: 13176, 758: ENSG00000101493,
759: FOSL1, 760: ENSG00000087510, 761: ENSG00000179388, 762: Entrez ID: 3237, 763:
HGNC: 5104, 764: NM_024016, 765: HGNC: 1836, 766: NM_001267043, 767: HGNC: 7134,
768: HGNC: 25597, 769: HGNC: 5105, 770: ENSG00000178229, 771: ENSG00000163132,
772: TUB, 773: ENSG00000125845, 774: ZXDB, 775: NM_001135610, 776: ZNF485, 777:
HGNC: 7836, 778: NM_139266, 779: ENSG00000167637, 780: ENSG00000276461, 781:
NM_001293233, 782: HGNC: 33115, 783: Entrez ID: 116113, 784: ZNF596, 785:
NM_001349617, 786: ENSG00000160224, 787: ZNF37A, 788: ENSG00000136826, 789:
HGNC: 1539, 790: Entrez ID: 64344, 791: ENSG00000141956, 792: ENSG00000043039,
793: SOX9, 794: ENSG00000281178, 795: ENSG00000118707, 796: HGNC: 10478, 797:
ENSG00000186766, 798: ENSG00000249961, 799: ZNF526, 800: NM_145044, 801:
HGNC: 8138, 802: NM_001042681, 803: Entrez ID: 84215, 804: Entrez ID: 162972, 805:
MXI1, 806: ENSG00000147180, 807: ENSG00000277015, 808: ENSG00000104447, 809:
HGNC: 5109, 810: HGNC: 29210, 811: HGNC: 7805, 812: NM_003709, 813:
ENSG00000159263, 814: ZNF326, 815: ENSG00000139800, 816: HGNC: 13994, 817:
ENSG00000178573, 818: ID3, 819: ENSG00000197360, 820: Entrez ID: 284346, 821:
NM_032788, 822: HGNC: 28854, 823: NM_013952, 824: ENSG00000235589, 825: Entrez
ID: 10664, 826: NM_015866, 827: ENSG00000061455, 828: NM_001394499, 829:
HGNC: 13016, 830: ENSG00000105880, 831: ENSG00000227858, 832: ENSG00000019549,
833: ENSG00000100968, 834: HGNC: 16747, 835: HGNC: 20152, 836: HGNC: 13672, 837:
ZNF791, 838: HGNC: 346, 839: ENSG00000143032, 840: HGNC: 13104, 841: Entrez ID:
56917, 842: DBP, 843: HGNC: 3017, 844: HGNC: 26328, 845: HGNC: 26629, 846:
HGNC: 33108, 847: ENSG00000186350, 848: NM_003036, 849: ENSG00000136535, 850:
HGNC: 11641, 851: HGNC: 29983, 852: ENSG00000134852, 853: HGNC: 6345, 854: ZNF16,
855: NM_001354606, 856: ENSG00000283009, 857: HGNC: 25669, 858: NM_002908, 859:
NM_001145098, 860: ENSG00000100426, 861: BCLAF1, 862: Entrez ID: 22807, 863:
NM_014620, 864: HGNC: 8618, 865: EHF, 866: ENSG00000204103, 867: KIAA1549, 868:
ENSG00000206315, 869: Entrez ID: 10522, 870: ENSG00000197714, 871: HGNC: 9984,
872: ENSG00000285493, 873: ENSG00000224941, 874: HGNC: 25594, 875: HGNC: 11729,
876: ENSG00000250312, 877: OTX2, 878: HGNC: 14008, 879: ENSG00000167157, 880:
POU3F3, 881: ZNF581, 882: HGNC: 12988, 883: ENSG00000197044, 884: ZNF678, 885:
ZNF175, 886: HGNC: 18609, 887: MYRF, 888: HGNC: 24050, 889: ENSG00000165495,
890: HGNC: 14097, 891: ENSG00000280774, 892: HGNC: 11753, 893: ENSG00000206454,
894: ENSG00000025293, 895: HGNC: 11811, 896: ENSG00000197050, 897:
NM_001313901, 898: ZNF549, 899: Entrez ID: 1389, 900: ENSG00000275746, 901:
NM_001204961, 902: ENSG00000072364, 903: HGNC: 6551, 904: ENSG00000171056, 905:
PRDM12, 906: NM_001320672, 907: HES6, 908: NM_001282856, 909:
ENSG00000280568, 910: HGNC: 16421, 911: TBX2, 912: NM_001184967, 913:
NM_020832, 914: ENSG00000122034, 915: NM_001076684, 916: ENSG00000108064, 917:
ENSG00000009709, 918: REST, 919: ENSG00000176399, 920: ENSG00000168795, 921:
MAFF, 922: SP100, 923: Entrez ID: 64764, 924: HGNC: 28200, 925: ENSG00000112837,
926: HGNC: 25777, 927: HGNC: 6594, 928: HGNC: 29147, 929: HGNC: 4930, 930: DMTF1,
931: ENSG00000165061, 932: NM_001291281, 933: ZNF280B, 934: ZNF30, 935:
ENSG00000205922, 936: ENSG00000095574, 937: HGNC: 32466, 938: HEY2, 939:
HGNC: 25919, 940: ENSG00000184677, 941: Entrez ID: 101060200, 942: HGNC: 17321,
943: ENSG00000106546, 944: NM_001330785, 945: HGNC: 30950, 946: HGNC: 26498, 947:
NM_012231, 948: NM_005427, 949: HGNC: 30461, 950: ENSG00000177732, 951:
ENSG00000105750, 952: ENSG00000277800, 953: HGNC: 1123, 954: POU2F1, 955:
ENSG00000273529, 956: NR1D1, 957: SOX2, 958: TERF1, 959: ENSG00000067082, 960:
ENSG00000142700, 961: ENSG00000134438, 962: HGNC: 29014, 963: ENSG00000263310,
964: Entrez ID: 3398, 965: NM_001142590, 966: POU4F3, 967: HGNC: 2935, 968:
HGNC: 8910, 969: ZSCAN25, 970: NM_152320, 971: ENSG00000124201, 972:
ENSG00000137709, 973: ENSG00000161405, 974: ENSG00000214513, 975: HGNC: 38705,
976: HGNC: 20814, 977: ENSG00000184937, 978: NM_033343, 979: HGNC: 37246, 980:
ESRRB, 981: HGNC: 26496, 982: ELK4, 983: ENSG00000138311, 984: MLXIP, 985:
NM_001145413, 986: ENSG00000281277, 987: NM_001934, 988: HGNC: 11366, 989:
ENSG00000156374, 990: MAEL, 991: NM_032292, 992: HGNC: 5103, 993: HGNC: 28043,
994: HGNC: 11742, 995: HGNC: 28071, 996: HGNC: 12993, 997: NM_001172682, 998:
ZNF670, 999: Entrez ID: 51085, 1000: HGNC: 20393, 1001: ZNF655, 1002:
ENSG00000125816, 1003: NM_001379414, 1004: ENSG00000285109, 1005: HGNC: 2349,
1006: HGNC: 14214, 1007: SPIC, 1008: NM_001349612, 1009: ZNF784, 1010: ZNF416,
1011: ENSG00000228628, 1012: ENSG00000184302, 1013: NFE2L3, 1014:
ENSG00000025156, 1015: HGNC: 4172, 1016: ENSG00000276876, 1017: HGNC: 18779,
1018: Entrez ID: 7699, 1019: HGNC: 30498, 1020: ZNF570, 1021: ENSG00000101190,
1022: HGNC: 6106, 1023: HGNC: 29003, 1024: Entrez ID: 57335, 1025: HGNC: 11368, 1026:
Entrez ID: 3234, 1027: ENSG00000280467, 1028: NM_001042682, 1029:
ENSG00000101096, 1030: NM_001353131, 1031: ZNF580, 1032: HGNC: 19196, 1033:
ZC3H3, 1034: Entrez ID: 195828, 1035: HGNC: 11205, 1036: Entrez ID: 3238, 1037:
NM_001204963, 1038: ENSG00000281526, 1039: HGNC: 8616, 1040: HGNC: 16682, 1041:
HGNC: 4317, 1042: ENSG00000227124, 1043: NM_001204191, 1044: FOXA2, 1045:
NM_002585, 1046: DACH2, 1047: HGNC: 37134, 1048: NM_001349623, 1049: Entrez ID:
162967, 1050: HGNC: 20690, 1051: FIZ1, 1052: ENSG00000173451, 1053:
ENSG00000141905, 1054: ENSG00000186812, 1055: HGNC: 28197, 1056:
ENSG00000141448, 1057: ENSG00000147596, 1058: HGNC: 24415, 1059: HGNC: 9982,
1060: HGNC: 27267, 1061: ENSG00000182903, 1062: ALX3, 1063: HGNC: 5724, 1064:
ZNF524, 1065: PROX2, 1066: CEBPZ, 1067: NM_000449, 1068: HGNC: 20877, 1069:
HGNC: 18855, 1070: LMX1A, 1071: Entrez ID: 4760, 1072: HGNC: 7764, 1073:
HGNC: 12958, 1074: HGNC: 3803, 1075: ENSG00000283034, 1076: ENSG00000283949,
1077: HGNC: 25883, 1078: Entrez ID: 105372481, 1079: NM_001485, 1080: Entrez ID:
51710, 1081: HGNC: 20629, 1082: HGNC: 21948, 1083: ENSG00000184895, 1084: PROX1,
1085: FOXH1, 1086: HGNC: 25105, 1087: FOXN3, 1088: ZNF724, 1089: Entrez ID: 649137,
1090: ZNF579, 1091: HGNC: 12887, 1092: ZNF792, 1093: HGNC: 6769, 1094:
ENSG00000043355, 1095: ENSG00000281518, 1096: HGNC: 33202, 1097: HGNC: 20994,
1098: KLF16, 1099: HGNC: 3811, 1100: HGNC: 12975, 1101: HGNC: 28160, 1102:
ENSG00000131116, 1103: ENSG00000135625, 1104: NM_198159, 1105: ZNF280A, 1106:
ENSG00000152192, 1107: ENSG00000269067, 1108: HGNC: 13019, 1109:
ENSG00000181444, 1110: HGNC: 28501, 1111: HGNC: 25932, 1112: ZNF585B, 1113:
ZNF808, 1114: HGNC: 3526, 1115: HGNC: 33519, 1116: HGNC: 13020, 1117:
ENSG00000140044, 1118: NKX1-1, 1119: ENSG00000234669, 1120: HGNC: 11714, 1121:
ENSG00000176182, 1122: HGNC: 6204, 1123: ENSG00000135638, 1124: TLX2, 1125:
OLIG3, 1126: ENSG00000171872, 1127: ENSG00000231321, 1128: ENSG00000198795,
1129: HGNC: 6767, 1130: ENSG00000198839, 1131: ENSG00000005801, 1132:
HGNC: 28730, 1133: HGNC: 5106, 1134: PRDM13, 1135: NM_004527, 1136: ZNF71, 1137:
HGNC: 10891, 1138: ENSG00000008196, 1139: NM_001261438, 1140: NM_001349621,
1141: ENSG00000179627, 1142: HGNC: 3816, 1143: NM_001384885, 1144: HGNC: 7960,
1145: HGNC: 20817, 1146: NM_024015, 1147: ENSG00000197921, 1148: HGNC: 12593,
1149: ZFP62, 1150: ZNF592, 1151: ENSG00000125820, 1152: ZFHX3, 1153: ADNP, 1154:
CUX2, 1155: ENSG00000281601, 1156: NM_001270944, 1157: ENSG00000182141, 1158:
HGNC: 12927, 1159: ENSG00000283868, 1160: GZF1, 1161: ENSG00000169953, 1162:
NM_001393988, 1163: ENSG00000168813, 1164: ENSG00000160229, 1165: HGNC: 20648,
1166: NM_001145511, 1167: NM_018952, 1168: HGNC: 23245, 1169: ENSG00000189180,
1170: NM_001261439, 1171: ENSG00000198815, 1172: HGNC: 12949, 1173:
ENSG00000111704, 1174: Entrez ID: 388559, 1175: DLX3, 1176: RORA, 1177:
HGNC: 28696, 1178: ZNF239, 1179: RC3H2, 1180: ENSG00000258873, 1181:
HGNC: 13155, 1182: ENSG00000151379, 1183: HGNC: 6116, 1184: NM_015265, 1185:
ENSG00000236515, 1186: HGNC: 14536, 1187: ENSG00000260428, 1188: HGNC: 12984,
1189: ENSG00000116016, 1190: ENSG00000101665, 1191: NM_004350, 1192: SPI1, 1193:
TFAP2D, 1194: NM_001349615, 1195: HGNC: 5108, 1196: ENSG00000282892, 1197:
ENSG00000103460, 1198: HGNC: 12980, 1199: NM_001364867, 1200: HGNC: 13222, 1201:
HGNC: 4880, 1202: HGNC: 21193, 1203: HGNC: 13806, 1204: Entrez ID: 353274, 1205:
NM_001301687, 1206: ZNF92, 1207: ENSG00000282899, 1208: ZNF517, 1209:
ENSG00000157557, 1210: NM_001354605, 1211: HGNC: 32284, 1212: ENSG00000229676,
1213: ZNF41, 1214: ENSG00000063515, 1215: ENSG00000130700, 1216:
ENSG00000274636, 1217: HGNC: 1107, 1218: ENSG00000100207, 1219:
ENSG00000262024, 1220: Entrez ID: 6720, 1221: HGNC: 26457, 1222: ENSG00000183850,
1223: ENSG00000197016, 1224: NM_001376588, 1225: ENSG00000113658, 1226:
NM_001330323, 1227: ENSG00000136944, 1228: ENSG00000156925, 1229:
ENSG00000284774, 1230: SHOX, 1231: TLX1, 1232: NKRF, 1233: HGNC: 29658, 1234:
ENSG00000160352, 1235: ENSG00000153560, 1236: ENSG00000197134, 1237:
ENSG00000153779, 1238: ENSG00000197124, 1239: ENSG00000276561, 1240:
NM_001379418, 1241: ENSG00000142611, 1242: DNAJC2, 1243: NM_001134673, 1244:
NM_001142568, 1245: NM_006164, 1246: HGNC: 23258, 1247: NKX2-1, 1248:
NM_001172509, 1249: ENSG00000170370, 1250: POU4F2, 1251: ENSG00000196693,
1252: ZMAT2, 1253: ENSG00000126767, 1254: HGNC: 7983, 1255: Entrez ID: 64375,
1256: Entrez ID: 6668, 1257: Entrez ID: 56978, 1258: ENSG00000233608, 1259: CEBPB,
1260: ENSG00000206510, 1261: ZFP90, 1262: ENSG00000261678, 1263:
ENSG00000278091, 1264: ZNF493, 1265: ZNF845, 1266: HGNC: 18122, 1267:
NM_001384887, 1268: HGNC: 25612, 1269: NM_007315, 1270: HGNC: 13644, 1271:
KDM4C, 1272: NM_001379416, 1273: NM_001282430, 1274: HGNC: 23385, 1275:
HGNC: 25173, 1276: ENSG00000170260, 1277: ENSG00000175691, 1278: HGNC: 9349,
1279: ZKSCAN5, 1280: ENSG00000146592, 1281: NM_005595, 1282: Entrez ID: 63946,
1283: HGNC: 18695, 1284: Entrez ID: 57215, 1285: HGNC: 25885, 1286: NM_005531, 1287:
ENSG00000105717, 1288: ENSG00000169083, 1289: HGNC: 1808, 1290: HGNC: 7980,
1291: HGNC: 15809, 1292: HGNC: 7553, 1293: HGNC: 13805, 1294: NM_001033082, 1295:
NM_001349618, 1296: ZNF547, 1297: NR5A2, 1298: NM_001204190, 1299:
ENSG00000172466, 1300: NM_178010, 1301: HGNC: 22213, 1302: ZNF624, 1303:
HGNC: 28632, 1304: ATF3, 1305: HGNC: 26897, 1306: ZNF782, 1307: HGNC: 26273, 1308:
ENSG00000232099, 1309: HGNC: 13363, 1310: Entrez ID: 4899, 1311: ATMIN, 1312:
HGNC: 25827, 1313: ENSG00000156273, 1314: ENSG00000198963, 1315: Entrez ID:
201516, 1316: FOXL1, 1317: ENSG00000159917, 1318: ENSG00000096401, 1319:
HGNC: 7802, 1320: NM_012183, 1321: HGNC: 26479, 1322: NM_001270942, 1323:
NM_001009812, 1324: HGNC: 27614, 1325: ENSG00000164379, 1326: HGNC: 7975, 1327:
NM_001384891, 1328: Entrez ID: 2115, 1329: HGNC: 7974, 1330: HGNC: 19066, 1331:
NM_001384883, 1332: ENSG00000182463, 1333: ENSG00000149050, 1334: HGNC: 4882,
1335: ERF, 1336: NM_001369397, 1337: NKX2-6, 1338: ENSG00000054598, 1339:
ENSG00000180438, 1340: ENSG00000285485, 1341: NM_001384884, 1342:
ENSG00000159216, 1343: NM_001258280, 1344: HGNC: 19883, 1345: LYL1, 1346:
ENSG00000173276, 1347: NM_001394497, 1348: ENSG00000237344, 1349:
ENSG00000126778, 1350: ASCL1, 1351: HGNC: 25226, 1352: ENSG00000116017, 1353:
NM_001365804, 1354: ENSG00000147118, 1355: HGNC: 29659, 1356: NM_001242701,
1357: ENSG00000056277, 1358: HGNC: 20811, 1359: HGNC: 29093, 1360: HGNC: 11599,
1361: ENSG00000176407, 1362: ENSG00000086102, 1363: ENSG00000102349, 1364:
SOHLH2, 1365: HGNC: 5101, 1366: ENSG00000103199, 1367: SP6, 1368:
ENSG00000171295, 1369: Entrez ID: 146050, 1370: HGNC: 13154, 1371: HGNC: 12902,
1372: HGNC: 12948, 1373: ENSG00000159882, 1374: HGNC: 12904, 1375: Entrez ID:
94039, 1376: NM_019089, 1377: ENSG00000137273, 1378: HGNC: 27713, 1379:
ENSG00000182968, 1380: ENSG00000115844, 1381: NM_001376589, 1382: ZNF787,
1383: ENSG00000142684, 1384: NM_001290403, 1385: HGNC: 30690, 1386: HGNC: 1092,
1387: NM_001114176, 1388: Entrez ID: 503834, 1389: ENSG00000171467, 1390: FEV,
1391: ZNF414, 1392: ENSG00000274352, 1393: NM_153207, 1394: ENSG00000133740,
1395: MAX, 1396: HGNC: 28053, 1397: Entrez ID: 9841, 1398: NM_001248008, 1399:
HGNC: 29297, 1400: NM_001394496, 1401: ZNF70, 1402: HGNC: 5102, 1403:
NM_001349627, 1404: NR1H4, 1405: NM_001134440, 1406: NM_001145512, 1407:
ENSG00000167771, 1408: HGNC: 13146, 1409: Entrez ID: 4825, 1410: HGNC: 13149, 1411:
Entrez ID: 25803, 1412: HGNC: 20564, 1413: ENSG00000109787, 1414: NM_006722, 1415:
HGNC: 11598, 1416: ENSG00000226858, 1417: ENSG00000039600, 1418: ZNF740, 1419:
MIER3, 1420: NM_152989, 1421: ZMAT1, 1422: DMRT3, 1423: HGNC: 24905, 1424:
NM_001395199, 1425: HGNC: 8518, 1426: TCF21, 1427: ZNF462, 1428:
ENSG00000016082, 1429: ENSG00000175395, 1430: Entrez ID: 58158, 1431: ZNF473,
1432: ZNF540, 1433: HGNC: 12856, 1434: ENSG00000233911, 1435: ENSG00000196357,
1436: HGNC: 32467, 1437: KDM5C, 1438: ENSG00000285328, 1439: NM_001349626,
1440: KLF2, 1441: HGNC: 13067, 1442: PHOX2A, 1443: ENSG00000106511, 1444:
NM_001206567, 1445: HGNC: 11605, 1446: FOXF1, 1447: ENSG00000224952, 1448:
ENSG00000150051, 1449: HGNC: 23712, 1450: ZIK1, 1451: HMX2, 1452: NM_001313902,
1453: NM_138350, 1454: Entrez ID: 3232, 1455: NM_001172681, 1456:
ENSG00000165259, 1457: TFDP1, 1458: HGNC: 16656, 1459: E2F6, 1460: HGNC: 21894,
1461: YEATS2, 1462: NM_001354607, 1463: Entrez ID: 284355, 1464: HGNC: 29009, 1465:
ENSG00000186496, 1466: ENSG00000186777, 1467: NM_001308114, 1468:
ENSG00000122145, 1469: HGNC: 33105, 1470: ENSG00000113196, 1471: ZNF695, 1472:
Entrez ID: 4861, 1473: SOX13, 1474: HGNC: 10524, 1475: NM_001076683, 1476:
ENSG00000139515, 1477: NM_001376592, 1478: NM_001282880, 1479: NM_002146,
1480: POU6F2, 1481: HGNC: 18160, 1482: ENSG00000160199, 1483: HGNC: 20812, 1484:
HGNC: 25879, 1485: HGNC: 3527, 1486: HGNC: 3506, 1487: HGNC: 12922, 1488:
HGNC: 25736, 1489: HGNC: 25953, 1490: HGNC: 23179, 1491: ENSG00000129173, 1492:
ENSG00000141946, 1493: HGNC: 3238, 1494: ENSG00000165030, 1495: NM_002144,
1496: ZNF174, 1497: ZNF213, 1498: Entrez ID: 80152, 1499: ENSG00000134595, 1500:
ENSG00000176887, 1501: ENSG00000173404, 1502: HGNC: 1081, 1503: HGNC: 797, 1504:
NM_001330375, 1505: Entrez ID: 253461, 1506: HGNC: 17925, 1507: HGNC: 27030, 1508:
NM_001007257, 1509: HGNC: 16876, 1510: ZNF134, 1511: NM_006352, 1512: ZNF221,
1513: ENSG00000170345, 1514: ZNF587B, 1515: DRGX, 1516: HGNC: 15489, 1517:
HGNC: 18854, 1518: HGNC: 951, 1519: ENSG00000171476, 1520: Entrez ID: 163071, 1521:
ENSG00000100625, 1522: NM_001195563, 1523: HGNC: 11604, 1524: Entrez ID: 2116,
1525: ZNF519, 1526: Entrez ID: 4212, 1527: HGNC: 450, 1528: TFAP4, 1529:
ENSG00000213999, 1530: Entrez ID: 5080, 1531: ENSG00000117000, 1532: HGNC: 28917,
1533: ZNF585A, 1534: ENSG00000274382, 1535: NM_001379412, 1536: NM_001313903,
1537: HGNC: 16403, 1538: ZEB1, 1539: HGNC: 9985, 1540: ZNF260, 1541: HGNC: 7788,
1542: HGNC: 31446, 1543: NM_001308115, 1544: NM_001384890, 1545: ZNF597, 1546:
ZNF28, 1547: ENSG00000101057, 1548: GFI1B, 1549: HOXB13, 1550:
ENSG00000198146, 1551: ZNF668, 1552: HGNC: 7969, 1553: HGNC: 27603, 1554:
ARID5B, 1555: ENSG00000124459, 1556: HGNC: 5227, 1557: HGNC: 9983, 1558: LHX9,
1559: ENSG00000184828, 1560: HGNC: 13843, 1561: MAFA, 1562: ENSG00000139083,
1563: Entrez ID: 4208, 1564: ENSG00000188785, 1565: EN2, 1566: Entrez ID: 93986, 1567:
HGNC: 13059, 1568: ENSG00000229544, 1569: Entrez ID: 27086, 1570: HGNC: 3115, 1571:
HGNC: 12999, 1572: ENSG00000276219, 1573: ENSG00000272602, 1574: HGNC: 3126,
1575: ENSG00000165804, 1576: ENSG00000237440, 1577: IRX2, 1578:
ENSG00000175329, 1579: HGNC: 21399, 1580: HGNC: 26367, 1581: HGNC: 30489, 1582:
Entrez ID: 7101, 1583: ENSG00000221994, 1584: ENSG00000280635, 1585: HGNC: 29161,
1586: HGNC: 13995, 1587: HGNC: 32462, 1588: HGNC: 12939, 1589: PRRX1, 1590: HIF1A,
1591: HGNC: 20711, 1592: ZNF560, 1593: TCF12, 1594: ENSG00000196705, 1595:
HGNC: 30571, 1596: ENSG00000119547, 1597: HGNC: 13906, 1598: ENSG00000237582,
1599: ENSG00000164853, 1600: NM_001282861, 1601: ENSG00000124813, 1602: ZBTB3,
1603: ENSG00000131668, 1604: NM_001349622, 1605: NM_002147, 1606: HGNC: 12978,
1607: ESRRA, 1608: ENSG00000064490, 1609: NM_001204187, 1610: HGNC: 5100, 1611:
ENSG00000154727, 1612: ATOH7, 1613: HGNC: 25872, 1614: HGNC: 6781, 1615:
NM_001349619, 1616: HGNC: 26995, 1617: ENSG00000224472, 1618: HGNC: 12875, 1619:
ENSG00000285048, 1620: ZNF251, 1621: NM_005253, 1622: ENSG00000171956, 1623:
ZNF813, 1624: ENSG00000157259, 1625: ZNF417, 1626: NM_001384889, 1627:
NM_005263, 1628: HGNC: 4320

### Example 13: Expression of Albumin Detected Following Induction of TF Recipes

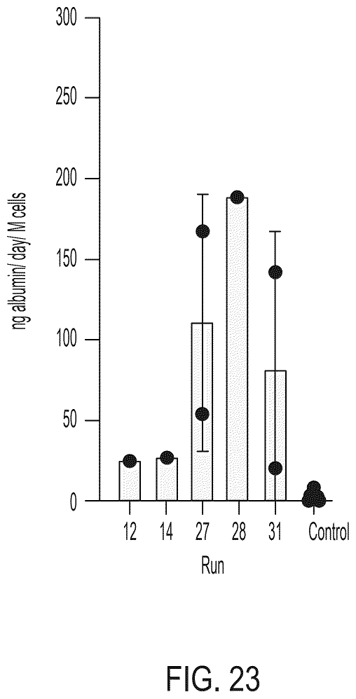

Cells were administered TF recipes as disclosed before and induction of differentiation was allowed to proceed for at least 96 hours. The amount of albumin secreted by the differentiated cells was measured by ELISA. Cell modified to express five TF combinations or recipes, namely, run 12, 14, 27, 28, and 31 (corresponding H0047, H0049, H0062, H0063, and H0066) resulted in detectable levels of albumin (FIG. 23).

### Example 14: Assessing TF Recipe H0011-Associated iPSC Differentiated Cells

A single cell RNA-sequencing experiment was designed to achieve two goals, (1) to gain additional insight into specific transcription factor combinations driving the differentiation of the iPSCs cells towards the hepatocyte lineage, (2) to characterize the hepatocyte-like cells and compare their gene expression to primary human hepatocytes. For both of these goals, the H0011 TF combination which is one of the key recipes with critical combination to drive iPSCs differentiation to hepatocytes was selected for further investigation.

The single cell RNA-sequencing experiment compared the transcriptomes of induced H0011 cells, uninduced H0011 cells, control iPSCs, and primary human hepatocytes. The H0011 cell line was created by transfecting iPSCs with seven transcription factors: CEBPA, FOXA1, FOXA2, HNF1A, HNF4A, ONECUT1/HNF6, and RBPJ.

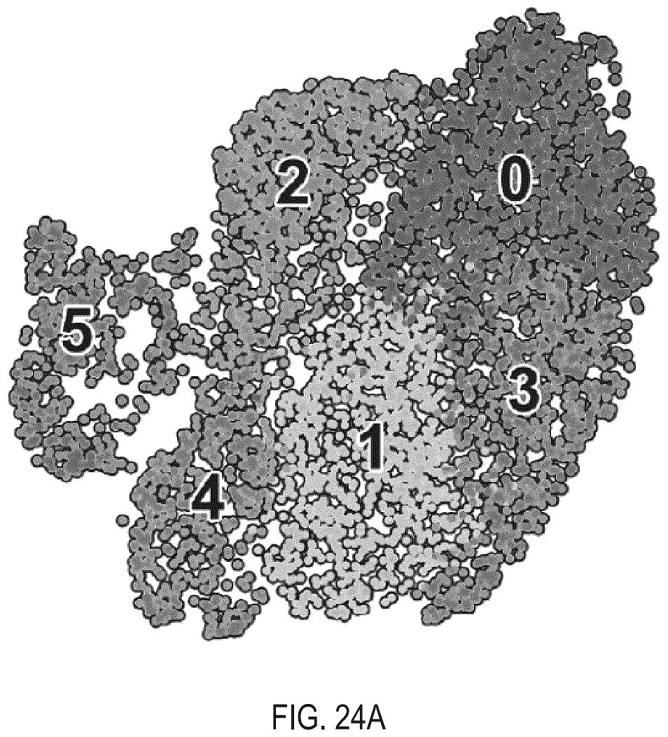

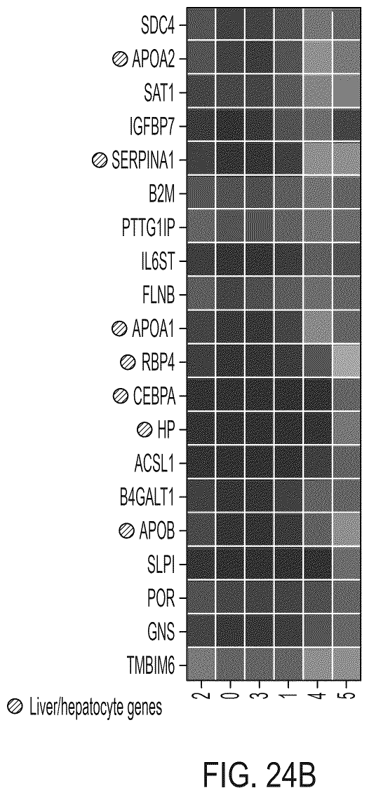

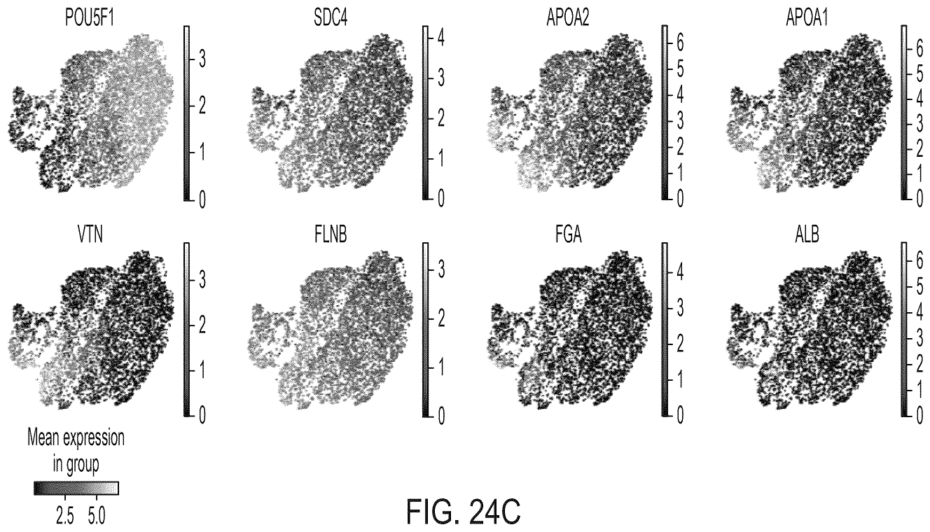

The induced H0011 cells could be categorized into six subclusters (FIG. 24A). UMAP of induced H0011 cells reveals two clusters of cells that express hepatocyte genes and have down regulated expression of pluripotent genes (FIG. 24A). This clustering of the induced H0011 cells revealed six clusters of gene expression profiles. A heat map of gene expression showed that clusters 4 and 5 had upregulated expression of hepatocyte genes (FIG. 24B), including APOA2, SERPINA1, APOA1, RBP4, CEBPA, HP, AND APOB. A map generated for the gene expression of selected genes onto the UMAP showed that clusters 4 and 5 had low expression of the stem cell marker POU5F1 and higher expression of hepatocyte markers including APOA2, fibrinogen (FGA), vitronectin (VTN), and albumin (ALB) (FIG. 24C). Clusters 4 and 5 had low expression of pluripotent markers such as POU5F1 (Oct4) and instead showed expression of hepatocyte-associated markers including SERPINA1, APOA1, APOA2, vitronectin (VTN), fibrinogen (FGA), and albumin (ALB) (FIG. 24B-24C), confirming that iPSCs had differentiated into hepatocytes.

### Example 15: Using Machine Learning to Understand iPSC Differentiation into Hepatocyte Lineage

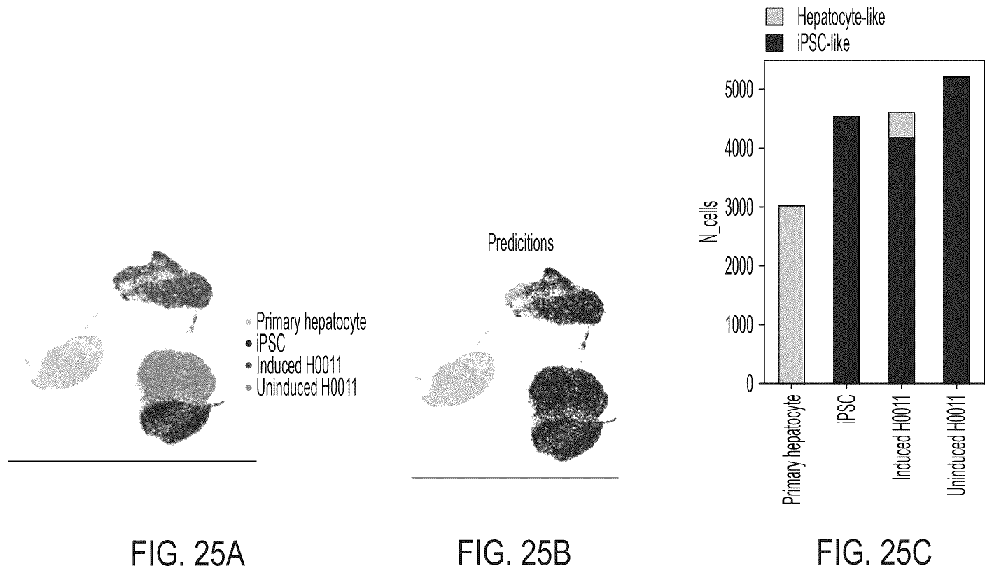

This study utilized a machine learning algorithm to assess the gene expression profiles of cells and categorize them as iPSC-like or hepatocyte-like. A UMAP showing the four samples studied: primary human hepatocytes, iPSCs, induced H0011 cells, and uninduced H0011 cells (FIG. 25A). Results from the classification (algorithm) exercise showed that primary human hepatocytes classified as hepatocyte-like and the iPSCs categorized as iPSC-like, while cells in which the H0011 transcription factor recipe was not activated (uninduced H0011) clustered more closely with the iPSCs than with the induced H0011 cells (FIG. 25A). Hepatocyte-like cells categorized by the classification algorithm included primary hepatocytes and iPSC-derived hepatocytes (FIG. 25B). The number of hepatocyte-like and iPSC-like cells observed in each sample is shown in FIG. 25C. These data confirmed the accuracy of the classification algorithm. Approximately 5% of the cells that had H0011 activated (induced H0011 cells) also classified as hepatocyte-like (FIG. 25C). These cells were found in clusters 4 and 5 that had been previously identified (FIG. 24A-C), which further supports their hepatocyte identity. These data further support the TF recipe H0011's role in iPSC differentiation into hepatocytes and importantly, highlights the accuracy of the classification algorithm which identified induced cells as hepatocytes or hepatocyte-like cells.

### Example 16: Characterizing Gene Expression of Induced H0011 Predicted Hepatocyte-Like Cells

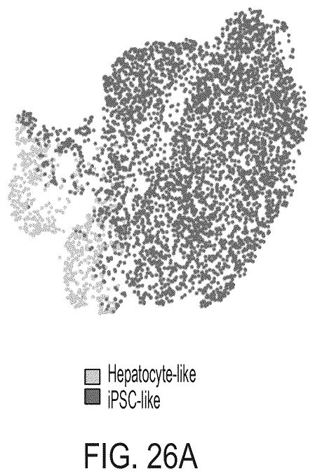

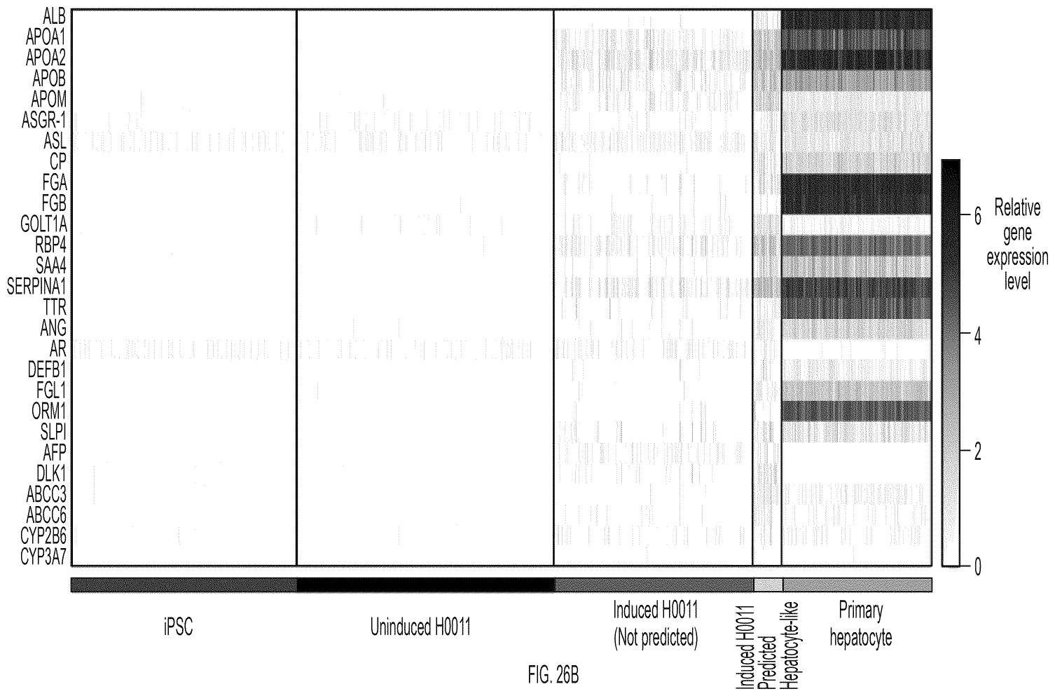

A further investigation was carried out on the 5% of induced H0011 cells that the classification algorithm predicted as hepatocyte-like cells (FIG. 25C). The assessment sought to establish gene expression profile among these predicted cells. The expression profile showed predicted cells expressed specific immature and mature hepatocyte markers; some immature hepatocyte markers included DLK1 and AFP and clustered together in the UMAP of the induced H0011 cells (FIG. 26A). The relative gene expression of a selected group of hepatocyte markers across all samples was evaluated (FIG. 26B) and again, the predicted cells had enriched expression of many key mature hepatocyte genes, including albumin, ASGR-1, apolipoproteins, and SERPINA1 (alpha-1-antitrypsin) (FIG. 26B) Expression of additional gene, for example, ABCC2, ABCC3, ABCC6, ACSL1, AFP, ALB, ALDH6A1, ANG, APOA1, APOA2, APOB, APOM, AR, ASGR1, ASL, CP, CYP2A7, CYP2B6, CYP3A5, CYP3A7, DEFB1, DLK1, FAH, FGA, FGB, FGL1, GOLT1A, HAL, HPR, LEPR, MTTP, ORM1, PLIN1, RBP4, SAA4, SERPINA1, SLPI, SULT1A1, TAT, and TTR was detected. Focusing on the predicted hepatocyte-like cells in the induced H0011 cells, it was observed that the predicted cells expressed many of the same hepatocyte-genes that are also expressed in the hepatocyte lineage.

### Example 17: H0011 Hepatocyte-Like Cells Gene Profile Enriched for Hepatocyte-Associated Genes

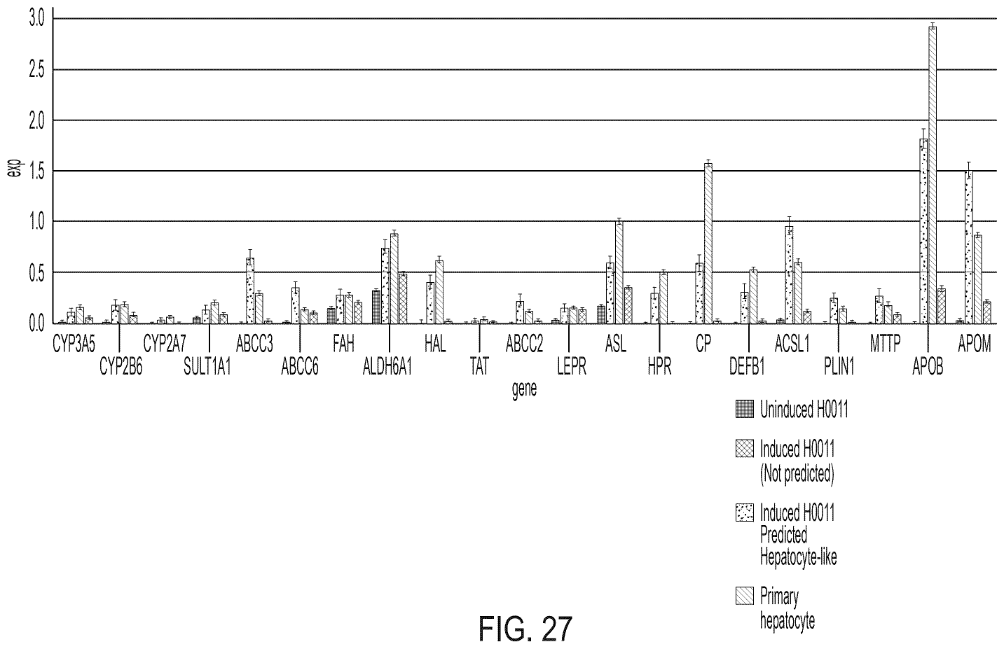

The predicted 5% hepatocyte-like cells in induced H0011 cells have enriched expression of many hepatocyte-associated genes, including CYP3A5, CYP2B6, CYP2A7, SULT1A1, ABCC3, ABCC6, FAH, ALDH6A1, HAL, TAT, ABCC2, LEPR, ASL, HPR, CP, DEFB1, ACSL1, PLIN1, MTTP, APOB, APOM (FIG. 27). In general, the transcriptome analysis showed that the quality and quantity of gene expression of individual genes in the induced H0011 cells predicted hepatocyte-like cells was similar to that observed in the primary hepatocytes (FIG. 26B and FIG. 27). These data confirm that iPSCs predicted to be hepatocyte-like had in fact differentiated into hepatocytes.

### Example 18: Assessing TFs that Drive iPSC Differentiation into Hepatocyte-Like Cells in Induced H0011 TF Combination

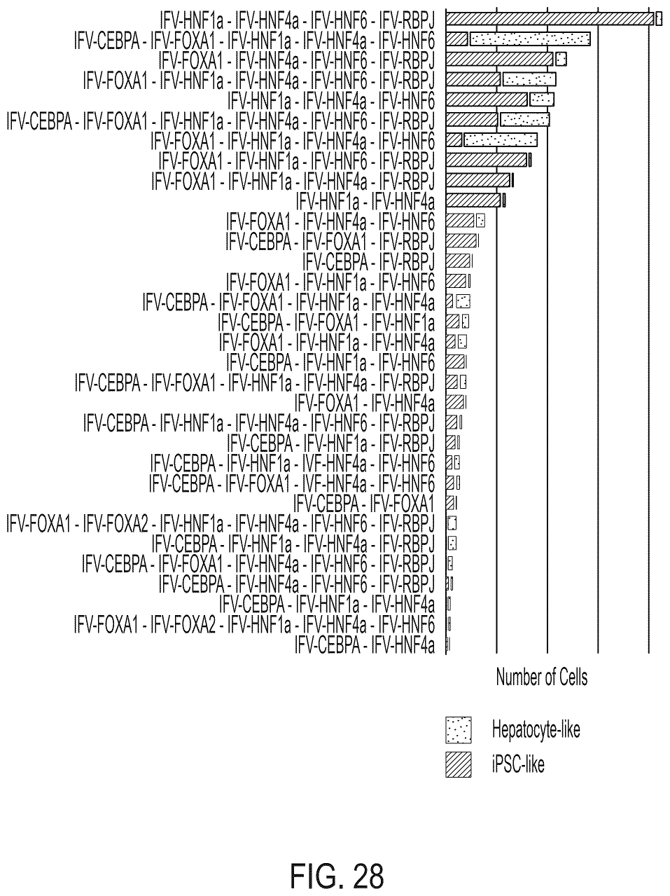

Analyses were conducted to identify transcription factor combinations that lead to high proportion of hepatocyte-like cells in induced H0011 cells. The exogenous transcription factor (TF) expression data was used to identify the combinations of transcription factors present in each individual cell in induced H0011 cells (FIG. 28). By plotting the number of predicted hepatocyte-like cells for every combination of transcription factors, example combinations of transcription factors (TF) were identified that associated with hepatocyte-like transcriptomes. For example, TF combinations or TF recipes (cocktails) were likely to be associated with hepatocyte-like transcriptomes when they included certain transcription factors (TF) such as, for example, CEBPA, FOXA1, HNF1A, HNF4A, FOXA1, HNF1A, HNF4A, HNF6, FOXA1, HNF1A, HNF4A, HNF6, RBPJ, CEBPA, FOXA1, HNF1A, HNF4A, HNF6, RBPJ, CEBPA, FOXA1, HNF1A, and HNF4A. Notably, all of these combinations included FOXA1, HNF1A, and HNF4A. When mapped onto the UMAP, the cells expressing these transcription factor combinations clustered near each other, suggesting that multiple transcription factor recipes can lead to similar hepatocyte-like gene expression in cells.

### Example 19: Effect of TF Recipes H0014-H0032A on iPSC Differentiation

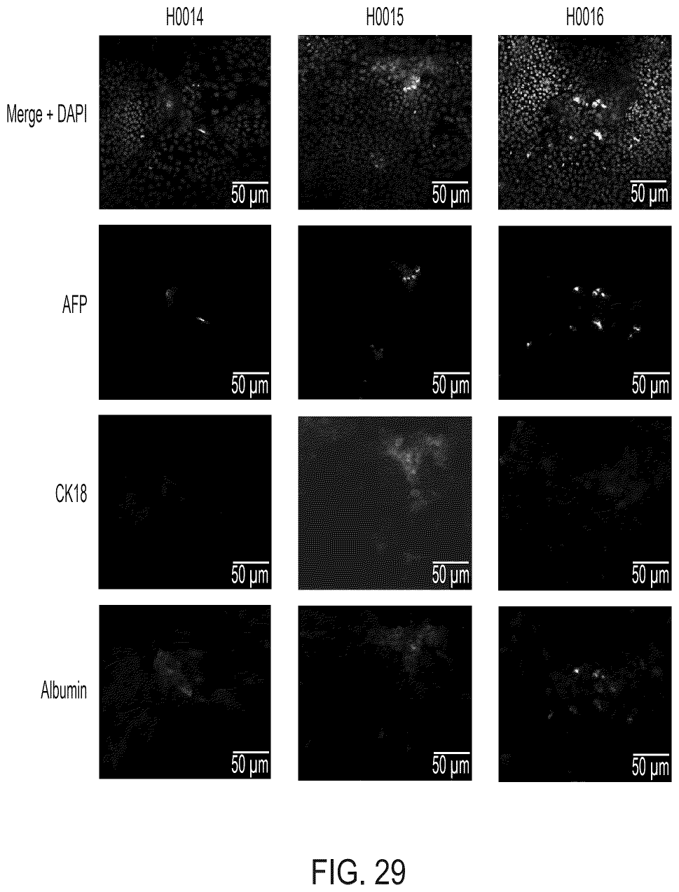

Experiments were conducted as described before, to induce differentiation. Briefly, iPSCs were administered TF recipes as disclosed before and induction of differentiation was allowed to proceed for at least 96 hours. Tests were conducted to evaluate cell lines modified to express H0014-H0019 on iPSC differentiation. Many of these TF combinations were smaller subsets of the transcription factor combinations in H0011 cells, including four of the specific TF combinations identified by the single cell RNA sequencing (scRNAseq) study (H0028, H0031, H0033, H0034). Over several experiments, cells were assessed by immunofluorescent staining for markers AFP, CK18, albumin (ALB), and in some experiments A1AT (FIG. 29-32). Induced cells from engineered cell lines H0014 to H0019 were stained with antibodies for alpha-fetoprotein (AFP), cytokeratin 18 (CK18), albumin, as well as DAPI to label nuclei. Scale bar: 50 μm. Different TF cocktails or combinations had cells at varying stages along the differentiation trajectory. In H0014 cells, for example, TF combinations induced expression of AFP and ALB positive cells, in H0015 cells, for example, TF combinations induced expression of AFP, CK18 and ALB positive cells and in H0016 cells, for example, TF combinations induced expression of AFP and ALB positive cells (FIG. 29).

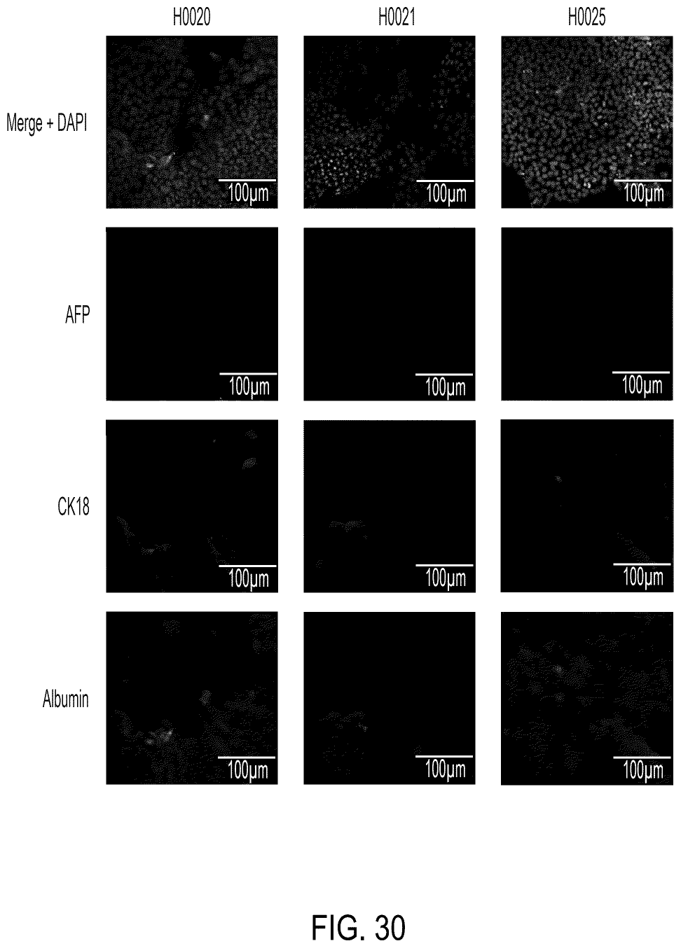

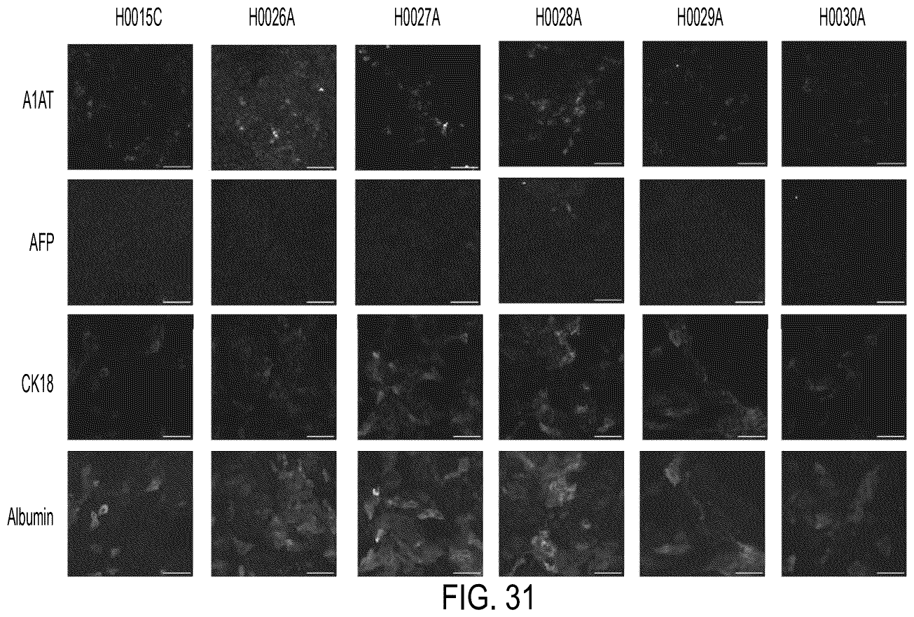

In H0020 cells, for example, TF combinations induced expression of CK18 and ALB positive cells, in H0021 cells, for example, TF combinations induced expression of CK18 and ALB positive cells and in H0025 cells, for example, TF combinations induced expression of ALB positive cells (FIG. 30). Induced cells from engineered cell lines H0020-25 were stained with antibodies for AFP, CK18, albumin, as well as DAPI to label nuclei. Scale bar: 100 μm. In additional staining that included alpha-1 anti-trypsin (A1AT), in H0015C cells, for example, TF combinations induced expression of A1AT, AFP, CK18 and ALB positive cells. In H0026A cells, for example, TF combinations induced expression of A1AT, CK18 and ALB positive cells, in H0027A cells, for example, TF combinations induced expression of A1AT, CK18 and ALB positive cells, in H0028A cells, for example, TF combinations induced expression of A1AT, AFP, CK18 and ALB positive cells, in H0029A cells, for example, TF combinations induced expression of A1AT, CK18 and ALB positive cells and in H0030A cells, for example, TF combinations induced expression of A1AT, AFP, CK18 and ALB positive cells (FIG. 31). Induced cells from engineered cells lines H0015C and H0026A-30A were stained with antibodies for A1AT, AFP, CK18, and albumin. Scale bar: 100 μm.

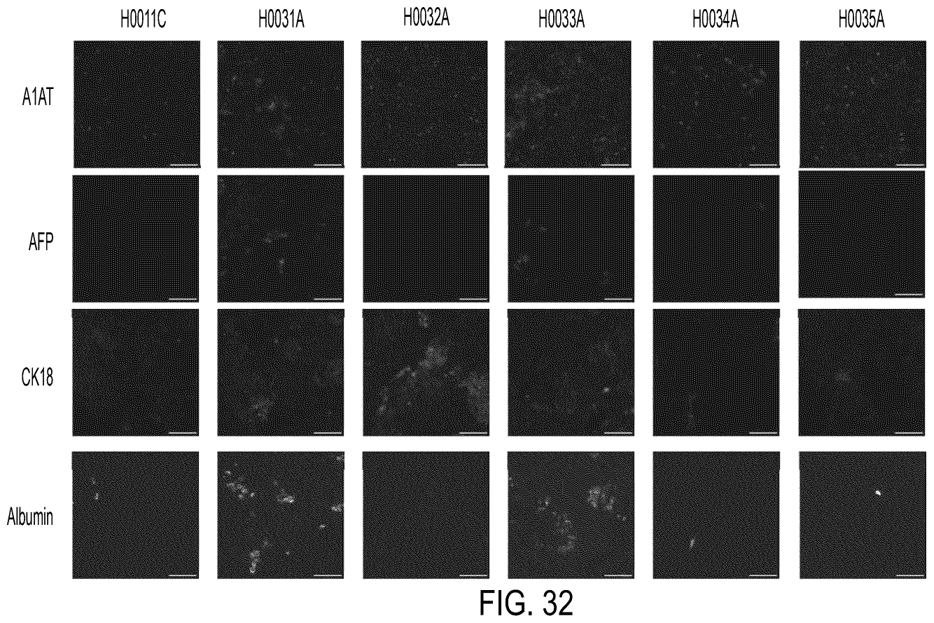

In H0011C cells, for example, TF combinations induced expression of A1AT, CK18, ALB positive cells, in H0031A cells, for example, TF combinations induced expression of A1AT, AFP, CK18, and ALB positive cells, in H0032A cells, for example, TF combinations induced expression of A1AT and CK18 positive cells, in H0033A cells, for example, TF combinations induced expression of A1AT, AFP, CK18 and ALB positive cells, in H0034A cells, for example, TF combinations induced expression of A1AT, CK18 and ALB positive cells, in H0035A cells, for example, TF combinations induced expression of A1AT, CK18 and ALB positive cells (FIG. 32). Induced cells from engineered lines H0011C and H0031A-35A cells were stained with antibodies for A1AT, AFP, CK18, and albumin. Scale bar: 100 μm.

### Example 20: Expression of Hepatocyte Markers Albumin, CK18 and ASGR1 in Induced Cells

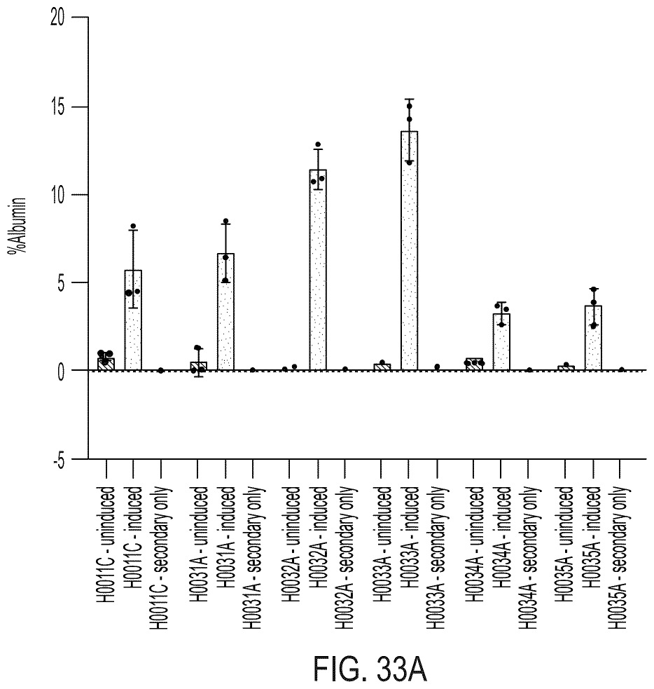

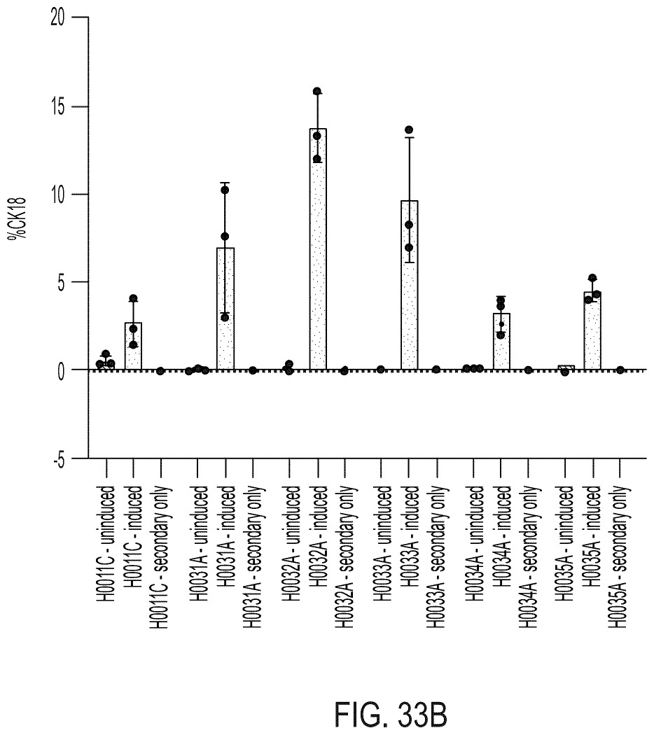

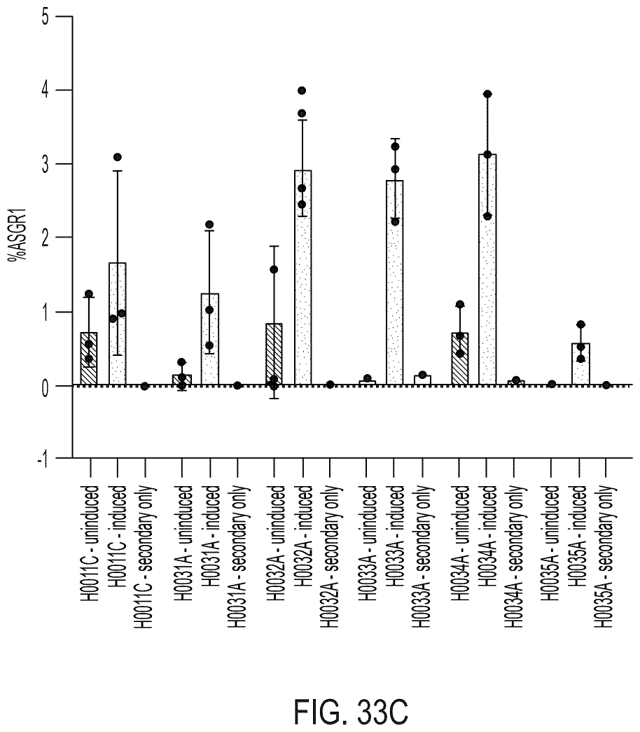

The percentage of induced cells from engineered lines H001C and H0031A-H0035A that expressed albumin, CK18 and ASGR1 markers was determined. Experiments were conducted as described before, to induce differentiation. Briefly, iPSCs were administered TF recipes as disclosed before and induction of differentiation was allowed to proceed for at least 96 hours. To quantify marker-expression, immunofluorescent staining on lines H0011C and H0031A-35A was conducted. Cells were stained with antibodies for albumin, CK18, and ASGR-1. The percentage of cells that were positive for a specific marker, as indicated by an average object intensity over a set threshold, were calculated. Using an automated imaging and quantification protocol for cell lines H0011C, and H0031A-35A following 96 hours of transcription factor (TF) induction, cells were albumin positive in up to ˜15% of H0033A cells, up to ˜13% of H0032A cells, up to 9% of H0031A, up to 9% of H0011C cells, up to ˜5% of H0035A cells and up to ˜4% of H0034A cells (FIG. 33A). After 96 hours of TF induction, cells were CK18 positive in up to ˜18% of H0032A cells, up to ˜14% of H0033A cells, up to ˜11% of H0031A cells, up to ˜5% of H0035A cells, up to 4% of H0034A cells and up to ˜4% of H0011C cells (FIG. 33B). After 96 hours of TF inductions, cells also expressed ASGR1 in up to ˜4% of H0034A cells, up to ˜4% of H0032A cells, up to ˜4% of H0033A cells, up to ˜3% of H0011C cells, up to ˜2% of H0031A cells, and up to ˜1% of H0035A cells (FIG. 33C).

### Example 21: Expression of Tra-1-60 Marker in Live Induced Cells

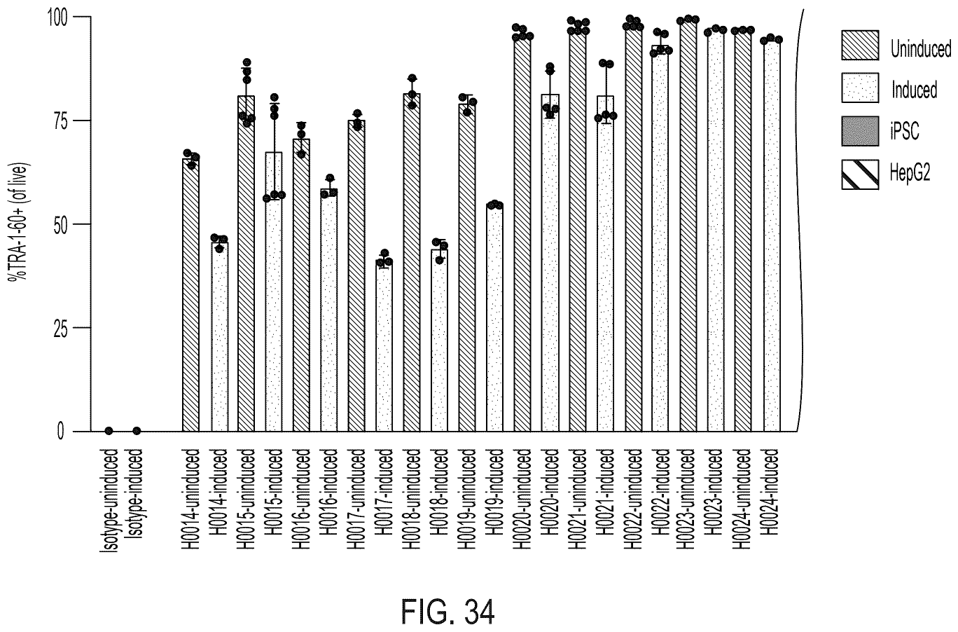

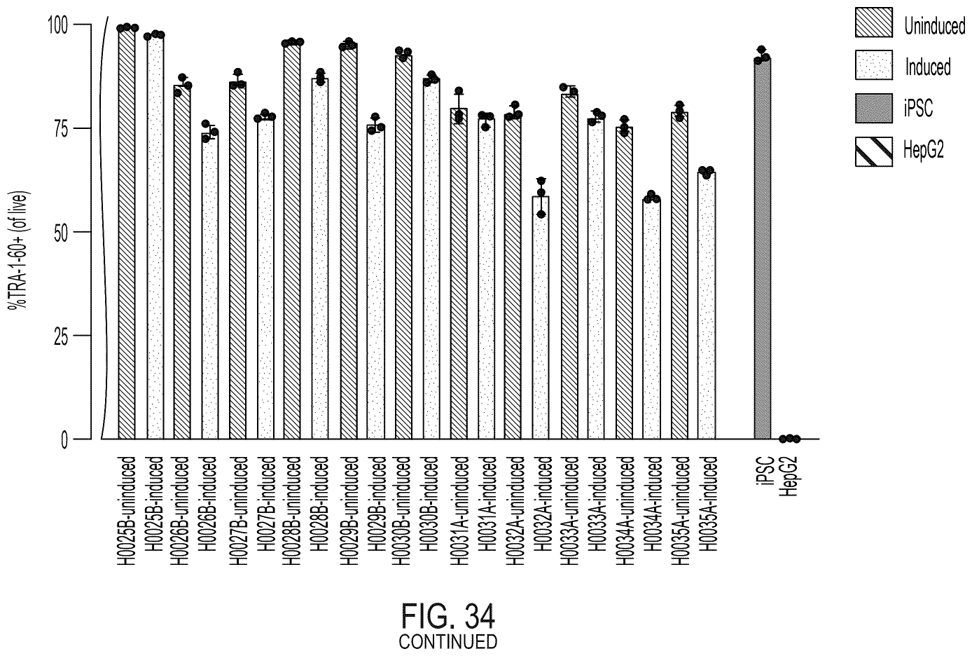

Expression of Tra-1-60 on cells that were induced to differentiate by the various TF recipes or cocktails was investigated. Following the induction experiment, a flow cytometry panel for ASGR-1, CXCR4, and Tra-1-60 was run on each line. Tra-1-60 is a cell surface marker expressed on e.g., human embryonic stem cells, iPSCs which is lost during cellular differentiation. TFs were induced in H0014-35 cells for 96 hours and then Tra-1-60 expression (a marker of pluripotency) was measured via flow cytometry. The isotype shows background fluorescence. Non-engineered iPSCs were a positive control and HepG2 cells were a negative control. The legend shows that induced cells have a light blue bar and uninduced cells have a dark blue bar. After 96 hours of transcription factor expression, virtually all live cells lost some Tra-1-60 expression compared to their uninduced controls (FIG. 34). This suggests that the live cells were in fact differentiating as a result of exogenous transcription factor (TF) expression and were becoming less stem cell-like.

### Example 22: Expression of ASGR-1 Marker in Induced Cells

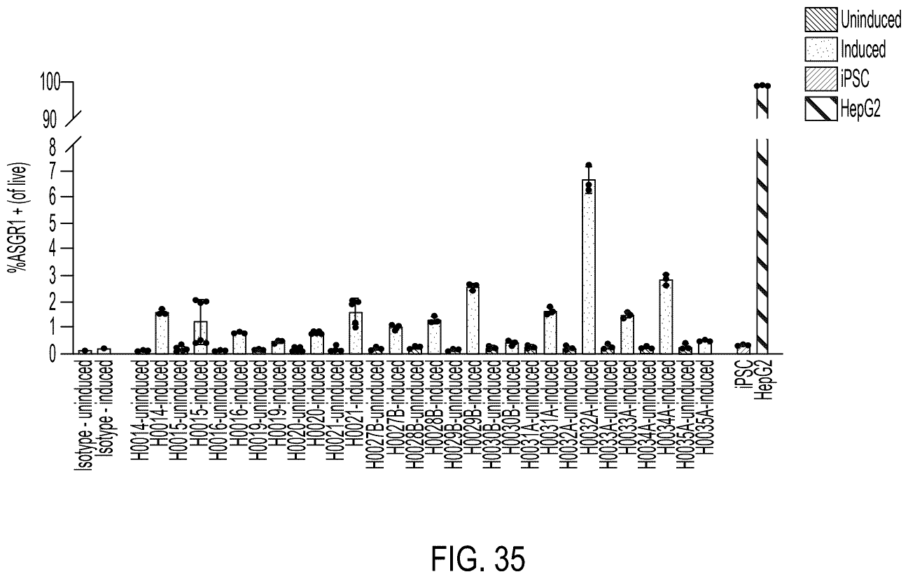

Experiments were conducted as described before, to induce differentiation. Briefly, iPSCs were administered TF recipes as disclosed before and induction of differentiation was allowed to proceed for at least 96 hours. TFs were induced in H0014-35 cells for 96 hours and then ASGR1 expression (a mature hepatocyte marker) was measured via flow cytometry. Several live cell lines expressed some amount of ASGR-1, a hepatocyte surface marker. The isotype shows background fluorescence. Non-engineered iPSCs are a negative control and HepG2 cells are a positive control. The legend shows that induced cells have a light blue bar and uninduced cells have a dark blue bar. Among the ASGR-1 positive cells, the most successful cell line was H0032A with 7% of live cells expressing ASGR-1 (FIG. 35). Up to ˜3% of H0034A cells, up to ˜3% of H0029B cells, up to ˜2% of H0014 cells, up to ˜2% of H0015 cells, up to ˜2% of H0021 cells, up to ˜2% of H0031A cells, up to ˜2% of H0033A cells, up to ˜1% of H0016 cells, up to ˜1% of H0019 cells, up to 1% of H0020 cells, up to ˜1% of H0027B cells, up to ˜1% of H0028B cells, up to ˜1% of H0030B cells, up to ˜1% of H0035A cells were ASGR1 positive (FIG. 35).

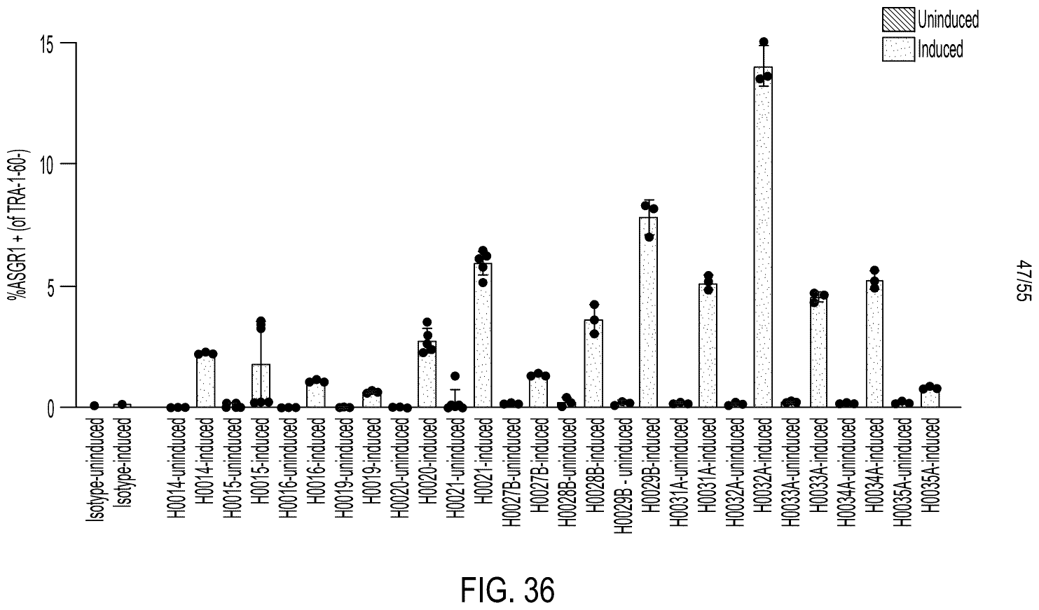

Among the Tra-1-60 negative cells, indicating that they have differentiated away from a stem cell phenotype, the percentage of ASGR1 positive cells increased for many cell lines. TFs were induced in H0014-35 cells for 96 hours and then ASGR1 and Tra-1-60 expression was measured via flow cytometry. This graph shows the percentage of ASGR1 positive cells of the Tra-1-60-population. The isotype shows background fluorescence. The legend shows that induced cells have a light blue bar and uninduced cells have a dark blue bar. ASGR1 increased with the highest again being H0032A with 13% of cells expressing ASGR-1 (FIG. 36). Up to ˜8% of H0029B cells, up to ˜7% of H0021 cells, up to ˜6% of H0031A cells, up to ˜6% of H0034A cells, up to ˜5% of H0033A cells, up to ˜4% of H0028B cells, up to ˜4% of H0015 cells, up to ˜4% of H0020 cells, up to ˜3% of H0014 cells, up to ˜1% of H0016 cells, up to ˜1% of H0019 cells, up to ˜1% of H0027B cells, and up to ˜1% of H0035A cells were ASGR1 positive among Tra-1-60 negative cells (FIG. 36).

### Example 23: Expression of CXCR-4 Marker in Induced Cells

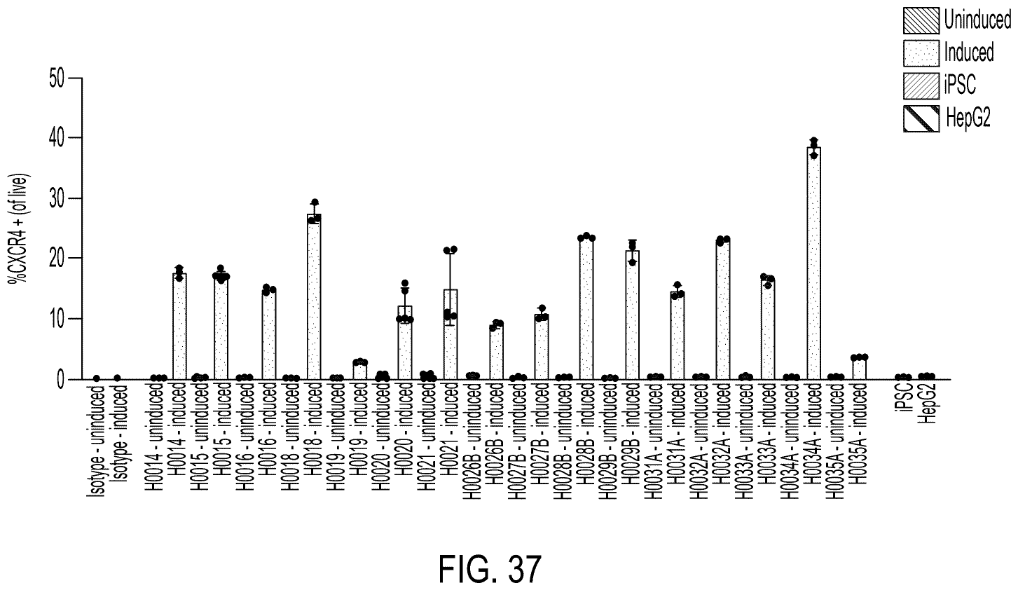

Expression of CXCR-4, chemokine receptor, is a marker of expressed in definitive endoderm and some other cell fates was investigated. Briefly, TFs were induced in H0014-35 cells for 96 hours and then CXCR4 expression (a marker of definitive endoderm) was measured via flow cytometry. The isotype in FIG. 37 shows background fluorescence. Non-engineered iPSCs were a negative control and HepG2 cells were a positive control. As shown in the legend, induced cells have a light blue bar and uninduced cells have a dark blue bar.

The CXCR-4 marker was expressed in many of the live cell lines, e.g., it was up to ˜40% of H0034A cells, up to ˜30% of H0018 cells, up to ˜25% of H0021 cells, up to ˜25% of H0028B cells, up to ˜25% of H0029B cells, up to ˜25% of H0032A cells, up to ˜20% of H0014 cells, up to ˜20% of H0015 cells, up to ˜20% of H0020 cells, up to ˜20% of H0031A cells, up to ˜20% of H0033A cells, up to ˜18% of H0016 cells, up to ˜10% of H0026B cells, up to ˜10% of H0027B cells, up to ˜5% of H0019 cells and up to ˜5% of H0035A cells (FIG. 37). Cells that express CXCR-4 but not ASGR1 positive may be differentiating into endoderm-lineage cells (e.g. H0018, H0026B, or H0032A cells).

### Example 24: Evaluating Differentiation of the Hepatocyte-Like Cells in H0021 Using RT-qPCR

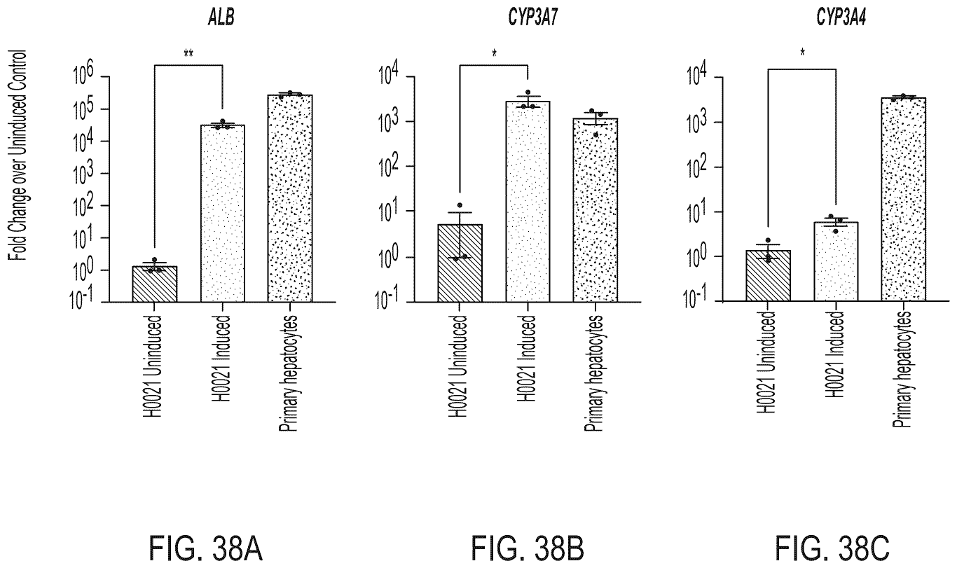

Differentiation of the hepatocyte-like cells was further investigated using an orthogonal readout using real time quantitative PCR (RT-qPCR) on uninduced and induced H0021 cells and compared expression of marker genes by hepatocyte-like cells to expression by primary human hepatocytes. RT-qPCR showed an increase in expression of liver specific markers after TF induction. RT-qPCR was performed on uninduced and induced engineered hepatocyte-like cells, as well as primary human hepatocytes normalized by a housekeeping gene. Fold-change expression was calculated relative to the median value of the uninduced control cells. Significance determined by unpaired t-test; * p<0.5, ** p<0.01. There was a significant increase in liver-specific markers tested, e.g., albumin (FIG. 38A), CYP3A7 (FIG. 38B), and CYP3A4 (FIG. 38C) in induced cells compared to the uninduced cells (FIG. 38).

### Example 25: Differentiation of the Hepatocyte-Like Cells H0027B, H0028B, H0031A and H0033A

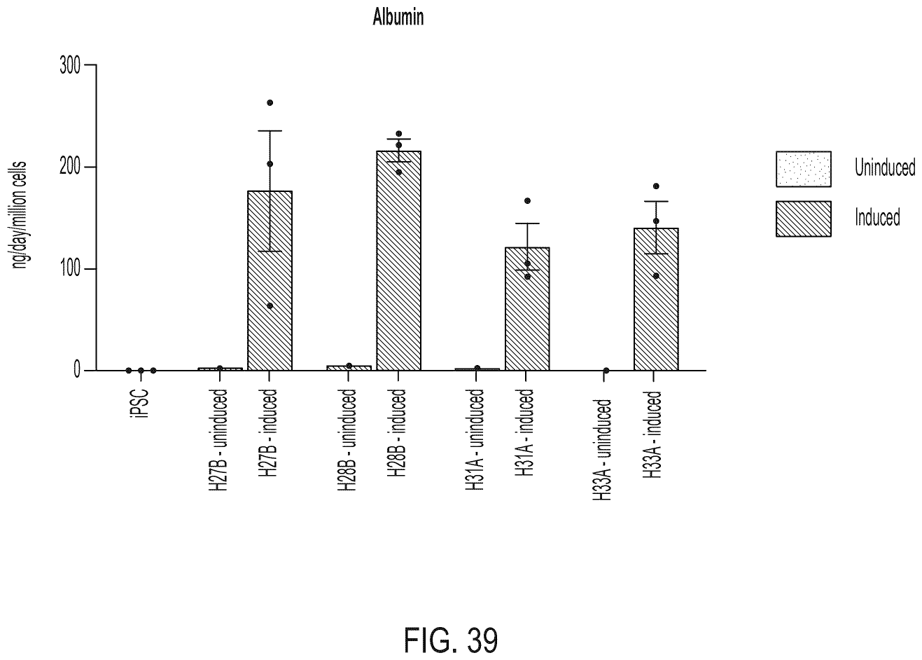

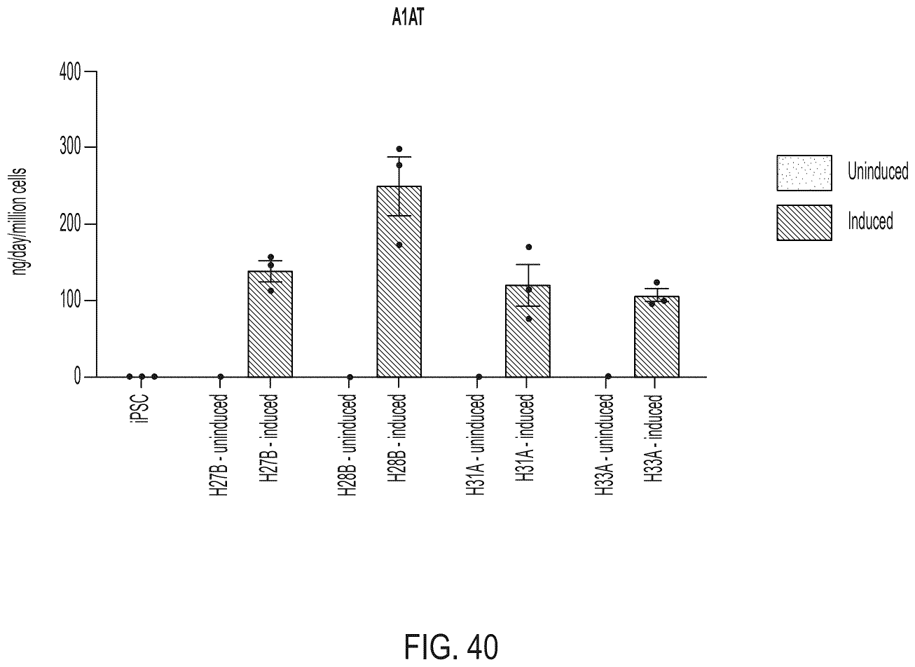

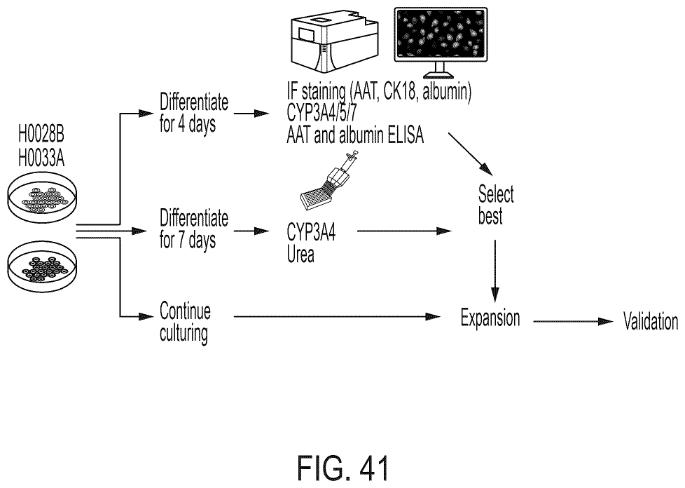

Following the screening of engineered hepatocyte-like cell lines by immunofluorescent staining and flow cytometry, cell line H0027B, H0028B, H0031A, and H0033A were investigated further. Studies were performed using ELISAs for albumin and A1AT on their conditioned media (FIGS. 39 and 40). The amount of albumin produced by uninduced and induced engineered cell lines H0027B, H0028B, H0031A, and H0033A was measured by ELISA and normalized for time and total number of cells. As well, the amount of A1AT produced by uninduced and induced engineered cell lines H0027B, H0028B, H0031A, and H0033A was measured by ELISA and normalized for time and total number of cells.

All of the induced cell lines produced both albumin and A1AT compared to uninduced cells, and there was no statistically significant difference between the induced cell lines. H0027B cells produced up to ˜250 ng of Albumin/day/million cells, H0028B cells produced up to ˜250 ng of Albumin/day/million cells, H0031A cells produced up to ˜200 ng of Albumin/day/million cells, and H0033A cells up to ˜200 ng of Albumin/day/million cells. H0027B cells produced up to 150 ng of A1AT/day/million cells, H0028B cells produced up to 300 ng of A1AT/day/million cells, H0031A cells produced up to ˜200 ng of A1AT/day/million cells, and H0033A cells produced up to ˜150 ng of A1AT/day/million cells.

### Example 26: Isolation of Most Hepatocyte-Like Cells from H0028B and H0033A

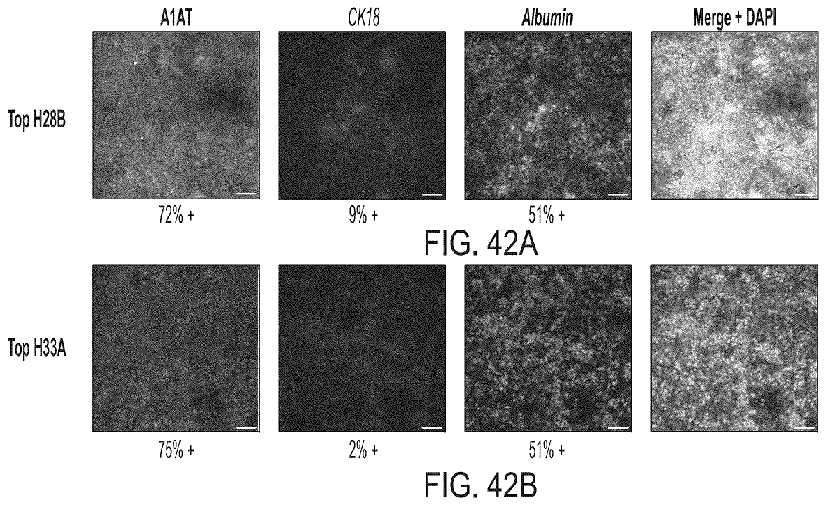

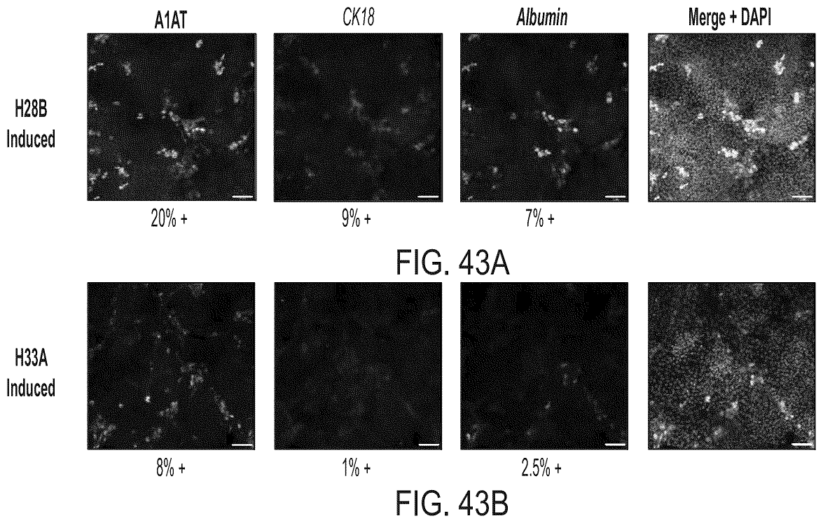

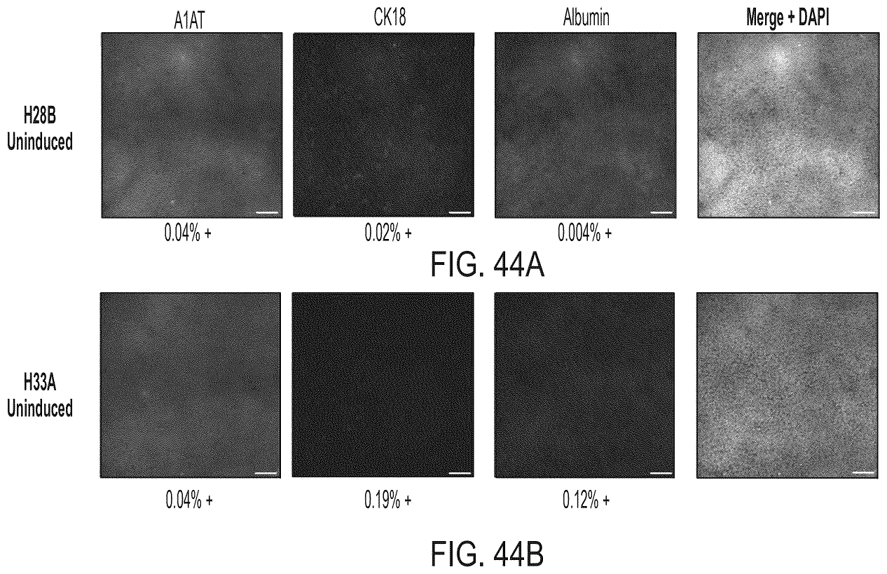

H0028B and H0033A had clear populations of hepatocyte-like cells but were composed of mixed populations. To isolate the most hepatocyte-like cells, cell lines H0028B and H0033A were isolated and differentiated for 96 hours or seven days and then analyzed by several metrics, including immunofluorescent staining (for A1AT, CK18, and albumin), ELISAs to detect secreted A1AT and albumin, chromogenic assay to detect secreted urea, and luminogenic substrate cleavage rate measurements of cytochrome p450 enzyme activity. The top performers that exhibited the most hepatocyte-like phenotype as assessed by these metrics were selected for expansion and further validation. Cells were analyzed by several metrics: immunofluorescent staining (for A1AT, CK18, and albumin), ELISAs to detect secreted A1AT and albumin, a chromogenic assay to detect secreted urea, and measurements of cytochrome p450 enzyme activity. Several performers functioned well across multiple assays, suggesting that they are hepatocyte-like (FIGS. 42A-42B; FIG. 43A-43B; FIG. 44A-44B). Twenty-four of the best performers were selected for expansion and further validation. H0028B and H0033A were compared for A1AT, CK18, and albumin to the original cells and the percentage of cells that express each marker was quantified. Higher percentages of cells expressed A1AT, CK18 and albumin compared to uninduced and parental cells (FIGS. 42A-42B, FIG. 43A-34B and FIG. 44A-44B).

1-162. (canceled)

163. A population of engineered cells configured to express at least three hepatocyte markers in a time period of 96 hours or less after induction of a plurality of transcription factors.

164. The population of claim 163, wherein the at least three hepatocyte markers comprise CK18, Albumin, ASGR-1 or CXCR4.

165. The population of claim 163, wherein 4% or more of the population is configured to express CK18.

166. The population of claim 163, wherein 2% or more of the population is configured to express Albumin.

167. The population of claim 163, wherein at least a portion of the population expressing Albumin also expresses CK18.

168. The population of claim 163, wherein 2% or more of the population is configured to express ASGR-1.

169. The population of claim 163, wherein 2% or more of the population is configured to express CXCR-4.

170. The population of claim 163, wherein the population has a reduced expression of a pluripotency marker.

171. The population of claim 170, wherein the pluripotency marker is TRA-1-60.

172. The population of claim 163, wherein at most 80% of the population is configured to express TRA-1-60.

173. The population of claim 163, wherein a portion of the population accumulates lipids.

174. The population of claim 163, wherein the population further upregulates expression of one or more hepatocyte genes or downregulates expression of one or more stem cell marker.

175. The population of claim 174, wherein the one or more hepatocyte genes comprises: ABCC2, ABCC3, ABCC6, ACSL1, AFP, ALB, ALDH6A1, ANG, APOA1, APOA2, APOB, APOM, AR, ASGR1, ASL, CP, CYP2A7, CYP2B6, CYP3A5, CYP3A7, DEFB1, DLK1, FAH, FGA, FGB, FGL1, GOLT1A, HAL, HPR, LEPR, MTTP, ORM1, PLIN1, RBP4, SAA4, SERPINA1, SLPI, SULT1A1, TAT, or TTR.

176. The population of claim 174, wherein the stem cell marker comprises POU5F1.

177. A method of generating a population of engineered cells comprising:
(a) delivering one or more transcription factors to one or more pluripotent stem cells (PSCs);
(b) generating said population of engineered cells from said one or more PSCs at least in part by inducing expression of the one or more transcription factors; and
(c) using said population of engineered cells to express at least three hepatocyte markers in a time period of 96 hours or less.

178. The method of claim 177, wherein at least one engineered cell of said population expresses CYP3A7 in 96 hours or less after said inducing.

179. The method of claim 177, wherein said at least one engineered cell of said population of engineered cells expressing CYP3A7 comprises higher CYP3A7 expression than a primary hepatocyte cell.

180. The method of claim 177, wherein at least one engineered cell of said population of engineered cells does not express TRA-1-60 within said time period.

181. The method of claim 177, wherein at least one engineered cell of said population of engineered cells has a higher capacity for a lipid storage function than a primary hepatocyte cell.

182. The method of claim 177, further comprising generating a population of hepatocytes or hepatic cells from said one or more PSCs in 30 days or less.
